(19) United States
(12) Patent Application Publication
(10) Pub. No.: US 2024/0092853 A1
(43) Pub. Date: Mar. 21, 2024

(54) PH-DEPENDENT MUTANT INTERLEUKIN-2 POLYPEPTIDES

(71) Applicant: Hoffmann-La Roche Inc., Little Falls, NJ (US)

(72) Inventors: Lorenzo DEHO, Zuerich (CH); Christian GASSNER, Penzberg (DE); Sylvia HERTER, Baden-Ruetihof (CH); Thomas HOFER, Zuerich (CH); Ralf HOSSE, Mettmenstetten (CH); Adrian HUGENMATTER, Zuerich (CH); Christian KLEIN, Bonstetten (CH); Florian LIMANI, Dietikon (CH); Ekkehard MOESSNER, Kreizlingen (CH); Melanie OBBA, Niederglatt (CH); Bianca SCHERER, Urdorf (CH); Pablo UMANA, Wollerau (CH)

(73) Assignee: Hoffmann-La Roche Inc., Little Falls, NJ (US)

(21) Appl. No.: 18/255,300

(22) PCT Filed: Dec. 2, 2021
(86) PCT No.: PCT/EP21/83864

§ 371 Date: May 31, 2023

(30) Foreign Application Priority Data
Dec. 4, 2020 (EP) 20211731.3

(51) Int. Cl. C07K 14/55; A61K 47/68

(52) U.S. Cl. CPC C07K 14/55; A61K 47/6851

(57) ABSTRACT

The present invention generally relates to pH-dependent mutant interleukin-2 polypeptides that exhibit reduced IL-2 receptor binding at neutral pH and retained IL-2 receptor binding at reduced pH. In addition, the invention relates to immunoconjugates comprising said pH-dependent mutant IL-2 polypeptides, polynucleotide molecules encoding the pH-dependent mutant IL-2 polypeptides or immunoconjugates, and vectors and host cells comprising such polynucleotide molecules. The invention further relates to methods for producing the pH-dependent mutant IL-2 polypeptides or immunoconjugates, pharmaceutical compositions comprising the same, and uses thereof.

### FIELD OF THE INVENTION

The present invention generally relates to pH-dependent mutant interleukin-2 polypeptides. More particularly, the inventions concerns mutant IL-2 polypeptides that exhibit improved properties for use as immunotherapeutic agents. In addition, the invention relates to immunoconjugates SpH-dependent mutant IL-2 polypeptides or immunoconjugates, and vectors and host cells comprising such polynucleotide molecules. The invention further relates to methods for producing the pH-dependent mutant IL-2 polypeptides or immunoconjugates, pharmaceutical compositions comprising the same, and uses thereof.

### BACKGROUND

Interleukin-2 (IL-2), also known as T cell growth factor (TCGF), is a 15.5 kDa globular glycoprotein playing a central role in lymphocyte generation, survival and homeostasis. It has a length of 133 amino acids and consists of four antiparallel, amphiphatic α-helices that form a quaternary structure indispensable of its function (Smith, Science 240, 1169-76 (1988); Bazan, Science 257, 410-413 (1992)). Sequences of IL-2 from different species are found under NCBI RefSeq Nos. NP000577 (human), NP032392 (mouse), NP446288 (rat) or NP517425 (chimpanzee).

IL-2 mediates its action by binding to IL-2 receptors (IL-2R), which consist of up to three individual subunits, the different association of which can produce receptor forms that differ in their affinity to IL-2. Association of the α (CD25), β (CD122), and γ (γ_{c}, CD132) subunits results in a trimeric, high-affinity receptor for IL-2. Dimeric IL-2 receptor consisting of the β and γ subunits is termed intermediate-affinity IL-2R. The a subunit forms the monomeric low affinity IL-2 receptor. Although the dimeric intermediate-affinity IL-2 receptor binds IL-2 with approximately 100-fold lower affinity than the trimeric high-affinity receptor, both the dimeric and the trimeric IL-2 receptor variants are able to transmit signal upon IL-2 binding (Minami et al., Annu Rev Immunol 11, 245-268 (1993)). Hence, the α-subunit, CD25, is not essential for IL-2 signalling. It confers high-affinity binding to its receptor, whereas the β subunit, CD122, and the γ-subunit are crucial for signal transduction (Krieg et al., Proc Natl Acad Sci 107, 11906-11 (2010)). Trimeric IL-2 receptors including CD25 are expressed by (resting) CD4+ forkhead box P3 (FoxP3)⁺ regulatory T (T_{reg}) cells. They are also transiently induced on conventional activated T cells, whereas in the resting state these cells express only dimeric IL-2 receptors. T_{reg} cells consistently express the highest level of CD25 in vivo (Fontenot et al., Nature Immunol 6, 1142-51 (2005)).

IL-2 is synthesized mainly by activated T-cells, in particular CD4+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (reviewed e.g. in Waldmann, Nat Rev Immunol 6, 595-601 (2009); Olejniczak and Kasprzak, Med Sci Monit 14, RA179-89 (2008); Malek, Annu Rev Immunol 26, 453-79 (2008)).

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma.

However, IL-2 has a dual function in the immune response in that it not only mediates expansion and activity of effector cells, but also is crucially involved in maintaining peripheral immune tolerance.

A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD3 complex, the expression of Fas ligand (FasL) and/or tumor necrosis factor (TNF) is induced, making the cells susceptible for Fas-mediated apoptosis. This process is IL-2 dependent (Lenardo, Nature 353, 858-61 (1991)) and mediated via STAT5. By the process of AICD in T lymphocytes tolerance can not only be established to self-antigens, but also to persistent antigens that are clearly not part of the host's makeup, such as tumor antigens.

Moreover, IL-2 is also involved in the maintenance of peripheral CD4⁺ CD25⁺ regulatory T (T_{reg}) cells (Fontenot et al., Nature Immunol 6, 1142-51 (2005); D'Cruz and Klein, Nature Immunol 6, 1152-59 (2005); Maloy and Powrie, Nature Immunol 6, 1171-72 (2005), which are also known as suppressor T cells. They suppress effector T cells from destroying their (self-)target, either through cell-cell contact by inhibiting T cell help and activation, or through release of immunosuppressive cytokines such as IL-10 or TGF-β. Depletion of T_{reg} cells was shown to enhance IL-2 induced anti-tumor immunity (Imai et al., Cancer Sci 98, 416-23 (2007)).

Therefore, IL-2 is not optimal for inhibiting tumor growth, because in the presence of IL-2 either the CTLs generated might recognize the tumor as self and undergo AICD or the immune response might be inhibited by IL-2 dependent T_{reg} cells.

A further concern in relation to IL-2 immunotherapy are the side effects produced by recombinant human IL-2 treatment. Patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g. pulmonary and cutaneous edema and liver cell damage) and intravascular fluid depletion (causing a drop in blood pressure and compensatory increase in heart rate). There is no treatment of VLS other than withdrawal of IL-2. Low-dose IL-2 regimens have been tested in patients to avoid VLS, however, at the expense of suboptimal therapeutic results. VLS was believed to be caused by the release of proinflammatory cytokines, such as tumor necrosis factor (TNF)-α from IL-2-activated NK cells, however it has recently been shown that IL-2-induced pulmonary edema resulted from direct binding of IL-2 to lung endothelial cells, which expressed low to intermediate levels of functional αβγ IL-2 receptors (Krieg et al., Proc Nat Acad Sci USA 107, 11906-11 (2010)).

Several approaches have been taken to overcome these problems associated with IL-2 immunotherapy. For example, it has been found that the combination of IL-2 with certain anti-IL-2 monoclonal antibodies enhances treatment effects of IL-2 in vivo (Kamimura et al., J Immunol 177, 306-14 (2006); Boyman et al., Science 311, 1924-27 (2006)). In an alternative approach, IL-2 has been mutated in various ways to reduce its toxicity and/or increase its efficacy. Hu et al. (Blood 101, 4853-4861 (2003), US Pat. Publ. No. 2003/0124678) have substituted the arginine residue in position 38 of IL-2 by tryptophan to eliminate IL-2's vasopermeability activity. Shanafelt et al. (Nature Biotechnol 18, 1197-1202 (2000)) have mutated asparagine 88 to arginine to enhance selectivity for T cells over NK cells. Heaton et al. (Cancer Res 53, 2597-602 (1993); U.S. Pat. No. 5,229,109) have introduced two mutations, Arg38Ala and Phe42Lys, to reduce the secretion of proinflammatory cytokines from NK cells. Gillies et al. (US Pat. Publ. No. 2007/0036752) have substituted three residues of IL-2 (Asp20Thr, Asn88Arg, and Gln126Asp) that contribute to affinity for the intermediate-affinity IL-2 receptor to reduce VLS. Gillies et al. (WO 2008/0034473) have also mutated the interface of IL-2 with CD25 by amino acid substitution Arg38Trp and Phe42Lys to reduce interaction with CD25 and activation of T_{reg} cells for enhancing efficacy. To the same aim, Wittrup et al. (WO 2009/061853) have produced IL-2 mutants that have enhanced affinity to CD25, but do not activate the receptor, thus act as antagonists. The mutations introduced were aimed at disrupting the interaction with the β- and/or γ-subunit of the receptor.

A particular mutant IL-2 polypeptide, designed to overcome the above-mentioned problems associated with IL-2 immunotherapy (toxicity caused by the induction of VLS, tumor tolerance caused by the induction of AICD, and immunosuppression caused by activation of T_{reg} cells), is described in WO 2012/107417. Substitution of the phenylalanine residue at position 42 by alanine, the tyrosine residue at position 45 by alanine and the leucine residue at position 72 of IL-2 by glycine essentially abolishes binding of this mutant IL-2 polypeptide to the α-subunit of the IL-2 receptor (CD25).

However, none of the known IL-2 mutants was shown to overcome all of the above-mentioned problems associated with IL-2 immunotherapy, namely toxicity caused by the induction of VLS, tumor tolerance caused by the induction of AICD, and immunosuppression caused by activation of T_{reg} cells.

Further to the above-mentioned approaches, IL-2 immunotherapy may be improved by selectively targeting IL-2 to tumors, e.g. in the form of immunoconjugates comprising an antibody that binds to an antigen expressed on tumor cells or that binds to effector cells in the tumor environment. Several such immunoconjugates have been described (see e.g. Ko et al., J Immunother (2004) 27, 232-239; Klein et al., Oncoimmunology (2017) 6(3), e1277306; WO 2018/184964 A1).

Given the clinical success and unprecedented efficacy of PD-1/PD-L1 check-point inhibitors there remains a major medical need to increase the response rate and duration in patients with pre-existing T cell immunity further. Recent reports have demonstrated that two populations of tumor-specific CD8 T cells are targeted by PD-1 antibodies: the exhausted TILs and, their newly described TCF1+ precursor with stem-like properties, TResource cells. Of the two the latter correlates with a favorable disease prognosis and response to anti-PD-1 therapy. Cytokines, like interleukin-2, have also been described to induce the proliferation/differentiation of the TResource cells towards functional effector T cells.

IL-2 has been the first effective cancer immunotherapy used to treat metastatic melanoma and renal cell carcinoma. Unfortunately, IL-2, at high concentrations, is toxic by inducing vascular leak syndrome (VLS) and detrimentally expands regulatory T cells and induces activation induced cell death due to binding to CD25. In order to overcome these limitations of wildtype IL2/Proleukin, IL-2v variants with abolished CD25 binding have been described. However, due to the mechanism of IL-2 signaling through the heterodimeric intermediate affinity IL-2Rbg complex IL2v and other IL2 variants automatically activate IL-2R signaling as soon as they encounter the IL-2R and as a consequence mediate unspecific and peripheral immune cell activation outside of the tumor in blood, the vasculature and lymphoid tissues resulting in dose limiting toxicities. As a consequence, it is not possible to administer to a patients as much IL-2 or IL2v as desired in order to achieve the maximal therapeutic benefit.

PD1-IL2v has been developed to overcome some of these limitations by delivering an IL-2 variant, with abrogated binding to CD25, to tumor-reactive PD-1 expressing T cells in cis on the same T cell (WO 2018/184964 A1). As a consequence PD1-IL2v is 10-40-fold more potent on PD-1 expressing T cells as compared to PD-1 negative T cells. In the tumor, PD1-IL2v promotes CD8 TResource cells differentiation in fully functional cytotoxic T cells,—called fresh and better effector T cells, resulting in an effective, long-term anti-tumor immune response and complete tumor eradication in a mouse model of pancreatic cancer.

Taken together by targeting PD1-IL2v in cis to PD-1+ T cells a stronger therapeutic effect of PD1-IL2v can be achieved. In fact, cis-targeting of PD1-IL2v to appropriate antigen specific T-cell subsets, together with PD-1/-L1 inhibition is a better way to exploit endogenous immunity therapeutically and one of the strongest immunomodulatory pathway known for unleashing endogenous immunity for cancer immunotherapy. However, due to the possibility of the IL2v moiety to trigger IL-2R signaling in the periphery also for PD1-IL2v not the maximally desired dose can be administered due to the peripheral non tumor specific IL-2R activation. Thus, the therapeutic index is believed to remain narrow with an anticipated MTD with a flat dose of >10-30 mg in man which may limit utilizing full pathway potential.

Therefore, it is critical to generate next generations of IL-2 molecules cis-targeted to antigen-experienced T cells but with wider therapeutic index.

Although current immunotherapies directed against the PD1/PDL1 axis have shown unprecedented efficacy in several cancer indications, there is a substantial portion of patients who do not respond to the treatment or relapse, while other tumor-types remain largely refractory to these therapies. Therefore, there is a clear, high unmet need of a considerable cancer patient population in patients that have some kind of pre-existing T cell immune response. Examples for indications where PD1 antagonism has resulted in objective responses are e.g. advanced or metastatic melanoma, Merkel cell carcinoma, NSCLC, SCLC, RCC, gastric cancer, hepatocellular cancer, head and neck carcinoma, breast cancer, ovarian cancer, mismatch repair deficient versus sufficient CRC and haematological malignancies like DLBCL and PMBCL after autologous stem cell transplant and HL (Editorial: PD-Loma: a cancer entity with a shared sensitivity to the PD-1/PD-L1 pathway blockade, British Journal of Cancer (2019) 120:3-5; https://doi.org/10.1038/s41416-018-0294-4).

PD-1 is an immune checkpoint that, upon binding to its ligands (PD-L1 and PD-L2), can attenuate the signalling downstream the TCR. PD-1 is expressed on antigen experienced T cells and to a lesser extent on NK cells and B cells. Its upregulation on T cell-surface follows strong and continuous TCR triggering and has been associated with T cell exhaustion/dysfunction in the context of chronic viral infections and cancer. Therefore, PD-1 marks those tumor infiltrating lymphocytes TILs which recognize tumor associated antigens (TAAs) (Simon et al, Oncoimmunology, 2015 Oct. 29; 5(1):e1104448; Simon et al, Oncoimmunology. 2018; 7(1): e1364828.) and therefore represents an appealing therapeutic target for cancer immunotherapy.

Recently it has been reported that PD-1 is not only expressed on terminally differentiated cells, like in the tumor microenvironment, but also on the surface of a newly identified (neo)antigen specific T cell population, called stem cell-like resource T cells (TResource). This cell subset is constituted by multipotent progenitors that can both self-renew and replenish more differentiated subsets of antigen-experienced T cells (Im S J et al, Nature, 2016 Sep. 15; 537(7620):417-421). Hence, PD-1-related exhaustion/dysfunction presents a more complex picture that comprises a reduction in effector functions of tumor specific TILs plus a decrease in self-renewal and differentiation ability of TResource. Interestingly, administration of IL-2 together with PD-1 blockade during chronic LCMV infection has been reported to induce striking synergistic effects (West E. E. et al, J Clin Invest, 2013 June; 123(6):2604-15) which might be the result of the dual therapeutic effect of both molecules on virus-specific effector T cells and TResource. IL-2 plays a central pleiotropic role in lymphocyte generation, activation, differentiation, proliferation, effector functions, survival and homeostasis, and it is mainly synthesized by activated T cells, specifically CD4 T helpers. IL-2 binds to the IL-2R which consists of 3 individual subunits, α (CD25), β (CD122), and γ (γc; CD132), also known as high-affinity IL-2R (10 μM). The dimeric IL-2βγ is an intermediate-affinity receptor which binds IL-2 with approximately 50-100-fold lower affinity than the high-affinity ones. The dimeric IL-2R is expressed on NK cells, memory and effector memory T cells, and at lower levels on resting CD4, CD8 T cells, and monocytes/macrophages. CD25 is transiently induced on conventionally activated T cells and NK cells whereas it is constitutively expressed at high levels on Tregs (naturally occurring). As a consequence of the IL-2 mode of action, anti-tumor efficacy may be compromised by induction of Tregs and activation induced cell death (AICD).

Tumors may be able, however, to escape such targeting by shedding, mutating or downregulating the target antigen of the antibody. Moreover, tumor-targeted IL-2 may not come into optimal contact with effector cells such as cytotoxic T lymphocytes (CTLs), in tumor microenvironments that actively exclude lymphocytes.

The physiological microenvironment of solid tumors is normally characterized by poor perfusion and high metabolic rates. In tumor and tumor stroma, the increased glucose catabolism results independently of the tissue oxygen status in significant production of lactate and H+ (Warburg effect). The disorganized tumor vasculature prevents an efficient wash-out of these H+ ions released into the extracellular medium but also favors the development of tumor hypoxic regions associated with a further shift towards glycolytic metabolism. A common consequence is the acidification of the extracellular pH in solid tumors, a hallmark condition known as tumor acidosis (Zhang X et al., J Nucl Med., 2010 August; 51(8):1167-70.; Corbet et al., Nat Rev Cancer 17, 577-593 (2017); Damgaci et al., Immunology. 2018 July; 154(3):354-362). Tumor cells and cancer associated fibroblasts acidify the tumor environment to pH values around pH 6.0-6.8 relative to healthy tissue with a pH average of pH 7.2-7.4 (Viklund J et al., Curr Med Chem. 2017; 24(26):2827-2845). Tumor acidosis has been shown to contribute in many aspects of cancer pathobiology, including invasion and metastasis, genetic instability, anchorage-independent growth, tumorigenesis, chemoresistance and resistance to apoptosis, angiogenesis, and evasion from the immune system (Viklund J et al., Curr Med Chem. 2017; 24(26):2827-2845; Kolosenko et al., Semin Cancer Biol. 2017 April; 43:119-133; Corbet et al., Nat Rev Cancer 17, 577-593 (2017)).

Some external pH conditional cancer immunotherapy approaches support that tumor acidosis can be utilized for conditional drug activity. Amongst other are CCT-301-59, a pH dependent anti-ROR2 conditionally active biologic—CarT cell therapy, and a pH dependent CTLA-4 check point inhibitor, both developed by BioAtla (https://www.bioatla.com/technology/cab/) and pH-sensitive liposomes delivering cancer antigens for vaccination (Yuba et al., Biomaterials. 2013 April; 34(12):3042-52).

Tumor acidosis is discussed in Corbet, C., Feron, O. Tumour acidosis: from the passenger to the driver's seat. Nat Rev Cancer 17, 577-593 (2017) or Damgaci et al., Immunology. 2018 July; 154(3):354-362.

Thus there remains a need in the art to further enhance the therapeutic usefulness of IL-2 proteins.

### SUMMARY OF THE INVENTION

The present invention is based, in part, on the recognition (i) that tumor microenvironments (TME) exhibit decreased pH levels compared to the neutral pH in the periphery, i.e. neutral systemic pH, and (ii) that pH-dependent mutant interleukin-2 polypeptides have a reduced or abolished systemic activity at neutral pH and full activity in the tumor microenvironment at acidic pH.

Accordingly, in a first aspect the invention provides a mutant interleukin-2 (IL-2) polypeptide comprising one or more amino acid substitutions, each compared to a wilde-type IL-2, preferably human IL-2 according to SEQ ID NO: 144, wherein the one or more amino acid substitutions abolishes or reduces binding to the IL-2 receptor, preferably to the intermediate-affinity IL-2 receptor (IL2Rβγ), at neutral pH and facilitate binding to the IL-2 receptor, preferably to the intermediate-affinity IL-2 receptor (IL2Rfβγ), at decreased pH. In one embodiment, the mutant IL-2 polypetide exhibits reduced or abolished IL-2 receptor binding, preferably intermediate-affinity IL-2 receptor binding, at pH 7.4 and/or pH 7.0 and retained IL-2 receptor binding, preferably intermediate-affinity IL-2 receptor binding, at a pH 6 and/or pH 6.5. In one embodiment said one or more amino acid substitutions is at a position selected from the group of positions corresponding to residue 6, 8, 11, 12, 13, 15, 16, 19, 20, 22, 23, 81, 84, 87, 91, 95, 120, 123, 126, 130, 133 of human IL-2 according to SEQ ID NO: 144. In one embodiment said one or more amino acid substitutions is selected from the group of S6Y, K8E, Q11E, Q11T, L12D, L12E, L12Q, L12S, L12T, Q13H, Q13R, E15Q, H16D, H16E, H16N, H16Q, L19D, L19Q, D20E, D20Q, Q22D, Q22E, Q22H, M23E, M23N, M23Q, R81D, R81E, R81H, R81N, R81Q, D84E, D84Q, S87D, S87E, 587N, S87Q, V91D, V91E, V91N, E95D, E95Q, R120E, R120H, T123E, T123Q, Q126E, Q126H, S130E, T133D, T133E, T133N, T133Q. In one embodiment the mutant IL-2 polypeptide comprises the amino acid substitutions (i) L12E, D20E, M23N, R81N, D84E, S87E, R120E, T123E, S130E, T133N; (ii) Q11E, D20Q, M23E, R81D, D84E, S87E, S130E, T133N; (iii) L12E, L19D, R81D, R120E, T123E, S130E, T133E; (iv) R81Q, S87E, V91D, T123E, S130E, T133D; (v) Q11E, L12E, M23Q, R81D, S87E, V91D, S130E, T133Q; (vi) Q11E, L19D, R81D, D84E, S130E, T133D; (vii) R81D, D84Q, S87D, V91N, T123Q, S130E, T133D; (viii) L19D, R81E, D84E, S87Q, R120H, S130E, T133E; (iix) L19D, M23N, R81D, T133E; (ix) Q11E, L12E, M23Q, R81Q, S87D, V91N, E95Q, R120H, T123E, S130E, T133E; (x) L19D, R81E, S130E, T133D; (xi) R81Q, S87E, V91D, R120E, S130E, T133D; (xii) L12Q, L19Q, R81H, V91E, T123E, S130E, T133E; (xiii) K8E, D20E, M23N, R81H, D84Q, S87E, R120H, S130E, T133D; (xiv) L12E, L19Q, R81H, R120E, T133D; (xv) H16E, L19D, Q22E, M23Q, R81D, D84E, S87D, R120H, S130E, T133E; (xvi) Q11E, L12E, H16Q, L19D, Q22E, M23N, R81E, D84E, S87E, R120H, S130E, T133E (SEQ ID NO: 21); (xvii) Q11E, H16E, L19D, M23E, R81D, S87E, R120H, Q126E, T133D; (xviii) Q11E, L12S, E15Q, H16N, L19D, M23E, R81E, D84E, S87D, R120H, S130E, T133E; (xix) Q11E, H16E, M23E, R81N, D84E, S87E, R120H, Q126E, S130E, T133E; (xx) Q11E, E15Q, H16E, Q22E, M23E, R81H, D84E, S87E, R120H, S130E, T133D; (xxi) Q11E, L12D, Q13H, E15Q, H16E, Q22E, M23E, R81N, D84E, S87E, R120H, S130E, T133E; (xxii) Q11E, E15Q, H16E, L19D, R81E, S87E, R120H, S130E, T133E; (xxiii) H16E, L19D, M23Q, R81N, D84E, S87D, R120H, S130E, T133D; (xxiv) Q11E, L12T, E15Q, H16E, L19D, Q22H, R81D, D84E, S87E, R120H, S130E, T133E; (xxv) Q11E, L12T, E15Q, H16E, L19D, Q22H, M23E, R81E, D84E, S87E, R120H, S130E, T133E; (xxvi) Q11E, E15Q, H16E, L19D, R81E, D84E, S87E, R120H, S130E, T133E; (xxvii) H16E, Q22E, M23Q, 587N, R120H, S130E, T133E; (xxviii) H16E, L19D, Q22D, M23Q, R81D, D84E, S87E, R120H, S130E, T133E; (xxiix) Q11E, L12E, Q13H, E15Q, H16N, L19D, Q22E, M23Q, R81E, D84E, S87D, E95D, R120H, T133E; (xxix) Q11E, L12T, H16E, L19D, Q22E, R81D, D84E, S87E, R120H, S130E, T133D; (xxx) Q11T, L12E, E15Q, H16E, L19D, R81D, D84E, S87E, R120E, S130E, T133D; (xxxi) Q11E, E15Q, H16E, L19D, R81D, D84E, S87E, R120H, S130E, T133E; (xxxii) Q11E, E15Q, H16E, L19D, R81Q, D84E, S87E, R120H, S130E, T133E; (xxxiii) Q11E, L12S, H16E, L19D, M23Q, R81D, D84E, S87E, R120H, S130E, T133E; (xxxiv) S6Y, L12E, Q13R, H16Q, Q22E, M23Q, R81N, D84E, S87E, R120H, S130E, T133D; (xxxv) H16D, M23N, R81D, D84E, R120H, S130E, T133E; (xxxvi) Q11E, L12TE, H16Q, L19D, M23E, R81D, D84E, S87E, R120H, S130E, T133E; (xxxvii) Q11E, L12E, H16NE, M23N, R81E, D84E, R120H, S130E, T133E; (xxxviii) E15Q, H16E, L19D, R81D, D84E, S87E; (xxxix) Q11E, R120H, S130E, T133D; or (xl) Q11E, R81D, D84E, S87E, R120H, S130E, T133D.

In one embodiment the mutant IL-2 polypeptide comprises the amino acid substitution Q11E (as in SEQ ID NO: 44). In one embodiment the mutant IL-2 polypeptide comprises the amino acid substitution E15Q (as in SEQ ID NO: 45). In one embodiment the mutant IL-2 polypeptide comprises the amino acid substitution H16E (as in SEQ ID NO: 46). In one embodiment the mutant IL-2 polypeptide comprises the amino acid substitution L19D (as in SEQ ID NO: 47). In one embodiment mutant IL-2 polypeptide comprises the amino acid substitution Q22E (as in SEQ ID NO: 48). In one embodiment the mutant IL-2 polypeptide comprises the amino acid substitution M23Q (as in SEQ ID NO: 49). In one embodiment the mutant IL-2 polypeptide comprises the amino acid substitution R81D (as in SEQ ID NO: 50). In one embodiment the mutant IL-2 polypeptide comprises the amino acid substitution D84E (as in SEQ ID NO: 51). In one embodiment the mutant IL-2 polypeptide comprises the amino acid substitution S87E (as in SEQ ID NO: 52). In one embodiment the mutant IL-2 polypeptide comprises the amino acid substitution R120H (as in SEQ ID NO: 53). In one embodiment the mutant IL-2 polypeptide comprises the amino acid substitution Q126E (as in SEQ ID NO: 54). In one embodiment the mutant IL-2 polypeptide comprises the amino acid substitution Q126H (as in SEQ ID NO: 55). In one embodiment the mutant IL-2 polypeptide comprises the amino acid substitution 5130E (as in SEQ ID NO: 56). In one embodiment the mutant IL-2 polypeptide comprises the amino acid substitution T133E (as in SEQ ID NO: 57). In one embodiment the mutant IL-2 polypeptide comprises the amino acid substitution T133D (as in SEQ ID NO: 58).

In one embodiment the mutant IL-2 polypeptide comprises the amino acid substitutions E15Q, H16E, L19D, R81D, D84E and S87E (as in SEQ ID NO: 59). In one embodiment the mutant IL-2 polypeptide comprises the amino acid substitutions Q11E, R120H, 5130E and T133D (as in SEQ ID NO: 60). In one embodiment the mutant IL-2 polypeptide comprises the amino acid substitutions Q11E, R81D, D84E, S87E, R120H, 5130E and T133D (as in SEQ ID NO: 61).

In one embodiment the mutant IL-2 polypetider comprises a first amino acid mutation that abolishes or reduces affinity of the mutant IL-2 polypeptide to the high-affinity IL-2 receptor and preserves affinity of the mutant IL-2 polypeptide to the intermediate-affinity IL-2 receptor, each compared to a wild-type IL-2 polypeptide. In one embodiment said first amino acid mutation is at a position corresponding to residue 72 of human IL-2. In one embodiment said amino acid mutation is an amino acid substitution, selected from the group of L72G, L72A, L72S, L72T, L72Q, L72E, L72N, L72D, L72R, and L72K. In a more specific embodiment said first amino acid mutation is the amino acid substitution L72G. In certain embodiments the mutant IL-2 polypeptide comprises a second amino acid mutation that abolishes or reduces affinity of the mutant IL-2 polypeptide to the high-affinity IL-2 receptor and preserves affinity of the mutant IL-2 polypeptide to the intermediate-affinity IL-2 receptor, each compared to a wild-type IL-2 polypeptide. In one embodiment said second amino acid mutation is at a position selected from the positions corresponding to residue 35, 38, 42, 43, and 45 of human IL-2. In a specific embodiment said second amino acid mutation is at a position corresponding to residue 42 of human IL-2. In a more specific embodiment said second amino acid mutation is an amino acid substitution, selected from the group of F42A, F42G, F42S, F42T, F42Q, F42E, F42N, F42D, F42R, and F42K. In an even more specific embodiment said second amino acid mutation is the amino acid substitution F42A. In certain embodiments the mutant interleukin-2 polypeptide comprises a third amino acid mutation that abolishes or reduces affinity of the mutant IL-2 polypeptide to the high-affinity IL-2 receptor and preserves affinity of the mutant IL-2 polypeptide to the intermediate-affinity IL-2 receptor, each compared to a wild-type IL-2 polypeptide. In a particular embodiment, the mutant interleukin-2 polypeptide comprises three amino acid mutations that abolish or reduce affinity of the mutant IL-2 polypeptide to the high-affinity IL-2 receptor and preserve affinity of the mutant IL-2 polypeptide to the intermediate-affinity IL-2 receptor, each compared to a wild-type IL-2 polypeptide, wherein said three amino acid mutations are at positions corresponding to residue 42, 45, and 72 of human IL-2. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are the amino acid substitutions F42A, Y45A and L72G. In certain embodiments the mutant interleukin-2 polypeptide further comprises an amino acid mutation which eliminates the 0-glycosylation site of IL-2 at a position corresponding to residue 3 of human IL-2. In one embodiment said amino acid mutation which eliminates the O-glycosylation site of IL-2 at a position corresponding to residue 3 of human IL-2 is an amino acid substitution selected from the group of T3A, T3G, T3Q, T3E, T3N, T3D, T3R, T3K, and T3P. In a specific embodiment the amino acid mutation which eliminates the O-glycosylation site of IL-2 at a position corresponding to residue 3 of human IL-2 is T3A. In certain embodiments the mutant IL-2 polypeptide further comprises an amino acid at a position corresponding to residue 125 of human IL-2. In a specific embodiment the amino acid at a position corresponding to residue 125 of human IL-2 is C125A. In certain embodiments the mutant IL-2 polypeptide is essentially a full-length IL-2 molecule, particularly a human full-length IL-2 molecule. In one embodiment the mutant IL-2 polypeptide comprises an amino acid substitution selected from the group T3A, F42A, Y45A, L72G, C125A. In one embodiment the mutant IL-2 polypeptide comprises the amino acid substitutions T3A, F42A, Y45A, L72G and C125A.

In one embodiment the mutant IL-2 polypeptide is linked to a non-IL-2 moiety. In one embodiment the mutant IL-2 polypeptide is linked to a first and a second non-IL-2 moiety. In one embodiment the mutant IL-2 polypeptide is linked to a first and a second non-IL-2 moiety. In one embodiment the mutant IL-2 polypeptide shares a carboxy-terminal peptide bond with said first non-IL-2 moiety and an amino-terminal peptide bond with said second non-IL-2 moiety. In certain embodiment the non-IL-2 moiety is a targeting moiety. In certain embodiments said non-IL-2 moiety is an antigen binding moiety or an effector cell binding moiety. In one embodiment said antigen binding moiety is an antibody. In another embodiment said antigen binding moiety or effector cell binding moiety is an antibody fragment. In a more specific embodiment said antigen binding moiety effector cell binding moiety is selected from a Fab molecule and a scFv molecule. In a particular embodiment said antigen binding moiety or effector cell binding moiety is a Fab molecule. In another embodiment said antigen binding moiety or effector cell binding moiety is a scFv molecule. In one embodiment said antigen binding moiety or effector cell binding moiety is an immunoglobulin molecule. In a more specific embodiment said antigen binding moiety or effector cell binding moiety is an IgG class, particularly an IgG₁ subclass, immunoglobulin molecule. In certain embodiments said antigen binding moiety is directed to an antigen presented on a tumor cell or in a tumor cell environment, particularly an antigen selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). In certain embodiments said effector cell binding moiety is directed to an effector cell present in the tumor cell environment in order to achieve cis-targeting, particularly a target selected from the group of CD8, PD-1, LAG3, TIM3, TIGIT, CD28, 4-1BB, OX40. GITR. ICOS, CXCR3, CXCR5.

In a further the aspect the invention provides an immunoconjugate comprising a mutant IL-2 polypeptide as described herein, and an antigen binding moiety and/or an effector cell binding moiety. In one embodiment of the immunoconjugate according to the invention the mutant IL-2 polypeptide shares an amino- or carboxy-terminal peptide bond with said antigen binding moiety or said effector cell binding moiety. In particular embodiments the immunoconjugate comprises as first and a second antigen binding moiety or a first and a second effector cell antigen binding moiety or an antigen binding moiety and an effector cell binding moiety. In one such embodiment the mutant IL-2 polypeptide comprised in the immunoconjugate according to the invention shares an amino- or carboxy-terminal peptide bond with said first antigen binding moiety and said second antigen binding moiety shares an amino- or carboxy-terminal peptide bond with either a) said mutant IL-2 polypeptide or b) said first antigen binding moiety. In another embodiment the mutant IL-2 polypeptide comprised in the immunoconjugate according to the invention shares an amino- or carboxy-terminal peptide bond with said first effector cell binding moiety and said second effector cell binding moiety shares an amino- or carboxy-terminal peptide bond with either a) said mutant IL-2 polypeptide or b) said first effector cell binding moiety. In another embodiment the mutant IL-2 polypeptide comprised in the immunoconjugate according to the invention shares an amino- or carboxy-terminal peptide bond with the antigen binding moiety and the effector cell binding moiety shares an amino- or carboxy-terminal peptide bond with either a) said mutant IL-2 polypeptide or b) said antigen binding moiety. In another embodiment the mutant IL-2 polypeptide comprised in the immunoconjugate according to the invention shares an amino- or carboxy-terminal peptide bond with the effector cell binding moiety and the antigen binding moiety shares an amino- or carboxy-terminal peptide bond with either a) said mutant IL-2 polypeptide or b) said effector cell binding moiety.

In one embodiment the antigen binding moiety and/or the effector cell binding moiety comprised in the immunoconjugate according to the invention is an antibody. In another embodiment said antigen binding moiety and/or the effector cell binding moiety is an antibody fragment. In a specific embodiment said antigen binding moiety and/or the effector cell binding moiety is selected from a Fab molecule and a scFv molecule. In a particular embodiment said antigen binding moiety and/or the effector cell binding moiety is a Fab molecule. In another particular embodiment said antigen binding moiety and/or the effector cell binding moiety is an immunoglobulin molecule. In a more specific embodiment said antigen binding moiety and/or the effector cell binding moiety is an IgG class, particularly an IgG₁ subclass, immunoglobulin molecule. In certain embodiments said antigen binding moiety is directed to an antigen presented on a tumor cell or in a tumor cell environment, particularly an antigen selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). In certain embodiments the effector cell binding moiety is directed to an effector cell present in a tumor cell environment in order to achieve cis-targeting, particularly a target selected from the group of CD8, PD-1, LAG3, TIM3, TIGIT, CD28, 4-1BB, OX40. GITR. ICOS, CXCR3, CXCR5.

The invention further provides isolated polynucleotides encoding a mutant IL-2 polypeptide or an immunoconjugate as described herein, expression vectors comprising said polynucleotides, and host cells comprising the polynucleotides or the expression vectors.

Also provided is a method of producing a mutant IL-2 polypeptide or an immunoconjugate as described herein, a pharmaceutical composition comprising a mutant IL-2 polypeptide or an immunoconjugate as described herein and a pharmaceutically acceptable carrier, and methods of using a mutant IL-2 polypeptide or an immunoconjugate as described herein.

In particular, the invention encompasses a mutant IL-2 polypeptide or an immunoconjugate as described herein for use in the treatment of a disease in an individual in need thereof. In a particular embodiment said disease is cancer. In a particular embodiment the individual is a human.

Also encompassed by the invention is the use of the mutant IL-2 polypeptide or immunoconjugate as described herein for the manufacture of a medicament for treating a disease in an individual in need thereof.

Further provided is a method of treating disease in an individual, comprising administering to said individual a therapeutically effective amount of a composition comprising a mutant IL-2 polypeptide or an immunoconjugate as described herein. Said disease preferably is cancer.

Also provided is a method of stimulating the immune system of an individual, comprising administering to said individual an effective amount of a composition comprising the mutant IL-2 polypeptide or immunoconjugate described herein in a pharmaceutically acceptable form.

### BRIEF DESCRIPTION OF THE DRAWINGS

FIG. 1. A schematic representation of the constructs comprising the extracellular domains (ECD) of the IL2R beta and gamma (IL2Rβγ) subunits.

FIG. 2. Analysis of IL2v binding to the receptor IL2Rfβγ at pH 6 and pH 7.4 by ELISA. Bacterial supernatant containing IL2v was incubated with previously coated IL2Rfβγ. Prior to the detection by an anti-Flag/HRP secondary antibody, extensive washing for 30 minutes was performed at pH 6 or pH 7.4.

FIG. 3. Binding analysis for the first set of selected pH-dependent IL2v variants to IL2Rfβγ at pH 6 and pH 7.4 by ELISA. Bacterial supernatants containing clonal pH-dependent IL2v variants were incubated with previously coated IL2Rfβγ. Prior to the detection by an anti-Flag/HRP secondary antibody, extensive washing for 30 minutes was performed at pH 6 or pH 7.4. The absorption at pH 6 was normalized to a value of 1 and the ratio of absorption levels was compared.

FIG. 4. Binding analysis for the second set of selected pH-dependent IL2v variants to IL2Rfβγ at pH 6, pH 6.5, pH 7 and pH 7.4 by ELISA. Bacterial supernatants containing clonal pH-dependent IL2v variants were incubated with previously coated IL2Rfβγ. Prior to the detection by an anti-Flag/HRP secondary antibody, extensive washing for 30 minutes was performed at pH 6 or pH 7.4. The absorption at pH 6 was normalized to a value of 1 and the ratio of absorption levels was compared.

FIGS. 5A-U. Binding analysis of selected pH-dependent IL2v variants to IL2Rfβγ at pH 6 and pH 7.4 by SPR. An exemplary binding analysis is provided in FIG. 5A. Selected clones as indicated in FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, FIG. 5O, FIG. 5P, FIG. 5Q, FIG. 5R FIG. 5S FIG. 5T and FIG. 5U were analyzed under the same conditions as indicated in FIG. 5A.

FIGS. 6A-B. Concentration-dependent pSTAT5 induction by anti-CD8-IL2v (pH) constructs in human NK cells at normal pH (FIG. 6A); or low pH (FIG. 6B)

FIG. 7. Comparison of pSTAT5 activation (AUC) by anti-CD8-IL2v (pH) constructs in human NK cells at normal and low pH.

FIG. 8. One-armed CD8-targeted IgG PG LALA with IL2v or pH-dependent variant thereof fused to the C-terminus of the empty Fc knob chain.

FIGS. 9A-D. Proliferation-induction of CD8+ T cells at pH 6.5 (FIG. 9A), NK cells at pH 6.5 (FIG. 9B), CD8+ T cells at pH 7.4 (FIG. 9C) and NK cells at pH 7.4 (FIG. 9D) by anti-CD8-IL2v (pH) constructs.

FIGS. 10A-F. Determination of pH-dependent proliferation at pH6.5 and pH7.5 of CD8+ T cells (FIG. 10A) or NK cells (FIG. 10B) and their activation by CD69 expression (FIG. 10C and FIG. 10D, respectively) and by CD25 expression (FIG. 10E and FIG. 10F, respectively) mediated by anti-CD8-IL2v (pH) constructs during 5 days of incubation in vitro.

FIG. 11. Determination of anti-CD8-IL2v (pH) construct activity ratios at pH6.5 to pH7.4 based on area under the curve (AUC) values of immune cell activation and proliferation.

FIG. 12. Bivalent PD1-targeted IgG PG LALA with IL2v or pH-dependent variants thereof fused to the C-terminus of the Fc knob chain.

FIGS. 13A-D. Determination of enhancement of aCD3-stimulated CD4+ T cell activation and CD8+ T cell activation by CD25 expression (FIG. 13A and FIG. 13B) and by CD69 expression (FIG. 13C and FIG. 13D) at pH6.5, pH6.8 and pH7.4 induced by anti-PD1-IL2v (pH) constructs in vitro.

FIG. 14: Non-targeted Fc PG LALA-fusions with wt IL2 or pH-dependent variants thereof fused to the C-terminus of the Fc knob chain.

FIGS. 15A-F. Activation of human CD8+ T cells (FIG. 15A, FIG. 15B and FIG. 15C) and NK cells (FIG. 15D, FIG. 15E and FIG. 15F) as determined by CD69 and proliferation induced by IL2 wt (pH) constructs at pH6.5 vs pH 7.4 in vitro upon 5 days of incubation.

### DETAILED DESCRIPTION OF THE INVENTION

### Definitions

Terms are used herein as generally used in the art, unless otherwise defined in the following.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 144.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 144). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as an IL-2 mutant peptide sequence, an IL-2 mutant polypeptide, IL-2 mutant protein or IL-2 mutant analog.

Designation of various forms of IL-2 is herein made with respect to the sequence shown in SEQ ID NO: 144. Various designations may be used herein to indicate the same mutation. For example a mutation from phenylalanine at position 42 to alanine can be indicated as 42A, A42, A42, F42A, or Phe42Ala.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to CD25. Amino acid sequence deletions and insertions include amino- and/or carboxy-terminal deletions and insertions of amino acids. An example of a terminal deletion is the deletion of the alanine residue in position 1 of full-length human IL-2. Preferred amino acid mutations are amino acid substitutions. For the purpose of altering e.g. the binding characteristics of an IL-2 polypeptide, non-conservative amino acid substitutions, i.e. replacing one amino acid with another amino acid having different structural and/or chemical properties, are particularly preferred. Preferred amino acid substations include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A. In certain embodiments according to the invention the wild-type IL-2 polypeptide to which the mutant IL-2 polypeptide is compared comprises the amino acid sequence of SEQ ID NO: 144.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25.

The term “high-affinity IL-2 receptor” as used herein refers to the heterotrimeric form of the IL-2 receptor, consisting of the receptor γ-subunit (also known as common cytokine receptor γ-subunit, γ_{c}, or CD132), the receptor β-subunit (also known as CD122 or p70) and the receptor α-subunit (also known as CD25 or p55). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein.

The affinity of the mutant or wild-type IL-2 polypeptide for various forms of the IL-2 receptor can be determined in accordance with the method set forth in the Examples by surface plasmon resonance (SPR), using standard instrumentation such as a BIAcore instrument (GE Healthcare) and receptor subunits such as may be obtained by recombinant expression (see e.g. Shanafelt et al., Nature Biotechnol 18, 1197-1202 (2000)). Alternatively, binding affinity of IL-2 mutants for different forms of the IL-2 receptor may be evaluated using cell lines known to express one or the other such form of the receptor. Specific illustrative and exemplary embodiments for measuring binding affinity are described hereinafter.

By “regulatory T cell” or “T_{reg} cell” is meant a specialized type of CD4⁺ T cell that can suppress the responses of other T cells. T_{reg} cells are characterized by expression of the α-subunit of the IL-2 receptor (CD25) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. T_{reg} cells require IL-2 for their function and development and induction of their suppressive characteristics.

As used herein, the term “effector cells” refers to a population of lymphocytes that mediate the cytotoxic effects of IL-2. Effector cells include effector T cells such as CD8⁺ cytotoxic T cells, NK cells, lymphokine-activated killer (LAK) cells and macrophages/monocytes.

As used herein, the term “antigen binding moiety” refers to a polypeptide molecule that specifically binds to an antigenic determinant. In one embodiment, an antigen binding moiety is able to direct the entity to which it is attached (e.g. a cytokine or a second antigen binding moiety) to a target site, for example to a specific type of tumor cell or tumor stroma bearing the antigenic determinant. Antigen binding moieties include antibodies and fragments thereof as further defined herein. Preferred antigen binding moieties include an antigen binding domain of an antibody, comprising an antibody heavy chain variable region and an antibody light chain variable region. In certain embodiments, the antigen binding moieties may include antibody constant regions as further defined herein and known in the art. Useful heavy chain constant regions include any of the five isotypes: α, δ, ε, γ, or μ. Useful light chain constant regions include any of the two isotypes: κ and λ.

By “specifically binds” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance technique (analyzed on a BIAcore instrument) (Liljeblad et al., Glyco J 17, 323-329 (2000)), and traditional binding assays (Heeley, Endocr Res 28, 217-229 (2002)).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM).

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a therapeutic polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode mutant IL-2 polypeptides or immunoconjugates of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode mutant IL-2 polypeptides or immunoconjugates of the invention or fragments thereof.

The term “artificial” refers to a synthetic, or non-host cell derived composition, e.g. a chemically-synthesized oligonucleotide.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen binding activity.

The terms “full length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and multispecific antibodies formed from antibody fragments. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Antibody fragments can be made by various techniques, including but not limited to proteolytic digestion of an intact antibody as well as production by recombinant host cells (e.g. E. coli or phage), as described herein.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g. γ₁ (IgG₁), γ2 (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present.

A “modification promoting heterodimerization” is a manipulation of the peptide backbone or the post-translational modifications of a polypeptide, e.g. an immunoglobulin heavy chain, that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two immunoglobulin heavy chains wherein further immunoconjugate components fused to each of the heavy chains (e.g. IL-2 polypeptide) are not the same. In the immunoconjugates of the present invention, the modification promoting heterodimerization is in the heavy chain(s), specifically in the Fc domain, of an immunoglobulin molecule. In some embodiments the modification promoting heterodimerziation comprises an amino acid mutation, specifically an amino acid substitution. In a particular embodiment, the modification promoting heterodimerization comprises a separate amino acid mutation, specifically an amino acid substitution, in each of the two immunoglobulin heavy chains.

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

As used herein, the term “immunoconjugate” refers to a polypeptide molecule that includes at least one IL-2 moiety and at least one antigen binding moiety. In certain embodiments, the immunoconjugate comprises at least one IL-2 moiety, and at least two antigen binding moieties. Particular immunoconjugates according to the invention essentially consist of one IL-2 moiety and two antigen binding moieties joined by one or more linker sequences. The antigen binding moiety can be joined to the IL-2 moiety by a variety of interactions and in a variety of configurations as described herein.

As used herein, the term “control antigen binding moiety” refers to an antigen binding moiety as it would exist free of other antigen binding moieties and effector moieties. For example, when comparing an Fab-IL2-Fab immunoconjugate of the invention with a control antigen binding moiety, the control antigen binding moiety is free Fab, wherein the Fab-IL2-Fab immunoconjugate and the free Fab molecule can both specifically bind to the same antigen determinant.

As used herein, the terms “first” and “second” with respect to antigen binding moieties etc., are used for convenience of distinguishing when there is more than one of each type of moiety. Use of these terms is not intended to confer a specific order or orientation of the immunoconjugate unless explicitly so stated.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Preferably, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “pH-dependent” refers to a property of a molecule, particularly an IL-2 variant, which is dependent on the pH of the environment, e.g. receptor binding is facilitated at a first pH range or pH value but not facilitated with the same quality at a second pH range or pH value, wherein the first and the second pH range do not overlap or the first and the second pH value are not identical.

As used herein, “pH-dependent IL-2 receptor binding” refers to the binding property of a molecule, particularly an IL-2 variant, wherein said molecule binds to the IL-2 receptor, specific IL-2 receptor subunits or IL-2 receptor subunit combinations in a pH-dependent manner. Said “pH-dependent IL-2 receptor binding” relates to, but this not limited to, the situation, wherein a pH-dependent IL-2 binds to the IL-2 receptor, IL-2 receptor subunits or IL-2 receptor subunit combinations at slightly acidic pH, for example at pH 6.0 and/or pH 6.5, and shows reduced or abolished receptor binding at systemic pH, for example at pH 7.0 and/or pH 7.4.

### DETAILED DESCRIPTION OF THE EMBODIMENTS

The present invention aims at providing a mutant IL-2 polypeptide having improved properties for immunotherapy. In particular the invention aims at eliminating pharmacological properties of IL-2 that contribute to toxicity but are not essential for efficacy of IL-2. As discussed above, different forms of the IL-2 receptor consist of different subunits and exhibit different affinities for IL-2. The intermediate-affinity IL-2 receptor, consisting of the β and γ receptor subunits, is expressed on resting effector cells and is sufficient for IL-2 signaling. The high-affinity IL-2 receptor, additionally comprising the α-subunit of the receptor, is mainly expressed on regulatory T (T_{reg}) cells as well as on activated effector cells where its engagement by IL-2 can promote T_{reg} cell-mediated immunosuppression or activation-induced cell death (AICD), respectively. Thus, without wishing to be bound by theory, reducing or abolishing the affinity of IL-2 to the α-subunit of the IL-2 receptor should reduce IL-2 induced downregulation of effector cell function by regulatory T cells and development of tumor tolerance by the process of AICD. On the other hand, maintaining the affinity to the intermediate-affinity IL-2 receptor should preserve the induction of proliferation and activation of effector cells like NK and T cells by IL-2.

Several IL-2 mutants already exist in the art, however, the inventors have found novel amino acid mutations of the IL-2 polypeptide and combinations thereof that are particularly suitable to confer to IL-2 the desired characteristics for immunotherapy.

Furthermore, the invention aims at providing pH-dependent IL-2 polypeptides having a reduced or abolished systemic activity at neutral pH and full activity in the tumor microenvironment at acidic pH.

In a first aspect the invention provides a mutant interleukin-2 (IL-2) polypeptide comprising one or more amino acid substitutions, each compared to a wilde-type IL-2, preferably human IL-2 according to SEQ ID NO: 144, wherein the one or more amino acid substitutions abolishes or reduces binding to the IL-2 receptor, preferably to the intermediate-affinity IL-2 receptor (IL2Rfβγ), at neutral pH and facilitate binding to the IL-2 receptor, preferably to the intermediate-affinity IL-2 receptor (IL2Rβγ), at decreased pH. In one embodiment, the mutant IL-2 polypetide comprises one or more amino acid substitutions, each compared to a human IL-2 according to SEQ ID NO: 144, wherein the one or more amino acid substitutions abolishes or reduces binding to the intermediate-affinity IL-2 receptor (IL2Rβγ) at neutral pH and facilitate binding to the intermediate-affinity IL-2 receptor (IL2Rfβγ) at decreased pH.

In one embodiment, the mutant IL-2 polypetide exhibits reduced or abolished IL-2 receptor binding, preferably intermediate-affinity IL-2 receptor binding, at pH 7.4 and/or pH 7.0 and retained IL-2 receptor binding, preferably intermediate-affinity IL-2 receptor binding, at a pH 6 and/or pH 6.5. In one embodiment said one or more amino acid substitutions is at a position selected from the group of positions corresponding to residue 6, 8, 11, 12, 13, 15, 16, 19, 20, 22, 23, 81, 84, 87, 91, 95, 120, 123, 126, 130, 133 of human IL-2 according to SEQ ID NO: 144. In one embodiment said one or more amino acid substitutions is selected from the group of S6Y, K8E, Q11E, Q11T, L12D, L12E, L12Q, L12S, L12T, Q13H, Q13R, E15Q, H16D, H16E, H16N, H16Q, L19D, L19Q, D20E, D20Q, Q22D, Q22E, Q22H, M23E, M23N, M23Q, R81D, R81E, R81H, R81N, R81Q, D84E, D84Q, S87D, S87E, 587N, S87Q, V91D, V91E, V91N, E95D, E95Q, R120E, R120H, T123E, T123Q, Q126E, Q126H, S130E, T133D, T133E, T133N, T133Q. In one embodiment the mutant IL-2 polypeptide comprises the amino acid substitutions (i) L12E, D20E, M23N, R81N, D84E, S87E, R120E, T123E, S130E, T133N; (ii) Q11E, D20Q, M23E, R81D, D84E, S87E, S130E, T133N; (iii) L12E, L19D, R81D, R120E, T123E, S130E, T133E; (iv) R81Q, S87E, V91D, T123E, S130E, T133D; (v) Q11E, L12E, M23Q, R81D, S87E, V91D, S130E, T133Q; (vi) Q11E, L19D, R81D, D84E, S130E, T133D; (vii) R81D, D84Q, S87D, V91N, T123Q, S130E, T133D; (viii) L19D, R81E, D84E, S87Q, R120H, S130E, T133E; (iix) L19D, M23N, R81D, T133E; (ix) Q11E, L12E, M23Q, R81Q, S87D, V91N, E95Q, R120H, T123E, S130E, T133E; (x) L19D, R81E, S130E, T133D; (xi) R81Q, S87E, V91D, R120E, S130E, T133D; (xii) L12Q, L19Q, R81H, V91E, T123E, S130E, T133E; (xiii) K8E, D20E, M23N, R81H, D84Q, S87E, R120H, S130E, T133D; (xiv) L12E, L19Q, R81H, R120E, T133D; (xv) H16E, L19D, Q22E, M23Q, R81D, D84E, S87D, R120H, S130E, T133E; (xvi) Q11E, L12E, H16Q, L19D, Q22E, M23N, R81E, D84E, S87E, R120H, S130E, T133E; (xvii) Q11E, H16E, L19D, M23E, R81D, S87E, R120H, Q126E, T133D; (xviii) Q11E, L12S, E15Q, H16N, L19D, M23E, R81E, D84E, S87D, R120H, S130E, T133E; (xix) Q11E, H16E, M23E, R81N, D84E, S87E, R120H, Q126E, S130E, T133E; (xx) Q11E, E15Q, H16E, Q22E, M23E, R81H, D84E, S87E, R120H, S130E, T133D; (xxi) Q11E, L12D, Q13H, E15Q, H16E, Q22E, M23E, R81N, D84E, S87E, R120H, S130E, T133E; (xxii) Q11E, E15Q, H16E, L19D, R81E, S87E, R120H, S130E, T133E; (xxiii) H16E, L19D, M23Q, R81N, D84E, S87D, R120H, S130E, T133D; (xxiv) Q11E, L12T, E15Q, H16E, L19D, Q22H, R81D, D84E, S87E, R120H, S130E, T133E; (xxv) Q11E, L12T, E15Q, H16E, L19D, Q22H, M23E, R81E, D84E, S87E, R120H, S130E, T133E; (xxvi) Q11E, E15Q, H16E, L19D, R81E, D84E, S87E, R120H, S130E, T133E; (xxvii) H16E, Q22E, M23Q, S87N, R120H, S130E, T133E; (xxviii) H16E, L19D, Q22D, M23Q, R81D, D84E, S87E, R120H, S130E, T133E; (xxiix) Q11E, L12E, Q13H, E15Q, H16N, L19D, Q22E, M23Q, R81E, D84E, S87D, E95D, R120H, T133E; (xxix) Q11E, L12T, H16E, L19D, Q22E, R81D, D84E, S87E, R120H, S130E, T133D; (xxx) Q11T, L12E, E15Q, H16E, L19D, R81D, D84E, S87E, R120E, S130E, T133D; (xxxi) Q11E, E15Q, H16E, L19D, R81D, D84E, S87E, R120H, S130E, T133E; (xxxii) Q11E, E15Q, H16E, L19D, R81Q, D84E, S87E, R120H, S130E, T133E; (xxxiii) Q11E, L12S, H16E, L19D, M23Q, R81D, D84E, S87E, R120H, S130E, T133E; (xxxiv) S6Y, L12E, Q13R, H16Q, Q22E, M23Q, R81N, D84E, S87E, R120H, S130E, T133D; (xxxv) H16D, M23N, R81D, D84E, R120H, S130E, T133E; (xxxvi) Q11E, L12TE, H16Q, L19D, M23E, R81D, D84E, S87E, R120H, S130E, T133E; (xxxvii) Q11E, L12E, H16NE, M23N, R81E, D84E, R120H, S130E, T133E; (xxxviii) E15Q, H16E, L19D, R81D, D84E, S87E; (xxxix) Q11E, R120H, S130E, T133D; or (xl) Q11E, R81D, D84E, S87E, R120H, S130E, T133D.

In one embodiment the mutant IL-2 polypeptide comprises the amino acid substitutions (i) L12E, D20E, M23N, R81N, D84E, S87E, R120E, T123E, S130E, T133N, F42A, Y45A, L72G; (ii) Q11E, D20Q, M23E, R81D, D84E, S87E, S130E, T133N, F42A, Y45A, L72G; (iii) L12E, L19D, R81D, R120E, T123E, S130E, T133E, F42A, Y45A, L72G; (iv) R81Q, S87E, V91D, T123E, S130E, T133D, F42A, Y45A, L72G; (v) Q11E, L12E, M23Q, R81D, S87E, V91D, S130E, T133Q, F42A, Y45A, L72G; (vi) Q11E, L19D, R81D, D84E, S130E, T133D, F42A, Y45A, L72G; (vii) R81D, D84Q, S87D, V91N, T123Q, S130E, T133D, F42A, Y45A, L72G; (viii) L19D, R81E, D84E, S87Q, R120H, S130E, T133E, F42A, Y45A, L72G; (iix) L19D, M23N, R81D, T133E, F42A, Y45A, L72G; (ix) Q11E, L12E, M23Q, R81Q, S87D, V91N, E95Q, R120H, T123E, S130E, T133E, F42A, Y45A, L72G; (x) L19D, R81E, S130E, T133D, F42A, Y45A, L72G; (xi) R81Q, S87E, V91D, R120E, S130E, T133D, F42A, Y45A, L72G; (xii) L12Q, L19Q, R81H, V91E, T123E, S130E, T133E, F42A, Y45A, L72G; (xiii) K8E, D20E, M23N, R81H, D84Q, S87E, R120H, S130E, T133D, F42A, Y45A, L72G; (xiv) L12E, L19Q, R81H, R120E, T133D, F42A, Y45A, L72G; (xv) H16E, L19D, Q22E, M23Q, R81D, D84E, S87D, R120H, S130E, T133E, F42A, Y45A, L72G; (xvi) Q11E, L12E, H16Q, L19D, Q22E, M23N, R81E, D84E, S87E, R120H, S130E, T133E, F42A, Y45A, L72G; (xvii) Q11E, H16E, L19D, M23E, R81D, S87E, R120H, Q126E, T133D, F42A, Y45A, L72G; (xviii) Q11E, L12S, E15Q, H16N, L19D, M23E, R81E, D84E, S87D, R120H, S130E, T133E, F42A, Y45A, L72G; (xix) Q11E, H16E, M23E, R81N, D84E, S87E, R120H, Q126E, S130E, T133E, F42A, Y45A, L72G; (xx) Q11E, E15Q, H16E, Q22E, M23E, R81H, D84E, S87E, R120H, S130E, T133D, F42A, Y45A, L72G; (xxi) Q11E, L12D, Q13H, E15Q, H16E, Q22E, M23E, R81N, D84E, S87E, R120H, S130E, T133E, F42A, Y45A, L72G; (xxii) Q11E, E15Q, H16E, L19D, R81E, S87E, R120H, S130E, T133E, F42A, Y45A, L72G; (xxiii) H16E, L19D, M23Q, R81N, D84E, S87D, R120H, S130E, T133D, F42A, Y45A, L72G; (xxiv) Q11E, L12T, E15Q, H16E, L19D, Q22H, R81D, D84E, S87E, R120H, S130E, T133E, F42A, Y45A, L72G; (xxv) Q11E, L12T, E15Q, H16E, L19D, Q22H, M23E, R81E, D84E, S87E, R120H, S130E, T133E, F42A, Y45A, L72G; (xxvi) Q11E, E15Q, H16E, L19D, R81E, D84E, S87E, R120H, S130E, T133E, F42A, Y45A, L72G; (xxvii) H16E, Q22E, M23Q, S87N, R120H, S130E, T133E, F42A, Y45A, L72G; (xxviii) H16E, L19D, Q22D, M23Q, R81D, D84E, S87E, R120H, S130E, T133E, F42A, Y45A, L72G; (xxiix) Q11E, L12E, Q13H, E15Q, H16N, L19D, Q22E, M23Q, R81E, D84E, S87D, E95D, R120H, T133E, F42A, Y45A, L72G; (xxix) Q11E, L12T, H16E, L19D, Q22E, R81D, D84E, S87E, R120H, S130E, T133D, F42A, Y45A, L72G; (xxx) Q11T, L12E, E15Q, H16E, L19D, R81D, D84E, S87E, R120E, S130E, T133D, F42A, Y45A, L72G; (xxxi) Q11E, E15Q, H16E, L19D, R81D, D84E, S87E, R120H, S130E, T133E, F42A, Y45A, L72G; (xxxii) Q11E, E15Q, H16E, L19D, R81Q, D84E, S87E, R120H, S130E, T133E, F42A, Y45A, L72G; (xxxiii) Q11E, L12S, H16E, L19D, M23Q, R81D, D84E, S87E, R120H, S130E, T133E, F42A, Y45A, L72G; (xxxiv) S6Y, L12E, Q13R, H16Q, Q22E, M23Q, R81N, D84E, S87E, R120H, S130E, T133D, F42A, Y45A, L72G; (xxxv) H16D, M23N, R81D, D84E, R120H, S130E, T133E, F42A, Y45A, L72G; (xxxvi) Q11E, L12TE, H16Q, L19D, M23E, R81D, D84E, S87E, R120H, S130E, T133E, F42A, Y45A, L72G; (xxxvii) Q11E, L12E, H16NE, M23N, R81E, D84E, R120H, S130E, T133E, F42A, Y45A, L72G; (xxxviii) E15Q, H16E, L19D, R81D, D84E, S87E, F42A, Y45A, L72G; (xxxix) Q11E, R120H, S130E, T133D F42A, Y45A, L72G; or (xl) Q11E, R81D, D84E, S87E, R120H, S130E, T133D, F42A, Y45A, L72G.

In one embodiment the mutant IL-2 polypeptide comprises the amino acid substitutions (i) L12E, D20E, M23N, R81N, D84E, S87E, R120E, T123E, S130E, T133N, F42A, Y45A, L72G, T3A, C125A; (ii) Q11E, D20Q, M23E, R81D, D84E, S87E, S130E, T133N, F42A, Y45A, L72G, T3A, C125A; (iii) L12E, L19D, R81D, R120E, T123E, S130E, T133E, F42A, Y45A, L72G, T3A, C125A; (iv) R81Q, S87E, V91D, T123E, S130E, T133D, F42A, Y45A, L72G, T3A, C125A; (v) Q11E, L12E, M23Q, R81D, S87E, V91D, S130E, T133Q, F42A, Y45A, L72G, T3A, C125A; (vi) Q11E, L19D, R81D, D84E, S130E, T133D, F42A, Y45A, L72G, T3A, C125A; (vii) R81D, D84Q, S87D, V91N, T123Q, S130E, T133D, F42A, Y45A, L72G, T3A, C125A; (viii) L19D, R81E, D84E, S87Q, R120H, S130E, T133E, F42A, Y45A, L72G, T3A, C125A; (iix) L19D, M23N, R81D, T133E, F42A, Y45A, L72G, T3A, C125A; (ix) Q11E, L12E, M23Q, R81Q, S87D, V91N, E95Q, R120H, T123E, S130E, T133E, F42A, Y45A, L72G, T3A, C125A; (x) L19D, R81E, S130E, T133D, F42A, Y45A, L72G, T3A, C125A; (xi) R81Q, S87E, V91D, R120E, S130E, T133D, F42A, Y45A, L72G, T3A, C125A; (xii) L12Q, L19Q, R81H, V91E, T123E, S130E, T133E, F42A, Y45A, L72G, T3A, C125A; (xiii) K8E, D20E, M23N, R81H, D84Q, S87E, R120H, S130E, T133D, F42A, Y45A, L72G, T3A, C125A; (xiv) L12E, L19Q, R81H, R120E, T133D, F42A, Y45A, L72G, T3A, C125A; (xv) H16E, L19D, Q22E, M23Q, R81D, D84E, S87D, R120H, S130E, T133E, F42A, Y45A, L72G, T3A, C125A; (xvi) Q11E, L12E, H16Q, L19D, Q22E, M23N, R81E, D84E, S87E, R120H, S130E, T133E, F42A, Y45A, L72G, T3A, C125A; (xvii) Q11E, H16E, L19D, M23E, R81D, S87E, R120H, Q126E, T133D, F42A, Y45A, L72G, T3A, C125A; (xviii) Q11E, L12S, E15Q, H16N, L19D, M23E, R81E, D84E, S87D, R120H, S130E, T133E, F42A, Y45A, L72G, T3A, C125A; (xix) Q11E, H16E, M23E, R81N, D84E, S87E, R120H, Q126E, S130E, T133E, F42A, Y45A, L72G, T3A, C125A; (xx) Q11E, E15Q, H16E, Q22E, M23E, R81H, D84E, S87E, R120H, S130E, T133D, F42A, Y45A, L72G, T3A, C125A; (xxi) Q11E, L12D, Q13H, E15Q, H16E, Q22E, M23E, R81N, D84E, S87E, R120H, S130E, T133E, F42A, Y45A, L72G, T3A, C125A; (xxii) Q11E, E15Q, H16E, L19D, R81E, S87E, R120H, S130E, T133E, F42A, Y45A, L72G, T3A, C125A; (xxiii) H16E, L19D, M23Q, R81N, D84E, S87D, R120H, S130E, T133D, F42A, Y45A, L72G, T3A, C125A; (xxiv) Q11E, L12T, E15Q, H16E, L19D, Q22H, R81D, D84E, S87E, R120H, S130E, T133E, F42A, Y45A, L72G, T3A, C125A; (xxv) Q11E, L12T, E15Q, H16E, L19D, Q22H, M23E, R81E, D84E, S87E, R120H, S130E, T133E, F42A, Y45A, L72G, T3A, C125A; (xxvi) Q11E, E15Q, H16E, L19D, R81E, D84E, S87E, R120H, S130E, T133E, F42A, Y45A, L72G, T3A, C125A; (xxvii) H16E, Q22E, M23Q, S87N, R120H, S130E, T133E, F42A, Y45A, L72G, T3A, C125A; (xxviii) H16E, L19D, Q22D, M23Q, R81D, D84E, S87E, R120H, S130E, T133E, F42A, Y45A, L72G, T3A, C125A; (xxiix) Q11E, L12E, Q13H, E15Q, H16N, L19D, Q22E, M23Q, R81E, D84E, S87D, E95D, R120H, T133E, F42A, Y45A, L72G, T3A, C125A; (xxix) Q11E, L12T, H16E, L19D, Q22E, R81D, D84E, S87E, R120H, S130E, T133D, F42A, Y45A, L72G, T3A, C125A; (xxx) Q11T, L12E, E15Q, H16E, L19D, R81D, D84E, S87E, R120E, S130E, T133D, F42A, Y45A, L72G, T3A, C125A; (xxxi) Q11E, E15Q, H16E, L19D, R81D, D84E, S87E, R120H, S130E, T133E, F42A, Y45A, L72G, T3A, C125A; (xxxii) Q11E, E15Q, H16E, L19D, R81Q, D84E, S87E, R120H, S130E, T133E, F42A, Y45A, L72G, T3A, C125A; (xxxiii) Q11E, L12S, H16E, L19D, M23Q, R81D, D84E, S87E, R120H, S130E, T133E, F42A, Y45A, L72G, T3A, C125A; (xxxiv) S6Y, L12E, Q13R, H16Q, Q22E, M23Q, R81N, D84E, S87E, R120H, S130E, T133D, F42A, Y45A, L72G, T3A, C125A; (xxxv) H16D, M23N, R81D, D84E, R120H, S130E, T133E, F42A, Y45A, L72G, T3A, C125A; (xxxvi) Q11E, L12TE, H16Q, L19D, M23E, R81D, D84E, S87E, R120H, S130E, T133E, F42A, Y45A, L72G, T3A, C125A; (xxxvii) Q11E, L12E, H16NE, M23N, R81E, D84E, R120H, S130E, T133E, F42A, Y45A, L72G, T3A, C125A; (xxxviii) E15Q, H16E, L19D, R81D, D84E, S87E, F42A, Y45A, L72G, T3A, C125A; (xxxix) Q11E, R120H, S130E, T133D, F42A, Y45A, L72G, T3A, T3A, C125A; or (xl) Q11E, R81D, D84E, S87E, R120H, S130E, T133D, F42A, Y45A, L72G, T3A, C125A.

In a specific embodiment, the mutant IL-2 polypeptide of the invention comprises a polypeptide sequence that is at least about 80%, 85%, 90%, 95%, 96%, 97%, 98% or 99% identical to a sequence selected from the group of SEQ ID NO: 5; SEQ ID NO: 6; SEQ ID NO:7; SEQ ID NO:8; SEQ ID NO:9; SEQ ID NO: 10; SEQ ID NO: 11; SEQ ID NO: 12; SEQ ID NO: 13; SEQ ID NO: 14; SEQ ID NO: 15; SEQ ID NO: 16); SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 59; SEQ ID NO: 60; SEQ ID NO: 61.

In a specific embodiment the mutant IL-2 polypeptide comprises the amino acid sequence selected from the group of SEQ ID NO: 5; SEQ ID NO: 6; SEQ ID NO:7; SEQ ID NO:8; SEQ ID NO:9; SEQ ID NO: 10; SEQ ID NO: 11; SEQ ID NO: 12; SEQ ID NO: 13; SEQ ID NO: 14; SEQ ID NO: 15; SEQ ID NO: 16); SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 59; SEQ ID NO: 60; SEQ ID NO: 61.

In one embodiment the mutant IL-2 polypeptide comprises the amino acid substitution Q11E. In one embodiment the mutant IL-2 polypeptide comprises the amino acid substitution E15Q. In one embodiment the mutant IL-2 polypeptide comprises the amino acid substitution H16E. In one embodiment the mutant IL-2 polypeptide comprises the amino acid substitution L19D. In one embodiment mutant IL-2 polypeptide comprises the amino acid substitution Q22E. In one embodiment the mutant IL-2 polypeptide comprises the amino acid substitution M23Q. In one embodiment the mutant IL-2 polypeptide comprises the amino acid substitution R81D. In one embodiment the mutant IL-2 polypeptide comprises the amino acid substitution D84E. In one embodiment the mutant IL-2 polypeptide comprises the amino acid substitution S87E. In one embodiment the mutant IL-2 polypeptide comprises the amino acid substitution R120H. In one embodiment the mutant IL-2 polypeptide comprises the amino acid substitution Q126E. In one embodiment the mutant IL-2 polypeptide comprises the amino acid substitution Q126H. In one embodiment the mutant IL-2 polypeptide comprises the amino acid substitution S130E. In one embodiment the mutant IL-2 polypeptide comprises the amino acid substitution T133E. In one embodiment the mutant IL-2 polypeptide comprises the amino acid substitution T133D.

In one embodiment the mutant IL-2 polypeptide comprises the amino acid substitutions Q11E, F42A, Y45A and L72G. In one embodiment the mutant IL-2 polypeptide comprises the amino acid substitutions E15Q, F42A, Y45A and L72G. In one embodiment the mutant IL-2 polypeptide comprises the amino acid substitutions H16E, F42A, Y45A and L72G. In one embodiment the mutant IL-2 polypeptide comprises the amino acid substitutions L19D, F42A, Y45A and L72G. In one embodiment mutant IL-2 polypeptide comprises the amino acid substitutions Q22E, F42A, Y45A and L72G. In one embodiment the mutant IL-2 polypeptide comprises the amino acid substitutions M23Q, F42A, Y45A and L72G. In one embodiment the mutant IL-2 polypeptide comprises the amino acid substitutions R81D, F42A, Y45A and L72G. In one embodiment the mutant IL-2 polypeptide comprises the amino acid substitutions D84E, F42A, Y45A and L72G. In one embodiment the mutant IL-2 polypeptide comprises the amino acid substitutions S87E, F42A, Y45A and L72G. In one embodiment the mutant IL-2 polypeptide comprises the amino acid substitutions R120H, F42A, Y45A and L72G. In one embodiment the mutant IL-2 polypeptide comprises the amino acid substitutions Q126E, F42A, Y45A and L72G. In one embodiment the mutant IL-2 polypeptide comprises the amino acid substitutions Q126H, F42A, Y45A and L72G.

In one embodiment the mutant IL-2 polypeptide comprises the amino acid substitutions S130E, F42A, Y45A and L72G. In one embodiment the mutant IL-2 polypeptide comprises the amino acid substitutions T133E, F42A, Y45A and L72G. In one embodiment the mutant IL-2 polypeptide comprises the amino acid substitutions T133D, F42A, Y45A and L72G.

In one embodiment the mutant IL-2 polypeptide comprises the amino acid substitutions Q11E, F42A, Y45A, L72G, T3A and C125A. In one embodiment the mutant IL-2 polypeptide comprises the amino acid substitutions E15Q, F42A, Y45A, L72G, T3A and C125A. In one embodiment the mutant IL-2 polypeptide comprises the amino acid substitutions H16E, F42A, Y45A, L72G, T3A and C125A. In one embodiment the mutant IL-2 polypeptide comprises the amino acid substitutions L19D, F42A, Y45A, L72G, T3A and C125A. In one embodiment mutant IL-2 polypeptide comprises the amino acid substitutions Q22E, F42A, Y45A, L72G, T3A and C125A. In one embodiment the mutant IL-2 polypeptide comprises the amino acid substitutions M23Q, F42A, Y45A, L72G, T3A and C125A. In one embodiment the mutant IL-2 polypeptide comprises the amino acid substitutions R81D, F42A, Y45A, L72G, T3A and C125A. In one embodiment the mutant IL-2 polypeptide comprises the amino acid substitutions D84E, F42A, Y45A, L72G, T3A and C125A. In one embodiment the mutant IL-2 polypeptide comprises the amino acid substitutions S87E, F42A, Y45A, L72G, T3A and C125A. In one embodiment the mutant IL-2 polypeptide comprises the amino acid substitutions R120H, F42A, Y45A, L72G. In one embodiment the mutant IL-2 polypeptide comprises the amino acid substitutions Q126E, F42A, Y45A, L72G, T3A and C125A. In one embodiment the mutant IL-2 polypeptide comprises the amino acid substitutions Q126H, F42A, Y45A, L72G, T3A and C125A. In one embodiment the mutant IL-2 polypeptide comprises the amino acid substitutions S130E, F42A, Y45A, L72G, T3A and C125A. In one embodiment the mutant IL-2 polypeptide comprises the amino acid substitutions T133E, F42A, Y45A, L72G, T3A and C125A. In one embodiment the mutant IL-2 polypeptide comprises the amino acid substitutions T133D, F42A, Y45A, L72G, T3A and C125A.

In one embodiment the mutant IL-2 polypeptide comprises the amino acid sequence selected from the group of SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 5; SEQ ID NO: 52; SEQ ID NO: 53; SEQ ID NO: 54; SEQ ID NO: 55; SEQ ID NO: 56; SEQ ID NO: 57; SEQ ID NO: 58.

In one embodiment the mutant IL-2 polypeptide comprises the amino acid substitutions E15Q, H16E, L19D, R81D, D84E and S87E. In one embodiment the mutant IL-2 polypeptide comprises the amino acid substitutions Q11E, R120H, S130E and T133D. In one embodiment the mutant IL-2 polypeptide comprises the amino acid substitutions Q11E, R81D, D84E, S87E, R120H, S130E and T133D.

In one embodiment the mutant IL-2 polypeptide comprises the amino acid substitutions E15Q, H16E, L19D, R81D, D84E, S87E, F42A, Y45A and L72G. In one embodiment the mutant IL-2 polypeptide comprises the amino acid substitutions Q11E, R120H, S130E, T133D, F42A, Y45A and L72G. In one embodiment the mutant IL-2 polypeptide comprises the amino acid substitutions Q11E, R81D, D84E, S87E, R120H, S130E, T133D, F42A, Y45A and L72G.

In one embodiment the mutant IL-2 polypeptide comprises the amino acid substitutions E15Q, H16E, L19D, R81D, D84E, S87E, F42A, Y45A, L72G, T3A and C125A. In one embodiment the mutant IL-2 polypeptide comprises the amino acid substitutions Q11E, R120H, S130E, T133D, F42A, Y45A, L72G, T3A and C125A. In one embodiment the mutant IL-2 polypeptide comprises the amino acid substitutions Q11E, R81D, D84E, S87E, R120H, S130E, T133D, F42A, Y45A, L72G, T3A and C125A.

In one embodiment the mutant IL-2 polypeptide comprises the amino acid sequence selected from the group of SEQ ID NO: 59; SEQ ID NO: 60; SEQ ID NO: 61.

In another aspect the invention provides a mutant interleukin-2 (IL-2) polypeptide comprising an amino acid mutation that abolishes or reduces affinity of the mutant IL-2 polypeptide to the α-subunit of the IL-2 receptor and preserves affinity of the mutant IL-2 polypeptide to the intermediate-affinity IL-2 receptor each compared to a wild-type IL-2 polypeptide. Mutants of human IL-2 (hIL-2) with decreased affinity to CD25 may for example be generated by amino acid substitution at amino acid position 35, 38, 42, 43, 45 or 72 or combinations thereof. Exemplary amino acid substitutions include K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. Particular IL-2 mutants according to the invention comprise a mutation at an amino acid position corresponding to residue 42, 45, or 72 of human IL-2, or a combination thereof. These mutants exhibit substantially similar binding affinity to the intermediate-affinity IL-2 receptor, and have substantially reduced affinity to the α-subunit of the IL-2 receptor and the high-affinity IL-2 receptor compared to a wild-type form of the IL-2 mutant.

Other characteristics of useful mutants may include the ability to induce proliferation of IL-2 receptor-bearing T and/or NK cells, the ability to induce IL-2 signaling in IL-2 receptor-bearing T and/or NK cells, the ability to generate interferon (IFN)-γ as a secondary cytokine by NK cells, a reduced ability to induce elaboration of secondary cytokines—particularly IL-10 and TNF-α—by peripheral blood mononuclear cells (PBMCs), a reduced ability to activate regulatory T cells, a reduced ability to induce apoptosis in T cells, and a reduced toxicity profile in vivo.

In one embodiment according to the invention, the amino acid mutation that abolishes or reduces affinity of the mutant IL-2 polypeptide to the high-affinity IL-2 receptor and preserves affinity of the mutant IL-2 polypeptide to the intermediate-affinity IL-2 receptor is at a position corresponding to residue 72 of human IL-2. In one embodiment said amino acid mutation is an amino acid substitution. In one embodiment said amino acid substitution is selected from the group of L72G, L72A, L72S, L72T, L72Q, L72E, L72N, L72D, L72R, and L72K. In a more specific embodiment said amino acid mutation is the amino acid substitution L72G.

In a particular aspect the invention provides a mutant IL-2 polypeptide comprising a first and a second amino acid mutation that abolishes or reduces affinity of the mutant IL-2 polypeptide to the α-subunit of the IL-2 receptor and preserves affinity of the mutant IL-2 polypeptide to the intermediate affinity IL-2 receptor. In one embodiment said first amino acid mutation is at a position corresponding to residue 72 of human IL-2. In one embodiment said first amino acid mutation is an amino acid substitution. In a specific embodiment said first amino acid mutation is an amino acid substitution selected from the group of L72G, L72A, L72S, L72T, L72Q, L72E, L72N, L72D, L72R, and L72K. In an even more specific embodiment said amino acid substitution is L72G. Said second amino acid mutation is at a different position than said first amino acid mutation. In one embodiment said second amino acid mutation is at a position selected from a position corresponding to residue 35, 38, 42, 43 and 45 of human IL-2. In one embodiment said second amino acid mutation is an amino acid substitution. In a specific embodiment said amino acid substitution is selected from the group of K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, and Y45K. In a particular embodiment said second amino acid mutation is at a position corresponding to residue 42 or 45 of human IL-2. In a specific embodiment said second amino acid mutation is an amino acid substitution, selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, and Y45K. In a more specific embodiment said second amino acid mutation is the amino acid substitution F42A or Y45A. In a more particular embodiment said second amino acid mutation is at the position corresponding to residue 42 of human IL-2. In a specific embodiment said second amino acid mutation is an amino acid substitution, selected from the group of F42A, F42G, F42S, F42T, F42Q, F42E, F42N, F42D, F42R, and F42K. In a more specific embodiment said amino acid substitution is F42A. In another embodiment said second amino acid mutation is at the position corresponding to residue 45 of human IL-2. In a specific embodiment said second amino acid mutation is an amino acid substitution, selected from the group of Y45A, Y45G, Y45S, Y45T, Y45Q, Y45E, Y45N, Y45D, Y45R, and Y45K. In a more specific embodiment said amino acid substitution is Y45A. In certain embodiments the mutant IL-2 polypeptide comprises a third amino acid mutation that abolishes or reduces affinity of the mutant IL-2 polypeptide to the α-subunit of the IL-2 receptor and preserves affinity of the mutant IL-2 polypeptide to the intermediate-affinity IL-2 receptor, each compared to a wild-type IL-2 polypeptide. Said third amino acid mutation is at a different position than said first and second amino acid mutations. In one embodiment said third amino acid mutation is at a position selected from a position corresponding to residue 35, 38, 42, 43 and 45 of human IL-2. In a preferred embodiment said third amino acid mutation is at a position corresponding to residue 42 or 45 of human IL-2. In one embodiment said third amino acid mutation is at a position corresponding to residue 42 of human IL-2. In another embodiment said third amino acid mutation is at a position corresponding to residue 45 of human IL-2. In one embodiment said third amino acid mutation is an amino acid substitution. In a specific embodiment said amino acid substitution is selected from the group of K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, and Y45K. In a more specific embodiment said amino acid substitution is selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, and Y45K. In an even more specific embodiment said amino acid substitution is F42A or Y45A. In one embodiment said amino acid substitution is F42A. In another embodiment said amino acid substitution is Y45A. In certain embodiments the mutant IL-2 polypeptide does not comprise an amino acid mutation at the position corresponding to residue 38 of human IL-2.

In an even more particular aspect of the invention is provided a mutant IL-2 polypeptide comprising three amino acid mutations that abolish or reduce affinity of the mutant IL-2 polypeptide to the α-subunit of the IL-2 receptor but preserve affinity of the mutant IL-2 polypeptide to the intermediate affinity IL-2 receptor. In one embodiment said three amino acid mutations are at positions corresponding to residue 42, 45 and 72 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are amino acid substitutions F42A, Y45A and L72G.

In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the mutant IL-2 polypeptide to the α-subunit of the IL-2 receptor, the combination of these amino acid mutations may reduce the affinity of the mutant IL-2 polypeptide to the α-subunit of the IL-2 receptor by at least 30-fold, at least 50-fold, or even at least 100-fold. In one embodiment said amino acid mutation or combination of amino acid mutations abolishes the affinity of the mutant IL-2 polypeptide to the α-subunit of the IL-2 receptor so that no binding is detectable by surface plasmon resonance as described hereinbelow.

Substantially similar binding to the intermediate-affinity receptor, i.e. preservation of the affinity of the mutant IL-2 polypeptide to said receptor, is achieved when the IL-2 mutant exhibits greater than about 70% of the affinity of a wild-type form of the IL-2 mutant to the intermediate-affinity IL-2 receptor. IL-2 mutants of the invention may exhibit greater than about 80% and even greater than about 90% of such affinity.

The inventors have found that a reduction of the affinity of IL-2 for the α-subunit of the IL-2 receptor in combination with elimination of the O-glycosylation of IL-2 results in an IL-2 protein with improved properties. For example, elimination of the O-glycosylation site results in a more homogenous product when the mutant IL-2 polypeptide is expressed in mammalian cells such as CHO or HEK cells.

Thus, in certain embodiments the mutant IL-2 polypeptide according to the invention comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.

In certain embodiments the mutant IL-2 polypeptide is essentially a full-length IL-2 molecule. In certain embodiments the mutant IL-2 polypeptide is a human IL-2 molecule. In one embodiment the mutant IL-2 polypeptide comprises the sequence of SEQ ID NO: 144 with at least one amino acid mutation that abolishes or reduces affinity of the mutant IL-2 polypeptide to the α-subunit of the IL-2 receptor but preserve affinity of the mutant IL-2 polypeptide to the intermediate affinity IL-2 receptor, compared to an IL-2 polypeptide comprising SEQ ID NO: 144 without said mutation.

In a specific embodiment, the mutant IL-2 polypeptide can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment the mutant IL-2 polypeptide has a reduced ability to induce IL-2 signaling in regulatory T cells, compared to a wild-type IL-2 polypeptide. In one embodiment the mutant IL-2 polypeptide induces less activation-induced cell death (AICD) in T cells, compared to a wild-type IL-2 polypeptide. In one embodiment the mutant IL-2 polypeptide has a reduced toxicity profile in vivo, compared to a wild-type IL-2 polypeptide. In one embodiment the mutant IL-2 polypeptide has a prolonged serum half-life, compared to a wild-type IL-2 polypeptide.

A particular mutant IL-2 polypeptide according to the invention comprises four amino acid substitutions at positions corresponding to residues 3, 42, 45 and 72 of human IL-2. Specific amino acid substitutions are T3A, F42A, Y45A and L72G. As demonstrated in the appended Examples, said quadruple mutant IL-2 polypeptide exhibits no detectable binding to CD25, reduced ability to induce apoptosis in T cells, reduced ability to induce IL-2 signaling in T_{reg} cells, and a reduced toxicity profile in vivo. However, it retains ability to activate IL-2 signaling in effector cells, to induce proliferation of effector cells, and to generate IFN-γ as a secondary cytokine by NK cells.

Moreover, said mutant IL-2 polypeptide has further advantageous properties, such as reduced surface hydrophobicity, good stability, and good expression yield, as described in the Examples. Unexpectedly, said mutant IL-2 polypeptide also provides a prolonged serum half-life, compared to wild-type IL-2.

IL-2 mutants of the invention, in addition to having mutations in the region of IL-2 that forms the interface of IL-2 with CD25 or the glycosylation site, also may have one or more mutations in the amino acid sequence outside these regions. Such additional mutations in human IL-2 may provide additional advantages such as increased expression or stability. For example, the cysteine at position 125 may be replaced with a neutral amino acid such as serine, alanine, threonine or valine, yielding C125S IL-2, C125A IL-2, C125T IL-2 or C125V IL-2 respectively, as described in U.S. Pat. No. 4,518,584. As described therein, one may also delete the N-terminal alanine residue of IL-2 yielding such mutants as des-A1 C125S or des-A1 C125A. Alternatively or conjunctively, the IL-2 mutant may include a mutation whereby methionine normally occurring at position 104 of wild-type human IL-2 is replaced by a neutral amino acid such as alanine (see U.S. Pat. No. 5,206,344). The resulting mutants, e.g., des-A1 M104A IL-2, des-A1 M104A C125S IL-2, M104A IL-2, M104A C125A IL-2, des-A1 M104A C125A IL-2, or M104A C125S IL-2 (these and other mutants may be found in U.S. Pat. No. 5,116,943 and in Weiger et al., Eur J Biochem 180, 295-300 (1989)) may be used in conjunction with the particular IL-2 mutations of the invention.

Thus, in certain embodiments the mutant IL-2 polypeptide according to the invention comprises an additional amino acid mutation at a position corresponding to residue 125 of human IL-2. In one embodiment said additional amino acid mutation is the amino acid substitution C125A.

The skilled person will be able to determine which additional mutations may provide additional advantages for the purpose of the invention. For example, he will appreciate that amino acid mutations in the IL-2 sequence that reduce or abolish the affinity of IL-2 to the intermediate-affinity IL-2 receptor, such as D20T, N88R or Q126D (see e.g. US 2007/0036752), may not be suitable to include in the mutant IL-2 polypeptide according to the invention.

Mutant IL-2 polypeptides of the invention are particularly useful in the context of IL-2 fusion proteins such as IL-2 bearing immunoconjugates. Such fusion proteins comprise a mutant IL-2 polypeptide of the invention fused to a non-IL-2 moiety. The non-IL-2 moiety can be a synthetic or natural protein or a portion or variant thereof. Exemplary non-IL-2 moieties include albumin, or antibody domains such as Fc domains or antigen binding domains of immunoglobulins.

IL-2 bearing immunoconjugates are fusion proteins comprising an antigen binding moiety and an IL-2 moiety. They significantly increase the efficacy of IL-2 therapy by directly targeting IL-2 e.g. into a tumor microenvironment. According to the invention, an antigen binding moiety can be a whole antibody or immunoglobulin, or a portion or variant thereof that has a biological function such as antigen specific binding affinity.

The benefits of immunoconjugate therapy are readily apparent. For example, an antigen binding moiety of an immunoconjugate recognizes a tumor-specific epitope and results in targeting of the immunoconjugate molecule to the tumor site. Therefore, high concentrations of IL-2 can be delivered into the tumor microenvironment, thereby resulting in activation and proliferation of a variety of immune effector cells mentioned herein using a much lower dose of the immunoconjugate than would be required for unconjugated IL-2. Moreover, since application of IL-2 in form of immunoconjugates allows lower doses of the cytokine itself, the potential for undesirable side effects of IL-2 is restricted, and targeting the IL-2 to a specific site in the body by means of an immunoconjugate may also result in a reduction of systemic exposure and thus less side effects than obtained with unconjugated IL-2. In addition, the increased circulating half-life of an immunoconjugate compared to unconjugated IL-2 contributes to the efficacy of the immunoconjugate. However, this characteristic of IL-2 immunoconjugates may again aggravate potential side effects of the IL-2 molecule: Because of the significantly longer circulating half-life of IL-2 immunoconjugate in the bloodstream relative to unconjugated IL-2, the probability for IL-2 or other portions of the fusion protein molecule to activate components generally present in the vasculature is increased. The same concern applies to other fusion proteins that contain IL-2 fused to another moiety such as Fc or albumin, resulting in an extended half-life of IL-2 in the circulation. Therefore an immunoconjugate comprising a mutant IL-2 polypeptide according to the invention, with reduced toxicity compared to wild-type forms of IL-2, is particularly advantageous.

Accordingly, the invention further provides a mutant IL-2 polypeptide as described hereinbefore, linked to at least one non-IL-2 moiety. In one embodiment the mutant IL-2 polypeptide and the non-IL-2 moiety form a fusion protein, i.e. the mutant IL-2 polypeptide shares a peptide bond with the non-IL-2 moiety. In one embodiment the mutant IL-2 polypeptide is linked to a first and a second non-IL-2 moiety. In one embodiment the mutant IL-2 polypeptide shares an amino- or carboxy-terminal peptide bond with the first antigen binding moiety, and the second antigen binding moiety shares an amino- or carboxy-terminal peptide bond with either i) the mutant IL-2 polypeptide or ii) the first antigen binding moiety. In a specific embodiment the mutant IL-2 polypeptide shares a carboxy-terminal peptide bond with said first non-IL-2 moiety and an amino-terminal peptide bond with said second non-IL-2 moiety. In one embodiment said non-IL-2 moiety is a targeting moiety. In a particular embodiment said non-IL-2 moiety is an antigen binding moiety (thus forming an immunoconjugate with the mutant IL-2 polypeptide, as described in more detail hereinbelow). In certain embodiments the antigen binding moiety is an antibody or an antibody fragment. In one embodiment the antigen binding moiety is a full-length antibody. In one embodiment the antigen binding moiety is an immunoglobulin molecule, particularly an IgG class immunoglobulin molecule, more particularly an IgG₁ subclass immunoglobulin molecule. In one such embodiment, the mutant IL-2 polypeptide shares an amino-terminal peptide bond with one of the immunoglobulin heavy chains. In another embodiment the antigen binding moiety is an antibody fragment. In some embodiments said antigen binding moiety comprises an antigen binding domain of an antibody comprising an antibody heavy chain variable region and an antibody light chain variable region. In a more specific embodiment the antigen binding moiety is a Fab molecule or a scFv molecule. In a particular embodiment the antigen binding moiety is a Fab molecule. In another embodiment the antigen binding moiety is a scFv molecule. In one embodiment said antigen binding moiety is directed to an antigen presented on a tumor cell or in a tumor cell environment. In a preferred embodiment said antigen is selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). Where the mutant IL-2 polypeptide is linked to more than one antigen binding moiety, e.g. a first and a second antigen binding moiety, each antigen binding moiety can be independently selected from various forms of antibodies and antibody fragments. For example, the first antigen binding moiety can be a Fab molecule and the second antigen binding moiety can be a scFv molecule. In a specific embodiment each of said first and said second antigen binding moieties is a scFv molecule or each of said first and said second antigen binding moieties is a Fab molecule. In a particular embodiment each of said first and said second antigen binding moieties is a Fab molecule. Likewise, where the mutant IL-2 polypeptide is linked to more than one antigen binding moiety, e.g. a first and a second antigen binding moiety, the antigen to which each of the antigen binding moieties is directed can be independently selected. In one embodiment said first and said second antigen binding moieties are directed to different antigens. In another embodiment said first and said second antigen binding moieties are directed to the same antigen. As described above, the antigen is particularly an antigen presented on a tumor cell or in a tumor cell environment, more particularly an antigen selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). The antigen binding region may further incorporate any of the features, singly or in combination, described herein in relation to antigen binding domains of immunoconjugates.

Immunoconjugates

In a particular aspect the invention provides an immunoconjugate comprising a mutant IL-2 polypeptide, as disclosed herein, comprising one or more amino acid mutation, wherein the one or more amino acid substitutions abolishes or reduces binding to the IL-2 receptor, preferably to the intermediate-affinity IL-2 receptor (IL2Rβγ), at neutral pH and facilitate binding to the IL-2 receptor, preferably to the intermediate-affinity IL-2 receptor (IL2Rβγ), at decreased pH. In one embodiment, the immunoconjugate comprises a mutant IL-2 polypetide that exhibits reduced or abolished IL-2 receptor binding, preferably intermediate-affinity IL-2 receptor binding, at pH 7.4 and/or pH 7.0 and retained IL-2 receptor binding, preferably intermediate-affinity IL-2 receptor binding, at a pH 6 and/or pH 6.5, as disclosed herein.

In a particular aspect the invention provides an immunoconjugate comprising a mutant IL-2 polypeptide comprising one or more amino acid mutation that abolishes or reduces affinity of the mutant IL-2 polypeptide to the α-subunit of the IL-2 receptor and preserves affinity of the mutant IL-2 polypeptide to the intermediate-affinity IL-2 receptor, and at least one antigen binding moiety. In one embodiment according to the invention, the amino acid mutation that abolishes or reduces affinity of the mutant IL-2 polypeptide to the α-subunit of the IL-2 receptor and preserves affinity of the mutant IL-2 polypeptide to the intermediate affinity IL-2 receptor is at a position selected from a position corresponding to residue 42, 45 and 72 of human IL-2. In one embodiment said amino acid mutation is an amino acid substitution. In one embodiment said amino acid mutation is an amino acid substitution selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, more specifically an amino acid substitution selected from the group of F42A, Y45A and L72G. In one embodiment the amino acid mutation is at a position corresponding to residue 42 of human IL-2. In a specific embodiment said amino acid mutation is an amino acid substitution selected from the group of F42A, F42G, F42S, F42T, F42Q, F42E, F42N, F42D, F42R, and F42. In an even more specific embodiment said amino acid substitution is F42A. In another embodiment the amino acid mutation is at a position corresponding to residue 45 of human IL-2. In a specific embodiment said amino acid mutation is an amino acid substitution selected from the group of Y45A, Y45G, Y45S, Y45T, Y45Q, Y45E, Y45N, Y45D, Y45R, and Y45K. In an even more specific embodiment said amino acid substitution is Y45A. In yet another embodiment the amino acid mutation is at a position corresponding to residue 72 of human IL-2. In a specific embodiment said amino acid mutation is an amino acid substitution selected from the group of L72G, L72A, L72S, L72T, L72Q, L72E, L72N, L72D, L72R, and L72K. In an even more specific embodiment said amino acid substitution is L72G. In certain embodiments, the mutant IL-2 polypeptide according to the invention does not comprise an amino acid mutation at a position corresponding to residue 38 of human IL-2. In a particular embodiment, the mutant IL-2 polypeptide comprised in the immunoconjugate of the invention comprises at least a first and a second amino acid mutation that abolishes or reduces affinity of the mutant IL-2 polypeptide to the α-subunit of the IL-2 receptor and preserves affinity of the mutant IL-2 polypeptide to the intermediate affinity IL-2 receptor. In one embodiment said first and second amino acid mutations are at two positions selected from the positions corresponding to residue 42, 45 and 72 of human IL-2. In one embodiment said first and second amino acid mutations are amino acid substitutions. In one embodiment said first and second amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a particular embodiment said first and second amino acid mutations are amino acid substitutions selected from the group of F42A, Y45A and L72G. The mutant IL-2 polypeptide may further incorporate any of the features, singly or in combination, described in the preceding paragraphs in relation to the mutant IL-2 polypeptides of the invention. In one embodiment said mutant IL-2 polypeptide shares an amino- or carboxy-terminal peptide bond with said antigen binding moiety comprised in the immunoconjugate, i.e. the immunoconjugate is a fusion protein. In certain embodiments said antigen binding moiety is an antibody or an antibody fragment. In some embodiments said antigen binding moiety comprises an antigen binding domain of an antibody comprising an antibody heavy chain variable region and an antibody light chain variable region. The antigen binding region may incorporate any of the features, singly or in combination, described hereinabove or below in relation to antigen binding domains.

Immunoconjugate Formats

Particularly suitable immunoconjugate formats are described in PCT publication no. WO 2011/020783, which is incorporated herein by reference in its entirety. These immunoconjugates comprise at least two antigen binding domains. Thus, in one embodiment, the immunoconjugate according to the present invention comprises at least a first mutant IL-2 polypeptide as described herein, and at least a first and a second antigen binding moiety. In a particular embodiment, said first and second antigen binding moiety are independently selected from the group consisting of an Fv molecule, particularly a scFv molecule, and a Fab molecule. In a specific embodiment, said first mutant IL-2 polypeptide shares an amino- or carboxy-terminal peptide bond with said first antigen binding moiety and said second antigen binding moiety shares an amino- or carboxy-terminal peptide bond with either i) the first mutant IL-2 polypeptide or ii) the first antigen binding moiety. In a particular embodiment, the immunoconjugate consists essentially of a first mutant IL-2 polypeptide and first and second antigen binding moieties, joined by one or more linker sequences. Such formats have the advantage that they bind with high affinity to the target antigen (such as a tumor antigen), but only monomeric binding to the IL-2 receptor, thus avoiding targeting the immunoconjugate to IL-2 receptor bearing immune cells at other locations than the target site. In a particular embodiment, a first mutant IL-2 polypeptide shares a carboxy-terminal peptide bond with a first antigen binding moiety and further shares an amino-terminal peptide bond with a second antigen binding moiety. In another embodiment, a first antigen binding moiety shares a carboxy-terminal peptide bond with a first mutant IL-2 polypeptide, and further shares an amino-terminal peptide bond with a second antigen binding moiety. In another embodiment, a first antigen binding moiety shares an amino-terminal peptide bond with a first mutant IL-2 polypeptide, and further shares a carboxy-terminal peptide with a second antigen binding moiety. In a particular embodiment, a mutant IL-2 polypeptide shares a carboxy-terminal peptide bond with a first heavy chain variable region and further shares an amino-terminal peptide bond with a second heavy chain variable region. In another embodiment a mutant IL-2 polypeptide shares a carboxy-terminal peptide bond with a first light chain variable region and further shares an amino-terminal peptide bond with a second light chain variable region. In another embodiment, a first heavy or light chain variable region is joined by a carboxy-terminal peptide bond to a first mutant IL-2 polypeptide and is further joined by an amino-terminal peptide bond to a second heavy or light chain variable region. In another embodiment, a first heavy or light chain variable region is joined by an amino-terminal peptide bond to a first mutant IL-2 polypeptide and is further joined by a carboxy-terminal peptide bond to a second heavy or light chain variable region. In one embodiment, a mutant IL-2 polypeptide shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a first mutant IL-2 polypeptide and further shares an amino-terminal peptide bond with a second Fab heavy or light chain. In other embodiments, a first Fab heavy or light chain shares an amino-terminal peptide bond with a first mutant IL-2 polypeptide and further shares a carboxy-terminal peptide bond with a second Fab heavy or light chain. In one embodiment, the immunoconjugate comprises at least a first mutant IL-2 polypeptide sharing an amino-terminal peptide bond with one or more scFv molecules and further sharing a carboxy-terminal peptide bond with one or more scFv molecules.

Other particularly suitable immunoconjugate formats comprise an immunoglobulin molecule as antigen binding moiety. In one such embodiment, the immunoconjugate comprises at least one mutant IL-2 polypeptide as described herein and an immunoglobulin molecule, particularly an IgG molecule, more particularly an IgG₁ molecule. In one embodiment the immunoconjugate comprises not more than one mutant IL-2 polypeptide. In one embodiment the immunoglobulin molecule is human. In one embodiment the mutant IL-2 polypeptide shares an amino- or carboxy-terminal peptide bond with the immunoglobulin molecule. In one embodiment, the immunoconjugate essentially consists of a mutant IL-2 polypeptide and an immunoglobulin molecule, particularly an IgG molecule, more particularly an IgG₁ molecule, joined by one or more linker sequences. In a specific embodiment the mutant IL-2 polypeptide is joined at its amino-terminal amino acid to the carboxy-terminal amino acid of one of the immunoglobulin heavy chains. In certain embodiments, the immunoglobulin molecule comprises in the Fc domain a modification promoting heterodimerization of two non-identical immunoglobulin heavy chains. The site of most extensive protein-protein interaction between the two polypeptide chains of a human IgG Fc domain is in the CH3 domain of the Fc domain. Thus, in one embodiment said modification is in the CH3 domain of the Fc domain. In a specific embodiment said modification is a knob-into-hole modification, comprising a knob modification in one of the immunoglobulin heavy chains and a hole modification in the other one of the immunoglobulin heavy chains. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two immunoglobulin heavy chains, and the hole modification comprises the amino acid substitutions T366S, L368A and Y407V in the other one of the two immunoglobulin heavy chains. In a further specific embodiment, immunoglobulin heavy chain comprising the knob modification additionally comprises the amino acid substitution S354C, and the immunoglobulin heavy chain comprising the hole modification additionally comprises the amino acid substitution Y349C. Introduction of these two cysteine residues results in formation of a disulfide bridge between the two heavy chains, further stabilizing the dimer (Carter, J Immunol Methods 248, 7-(2001)).

In a particular embodiment the mutant IL-2 polypeptide is joined to the carboxy-terminal amino acid of the immunoglobulin heavy chain comprising the knob modification.

In an alternative embodiment a modification promoting heterodimerization of two non-identical polypeptide chains comprises a modification mediating electrostatic steering effects, e.g. as described in PCT publication WO 2009/089004. Generally, this method involves replacement of one or more amino acid residues at the interface of the two polypeptide chains by charged amino acid residues so that homodimer formation becomes electrostatically unfavorable but heterodimerization electrostatically favorable.

An Fc domain confers to the immunoconjugate favorable pharmacokinetic properties, including a long serum half-life which contributes to good accumulation in the target tissue and a favorable tissue-blood distribution ratio. At the same time it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-2 polypeptide and the long half-life of the immunoconjugate, results in excessive activation of cytokine receptors and severe side effects upon systemic administration. In line with this, conventional IgG-IL-2 immunoconjugates have been described to be associated with infusion reactions (see e.g. King et al., J Clin Oncol 22, 4463-4473 (2004)).

Accordingly, in certain embodiments the immunoglobulin molecule comprised in the immunoconjugate according to the invention is engineered to have reduced binding affinity to an Fc receptor. In one such embodiment the immunoglobulin comprises in its Fc domain one or more amino acid mutation that reduces the binding affinity of the immunoconjugate to an Fc receptor. Typically, the same one or more amino acid mutation is present in each of the two immunoglobulin heavy chains. In one embodiment said amino acid mutation reduces the binding affinity of the immunoconjugate to the Fc receptor by at least 2-fold, at least 5-fold, or at least 10-fold. In embodiments where there is more than one amino acid mutation that reduces the binding affinity of the immunoconjugate to the Fc receptor, the combination of these amino acid mutations may reduce the binding affinity of the Fc domain to the Fc receptor by at least 10-fold, at least 20-fold, or even at least 50-fold. In one embodiment the immunoconjugate comprising an engineered immunoglobulin molecule exhibits less than 20%, particularly less than 10%, more particularly less than 5% of the binding affinity to an Fc receptor as compared to an immunoconjugate comprising a non-engineered immunoglobulin molecule. In one embodiment the Fc receptor is an activating Fc receptor. In a specific embodiment the Fc receptor is an Fcγ receptor, more specifically an FcγRIIIa, FcγRI or FcγRIIa receptor. Preferably, binding to each of these receptors is reduced. In some embodiments binding affinity to a complement component, specifically binding affinity to C1 q, is also reduced. In one embodiment binding affinity to neonatal Fc receptor (FcRn) is not reduced. Substantially similar binding to FcRn, i.e. preservation of the binding affinity of the immunoglobulin to said receptor, is achieved when the immunoglobulin (or the immunoconjugate comprising said immunoglobulin) exhibits greater than about 70% of the binding affinity of a non-engineered form of the immunoglobulin (or the immunoconjugate comprising said non-engineered form of the immunoglobulin) to FcRn.

Immunoglobulins, or immunoconjugates comprising said immunoglobulins, may exhibit greater than about 80% and even greater than about 90% of such affinity. In one embodiment the amino acid mutation is an amino acid substitution. In one embodiment the immunoglobulin comprises an amino acid substitution at position P329 of the immunoglobulin heavy chain (Kabat numbering). In a more specific embodiment the amino acid substitution is P329A or P329G, particularly P329G. In one embodiment the immunoglobulin comprises a further amino acid substitution at a position selected from 5228, E233, L234, L235, N297 and P331 of the immunoglobulin heavy chain. In a more specific embodiment the further amino acid substitution is S228P, E233P, L234A, L235A, L235E, N297A, N297D or P331S. In a particular embodiment the immunoglobulin comprises amino acid substitutions at positions P329, L234 and L235 of the immunoglobulin heavy chain. In a more particular embodiment the immunoglobulin comprises the amino acid mutations L234A, L235A and P329G (LALA P329G). This combination of amino acid substitutions almost completely abolishes Fcγ receptor binding of a human IgG molecule, and hence decreases effector function including antibody-dependent cell-mediated cytotoxicity (ADCC).

In certain embodiments, the immunoconjugate comprises one or more proteolytic cleavage sites located between mutant IL-2 polypeptide and antigen binding moieties.

Components of the immunoconjugate (e.g. antigen binding moieties and/or mutant IL-2 polypeptide) may be linked directly or through various linkers, particularly peptide linkers comprising one or more amino acids, typically about 2-20 amino acids, that are described herein or are known in the art. Suitable, non-immunogenic linker peptides include, for example, (G4S)ₙ, (SG4)ₙ or G4(SG4)ₙ linker peptides, wherein n is generally a number between 1 and 10, typically between 2 and 4.

Antigen Binding Moieties

The antigen binding moiety of the immunoconjugate of the invention is generally a polypeptide molecule that binds to a specific antigenic determinant and is able to direct the entity to which it is attached (e.g. a mutant IL-2 polypeptide or a second antigen binding moiety) to a target site, for example to a specific type of tumor cell or tumor stroma that bears the antigenic determinant. The immunoconjugate can bind to antigenic determinants found, for example, on the surfaces of tumor cells, on the surfaces of virus-infected cells, on the surfaces of other diseased cells, free in blood serum, and/or in the extracellular matrix (ECM).

Non-limiting examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, ε-cadherin, α-catenin, β-catenin and γ-catenin, p120ctn, gp100 Pme1117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Non-limiting examples of viral antigens include influenza virus hemagglutinin, Epstein-Barr virus LMP-1, hepatitis C virus E2 glycoprotein, HIV gp160, and HIV gp120.

Non-limiting examples of ECM antigens include syndecan, heparanase, integrins, osteopontin, link, cadherins, laminin, laminin type EGF, lectin, fibronectin, notch, tenascin, and matrixin.

The immunoconjugates of the invention can bind to the following specific non-limiting examples of cell surface antigens: FAP, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R (IL6 receptor), CD20, MCSP, and PDGFβR (β platelet-derived growth factor receptor).

In one embodiment, the immunoconjugate of the invention comprises two or more antigen binding moieties, wherein each of these antigen binding moieties specifically binds to the same antigenic determinant. In another embodiment, the immunoconjugate of the invention comprises two or more antigen binding moieties, wherein each of these antigen binding moieties specifically binds to different antigenic determinants.

The antigen binding moiety can be any type of antibody or fragment thereof that retains specific binding to an antigenic determinant. Antibody fragments include, but are not limited to, V_{H} fragments, V_{L} fragments, Fab fragments, F(ab′)₂ fragments, scFv fragments, Fv fragments, minibodies, diabodies, triabodies, and tetrabodies (see e.g. Hudson and Souriau, Nature Med 9, 129-134 (2003)).

Particularly suitable antigen binding moieties are described in PCT publication no. WO 2011/020783, which is incorporated herein by reference in its entirety.

In one embodiment, the immunoconjugate comprises at least one, typically two or more antigen binding moieties that are specific for the Extra Domain B of fibronectin (EDB). In another embodiment, the immunoconjugate comprises at least one, typically two or more antigen binding moieties that can compete with monoclonal antibody L19 for binding to an epitope of EDB. See, e.g., PCT publication WO 2007/128563 A1 (incorporated herein by reference in its entirety). In yet another embodiment, the immunoconjugate comprises a polypeptide sequence wherein a first Fab heavy chain derived from the L19 monoclonal antibody shares a carboxy-terminal peptide bond with a mutant IL-2 polypeptide which in turn shares a carboxy-terminal peptide bond with a second Fab heavy chain derived from the L19 monoclonal antibody. In yet another embodiment, the immunoconjugate comprises a polypeptide sequence wherein a first Fab light chain derived from the L19 monoclonal antibody shares a carboxy-terminal peptide bond with a mutant IL-2 polypeptide which in turn shares a carboxy-terminal peptide bond with a second Fab light chain derived from the L19 monoclonal antibody. In a further embodiment, the immunoconjugate comprises a polypeptide sequence wherein a first scFv derived from the L19 monoclonal antibody shares a carboxy-terminal peptide bond with a mutant IL-2 polypeptide which in turn shares a carboxy-terminal peptide bond with a second scFv derived from the L19 monoclonal antibody.

In a specific embodiment, the immunoconjugate comprises a polypeptide sequence that is at least about 80%, 85%, 90%, 95%, 96%, 97%, 98%, 99% or 100% identical to a polypeptide sequence selected from the group of SEQ ID NOs 64-79 and SEQ ID NOs 82-143.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 64, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 65, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 66, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 67, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 68, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 69, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 70, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 71, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 72, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 73, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 74, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 75, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 76, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 77, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 78, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 63 and SEQ ID NO: 79, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 81, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 82, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 83, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 84, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 85, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 86, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 87, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 88, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 89, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 90, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 91, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 92, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 93, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 94, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 95, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 96, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 97, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 98, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 99, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 100, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 101, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 102, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 103, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 104, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 105, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 106, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 107, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 108, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 109, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 110, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 111, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 112, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 113, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 114, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 115, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 116, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 117, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 118, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 119, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 120, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 121, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 122, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 123, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 124, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 125, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 126, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 127, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 128, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 129, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 130, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 131, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 132, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 133, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 134, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 135, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 136, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 137, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 138, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 139, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 140, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 141, or

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 142, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 62, SEQ ID NO: 80 and SEQ ID NO: 143, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 147, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 148, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 149, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 150, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 151, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 152, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 153, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 154, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 155, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 156, or variants thereof that retain functionality.

In a specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 145, SEQ ID NO: 146 and SEQ ID NO: 157, or variants thereof that retain functionality.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 64.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 65.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 66.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 67.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 68.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 69.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 70.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 71.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 72.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 73.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 74.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 75.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 76.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 77.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 78.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 63, and a polypetide comprising an amino acid sequence that is at least about 80%, 85%, 90%, 95%, 96%, 97%, 98%, 99% or 100% identical to the sequence of SEQ ID NO: 79.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 81.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 82.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 83.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 84.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 85.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 86.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 87.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 88.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 89.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 90.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% sequence that is at least about 80%, 85%, 90%, 95%, 96%, 97%, 98%, 99% or 100% identical to the sequence of SEQ ID NO: 91.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 92.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 93.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 94.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 95.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 96.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 97.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 98.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 99.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 100.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 101.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 102.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 103.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% sequence that is at least about 80%, 85%, 90%, 95%, 96%, 97%, 98%, 99% or 100% identical to the sequence of SEQ ID NO: 104.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 105.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 106.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 107.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 108.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 109.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 110.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 111.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 112.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 113.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 114.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 115.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 116.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% sequence that is at least about 80%, 85%, 90%, 95%, 96%, 97%, 98%, 99% or 100% identical to the sequence of SEQ ID NO: 117.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 118.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 119.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 120.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 121.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 122.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 123.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 124.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 125.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 126.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 127.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 128.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 129.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% sequence that is at least about 80%, 85%, 90%, 95%, 96%, 97%, 98%, 99% or 100% identical to the sequence of SEQ ID NO: 130.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 131.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 132.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 133.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 134.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 135.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 136.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 137.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 138.

In a specific embodiment, the invention provides an immunoconjugate comprising a polypeptide 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 139.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 140.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 141.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% identical to the sequence of SEQ ID NO: 80, and a polypetide comprising an amino acid sequence that is at least about 80%, 85%, 90%, 95%, 96%, 97%, 98%, 99% or 100% identical to the sequence of SEQ ID NO: 142.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 62, a polypetide comprising an amino acid sequence that is at least about 80%, 85%, 90%, 95%, 96%, 97%, 98%, 99% or 100% sequence that is at least about 80%, 85%, 90%, 95%, 96%, 97%, 98%, 99% or 100% identical to the sequence of SEQ ID NO: 143.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 147.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 148.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 149.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 150.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 151.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 152.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 153.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 154.

In a specific embodiment, the invention provides an immunoconjugate comprising a polypeptide 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 155.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 156.

In a specific embodiment, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO: 145, a polypetide comprising an amino acid sequence that is at least about 80%, 85%, 90%, 95%, 96%, 97%, 98%, 99% or 100% identical to the sequence of SEQ ID NO: 146, and a polypetide comprising an amino acid sequence that is at least about 80%, 85%, 90%, 95%, 96%, 97%, 98%, 99% or 100% identical to the sequence of SEQ ID NO: 157.

Polynucleotides

The invention further provides isolated polynucleotides encoding a mutant IL-2 polypeptide or an immunoconjugate comprising a mutant IL-2 polypeptide as described herein.

Polynucleotides of the invention include those that encode for polypeptides which are at least about 80%, 85%, 90%, 95%, 96%, 97%, 98%, 99%, or 100% identical to the sequences set forth in SEQ ID NOs 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 145, 146, 147, 148, 149, 150, 151, 152, 153, 154, 155, 156, 157 including functional fragments or variants thereof.

The polynucleotides encoding mutant IL-2 polypeptides not linked to a non-IL-2 moiety are generally expressed as single polynucleotide that encodes the entire polypeptide.

The polynucleotides encoding immunoconjugates of the invention may be expressed as a single polynucleotide that encodes the entire immunoconjugate or as multiple (e.g., two or more) polynucleotides that are co-expressed. Polypeptides encoded by polynucleotides that are co-expressed may associate through, e.g., disulfide bonds or other means to form a functional immunoconjugate. For example, the heavy chain portion of an antigen binding moiety may be encoded by a separate polynucleotide from the portion of the immunoconjugate comprising the light chain portion of the antigen binding moiety and the mutant IL-2 polypeptide. When co-expressed, the heavy chain polypeptides will associate with the light chain polypeptides to form the antigen binding moiety. Alternatively, in another example, the light chain portion of the antigen binding moiety could be encoded by a separate polynucleotide from the portion of the immunoconjugate comprising the heavy chain portion of the antigen binding moiety and the mutant IL-2 polypeptide. In one embodiment, an isolated polynucleotide of the invention encodes a fragment of an immunoconjugate comprising a mutant IL-2 polypeptide and an antigen binding moiety. In one embodiment, an isolated polynucleotide of the invention encodes the heavy chain of an antigen binding moiety and a mutant IL-2 polypeptide. In another embodiment, an isolated polynucleotide of the invention encodes the light chain of an antigen binding moiety and a mutant IL-2 polypeptide.

In a specific embodiment, an isolated polynucleotide of the invention encodes a fragment of an immunoconjugate comprising at least one mutant IL-2 polypeptide, and at least one, preferably two or more antigen binding moieties, wherein a first mutant IL-2 polypeptide shares an amino- or carboxy-terminal peptide bond with a first antigen binding moiety and a second antigen binding moiety shares an amino- or carboxy-terminal peptide bond with either the first mutant IL-2 polypeptide or the first antigen binding moiety. In a one embodiment, the antigen binding moieties are independently selected from the group consisting of a Fv molecule, particularly a scFv molecule, and a Fab molecule. In another specific embodiment, the polynucleotide encodes the heavy chains of two of the antigen binding moieties and one mutant IL-2 polypeptide. In another specific embodiment, the polynucleotide encodes the light chains of two of the antigen binding moieties and one mutant IL-2 polypeptide. In another specific embodiment, the polynucleotide encodes one light chain of one of the antigen binding moieties, one heavy chain of a second antigen binding moiety and one mutant IL-2 polypeptide.

In another specific embodiment, an isolated polynucleotide of the invention encodes a fragment of an immunoconjugate, wherein the polynucleotide encodes the heavy chains of two Fab molecules and a mutant IL-2 polypeptide. In another specific embodiment, an isolated polynucleotide of the invention encodes a fragment of an immunoconjugate, wherein the polynucleotide encodes the light chains of two Fab molecules and a mutant IL-2 polypeptide. In another specific embodiment an isolated polynucleotide of the invention encodes a fragment of an immunoconjugate, wherein the polynucleotide encodes the heavy chain of one Fab molecule, the light chain of second Fab molecule and a mutant IL-2 polypeptide.

In one embodiment, an isolated polynucleotide of the invention encodes an immunoconjugate comprising at least one mutant IL-2 polypeptide, joined at its amino- and carboxy-terminal amino acids to one or more scFv molecules.

In one embodiment, an isolated polynucleotide of the invention encodes a fragment of an immunoconjugate, wherein the polynucleotide encodes the heavy chain of an immunoglobulin molecule, particularly an IgG molecule, more particularly an IgG₁ molecule, and a mutant IL-2 polypeptide. In a more specific embodiment, the isolated polynucleotide encodes a the heavy chain of an immunoglobulin molecule and a mutant IL-2 polypeptide, wherein the mutant IL-2 polypeptide shares a amino-terminal peptide bond with the immunoglobulin heavy chain.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Mutant IL-2 polypeptides of the invention can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. In this regard, the nucleotide sequence of native IL-2 has been described by Taniguchi et al. (Nature 302, 305-10(1983)) and nucleic acid encoding human IL-2 is available from public depositories such as the American Type Culture Collection (Rockville MD). The sequence of native human IL-2 is shown in SEQ ID NO: 144. Substitution or insertion may involve natural as well as non-natural amino acid residues. Amino acid modification includes well known methods of chemical modification such as the addition of glycosylation sites or carbohydrate attachments, and the like.

Mutant IL-2 polypeptides and immunoconjugates of the invention may be obtained, for example, by solid-state peptide synthesis or recombinant production. For recombinant production one or more polynucleotide encoding said mutant IL-2 polypeptide or immunoconjugate (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a mutant IL-2 polypeptide or immunoconjugate (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the IL-2 mutant or the immunoconjugate (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polyproteins, which are post- or co-translationally separated into the final proteins via proteolytic cleavage. In addition, a vector, polynucleotide, or nucleic acid of the invention may encode heterologous coding regions, either fused or unfused to a first or second polynucleotide encoding the polypeptides of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclines). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the mutant IL-2 polypeptide is desired, DNA encoding a signal sequence may be placed upstream of the nucleic acid encoding the mature amino acids of the mutant IL-2. The same applies to immunoconjugates of the invention or fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. For example, human IL-2 is translated with a 20 amino acid signal sequence at the N-terminus of the polypeptide, which is subsequently cleaved off to produce the mature, 133 amino acid human IL-2. In certain embodiments, the native signal peptide, e.g. the IL-2 signal peptide or an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the IL-2 mutant or immunoconjugate may be included within or at the ends of the IL-2 mutant or immunoconjugate (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes an amino acid sequence comprising the mutant IL-2 polypeptide of the invention. As used herein, the term “host cell” refers to any kind of cellular system which can be engineered to generate the mutant IL-2 polypeptides or immunoconjugates of the invention or fragments thereof. Host cells suitable for replicating and for supporting expression of mutant IL-2 polypeptides or immunoconjugates are well known in the art. Such cells may be transfected or transduced as appropriate with the particular expression vector and large quantities of vector containing cells can be grown for seeding large scale fermenters to obtain sufficient quantities of the IL-2 mutant or immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or lymphoid cell (e.g., YO, NS0, Sp20 cell).

Standard technologies are known in the art to express foreign genes in these systems. Cells expressing a mutant-IL-2 polypeptide fused to either the heavy or the light chain of an antigen binding domain such as an antibody, may be engineered so as to also express the other of the antibody chains such that the expressed mutant IL-2 fusion product is an antibody that has both a heavy and a light chain.

In one embodiment, a method of producing a mutant IL-2 polypeptide or an immunoconjugate according to the invention is provided, wherein the method comprises culturing a host cell comprising a polynucleotide encoding the mutant IL-2 polypeptide or immunoconjugate, as provided herein, under conditions suitable for expression of the mutant IL-2 polypeptide or immunoconjugate, and optionally recovering the mutant IL-2 polypeptide or immunoconjugate from the host cell (or host cell culture medium).

In certain embodiments according to the invention the mutant IL-2 polypeptide is linked to at least one non-IL-2 moiety. An IL-2 mutant can be prepared where the mutant IL-2 polypeptide segment is linked to one or more molecules such as a polypeptide, protein, carbohydrate, lipid, nucleic acid, polynucleotide or molecules that are combinations of these molecules (e.g. glycoproteins, glycolipids etc.). The mutant IL-2 polypeptide also may be linked to an organic moiety, inorganic moiety or pharmaceutical drug. As used herein, a pharmaceutical drug is an organic containing compound of about 5,000 daltons or less. The mutant IL-2 polypeptide also may be linked to any biological agent including therapeutic compounds such as anti-neoplastic agents, anti-microbial agents, hormones, immunomodulators, anti-inflammatory agents and the like. Also included are radioisotopes such as those useful for imaging as well as for therapy.

The mutant IL-2 polypeptide may also be linked to multiple molecules of the same type or to more than one type of molecule. In certain embodiments, the molecule that is linked to IL-2 can confer the ability to target the IL-2 to specific tissues or cells in an animal, and is referred to herein as a “targeting moiety”. In these embodiments, the targeting moiety may have affinity for a ligand or receptor in the target tissue or cell, thereby directing the IL-2 to the target tissue or cell. In a particular embodiment the targeting moiety directs the IL-2 to a tumor. Targeting moieties include, for example, antigen binding moieties (e.g. antibodies and fragments thereof) specific for cell surface or intracellular proteins, ligands of biological receptors, and the like. Such antigen binding moieties may be specific for tumor associated antigens such as the ones described herein.

A mutant IL-2 polypeptide may be genetically fused to another polypeptide, e.g. a single chain antibody, or (part of) an antibody heavy or light chains, or may be chemically conjugated to another molecule. Fusion of a mutant IL-2 polypeptide to part of an antibody heavy chain is described in the Examples. An IL-2 mutant which is a fusion between a mutant IL-2 polypeptide and another polypeptide can be designed such that the IL-2 sequence is fused directly to the polypeptide or indirectly through a linker sequence. The composition and length of the linker may be determined in accordance with methods well known in the art and may be tested for efficacy. Example of a linker sequence between IL-2 and an antibody heavy chain is found in the sequences SEQ ID NOs 209, 211, 213 of WO 2012/107417 A1. Additional sequences may also be included to incorporate a cleavage site to separate the individual components of the fusion if desired, for example an endopeptidase recognition sequence. In addition, an IL-2 mutant or fusion protein thereof may also be synthesized chemically using methods of polypeptide synthesis as is well known in the art (e.g. Merrifield solid phase synthesis). Mutant IL-2 polypeptides may be chemically conjugated to other molecules, e.g. another polypeptide, using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 and can readily be identified by those skilled in the art. Alternatively, or in addition, mutant IL-2 and/or the molecule to which it is intended to be conjugated may be chemically derivatized such that the two can be conjugated in a separate reaction as is also well known in the art.

In certain embodiments the mutant IL-2 polypeptide is linked to one or more antigen binding moieties (i.e. is part of an immunoconjugate) comprising at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Immunoconjugates, antigen binding moieties and methods for producing the same are also described in detail in PCT publication no. WO 2011/020783, the entire content of which is incorporated herein by reference.

Any animal species of antibody, antibody fragment, antigen binding domain or variable region can be linked to a mutant IL-2 polypeptide. Non-limiting antibodies, antibody fragments, antigen binding domains or variable regions useful in the present invention can be of murine, primate, or human origin. If the mutant IL-2/antibody conjugate or fusion is intended for human use, a chimeric form of the antibody may be used wherein the constant regions of the antibody are from a human. A humanized or fully human form of the antibody can also be prepared in accordance with methods well known in the art (see e.g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or α-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N J, 2001); and McCafferty et al., Nature 348, 552-554; Clackson et al., Nature 352, 624-628 (1991)). Phage typically display antibody fragments, either as single-chain Fv (scFv) fragments or as Fab fragments. A detailed description of the preparation of antigen binding moieties for immunoconjugates by phage display can be found in the Examples appended to PCT publication no. WO 2011/020783.

In certain embodiments, the antigen binding moieties useful in the present invention are engineered to have enhanced binding affinity according to, for example, the methods disclosed in PCT publication no. WO 2011/020783 (see Examples relating to affinity maturation) or U.S. Pat. Appl. Publ. No. 2004/0132066, the entire contents of which are hereby incorporated by reference. The ability of the immunoconjugate of the invention to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g. an antibody that competes with the L19 antibody for binding to the Extra Domain B of fibronectin (EDB). In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). In an exemplary competition assay, immobilized antigen (e.g. EDB) is incubated in a solution comprising a first labeled antibody that binds to the antigen (e.g. L19 antibody) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, NY).

Further chemical modification of the mutant IL-2 mutant or immunoconjugate of the invention may be desirable. For example, problems of immunogenicity and short half-life may be improved by conjugation to substantially straight chain polymers such as polyethylene glycol (PEG) or polypropylene glycol (PPG) (see e.g. WO 87/00056).

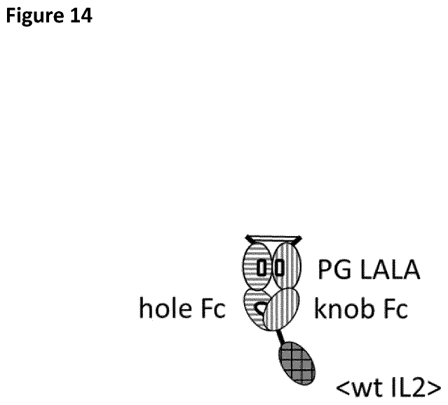

IL-2 mutants and immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the mutant IL-2 polypeptide or immunoconjugate binds. For example, an antibody which specifically binds the mutant IL-2 polypeptide may be used. For affinity chromatography purification of immunoconjugates of the invention, a matrix with protein A or protein G may be used. For example, sequential Protein A or G affinity chromatography and size exclusion chromatography can be used to isolate an immunoconjugate essentially as described in the Examples. The purity of the mutant IL-2 polypeptides and fusion proteins thereof can be determined by any of a variety of well known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see e.g. FIG. 14). Two bands were resolved at approximately Mr 25,000 and Mr 60,000, corresponding to the predicted molecular weights of the immunoglobulin light chain and heavy chain/IL-2 fusion protein.

Assays

Mutant IL-2 polypeptides and immunoconjugates provided herein may be identified, screened for, or characterized for their physical/chemical properties and/or biological activities by various assays known in the art.

Affinity Assays

The affinity of the mutant or wild-type IL-2 polypeptide for various forms of the IL-2 receptor can be determined in accordance with the method set forth in the Examples by surface plasmon resonance (SPR), using standard instrumentation such as a BIAcore instrument (GE Healthcare), and receptor subunits such as may be obtained by recombinant expression (see e.g. Shanafelt et al., Nature Biotechnol 18, 1197-1202 (2000)). A recombinant IL-2 receptor β/γ-subunit heterodimer can be generated by fusing each of the subunits to an antibody Fc domain monomer modified by the knobs-into-holes technology (see e.g. U.S. Pat. No. 5,731,168) to promote heterodimerization of the appropriate receptor subunit/Fc fusion proteins (see SEQ ID NOs 102 and 103). Alternatively, binding affinity of IL-2 mutants for different forms of the IL-2 receptor may be evaluated using cell lines known to express one or the other such form of the receptor. A specific illustrative and exemplary embodiment for measuring binding affinity is described in the following and in the Examples below. According to one embodiment, K_{D} is measured by surface plasmon resonance using a BIACORE® T100 machine (GE Healthcare) at 25° C. with IL-2 receptors immobilized on CMS chips. Briefly, carboxymethylated dextran biosensor chips (CMS, GE Healthcare) are activated with N-ethyl-N′-(3-dimethylaminopropyl)-carbodiimide hydrochloride (EDC) and N-hydroxysuccinimide (NETS) according to the supplier's instructions. Recombinant IL-2 receptor is diluted with 10 mM sodium acetate, pH 5.5, to 0.5-30 μg/ml before injection at a flow rate of 10 μl/minute to achieve approximately 200-1000 (for IL-2R α-subunit) or 500-3000 (for IL-2R βγ knobs-into-holes heterodimer) response units (RU) of coupled protein. Following the injection of IL-2 receptor, 1 M ethanolamine is injected to block unreacted groups. For kinetics measurements, three-fold serial dilutions of mutant IL-2 polypeptide or immunoconjugate (range between −0.3 nM to 300 nM) are injected in HBS-EP+(GE Healthcare, 10 mM HEPES, 150 mM NaCl, 3 mM EDTA, 0.05% Surfactant P20, pH 7.4) at 25° C. at a flow rate of approximately 30 μl/min. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

Binding of immunoconjugates of the invention to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or immunoconjugates comprising an Fc domain for Fc receptors may be evaluated using cell lines known to express particular Fc receptors, such as NK cells expressing FcγIIIa receptor. According to one embodiment, K_{D} is measured by surface plasmon resonance using a BIACORE® T100 machine (GE Healthcare) at 25° C. with Fc receptors immobilized on CMS chips. Briefly, carboxymethylated dextran biosensor chips (CMS, GE Healthcare) are activated with N-ethyl-N′-(3-dimethylaminopropyl)-carbodiimide hydrochloride (EDC) and N-hydroxysuccinimide (NETS) according to the supplier's instructions. Recombinant Fc receptor is diluted with 10 mM sodium acetate, pH 5.5, to 0.5-30 μg/ml before injection at a flow rate of 10 μl/minute to achieve approximately 100-5000 response units (RU) of coupled protein. Following the injection of the Fc receptor, 1 M ethanolamine is injected to block unreacted groups. For kinetics measurements, three- to five-fold serial dilutions of immunoconjugate (range between −0.01 nM to 300 nM) are injected in HBS-EP+(GE Healthcare, 10 mM HEPES, 150 mM NaCl, 3 mM EDTA, 0.05% Surfactant P20, pH 7.4) at 25° C. at a flow rate of approximately 30-50 μl/min. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

Activity Assays

The ability of an IL-2 mutant to bind to IL-2 receptors may be indirectly measured by assaying the effects of immune activation that occur downstream of receptor binding.

In one aspect, assays are provided for identifying mutant IL-2 polypeptides having biological activity. Biological activities may include, e.g., the ability to induce proliferation of IL-2 receptor-bearing T and/or NK cells, the ability to induce IL-2 signaling in IL-2 receptor-bearing T and/or NK cells, the ability to generate interferon (IFN)-γ as a secondary cytokine by NK cells, a reduced ability to induce elaboration of secondary cytokines, particularly IL-10 and TNF-α, by peripheral blood mononuclear cells (PBMCs), a reduced ability to induce apoptosis in T cells, the ability to induce tumor regression and/or improve survival, and a reduced toxicity profile, particularly reduced vasopermeability, in vivo. Mutant IL-2 polypeptides having such biological activity in vivo and/or in vitro are also provided.

In certain embodiments, a mutant IL-2 polypeptide of the invention is tested for such biological activity. A variety of methods are well known the art for determining biological activities of IL-2, and also details for many of these methods are disclosed in the Examples appended herewith. The Examples provide a suitable assay for testing IL-2 mutants of the invention for their ability to generate IFN-γ by NK cells. Cultured NK cells are incubated with the mutant IL-2 polypeptide or immunoconjugates of the invention, and IFN-γ concentration in the culture medium is subsequently measured by ELISA.

IL-2 induced signaling induces several signaling pathways, and involves JAK (Janus kinase) and STAT (signal transducer and activator of transcription) signaling molecules. The interaction of IL-2 with the receptor β- and γ-subunits leads to phosphorylation of the receptor and of JAK1 and JAK3, which are associated with the β- and γ-subunit, respectively. STAT5 then associates with the phosphorylated receptor and is phosphorylated itself on a crucial tyrosin residue. This results in the dissociation of STAT5 from the receptor, dimerization of STAT5 and translocation of the STAT5 dimers to the nucleus where they promote the transcription of target genes. The ability of mutant IL-2 polypeptides to induce signaling through the IL-2 receptor can thus be assessed, for example, by measuring phosphorylation of STAT5. Details of this method are disclosed in the Examples. PBMCs are treated with mutant IL-2 polypeptides or immunoconjugates of the invention and levels of phosphorylated STAT5 are determined by flow cytometry.

Proliferation of T cells or NK cells in response to IL-2 may be measured by incubating T cells or NK cells isolated from blood with mutant IL-2 polypeptides or immunoconjugates of the invention, followed by determination of the ATP content in lysates of the treated cells. Before treatment, T cells may be pre-stimulated with phytohemagglutinin (PHA-M). This assay, described in the Examples, allows sensitive quantitation of the number of viable cells, however there are numerous suitable alternative assays known in the art (e.g. [³H]-thymidine incorporation assay, Cell Titer Glo ATP assays, Alamar Blue assay, WST-1 assay, MTT assay).

An assay for determination of apoptosis of T cells and AICD is also provided in the Examples, wherein T cells are treated with an apoptosis-inducing antibody after the incubation with the mutant IL-2 polypeptides or immunoconjugates of the invention and apoptotic cells are quantified by flow cytometric detection of phosphatidyl serine/annexin exposure. Other assays are known in the art.

The effects of mutant IL-2 on tumor growth and survival can be assessed in a variety of animal tumor models known in the art. For example, xenografts of human cancer cell lines can be implanted to immunodeficient mice, and treated with mutant IL-2 polypeptides or immunoconjugates of the invention, as described in the Examples.

Toxicity of mutant IL-2 polypeptides and immunoconjugates of the invention in vivo can be determined based on mortality, in-life observations (visible symptoms of adverse effects, e.g. behaviour, body weight, body temperature) and clinical and anatomical pathology (e.g. measurements of blood chemistry values and/or histopathological analyses).

Vasopermeability induced by treatment with IL-2 can be examined in a pretreatment vasopermeability animal model. In general, the IL-2 mutant or immunoconjugate of the invention is administered to a suitable animal, e.g. a mouse, and at a later time the animal is injected with a vascular leak reporter molecule whose dissemination from the vasculature reflects the extent of vascular permeability. The vascular leak reporter molecule is preferably large enough to reveal permeability with the wild-type form of IL-2 used for pretreatment. An example of a vascular leak reporter molecule can be a serum protein such as albumin or an immunoglobulin. The vascular leak reporter molecule preferably is detectably labeled such as with a radioisotope to facilitate quantitative determination of the molecule's tissue distribution. Vascular permeability may be measured for vessels present in any of a variety of internal body organs such as liver, lung, and the like, as well as a tumor, including a tumor that is xenografted. Lung is a preferred organ for measuring vasopermeability of full-length IL-2 mutants.

Compositions, Formulations, and Routes of Administration

In a further aspect, the invention provides pharmaceutical compositions comprising any of the mutant IL-2 polypeptides or immunoconjugates provided herein, e.g., for use in any of the below therapeutic methods. In one embodiment, a pharmaceutical composition comprises any of the mutant IL-2 polypeptides or immunoconjugates provided herein and a pharmaceutically acceptable carrier. In another embodiment, a pharmaceutical composition comprises any of the mutant IL-2 polypeptides or immunoconjugates provided herein and at least one additional therapeutic agent, e.g., as described below.

Further provided is a method of producing a mutant IL-2 polypeptide or an immunoconjugate of the invention in a form suitable for administration in vivo, the method comprising (a) obtaining a mutant IL-2 polypeptide or immunoconjugate according to the invention, and (b) formulating the mutant IL-2 polypeptide or immunoconjugate with at least one pharmaceutically acceptable carrier, whereby a preparation of mutant IL-2 polypeptide or immunoconjugate is formulated for administration in vivo.

Pharmaceutical compositions of the present invention comprise a therapeutically effective amount of one or more mutant IL-2 polypeptide or immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one mutant IL-2 polypeptide or immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. Exemplary IL-2 compositions are described in U.S. Pat. Nos. 4,604,377 and 4,766,106. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. Mutant IL-2 polypeptides or immunoconjugates of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the mutant IL-2 polypeptides and immunoconjugates of the invention.

Parenteral compositions include those designed for administration by injection, e.g. subcutaneous, intradermal, intralesional, intravenous, intraarterial intramuscular, intrathecal or intraperitoneal injection. For injection, the mutant IL-2 polypeptides and immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the mutant IL-2 polypeptides and immunoconjugates may be in powder form for constitution with a suitable vehicle, e.g., sterile pyrogen-free water, before use. Sterile injectable solutions are prepared by incorporating the IL-2 polypeptides or immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the immunoconjugates may also be formulated as a depot preparation. Such long acting formulations may be administered by implantation (for example subcutaneously or intramuscularly) or by intramuscular injection. Thus, for example, the mutant IL-2 polypeptides and immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the mutant IL-2 polypeptides and immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The mutant IL-2 polypeptides and immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

Therapeutic Methods and Compositions

Any of the mutant IL-2 polypeptides and immunoconjugates provided herein may be used in therapeutic methods. Mutant IL-2 polypeptides and immunoconjugates of the invention can be used as immunotherapeutic agents, for example in the treatment of cancers.

For use in therapeutic methods, mutant IL-2 polypeptides and immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Mutant IL-2 polypeptides and immunoconjugates of the invention are useful in treating disease states where stimulation of the immune system of the host is beneficial, in particular conditions where an enhanced cellular immune response is desirable. These may include disease states where the host immune response is insufficient or deficient. Disease states for which the mutant IL-2 polypeptides or immunoconjugates of the invention can be administered comprise, for example, a tumor or infection where a cellular immune response would be a critical mechanism for specific immunity. Specific disease states for which IL-2 mutants of the present invention can be employed include cancer, for example renal cell carcinoma or melanoma; immune deficiency, specifically in HIV-positive patients, immunosuppressed patients, chronic infection and the like. The mutant IL-2 polypeptides or immunoconjugates of the invention may be administered per se or in any suitable pharmaceutical composition.

In one aspect, mutant IL-2 polypeptides and immunoconjugates of the invention for use as a medicament are provided. In further aspects, mutant IL-2 polypeptides and immunoconjugates of the invention for use in treating a disease are provided. In certain embodiments, mutant IL-2 polypeptides and immunoconjugates of the invention for use in a method of treatment are provided. In one embodiment, the invention provides a mutant IL-2 polypeptide or an immunoconjugate as described herein for use in the treatment of a disease in an individual in need thereof. In certain embodiments, the invention provides a mutant IL-2 polypeptide or an immunoconjugate for use in a method of treating an individual having a disease comprising administering to the individual a therapeutically effective amount of the mutant IL-2 polypeptide or the immunoconjugate. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a mutant IL-2 polypeptide or an immunoconjugate for use in stimulating the immune system. In certain embodiments, the invention provides a mutant IL-2 polypeptide or an immunoconjugate for use in a method of stimulating the immune system in an individual comprising administering to the individual an effective amount of the mutant IL-2 polypeptide or immunoconjugate to stimulate the immune system. An “individual” according to any of the above embodiments is a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides for the use of a mutant IL-2 polypeptide or an immunoconjugate of the invention in the manufacture or preparation of a medicament for the treatment of a disease in an individual in need thereof. In one embodiment, the medicament is for use in a method of treating a disease comprising administering to an individual having the disease a therapeutically effective amount of the medicament. In certain embodiments the disease to be treated is a proliferative disorder. In a preferred embodiment the disease is cancer. In one such embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in an individual comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides a method for treating a disease in an individual, comprising administering to said individual a therapeutically effective amount of a mutant IL-2 polypeptide or an immunoconjugate of the invention. In one embodiment a composition is administered to said individual, comprising the mutant IL-2 polypeptide or the immunoconjugate of the invention in a pharmaceutically acceptable form. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of a mutant IL-2 polypeptide or an immunoconjugate to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

It is understood that any of the above therapeutic methods may be carried out using an immunoconjugate of the invention in place of or in addition to a mutant IL-2 polypeptide.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a mutant IL-2 polypeptide or an immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. Similarly, other cell proliferation disorders can also be treated by the mutant IL-2 polypeptides and immunoconjugates of the present invention. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above. In another embodiment, the disease is related to autoimmunity, transplantation rejection, post-traumatic immune responses and infectious diseases (e.g. HIV). More specifically, the mutant IL-2 polypeptides and immunoconjugates may be used in eliminating cells involved in immune cell-mediated disorders, including lymphoma; autoimmunity, transplantation rejection, graft-versus-host disease, ischemia and stroke. A skilled artisan readily recognizes that in many cases the mutant IL-2 polypeptides or immunoconjugates may not provide a cure but may only provide partial benefit. In some embodiments, a physiological change having some benefit is also considered therapeutically beneficial. Thus, in some embodiments, an amount of mutant IL-2 polypeptide or immunoconjugate that provides a physiological change is considered an “effective amount” or a “therapeutically effective amount”. The subject, patient, or individual in need of treatment is typically a mammal, more specifically a human.

The immunoconjugates of the invention are also useful as diagnostic reagents. The binding of an immunoconjugate to an antigenic determinant can be readily detected by using a secondary antibody specific for the IL-2 polypeptide. In one embodiment, the secondary antibody and the immunoconjugate facilitate the detection of binding of the immunoconjugate to an antigenic determinant located on a cell or tissue surface.

In some embodiments, an effective amount of the mutant IL-2 polypeptides or immunoconjugates of the invention is administered to a cell. In other embodiments, a therapeutically effective amount of the mutant IL-2 polypeptides or immunoconjugates of the invention is administered to an individual for the treatment of disease.

For the prevention or treatment of disease, the appropriate dosage of a mutant IL-2 polypeptide or immunoconjugate of the invention (when used alone or in combination with one or more other additional therapeutic agents) will depend on the type of disease to be treated, the route of administration, the body weight of the patient, the type of polypeptide (e.g. unconjugated IL-2 or immunoconjugate), the severity and course of the disease, whether the antibody is administered for preventive or therapeutic purposes, previous or concurrent therapeutic interventions, the patient's clinical history and response to the mutant IL-2 polypeptide or immunoconjugate, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The mutant IL-2 polypeptides and immunoconjugates of the invention will generally be used in an amount effective to achieve the intended purpose. For use to treat or prevent a disease condition, the mutant IL-2 polypeptides and immunoconjugates of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

In cases of local administration or selective uptake, the effective local concentration of the immunoconjugates may not be related to plasma concentration. One having skill in the art will be able to optimize therapeutically effective local dosages without undue experimentation.

A therapeutically effective dose of the mutant IL-2 polypeptides or immunoconjugates described herein will generally provide therapeutic benefit without causing substantial toxicity. Toxicity and therapeutic efficacy of an IL-2 mutant or immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. IL-2 mutants and immunoconjugates that exhibit large therapeutic indices are preferred. In one embodiment, the mutant IL-2 polypeptide or the immunoconjugate according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with IL-2 mutants or immunoconjugates of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The maximum therapeutic dose of a mutant IL-2 polypeptide or immunoconjugate comprising said polypeptide may be increased from those used for wild-type IL-2 or an immunoconjugate comprising wild-type IL-2, respectively.

Other Agents and Treatments

The mutant IL-2 polypeptides and the immunoconjugates according to the invention may be administered in combination with one or more other agents in therapy. For instance, a mutant IL-2 polypeptide or immunoconjugate of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of mutant IL-2 polypeptide or immunoconjugate used, the type of disorder or treatment, and other factors discussed above. The mutant IL-2 polypeptides and immunoconjugates are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the mutant IL-2 polypeptide or immunoconjugate of the invention can occur prior to, simultaneously, and/or following, administration of the additional therapeutic agent and/or adjuvant. Mutant IL-2 polypeptides and immunoconjugates of the invention can also be used in combination with radiation therapy.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a mutant IL-2 polypeptide of the invention. The label or package insert indicates that the composition is used for treating the condition of choice. Moreover, the article of manufacture may comprise (a) a first container with a composition contained therein, wherein the composition comprises a mutant IL-2 polypeptide of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to a mutant IL-2 polypeptide.

Sequences

| | SEQ ID | |
|---|---|---|
| Name | NO | Sequence |
| human IL2- | 1 | PTSSSTKKTQLQLEHLLLDLQMILNGINNYKNPKLTRMLTFKFYMPKKATEL |
| proleukin | | KHLQCLEEELKPLEEVLNLAQSKNFHLRPRDLISNINVIVLELKGSETTFMCE |
| | | YADETATIVEFLNRWITFSQSIISTLT |
| Human | 2 | LNTTILTPNGNEDTTADFFLTTMPTDSLSVSTLPLPEVQCFVFNVEYMNCTW |
| IL2Rgamma extra | | NSSSEPQPTNLTLHYWYKNSDNDKVQKCSHYLFSEEITSGCQLQKKEIHLYQ |
| cellular domain | | TFVVQLQDPREPRRQATQMLKLQNLVIPWAPENLTLHKLSESQLELNWNNR |
| fused to Fc(hole) | | FLNHCLEHLVQYRTDWDHSWTEQSVDYRHKFSLPSVDGQKRYTFRVRSRF |
| | | NPLCGSAQHWSEWSHPIHWGSNTSKENPFLFALEAGAQDKTHTCPPCPAPEL |
| | | LGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTI |
| | | SKAKGQPREPQVCTLPPSRDELTKNQVSLSCAVKGFYPSDIAVEWESNGQPE |
| | | NNYKTTPPVLDSDGSFFLVSKLTVDKSRWQQGNVFSCSVMHEALHNHYTQ |
| | | KSLSLSPGK |
| Human IL2Rbeta | 3 | AVNGTSQFTCFYNSRANISCVWSQDGALQDTSCQVHAWPDRRRWNQTCEL |
| extra cellular | | LPVSQASWACNLILGAPDSQKLTTVDIVTLRVLCREGVRWRVMAIQDFKPFE |
| domain fused to | | NLRLMAPISLQVVHVETHRCNISWEISQASHYFERHLEFEARTLSPGHTWEE |
| Fc(knob) | | APLLTLKQKQEWICLETLTPDTQYEFQVRVKPLQGEFTTWSPWSQPLAFRTK |
| | | PAALGKDTGAQDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPCRDELTKNQVS |
| | | LWCLVKGFYPSDIAVEWESNGQPENNYKTTPPVLDSDGSFFLYSKLTVDKSR |
| | | WQQGNVFSCSVMHEALHNHYTQKSLSLSPGKSGGLNDIFEAQKIEWHE |
| IL2v | 4 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIISTLT |
| ExplResIEE.P026. | 5 | APASSSTKKTQEQLEHLLLELQNILNGINNYKNPKLTRMLTAKFAMPKKATE |
| 099 | | LKHLQCLEEELKPLEEVLNGAQSKNFHLNPRELIENINVIVLELKGSETTFMC |
| | | EYADETATIVEFLNEWIEFAQSIIETLN |
| ExplResIEE.P026. | 6 | APASSSTKKTELQLEHLLLQLQEILNGINNYKNPKLTRMLTAKFAMPKKATE |
| 175 | | LKHLQCLEEELKPLEEVLNGAQSKNFHLDPRELIENINVIVLELKGSETTFMC |
| | | EYADETATIVEFLNRWITFAQSIIETLN |
| ExplResIEE.P026. | 7 | APASSSTKKTQEQLEHLLDDLQMILNGINNYKNPKLTRMLTAKFAMPKKAT |
| 191 | | ELKHLQCLEEELKPLEEVLNGAQSKNFHLDPRDLISNINVIVLELKGSETTFM |
| | | CEYADETATIVEFLNEWIEFAQSIIETLE |
| ExplResIEE.P026. | 8 | APASSSTKKTQLQLEHLLLDLQMILNGINNYKNPKLTRMLTAKFAMPKKAT |
| 237 | | ELKHLQCLEEELKPLEEVLNGAQSKNFHLQPRDLIENINDIVLELKGSETTFM |
| | | CEYADETATIVEFLNRWIEFAQSIIETLD |
| ExplResIEE.P026. | 9 | APASSSTKKTEEQLEHLLLDLQQILNGINNYKNPKLTRMLTAKFAMPKKATE |
| 355 | | LKHLQCLEEELKPLEEVLNGAQSKNFHLDPRDLIENINDIVLELKGSETTFMC |
| | | EYADETATIVEFLNRWITFAQSIIETLQ |
| ExplResIEE.P027. | 10 | APASSSTKKTELQLEHLLDDLQMILNGINNYKNPKLTRMLTAKFAMPKKAT |
| 359 | | ELKHLQCLEEELKPLEEVLNGAQSKNFHLDPRELISNINVIVLELKGSETTFM |
| | | CEYADETATIVEFLNRWITFAQSIIETLD |
| ExplResIEE.P030. | 11 | APASSSTKKTQLQLEHLLLDLQMILNGINNYKNPKLTRMLTAKFAMPKKAT |
| 006 | | ELKHLQCLEEELKPLEEVLNGAQSKNFHLDPRQLIDNINNIVLELKGSETTFM |
| | | CEYADETATIVEFLNRWIQFAQSIIETLD |
| ExplResIEE.P030. | 12 | APASSSTKKTQLQLEHLLDDLQMILNGINNYKNPKLTRMLTAKFAMPKKAT |
| 045 | | ELKHLQCLEEELKPLEEVLNGAQSKNFHLEPRELIQNINVIVLELKGSETTFM |
| | | CEYADETATIVEFLNHWITFAQSIIETLE |
| ExplResIEE.P031. | 13 | APASSSTKKTQLQLEHLLDDLQNILNGINNYKNPKLTRMLTAKFAMPKKAT |
| 226 | | ELKHLQCLEEELKPLEEVLNGAQSKNFHLDPRDLISNINVIVLELKGSETTFM |
| | | CEYADETATIVEFLNRWITFAQSIISTLE |
| ExplResIEE.P041. | 14 | APASSSTKKTEEQLEHLLLDLQQILNGINNYKNPKLTRMLTAKFAMPKKATE |
| 017 | | LKHLQCLEEELKPLEEVLNGAQSKNFHLQPRDLIDNINNIVLQLKGSETTFMC |
| | | EYADETATIVEFLNHWIEFAQSIIETLE |
| ExplResIEE.P041. | 15 | APASSSTKKTQLQLEHLLDDLQMILNGINNYKNPKLTRMLTAKFAMPKKAT |
| 242 | | ELKHLQCLEEELKPLEEVLNGAQSKNFHLEPRDLISNINVIVLELKGSETTFM |
| | | CEYADETATIVEFLNRWITFAQSIIETLD |
| ExplResIEE.P041. | 16 | APASSSTKKTQLQLEHLLLDLQMILNGINNYKNPKLTRMLTAKFAMPKKAT |
| 449 | | ELKHLQCLEEELKPLEEVLNGAQSKNFHLQPRDLIENINDIVLELKGSETTFM |
| | | CEYADETATIVEFLNEWITFAQSIIETLD |
| ExplResIEE.P041. | 17 | APASSSTKKTQQQLEHLLQDLQMILNGINNYKNPKLTRMLTAKFAMPKKAT |
| 554 | | ELKHLQCLEEELKPLEEVLNGAQSKNFHLHPRDLISNINEIVLELKGSETTFM |
| | | CEYADETATIVEFLNRWIEFAQSIIETLE |
| ExplResIEE.P042. | 18 | APASSSTEKTQLQLEHLLLELQNILNGINNYKNPKLTRMLTAKFAMPKKATE |
| 107 | | LKHLQCLEEELKPLEEVLNGAQSKNFHLHPRQLIENINVIVLELKGSETTFMC |
| | | EYADETATIVEFLNHWITFAQSIIETLD |
| ExplResIEE.P043. | 19 | APASSSTKKTQEQLEHLLQDLQMILNGINNYKNPKLTRMLTAKFAMPKKAT |
| 217 | | ELKHLQCLEEELKPLEEVLNGAQSKNFHLHPRDLISNINVIVLELKGSETTFM |
| | | CEYADETATIVEFLNEWITFAQSIISTLD |
| ExplResOnc.P173. | 20 | APASSSTKKTQLQLEELLDDLEQILNGINNYKNPKLTRMLTAKFAMPKKATE |
| 0079 | | LKHLQCLEEELKPLEEVLNGAQSKNFHLDPRELIDNINVIVLELKGSETTFMC |
| | | EYADETATIVEFLNHWITFAQSIIETLE |
| ExplResOnc.P173. | 21 | APASSSTKKTEEQLEQLLDDLENILNGINNYKNPKLTRMLTAKFAMPKKATE |
| 0087 | | LKHLQCLEEELKPLEEVLNGAQSKNFHLEPRELIENINVIVLELKGSETTFMC |
| | | EYADETATIVEFLNHWITFAQSIIETLE |
| ExplResOnc.P173. | 22 | APASSSTKKTELQLEELLDDLQEILNGINNYKNPKLTRMLTAKFAMPKKATE |
| 0127 | | LKHLQCLEEELKPLEEVLNGAQSKNFHLDPRDLIENINVIVLELKGSETTFMC |
| | | EYADETATIVEFLNHWITFAESIISTLD |
| ExplResOnc.P173. | 23 | APASSSTKKTESQLQNLLDDLQEILNGINNYKNPKLTRMLTAKFAMPKKATE |
| 0156 | | LKHLQCLEEELKPLEEVLNGAQSKNFHLEPRELIDNINVIVLELKGSETTFMC |
| | | EYADETATIVEFLNHWITFAQSIIETLE |
| ExplResOnc.P173. | 24 | APASSSTKKTELQLEELLLDLQEILNGINNYKNPKLTRMLTAKFAMPKKATE |
| 0182 | | LKHLQCLEEELKPLEEVLNGAQSKNFHLNPRELIENINVIVLELKGSETTFMC |
| | | EYADETATIVEFLNHWITFAESIIETLE |
| ExplResOnc.P173. | 25 | APASSSTKKTELQLQELLLDLEEILNGINNYKNPKLTRMLTAKFAMPKKATE |
| 0239 | | LKHLQCLEEELKPLEEVLNGAQSKNFHLHPRELIENINVIVLELKGSETTFMC |
| | | EYADETATIVEFLNHWITFAQSIIETLD |
| ExplResOnc.P173. | 26 | APASSSTKKTEDHLQELLLDLEEILNGINNYKNPKLTRMLTAKFAMPKKATE |
| 0255 | | LKHLQCLEEELKPLEEVLNGAQSKNFHLNPRELIENINVIVLELKGSETTFMC |
| | | EYADETATIVEFLNHWITFAQSIIETLE |
| ExplResOnc.P173. | 27 | APASSSTKKTELQLQELLDDLQMILNGINNYKNPKLTRMLTAKFAMPKKAT |
| 0259 | | ELKHLQCLEEELKPLEEVLNGAQSKNFHLEPRDLIENINVIVLELKGSETTFM |
| | | CEYADETATIVEFLNHWITFAQSIIETLE |
| ExplResOnc.P173. | 28 | APASSSTKKTQLQLEELLDDLQQILNGINNYKNPKLTRMLTAKFAMPKKATE |
| 0371 | | LKHLQCLEEELKPLEEVLNGAQSKNFHLNPRELIDNINVIVLELKGSETTFMC |
| | | EYADETATIVEFLNHWITFAQSIIETLD |
| ExplResOnc.P174. | 29 | APASSSTKKTETQLQELLDDLHMILNGINNYKNPKLTRMLTAKFAMPKKAT |
| 0173 | | ELKHLQCLEEELKPLEEVLNGAQSKNFHLDPRELIENINVIVLELKGSETTFM |
| | | CEYADETATIVEFLNHWITFAQSIIETLE |
| ExplResOnc.P174. | 30 | APASSSTKKTETQLQELLDDLHEILNGINNYKNPKLTRMLTAKFAMPKKATE |
| 0238 | | LKHLQCLEEELKPLEEVLNGAQSKNFHLEPRELIENINVIVLELKGSETTFMC |
| | | EYADETATIVEFLNHWITFAQSIIETLE |
| ExplResOnc.P174. | 31 | APASSSTKKTELQLQELLDDLQMILNGINNYKNPKLTRMLTAKFAMPKKAT |
| 0277 | | ELKHLQCLEEELKPLEEVLNGAQSKNFHLEPRELIENINVIVLELKGSETTFM |
| | | CEYADETATIVEFLNHWITFAQSIIETLE |
| ExplResOnc.P174. | 32 | APASSSTKKTQLQLEELLLDLEQILNGINNYKNPKLTRMLTAKFAMPKKATE |
| 0281 | | LKHLQCLEEELKPLEEVLNGAQSKNFHLRPRDLINNINVIVLELKGSETTFMC |
| | | EYADETATIVEFLNHWITFAQSIIETLE |
| ExplResOnc.P174. | 33 | APASSSTKKTQLQLEELLDDLDQILNGINNYKNPKLTRMLTAKFAMPKKATE |
| 0326 | | LKHLQCLEEELKPLEEVLNGAQSKNFHLDPRELIENINVIVLELKGSETTFMC |
| | | EYADETATIVEFLNHWITFAQSIIETLE |
| ExplResOnc.P174. | 34 | APASSSTKKTEEHLQNLLDDLEQILNGINNYKNPKLTRMLTAKFAMPKKATE |
| 0327 | | LKHLQCLEEELKPLEEVLNGAQSKNFHLEPRELIDNINVIVLDLKGSETTFMC |
| | | EYADETATIVEFLNHWITFAQSIISTLE |
| ExplResOnc.P175. | 35 | APASSSTKKTETQLEELLDDLEMILNGINNYKNPKLTRMLTAKFAMPKKATE |
| 0125 | | LKHLQCLEEELKPLEEVLNGAQSKNFHLDPRELIENINVIVLELKGSETTFMC |
| | | EYADETATIVEFLNHWITFAQSIIETLD |
| ExplResOnc.P175. | 36 | APASSSTKKTTEQLQELLDDLQMILNGINNYKNPKLTRMLTAKFAMPKKAT |
| 0368 | | ELKHLQCLEEELKPLEEVLNGAQSKNFHLDPRELIENINVIVLELKGSETTFM |
| | | CEYADETATIVEFLNEWITFAQSIIETLD |
| ExplResOnc.P177. | 37 | APASSSTKKTELQLQELLDDLQMILNGINNYKNPKLTRMLTAKFAMPKKAT |
| 0035 (IL2v | | ELKHLQCLEEELKPLEEVLNGAQSKNFHLDPRELIENINVIVLELKGSETTFM |
| (consensus all)) | | CEYADETATIVEFLNHWITFAQSIIETLE |
| ExplResOnc.P177. | 38 | APASSSTKKTELQLQELLDDLQMILNGINNYKNPKLTRMLTAKFAMPKKAT |
| 0036 | | ELKHLQCLEEELKPLEEVLNGAQSKNFHLQPRELIENINVIVLELKGSETTFM |
| | | CEYADETATIVEFLNHWITFAQSIIETLE |
| ExplResOnc.P177. | 39 | APASSSTKKTESQLEELLDDLQQILNGINNYKNPKLTRMLTAKFAMPKKATE |
| 0156 | | LKHLQCLEEELKPLEEVLNGAQSKNFHLDPRELIENINVIVLELKGSETTFMC |
| | | EYADETATIVEFLNHWITFAQSIIETLE |
| ExplResOnc.P172. | 40 | APASSYTKKTQERLEQLLLDLEQILNGINNYKNPKLTRMLTAKFAMPKKATE |
| 0344 | | LKHLQCLEEELKPLEEVLNGAQSKNFHLNPRELIENINVIVLELKGSETTFMC |
| | | EYADETATIVEFLNHWITFAQSIIETLD |
| ExplResOnc.P173. | 41 | APASSSTKKTQLQLEDLLLDLQNILNGINNYKNPKLTRMLTAKFAMPKKATE |
| 0364 | | LKHLQCLEEELKPLEEVLNGAQSKNFHLDPRELISNINVIVLELKGSETTFMC |
| | | EYADETATIVEFLNHWITFAQSIIETLE |
| ExplResOnc.P174. | 42 | APASSSTKKTETQLEQLLDDLQEILNGINNYKNPKLTRMLTAKFAMPKKATE |
| 0040 | | LKHLQCLEEELKPLEEVLNGAQSKNFHLDPRELIENINVIVLELKGSETTFMC |
| | | EYADETATIVEFLNHWITFAQSIIETLE |
| ExplResOnc.P178. | 43 | APASSSTKKTEEQLENLLLDLQNILNGINNYKNPKLTRMLTAKFAMPKKATE |
| 0145 | | LKHLQCLEEELKPLEEVLNGAQSKNFHLEPRELISNINVIVLELKGSETTFMC |
| | | EYADETATIVEFLNHWITFAQSIIETLE |
| IL2v (Q11E) | 44 | APASSSTKKTELQLEHLLLDLQMILNGINNYKNPKLTRMLTAKFAMPKKATE |
| | | LKHLQCLEEELKPLEEVLNGAQSKNFHLRPRDLISNINVIVLELKGSETTFMC |
| | | EYADETATIVEFLNRWITFAQSIISTLT |
| IL2v (E15Q) | 45 | APASSSTKKTQLQLQHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIISTLT |
| IL2v (H16E) | 46 | APASSSTKKTQLQLEELLLDLQMILNGINNYKNPKLTRMLTAKFAMPKKATE |
| | | LKHLQCLEEELKPLEEVLNGAQSKNFHLRPRDLISNINVIVLELKGSETTFMC |
| | | EYADETATIVEFLNRWITFAQSIISTLT |
| IL2v (L19D) | 47 | APASSSTKKTQLQLEHLLDDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIISTLT |
| IL2v (Q22E) | 48 | APASSSTKKTQLQLEHLLLDLEMILNGINNYKNPKLTRMLTAKFAMPKKATE |
| | | LKHLQCLEEELKPLEEVLNGAQSKNFHLRPRDLISNINVIVLELKGSETTFMC |
| | | EYADETATIVEFLNRWITFAQSIISTLT |
| IL2v (M23Q) | 49 | APASSSTKKTQLQLEHLLLDLQQILNGINNYKNPKLTRMLTAKFAMPKKATE |
| | | LKHLQCLEEELKPLEEVLNGAQSKNFHLRPRDLISNINVIVLELKGSETTFMC |
| | | EYADETATIVEFLNRWITFAQSIISTLT |
| IL2v (R81D) | 50 | APASSSTKKTQLQLEHLLLDLQMILNGINNYKNPKLTRMLTAKFAMPKKAT |
| | | ELKHLQCLEEELKPLEEVLNGAQSKNFHLDPRDLISNINVIVLELKGSETTFM |
| | | CEYADETATIVEFLNRWITFAQSIISTLT |
| IL2v (D84E) | 51 | APASSSTKKTQLQLEHLLLDLQMILNGINNYKNPKLTRMLTAKFAMPKKAT |
| | | ELKHLQCLEEELKPLEEVLNGAQSKNFHLRPRELISNINVIVLELKGSETTFM |
| | | CEYADETATIVEFLNRWITFAQSIISTLT |
| IL2v (S87E) | 52 | APASSSTKKTQLQLEHLLLDLQMILNGINNYKNPKLTRMLTAKFAMPKKAT |
| | | ELKHLQCLEEELKPLEEVLNGAQSKNFHLRPRDLIENINVIVLELKGSETTFM |
| | | CEYADETATIVEFLNRWITFAQSIISTLT |
| IL2v (R120H) | 53 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNHWITFAQSIISTLT |
| IL2v (Q126E) | 54 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAESIISTLT |
| IL2v (Q126H) | 55 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAHSIISTLT |
| IL2v (S130E) | 56 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIIETLT |
| IL2v (T133E) | 57 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIISTLE |
| IL2v (T133D) | 58 | APASSSTKKTQLQLEHLLLDLQMILNGINNYKNPKLTRMLTAKFAMPKKAT |
| | | ELKHLQCLEEELKPLEEVLNGAQSKNFHLRPRDLISNINVIVLELKGSETTFM |
| | | CEYADETATIVEFLNRWITFAQSIISTLD |
| IL2v (consensus | 59 | APASSSTKKTQLQLQELLDDLQMILNGINNYKNPKLTRMLTAKFAMPKKAT |
| beta) | | ELKHLQCLEEELKPLEEVLNGAQSKNFHLDPRELIENINVIVLELKGSETTFM |
| | | CEYADETATIVEFLNRWITFAQSIISTLT |
| IL2v (consensus | 60 | APASSSTKKTELQLEHLLLDLQMILNGINNYKNPKLTRMLTAKFAMPKKATE |
| gamma) | | LKHLQCLEEELKPLEEVLNGAQSKNFHLRPRDLISNINVIVLELKGSETTFMC |
| | | EYADETATIVEFLNHWITFAQSIIETLD |
| template for 2^{nd} | 61 | APASSSTKKTELQLEHLLLDLQMILNGINNYKNPKLTRMLTAKFAMPKKATE |
| library | | LKHLQCLEEELKPLEEVLNGAQSKNFHLDPRELIENINVIVLELKGSETTFMC |
| | | EYADETATIVEFLNHWITFAQSIIETLD |
| LC for CD8- | 62 | DVQITQSPSSLSASVGDRVTITCRTSRSISQYLAWYQEKPGKTNKLLIYS |
| targeted format(1^{st} | | GSTLQSGIPSRFSGSGSGTDFTLTISSLQPEDFATYYCQQHNENPLTFGQ |
| and 2^{nd} set of pH- | | GTKVEIKRTVAAPSVFIFPPSDEQLKSGTASVVCLLNNFYPREAKVQWKV |
| dep IL2v variants) | | DNALQSGNSQESVTEQDSKDSTYSLSSTLTLSKADYEKHKVYACEVTHQG |
| | | LSSPVTKSFNRGEC |
| ‘empty’ HC for | 63 | DKTHTCPPCPAPEAAGGPSVFLFPPKPKDTLMISRTPEVTCVVVDVSHED |
| CD8-targeted | | PEVKFNWYVDGVEVHNAKTKPREEQYNSTYRVVSVLTVLHQDWLNGKEY |
| format (1^{st} set of | | KCKVSNKALGAPIEKTISKAKGQPREPQVCTLPPSRDELTKNQVSLSCAVK |
| pH-dep IL2v | | GFYPSDIAVEWESNGQPENNYKTTPPVLDSDGSFFLVSKLTVDKSRWQQG |
| variants) | | NVFSCSVMHEALHNRFTQKSLSLSPGK |
| ‘long’ HC for 1^{st} | 64 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (OA | | FPEPVTVSWNSGALTSGVHTFPAVLQSSGLYSLSSVVTVPSSSLGTQTYI |
| CD8 IgG IL2v) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLLDLQMILNGINNYKNPKLTRMLT |
| | | AKFAMPKKATELKHLQCLEEELKPLEEVLNGAQSKNFHLRPRDLISNINVIVL |
| | | ELKGSETTFMCEYADETATIVEFLNRWITFAQSIISTLT |
| ‘long’ HC for 1^{st} | 65 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P026. | | CNVNHKPSNTKVDKKVEPKSCDKTHTCPPCPAPEAAGGPSVFLFPPKPKD |
| 099 (OA CD8 IgG | | TLMISRTPEVTCVVVDVSHEDPEVKFNWYVDGVEVHNAKTKPREEQYNST |
| IL2v P026.099) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQEQLEHLLLELQNILNGINNYKNPKLTRMLT |
| | | AKFAMPKKATELKHLQCLEEELKPLEEVLNGAQSKNFHLNPRELIENINVIVL |
| | | ELKGSETTFMCEYADETATIVEFLNEWIEFAQSIIETLN |
| ‘long’ HC for 1^{st} | 66 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P026. | | CNVNHKPSNTKVDKKVEPKSCDKTHTCPPCPAPEAAGGPSVFLFPPKPKD |
| 175 (OA CD8 IgG | | TLMISRTPEVTCVVVDVSHEDPEVKFNWYVDGVEVHNAKTKPREEQYNST |
| IL2v P026.175) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTELQLEHLLLQLQEILNGINNYKNPKLTRMLT |
| | | AKFAMPKKATELKHLQCLEEELKPLEEVLNGAQSKNFHLDPRELIENINVIVL |
| | | ELKGSETTFMCEYADETATIVEFLNRWITFAQSIIETLN |
| ‘long’ HC for 1^{st} | 67 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P026. | | CNVNHKPSNTKVDKKVEPKSCDKTHTCPPCPAPEAAGGPSVFLFPPKPKD |
| 191 (OA CD8 IgG | | TLMISRTPEVTCVVVDVSHEDPEVKFNWYVDGVEVHNAKTKPREEQYNST |
| IL2v P026.191) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQEQLEHLLDDLQMILNGINNYKNPKLTRML |
| | | TAKFAMPKKATELKHLQCLEEELKPLEEVLNGAQSKNFHLDPRDLISNINVI |
| | | VLELKGSETTFMCEYADETATIVEFLNEWIEFAQSIIETLE |
| ‘long’ HC for 1^{st} | 68 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P026. | | CNVNHKPSNTKVDKKVEPKSCDKTHTCPPCPAPEAAGGPSVFLFPPKPKD |
| 237 (OA CD8 IgG | | TLMISRTPEVTCVVVDVSHEDPEVKFNWYVDGVEVHNAKTKPREEQYNST |
| IL2v P026.237) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLLDLQMILNGINNYKNPKLTRMLT |
| | | AKFAMPKKATELKHLQCLEEELKPLEEVLNGAQSKNFHLQPRDLIENINDIV |
| | | LELKGSETTFMCEYADETATIVEFLNRWIEFAQSIIETLD |
| ‘long’ HC for 1^{st} | 69 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P026. | | CNVNHKPSNTKVDKKVEPKSCDKTHTCPPCPAPEAAGGPSVFLFPPKPKD |
| 355 (OA CD8 IgG | | TLMISRTPEVTCVVVDVSHEDPEVKFNWYVDGVEVHNAKTKPREEQYNST |
| IL2v P026.355) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTEEQLEHLLLDLQQILNGINNYKNPKLTRMLT |
| | | AKFAMPKKATELKHLQCLEEELKPLEEVLNGAQSKNFHLDPRDLIENINDIV |
| | | LELKGSETTFMCEYADETATIVEFLNRWITFAQSIIETLQ |
| ‘long’ HC for 1^{st} | 70 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P027. | | CNVNHKPSNTKVDKKVEPKSCDKTHTCPPCPAPEAAGGPSVFLFPPKPKD |
| 359 (OA CD8 IgG | | TLMISRTPEVTCVVVDVSHEDPEVKFNWYVDGVEVHNAKTKPREEQYNST |
| IL2v P027.359) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTELQLEHLLDDLQMILNGINNYKNPKLTRMLT |
| | | AKFAMPKKATELKHLQCLEEELKPLEEVLNGAQSKNFHLDPRELISNINVIVL |
| | | ELKGSETTFMCEYADETATIVEFLNRWITFAQSIIETLD |
| ‘long’ HC for 1^{st} | 71 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P030. | | CNVNHKPSNTKVDKKVEPKSCDKTHTCPPCPAPEAAGGPSVFLFPPKPKD |
| 006 (OA CD8 IgG | | TLMISRTPEVTCVVVDVSHEDPEVKFNWYVDGVEVHNAKTKPREEQYNST |
| IL2v P030.006) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLLDLQMILNGINNYKNPKLTRMLT |
| | | AKFAMPKKATELKHLQCLEEELKPLEEVLNGAQSKNFHLDPRQLIDNINNIV |
| | | LELKGSETTFMCEYADETATIVEFLNRWIQFAQSIIETLD |
| ‘long’ HC for 1^{st} | 72 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P030. | | CNVNHKPSNTKVDKKVEPKSCDKTHTCPPCPAPEAAGGPSVFLFPPKPKD |
| 045 (OA CD8 IgG | | TLMISRTPEVTCVVVDVSHEDPEVKFNWYVDGVEVHNAKTKPREEQYNST |
| IL2v P030.045) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLDDLQMILNGINNYKNPKLTRML |
| | | TAKFAMPKKATELKHLQCLEEELKPLEEVLNGAQSKNFHLEPRELIQNINVIV |
| | | LELKGSETTFMCEYADETATIVEFLNHWITFAQSIIETLE |
| ‘long’ HC for 1^{st} | 73 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P031. | | CNVNHKPSNTKVDKKVEPKSCDKTHTCPPCPAPEAAGGPSVFLFPPKPKD |
| 226 (OA CD8 IgG | | TLMISRTPEVTCVVVDVSHEDPEVKFNWYVDGVEVHNAKTKPREEQYNST |
| IL2v P031.226) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLDDLQNILNGINNYKNPKLTRMLT |
| | | AKFAMPKKATELKHLQCLEEELKPLEEVLNGAQSKNFHLDPRDLISNINVIV |
| | | LELKGSETTFMCEYADETATIVEFLNRWITFAQSIISTLE |
| ‘long’ HC for 1^{st} | 74 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P041. | | CNVNHKPSNTKVDKKVEPKSCDKTHTCPPCPAPEAAGGPSVFLFPPKPKD |
| 017 (OA CD8 IgG | | TLMISRTPEVTCVVVDVSHEDPEVKFNWYVDGVEVHNAKTKPREEQYNST |
| IL2v P041.017) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTEEQLEHLLLDLQQILNGINNYKNPKLTRMLT |
| | | AKFAMPKKATELKHLQCLEEELKPLEEVLNGAQSKNFHLQPRDLIDNINNIV |
| | | LQLKGSETTFMCEYADETATIVEFLNHWIEFAQSIIETLE |
| ‘long’ HC for 1^{st} | 75 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P041. | | CNVNHKPSNTKVDKKVEPKSCDKTHTCPPCPAPEAAGGPSVFLFPPKPKD |
| 242 (OA CD8 IgG | | TLMISRTPEVTCVVVDVSHEDPEVKFNWYVDGVEVHNAKTKPREEQYNST |
| IL2v P041.242) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLDDLQMILNGINNYKNPKLTRML |
| | | TAKFAMPKKATELKHLQCLEEELKPLEEVLNGAQSKNFHLEPRDLISNINVIV |
| | | LELKGSETTFMCEYADETATIVEFLNRWITFAQSIIETLD |
| ‘long’ HC for 1^{st} | 76 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P041. | | CNVNHKPSNTKVDKKVEPKSCDKTHTCPPCPAPEAAGGPSVFLFPPKPKD |
| 449 (OA CD8 IgG | | TLMISRTPEVTCVVVDVSHEDPEVKFNWYVDGVEVHNAKTKPREEQYNST |
| IL2v P041.449) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQLQLEHLLLDLQMILNGINNYKNPKLTRMLT |
| | | AKFAMPKKATELKHLQCLEEELKPLEEVLNGAQSKNFHLQPRDLIENINDIV |
| | | LELKGSETTFMCEYADETATIVEFLNEWITFAQSIIETLD |
| ‘long’ HC for 1^{st} | 77 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P041. | | CNVNHKPSNTKVDKKVEPKSCDKTHTCPPCPAPEAAGGPSVFLFPPKPKD |
| 554 (OA CD8 IgG | | TLMISRTPEVTCVVVDVSHEDPEVKFNWYVDGVEVHNAKTKPREEQYNST |
| IL2v P041.554) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQQQLEHLLQDLQMILNGINNYKNPKLTRML |
| | | TAKFAMPKKATELKHLQCLEEELKPLEEVLNGAQSKNFHLHPRDLISNINEIV |
| | | LELKGSETTFMCEYADETATIVEFLNRWIEFAQSIIETLE |
| ‘long’ HC for 1^{st} | 78 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P042. | | CNVNHKPSNTKVDKKVEPKSCDKTHTCPPCPAPEAAGGPSVFLFPPKPKD |
| 107 (OA CD8 IgG | | TLMISRTPEVTCVVVDVSHEDPEVKFNWYVDGVEVHNAKTKPREEQYNST |
| IL2v P042.107) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTEKTQLQLEHLLLELQNILNGINNYKNPKLTRMLT |
| | | AKFAMPKKATELKHLQCLEEELKPLEEVLNGAQSKNFHLHPRQLIENINVIV |
| | | LELKGSETTFMCEYADETATIVEFLNHWITFAQSIIETLD |
| ‘long’ HC for 1^{st} | 79 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResIEE.P043. | | CNVNHKPSNTKVDKKVEPKSCDKTHTCPPCPAPEAAGGPSVFLFPPKPKD |
| 217 (OA CD8 IgG | | TLMISRTPEVTCVVVDVSHEDPEVKFNWYVDGVEVHNAKTKPREEQYNST |
| IL2v P043.217) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGSAPASSSTKKTQEQLEHLLQDLQMILNGINNYKNPKLTRML |
| | | TAKFAMPKKATELKHLQCLEEELKPLEEVLNGAQSKNFHLHPRDLISNINVI |
| | | VLELKGSETTFMCEYADETATIVEFLNEWITFAQSIISTLD |
| ‘empty’ HC for | 80 | DKTHTCPPCPAPEAAGGPSVFLFPPKPKDTLMISRTPEVTCVVVDVSHED |
| CD8-targeted | | PEVKFNWYVDGVEVHNAKTKPREEQYNSTYRVVSVLTVLHQDWLNGKEY |
| format (2nd set of | | KCKVSNKALGAPIEKTISKAKGQPREPQVCTLPPSRDELTKNQVSLSCAVK |
| pH-dep IL2v | | GFYPSDIAVEWESNGQPENNYKTTPPVLDSDGSFFLVSKLTVDKSRWQQG |
| variants) | | NVFSCSVMHEALHNHYTQKSLSLSPGK |
| ‘long’ HC for 2nd | 81 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0079 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLDDLEQILNGINNYKNPKLTRMLT |
| | | AKFAMPKKATELKHLQCLEEELKPLEEVLNGAQSKNFHLDPRELIDNINVIV |
| | | LELKGSETTFMCEYADETATIVEFLNHWITFAQSIIETLE |
| ‘long’ HC for 2nd | 82 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0087 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTEEQLEQLLDDLENILNGINNYKNPKLTRMLT |
| | | AKFAMPKKATELKHLQCLEEELKPLEEVLNGAQSKNFHLEPRELIENINVIVL |
| | | ELKGSETTFMCEYADETATIVEFLNHWITFAQSIIETLE |
| ‘long’ HC for 2nd | 83 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173 | | CNVNHKPSNTKVDKKVEPKSCDKTHTCPPCPAPEAAGGPSVFLFPPKPKD |
| 0127. | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEELLDDLQEILNGINNYKNPKLTRMLT |
| | | AKFAMPKKATELKHLQCLEEELKPLEEVLNGAQSKNFHLDPRDLIENINVIV |
| | | LELKGSETTFMCEYADETATIVEFLNHWITFAESIISTLD |
| ‘long’ HC for 2nd | 84 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0156 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTESQLQNLLDDLQEILNGINNYKNPKLTRMLT |
| | | AKFAMPKKATELKHLQCLEEELKPLEEVLNGAQSKNFHLEPRELIDNINVIVL |
| | | ELKGSETTFMCEYADETATIVEFLNHWITFAQSIIETLE |
| ‘long’ HC for 2nd | 85 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0182 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEELLLDLQEILNGINNYKNPKLTRMLT |
| | | AKFAMPKKATELKHLQCLEEELKPLEEVLNGAQSKNFHLNPRELIENINVIVL |
| | | ELKGSETTFMCEYADETATIVEFLNHWITFAESIIETLE |
| ‘long’ HC for 2nd | 86 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0239 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLLDLEEILNGINNYKNPKLTRMLT |
| | | AKFAMPKKATELKHLQCLEEELKPLEEVLNGAQSKNFHLHPRELIENINVIVL |
| | | ELKGSETTFMCEYADETATIVEFLNHWITFAQSIIETLD |
| ‘long’ HC for 2nd | 87 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0255 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTEDHLQELLLDLEEILNGINNYKNPKLTRMLT |
| | | AKFAMPKKATELKHLQCLEEELKPLEEVLNGAQSKNFHLNPRELIENINVIVL |
| | | ELKGSETTFMCEYADETATIVEFLNHWITFAQSIIETLE |
| ‘long’ HC for 2nd | 88 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0259 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLDDLQMILNGINNYKNPKLTRML |
| | | TAKFAMPKKATELKHLQCLEEELKPLEEVLNGAQSKNFHLEPRDLIENINVIV |
| | | LELKGSETTFMCEYADETATIVEFLNHWITFAQSIIETLE |
| ‘long’ HC for 2nd | 89 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173. | | CNVNHKPSNTKVDKKVEPKSCDKTHTCPPCPAPEAAGGPSVFLFPPKPKD |
| 0371 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLDDLQQILNGINNYKNPKLTRMLT |
| | | AKFAMPKKATELKHLQCLEEELKPLEEVLNGAQSKNFHLNPRELIDNINVIV |
| | | LELKGSETTFMCEYADETATIVEFLNHWITFAQSIIETLD |
| ‘long’ HC for 2nd | 90 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0173 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTETQLQELLDDLHMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNHWITFAQSIIETLE |
| ‘long’ HC for 2nd | 91 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0238 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTETQLQELLDDLHEILNGINNYKNPKLTRMLT |
| | | AKFAMPKKATELKHLQCLEEELKPLEEVLNGAQSKNFHLEPRELIENINVIVL |
| | | ELKGSETTFMCEYADETATIVEFLNHWITFAQSIIETLE |
| ‘long’ HC for 2nd | 92 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0277 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLDDLQMILNGINNYKNPKLTRML |
| | | TAKFAMPKKATELKHLQCLEEELKPLEEVLNGAQSKNFHLEPRELIENINVIV |
| | | LELKGSETTFMCEYADETATIVEFLNHWITFAQSIIETLE |
| ‘long’ HC for 2nd | 93 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0281 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLLDLEQILNGINNYKNPKLTRMLT |
| | | AKFAMPKKATELKHLQCLEEELKPLEEVLNGAQSKNFHLRPRDLINNINVIV |
| | | LELKGSETTFMCEYADETATIVEFLNHWITFAQSIIETLE |
| ‘long’ HC for 2nd | 94 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0326 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLDDLDQILNGINNYKNPKLTRMLT |
| | | AKFAMPKKATELKHLQCLEEELKPLEEVLNGAQSKNFHLDPRELIENINVIVL |
| | | ELKGSETTFMCEYADETATIVEFLNHWITFAQSIIETLE |
| ‘long’ HC for 2nd | 95 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0327 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTEEHLQNLLDDLEQILNGINNYKNPKLTRMLT |
| | | AKFAMPKKATELKHLQCLEEELKPLEEVLNGAQSKNFHLEPRELIDNINVIVL |
| | | DLKGSETTFMCEYADETATIVEFLNHWITFAQSIISTLE |
| ‘long’ HC for 2nd | 96 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P175. | | CNVNHKPSNTKVDKKVEPKSCDKTHTCPPCPAPEAAGGPSVFLFPPKPKD |
| 0125 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTETQLEELLDDLEMILNGINNYKNPKLTRMLT |
| | | AKFAMPKKATELKHLQCLEEELKPLEEVLNGAQSKNFHLDPRELIENINVIVL |
| | | ELKGSETTFMCEYADETATIVEFLNHWITFAQSIIETLD |
| ‘long’ HC for 2nd | 97 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P175. | | CNVNHKPSNTKVDKKVEPKSCDKTHTCPPCPAPEAAGGPSVFLFPPKPKD |
| 0368 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTTEQLQELLDDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNEWITFAQSIIETLD |
| ‘long’ HC for 2nd | 98 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P177. | | CNVNHKPSNTKVDKKVEPKSCDKTHTCPPCPAPEAAGGPSVFLFPPKPKD |
| 0035 (IL2v | | TLMISRTPEVTCVVVDVSHEDPEVKFNWYVDGVEVHNAKTKPREEQYNST |
| (consensus all)) | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLDDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNHWITFAQSIIETLE |
| ‘long’ HC for 2nd | 99 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P177. | | CNVNHKPSNTKVDKKVEPKSCDKTHTCPPCPAPEAAGGPSVFLFPPKPKD |
| 0036 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLDDLQMILNGINNYKNPKLTRML |
| | | TAKFAMPKKATELKHLQCLEEELKPLEEVLNGAQSKNFHLQPRELIENINVIV |
| | | LELKGSETTFMCEYADETATIVEFLNHWITFAQSIIETLE |
| ‘long’ HC for 2nd | 100 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P177. | | CNVNHKPSNTKVDKKVEPKSCDKTHTCPPCPAPEAAGGPSVFLFPPKPKD |
| 0156 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTESQLEELLDDLQQILNGINNYKNPKLTRMLT |
| | | AKFAMPKKATELKHLQCLEEELKPLEEVLNGAQSKNFHLDPRELIENINVIVL |
| | | ELKGSETTFMCEYADETATIVEFLNHWITFAQSIIETLE |
| ‘long’ HC for 2nd | 101 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P172. | | CNVNHKPSNTKVDKKVEPKSCDKTHTCPPCPAPEAAGGPSVFLFPPKPKD |
| 0344 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSYTKKTQERLEQLLLDLEQILNGINNYKNPKLTRMLT |
| | | AKFAMPKKATELKHLQCLEEELKPLEEVLNGAQSKNFHLNPRELIENINVIVL |
| | | ELKGSETTFMCEYADETATIVEFLNHWITFAQSIIETLD |
| ‘long’ HC for 2nd | 102 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P173 | | CNVNHKPSNTKVDKKVEPKSCDKTHTCPPCPAPEAAGGPSVFLFPPKPKD |
| 0364 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEDLLLDLQNILNGINNYKNPKLTRMLT |
| | | AKFAMPKKATELKHLQCLEEELKPLEEVLNGAQSKNFHLDPRELISNINVIVL |
| | | ELKGSETTFMCEYADETATIVEFLNHWITFAQSIIETLE |
| ‘long’ HC for 2nd | 103 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P174. | | CNVNHKPSNTKVDKKVEPKSCDKTHTCPPCPAPEAAGGPSVFLFPPKPKD |
| 0040 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTETQLEQLLDDLQEILNGINNYKNPKLTRMLT |
| | | AKFAMPKKATELKHLQCLEEELKPLEEVLNGAQSKNFHLDPRELIENINVIVL |
| | | ELKGSETTFMCEYADETATIVEFLNHWITFAQSIIETLE |
| ‘long’ HC for 2nd | 104 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P178. | | CNVNHKPSNTKVDKKVEPKSCDKTHTCPPCPAPEAAGGPSVFLFPPKPKD |
| 0145 | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTEEQLENLLLDLQNILNGINNYKNPKLTRMLT |
| | | AKFAMPKKATELKHLQCLEEELKPLEEVLNGAQSKNFHLEPRELISNINVIVL |
| | | ELKGSETTFMCEYADETATIVEFLNHWITFAQSIIETLE |
| ‘long’ HC for 2nd | 105 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (Q11E) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 106 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (E15Q) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLQHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 107 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (H16E) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 108 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (L19D) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLDDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 109 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (Q22E) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLEMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 110 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (M23Q) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQQILNGINNYKNPKLTRMLT |
| | | AKFAMPKKATELKHLQCLEEELKPLEEVLNGAQSKNFHLRPRDLISNINVIVL |
| | | ELKGSETTFMCEYADETATIVEFLNRWITFAQSIISTLT |
| ‘long’ HC for 2nd | 111 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (R81D) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLDPRDLISNINVI |
| | | VLELKGSETTFMCEYADETATIVEFLNRWITFAQSIISTLT |
| ‘long’ HC for 2nd | 112 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (D84E) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRELISNINVIV |
| | | LELKGSETTFMCEYADETATIVEFLNRWITFAQSIISTLT |
| ‘long’ HC for 2nd | 113 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (S87E) | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLIENINVI |
| | | VLELKGSETTFMCEYADETATIVEFLNRWITFAQSIISTLT |
| ‘long’ HC for 2nd | 114 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (R120H) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNHWITFAQSIISTLT |
| ‘long’ HC for 2nd | 115 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (Q126E) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAESIISTLT |
| ‘long’ HC for 2nd | 116 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (Q126H) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAHSIISTLT |
| ‘long’ HC for 2nd | 117 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (S130E) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIIETLT |
| ‘long’ HC for 2nd | 118 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (T133E) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLE |
| ‘long’ HC for 2nd | 119 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (T133D) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLD |
| ‘long’ HC for 2nd | 120 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (consensus beta) | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLQELLDDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNRWITFAQSIISTLT |
| ‘long’ HC for 2nd | 121 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (consensus | | CNVNHKPSNTKVDKKVEPKSCDKTHTCPPCPAPEAAGGPSVFLFPPKPKD |
| gamma) | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNHWITFAQSIIETLD |
| ‘long’ HC for 2nd | 122 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with template for | | FPEPVTVSWNSGALTSGVHTFPAVLQSSGLYSLSSVVTVPSSSLGTQTYI |
| 2^{nd} library | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNHWITFAQSIIETLD |
| ‘long’ HC for 2nd | 123 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLT |
| ‘long’ HC for 2nd | 124 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (Q11E) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| ‘long’ HC for 2nd | 125 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (E15Q) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLQHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| ‘long’ HC for 2nd | 126 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (H16E) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEELLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| ‘long’ HC for 2nd | 127 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (L19D) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLDDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| ‘long’ HC for 2nd | 128 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (Q22E) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLEMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| ‘long’ HC for 2nd | 129 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (M23Q) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQQILNGINNYKNPKLTRMLT |
| | | AKFAMPKKATELKHLQCLEEELKPLEEVLNGAQSKNFHLRPRDLISNINVIVL |
| | | ELKGSETTFMCEYADETATIVEFLNRWITFAQSIISTLTA |
| ‘long’ HC for 2nd | 130 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (R81D) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLDPRDLISNINVI |
| | | VLELKGSETTFMCEYADETATIVEFLNRWITFAQSIISTLTA |
| ‘long’ HC for 2nd | 131 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (D84E) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRELISNINVIV |
| | | LELKGSETTFMCEYADETATIVEFLNRWITFAQSIISTLTA |
| ‘long’ HC for 2nd | 132 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v (S87E) | | FPEPVTVSWNSGALTSGVHTFPAVLQSSGLYSLSSVVTVPSSSLGTQTYI |
| w/ C-terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLIENINVI |
| | | VLELKGSETTFMCEYADETATIVEFLNRWITFAQSIISTLTA |
| ‘long’ HC for 2nd | 133 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (R120H) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNHWITFAQSIISTLTA |
| ‘long’ HC for 2nd | 134 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (Q126E) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAESIISTLTA |
| ‘long’ HC for 2nd | 135 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (Q126H) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAHSIISTLTA |
| ‘long’ HC for 2nd | 136 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (S130E) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIIETLTA |
| ‘long’ HC for 2nd | 137 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (T133E) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLEA |
| ‘long’ HC for 2nd | 138 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (T133D) w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLDA |
| ‘long’ HC for 2nd | 139 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (consensus beta) | | CNVNHKPSNTKVDKKVEPKSCDKTHTCPPCPAPEAAGGPSVFLFPPKPKD |
| w/ C-terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLQELLDDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNRWITFAQSIISTLTA |
| ‘long’ HC for 2nd | 140 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v | | FPEPVTVSWNSGALTSGVHTFPAVLQSSGLYSLSSVVTVPSSSLGTQTYI |
| (consensus | | CNVNHKPSNTKVDKKVEPKSCDKTHTCPPCPAPEAAGGPSVFLFPPKPKD |
| gamma) w/ C- | | TLMISRTPEVTCVVVDVSHEDPEVKFNWYVDGVEVHNAKTKPREEQYNST |
| terminal ala | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNHWITFAQSIIETLDA |
| ‘long’ HC for 2nd | 141 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with template for | | FPEPVTVSWNSGALTSGVHTFPAVLQSSGLYSLSSVVTVPSSSLGTQTYI |
| 2^{nd} library w/ C- | | CNVNHKPSNTKVDKKVEPKSCDKTHTCPPCPAPEAAGGPSVFLFPPKPKD |
| terminal ala | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLEHLLLDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNHWITFAQSIIETLDA |
| ‘long’ HC for 2nd | 142 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with | | FPEPVTVSWNSGALTSGVHTFPAVLQSSGLYSLSSVVTVPSSSLGTQTYI |
| ExplResOnc.P177. | | CNVNHKPSNTKVDKKVEPKSCDKTHTCPPCPAPEAAGGPSVFLFPPKPKD |
| 0035 (IL2v | | TLMISRTPEVTCVVVDVSHEDPEVKFNWYVDGVEVHNAKTKPREEQYNST |
| (consensus all)) w/ | | YRVVSVLTVLHQDWLNGKEYKCKVSNKALGAPIEKTISKAKGQPREPQVY |
| C-terminal ala | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTELQLQELLDDLQMILNGINNYKNPKLTRML |
| | | TAKFAMPKKATELKHLQCLEEELKPLEEVLNGAQSKNFHLDPRELIENINVIV |
| | | LELKGSETTFMCEYADETATIVEFLNHWITFAQSIIETLEA |
| ‘long’ HC for 2nd | 143 | EVQLVQSGAEVKKPGASVKVSCKASGFNIKDTYIHWVRQAPGQGLEWIGR |
| set of CD8- | | IDPANDNTLYASKFQGRATITADTSTSTAYLELSSLRSEDTAVYYCGRGY |
| targeted formats | | GYYVFDHWGQGTLVTVSSASTKGPSVFPLAPSSKSTSGGTAALGCLVKDY |
| with IL2v w/ C- | | FPEPVTVSWNSGALTSGVHTFPAVLQSSGLYSLSSVVTVPSSSLGTQTYI |
| terminal ala | | CNVNHKPSNTKVDKKVEPKSCDKTHTCPPCPAPEAAGGPSVFLFPPKPKD |
| | | TLMISRTPEVTCVVVDVSHEDPEVKFNWYVDGVEVHNAKTKPREEQYNST |
| | | YRVVSVLTVLHQDWLNGKEYKCKVSNKALGAPIEKTISKAKGQPREPQVY |
| | | TLPPCRDELTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGGGGG |
| | | SGGGGSGGGGGAPASSSTKKTQLQLEHLLLDLQMILNGINNYKNPKLTRML |
| | | TAKFAMPKKATELKHLQCLEEELKPLEEVLNGAQSKNFHLRPRDLISNINVIV |
| | | LELKGSETTFMCEYADETATIVEFLNRWITFAQSIISTLTA |
| human IL-2 wild | 144 | APTSSSTKKTQLQLEHLLLDLQMILNGINNYKNPKLTRMLTFKFYMPKKATE |
| type | | LKHLQCLEEELKPLEEVLNLAQSKNFHLRPRDLISNINVIVLELKGSETTFMC |
| | | EYADETATIVEFLNRWITFCQSIISTLT |
| LC for PD1- | 145 | DIVMTQSPDSLAVSLGERATINCKASESVDTSDNSFIHWYQQKPGQSPKLLIY |
| targeted | | RSSTLESGVPDRFSGSGSGTDFTLTISSLQAEDVAVYYCQQNYDVPWTFGQG |
| format(short-listed | | TKVEIKRTVAAPSVFIFPPSDEQLKSGTASVVCLLNNFYPREAKVQWKVDNA |
| from 2nd set of | | LQSGNSQESVTEQDSKDSTYSLSSTLTLSKADYEKHKVYACEVTHQGLSSPV |
| pH-dep IL2v | | TKSFNRGEC |
| variants) | | |
| ‘hole’ HC of PD1- | 146 | EVQLLESGGGLVQPGGSLRLSCAASGFSFSSYTMSWVRQAPGKGLEWVATI |
| targeted format | | SGGGRDIYYPDSVKGRFTISRDNSKNTLYLQMNSLRAEDTAVYYCVLLTGR |
| short-listed from | | VYFALDSWGQGTLVTVSSASTKGPSVFPLAPSSKSTSGGTAALGCLVKDYFP |
| 2nd set of pH-dep | | EPVTVSWNSGALTSGVHTFPAVLQSSGLYSLSSVVTVPSSSLGTQTYICNVN |
| IL2v variants | | HKPSNTKVDKKVEPKSCDKTHTCPPCPAPEAAGGPSVFLFPPKPKDTLMISRT |
| | | PEVTCVVVDVSHEDPEVKFNWYVDGVEVHNAKTKPREEQYNSTYRVVSVL |
| | | TVLHQDWLNGKEYKCKVSNKALGAPIEKTISKAKGQPREPQVCTLPPSRDEL |
| | | TKNQVSLSCAVKGFYPSDIAVEWESNGQPENNYKTTPPVLDSDGSFFLVSKL |
| | | TVDKSRWQQGNVFSCSVMHEALHNHYTQKSLSLSPG |
| ‘knob’ HC of | 147 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P172.0344) | | TVLHQDWLNGKEYKCKVSNKALGAPIEKTISKAKGQPREPQVYTLPPCRDE |
| P1AG6055 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSYTKKTQERLEQLLLDLEQILNGINNYKNPKLTRMLTAKFAMPKK |
| | | ATELKHLQCLEEELKPLEEVLNGAQSKNFHLNPRELIENINVIVLELKGSETTF |
| | | MCEYADETATIVEFLNHWITFAQSIIETLD |
| ‘knob’ HC of | 148 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P173.0364) | | TVLHQDWLNGKEYKCKVSNKALGAPIEKTISKAKGQPREPQVYTLPPCRDE |
| P1AG6057 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTQLQLEDLLLDLQNILNGINNYKNPKLTRMLTAKFAMPKK |
| | | ATELKHLQCLEEELKPLEEVLNGAQSKNFHLDPRELISNINVIVLELKGSETTF |
| | | MCEYADETATIVEFLNHWITFAQSIIETLE |
| ‘knob’ HC of | 149 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0040) | | TVLHQDWLNGKEYKCKVSNKALGAPIEKTISKAKGQPREPQVYTLPPCRDE |
| P1AG6061 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTETQLEQLLDDLQEILNGINNYKNPKLTRMLTAKFAMPKK |
| | | ATELKHLQCLEEELKPLEEVLNGAQSKNFHLDPRELIENINVIVLELKGSETTF |
| | | MCEYADETATIVEFLNHWITFAQSIIETLE |
| ‘knob’ HC of | 150 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0173) | | TVLHQDWLNGKEYKCKVSNKALGAPIEKTISKAKGQPREPQVYTLPPCRDE |
| P1AG6051 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTETQLQELLDDLHMILNGINNYKNPKLTRMLTAKFAMPKK |
| | | ATELKHLQCLEEELKPLEEVLNGAQSKNFHLDPRELIENINVIVLELKGSETTF |
| | | MCEYADETATIVEFLNHWITFAQSIIETLE |
| ‘knob’ HC of | 151 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0238) | | TVLHQDWLNGKEYKCKVSNKALGAPIEKTISKAKGQPREPQVYTLPPCRDE |
| P1AG6058 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTETQLQELLDDLHEILNGINNYKNPKLTRMLTAKFAMPKK |
| | | ATELKHLQCLEEELKPLEEVLNGAQSKNFHLEPRELIENINVIVLELKGSETTF |
| | | MCEYADETATIVEFLNHWITFAQSIIETLE |
| ‘knob' HC of | 152 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0281) | | TVLHQDWLNGKEYKCKVSNKALGAPIEKTISKAKGQPREPQVYTLPPCRDE |
| P1AG6059 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTQLQLEELLLDLEQILNGINNYKNPKLTRMLTAKFAMPKKA |
| | | TELKHLQCLEEELKPLEEVLNGAQSKNFHLRPRDLINNINVIVLELKGSETTF |
| | | MCEYADETATIVEFLNHWITFAQSIIETLE |
| ‘knob’ HC of | 153 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0326) | | TVLHQDWLNGKEYKCKVSNKALGAPIEKTISKAKGQPREPQVYTLPPCRDE |
| P1AG6054 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTQLQLEELLDDLDQILNGINNYKNPKLTRMLTAKFAMPKK |
| | | ATELKHLQCLEEELKPLEEVLNGAQSKNFHLDPRELIENINVIVLELKGSETTF |
| | | MCEYADETATIVEFLNHWITFAQSIIETLE |
| ‘knob’ HC of | 154 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P174.0327) | | TVLHQDWLNGKEYKCKVSNKALGAPIEKTISKAKGQPREPQVYTLPPCRDE |
| P1AG6053 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTEEHLQNLLDDLEQILNGINNYKNPKLTRMLTAKFAMPKK |
| | | ATELKHLQCLEEELKPLEEVLNGAQSKNFHLEPRELIDNINVIVLDLKGSETT |
| | | FMCEYADETATIVEFLNHWITFAQSIISTLE |
| ‘knob’ HC of | 155 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P175.0125) | | TVLHQDWLNGKEYKCKVSNKALGAPIEKTISKAKGQPREPQVYTLPPCRDE |
| P1AG6052 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTETQLEELLDDLEMILNGINNYKNPKLTRMLTAKFAMPKK |
| | | ATELKHLQCLEEELKPLEEVLNGAQSKNFHLDPRELIENINVIVLELKGSETTF |
| | | MCEYADETATIVEFLNHWITFAQSIIETLD |
| ‘knob’ HC of | 156 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P177.0035 (IL2v | | TVLHQDWLNGKEYKCKVSNKALGAPIEKTISKAKGQPREPQVYTLPPCRDE |
| (consensus all))) | | LTKNQVSLWCLVKGFYPSDIAVEWESNGQPENNYKTTPPVLDSDGSFFLYSK |
| P1AG6060 | | LTVDKSRWQQGNVFSCSVMHEALHNHYTQKSLSLSPGGGGGSGGGGSGGG |
| | | GGAPASSSTKKTELQLQELLDDLQMILNGINNYKNPKLTRMLTAKFAMPKK |
| | | ATELKHLQCLEEELKPLEEVLNGAQSKNFHLDPRELIENINVIVLELKGSETTF |
| | | MCEYADETATIVEFLNHWITFAQSIIETLE |
| ‘knob’ HC of | 157 | EVQLLESGGGLVQPGGSLRLSCAASGFSFSSYTMSWVRQAPGKGLEWVATI |
| PD1-targeted | | SGGGRDIYYPDSVKGRFTISRDNSKNTLYLQMNSLRAEDTAVYYCVLLTGR |
| format short- | | VYFALDSWGQGTLVTVSSASTKGPSVFPLAPSSKSTSGGTAALGCLVKDYFP |
| listed from 2nd set | | EPVTVSWNSGALTSGVHTFPAVLQSSGLYSLSSVVTVPSSSLGTQTYICNVN |
| of pH-dep IL2v | | HKPSNTKVDKKVEPKSCDKTHTCPPCPAPEAAGGPSVFLFPPKPKDTLMISRT |
| variants with IL2v | | PEVTCVVVDVSHEDPEVKFNWYVDGVEVHNAKTKPREEQYNSTYRVVSVL |
| (P178.0145) | | TVLHQDWLNGKEYKCKVSNKALGAPIEKTISKAKGQPREPQVYTLPPCRDE |
| P1AG6056 | | LTKNQVSLWCLVKGFYPSDIAVEWESNGQPENNYKTTPPVLDSDGSFFLYSK |
| | | LTVDKSRWQQGNVFSCSVMHEALHNHYTQKSLSLSPGGGGGSGGGGSGGG |
| | | GGAPASSSTKKTEEQLENLLLDLQNILNGINNYKNPKLTRMLTAKFAMPKK |
| | | ATELKHLQCLEEELKPLEEVLNGAQSKNFHLEPRELISNINVIVLELKGSETTF |
| | | MCEYADETATIVEFLNHWITFAQSIIETLE |
| ‘empty’ HC hole | 158 | DKTHTCPPCPAPEAAGGPSVFLFPPKPKDTLMISRTPEVTCVVVDVSHEDPEV |
| for non-targeted | | KFNWYVDGVEVHNAKTKPREEQYNSTYRVVSVLTVLHQDWLNGKEYKCK |
| Fc-wild-type IL2 | | VSNKALGAPIEKTISKAKGQPREPQVCTLPPSRDELTKNQVSLSCAVKGFYPS |
| fusions | | DIAVEWESNGQPENNYKTTPPVLDSDGSFFLVSKLTVDKSRWQQGNVFSCS |
| | | VMHEALHNHYTQKSLSLSPG |
| HC knob for non- | 159 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc- | | SDIAVEWESNGQPENNYKTTPPVLDSDGSFFLYSKLTVDKSRWQQGNVFSCS |
| IL2wt_pH_P174- | | VMHEALHNHYTQKSLSLSPGGGGGSGGGGSGGGGGAPASSSTKKTETQLEQ |
| 0040) P1AG7461 | | LLDDLQEILNGINNYKNPKLTRMLTFKFYMPKKATELKHLQCLEEELKPLEE |
| | | VLNLAQSKNFHLDPRELIENINVIVLELKGSETTFMCEYADETATIVEFLNHW |
| | | ITFAQSIIETLE |
| HC knob for non- | 160 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc- | | SDIAVEWESNGQPENNYKTTPPVLDSDGSFFLYSKLTVDKSRWQQGNVFSCS |
| IL2wt_pH_P174- | | VMHEALHNHYTQKSLSLSPGGGGGSGGGGSGGGGGAPASSSTKKTQLQLEE |
| 0326) P1AG7462 | | LLDDLDQILNGINNYKNPKLTRMLTFKFYMPKKATELKHLQCLEEELKPLEE |
| | | VLNLAQSKNFHLDPRELIENINVIVLELKGSETTFMCEYADETATIVEFLNHW |
| | | ITFAQSIIETLE |
| HC knob for non- | 161 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc-wild- | | SDIAVEWESNGQPENNYKTTPPVLDSDGSFFLYSKLTVDKSRWQQGNVFSCS |
| type IL2) | | VMHEALHNHYTQKSLSLSPGGGGGSGGGGSGGGGGAPASSSTKKTQLQLE |
| P1AG7463 | | HLLLDLQMILNGINNYKNPKLTRMLTFKFYMPKKATELKHLQCLEEELKPLE |
| | | EVLNLAQSKNFHLRPRDLISNINVIVLELKGSETTFMCEYADETATIVEFLNR |
| | | WITFAQSIISTLT |
| HC knob for non- | 162 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc- | | SDIAVEWESNGQPENNYKTTPPVLDSDGSFFLYSKLTVDKSRWQQGNVFSCS |
| IL2wt_pH_P172- | | VMHEALHNHYTQKSLSLSPGGGGGSGGGGSGGGGGAPASSYTKKTQERLE |
| 0344) P1AG7464 | | QLLLDLEQILNGINNYKNPKLTRMLTFKFYMPKKATELKHLQCLEEELKPLE |
| | | EVLNLAQSKNFHLNPRELIENINVIVLELKGSETTFMCEYADETATIVEFLNH |
| | | WITFAQSIIETLD |
| HC knob for non- | 163 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc- | | SDIAVEWESNGQPENNYKTTPPVLDSDGSFFLYSKLTVDKSRWQQGNVFSCS |
| IL2wt_pH_P175- | | VMHEALHNHYTQKSLSLSPGGGGGSGGGGSGGGGGAPASSSTKKTETQLEE |
| 0125) P1AG7465 | | LLDDLEMILNGINNYKNPKLTRMLTFKFYMPKKATELKHLQCLEEELKPLEE |
| | | VLNLAQSKNFHLDPRELIENINVIVLELKGSETTFMCEYADETATIVEFLNHW |
| | | ITFAQSIIETLD |
| HC knob for non- | 164 | DKTHTCPPCPAPEAAGGPSVFLFPPKPKDTLMISRTPEVTCVVVDVSHEDPEV |
| targeted Fc- | | KFNWYVDGVEVHNAKTKPREEQYNSTYRVVSVLTVLHQDWLNGKEYKCK |
| wild-type IL2 | | VSNKALGAPIEKTISKAKGQPREPQVYTLPPCRDELTKNQVSLWCLVKGFYP |
| fusions (Fc- | | SDIAVEWESNGQPENNYKTTPPVLDSDGSFFLYSKLTVDKSRWQQGNVFSCS |
| IL2wt_pH_P174- | | VMHEALHNHYTQKSLSLSPGGGGGSGGGGSGGGGGAPASSSTKKTETQLQE |
| 0238) P1AG7466 | | LLDDLHEILNGINNYKNPKLTRMLTFKFYMPKKATELKHLQCLEEELKPLEE |
| | | VLNLAQSKNFHLEPRELIENINVIVLELKGSETTFMCEYADETATIVEFLNHW |
| | | ITFAQSIIETLE |

### EXAMPLES

The following are examples of methods and compositions of the invention. It is understood that various other embodiments may be practiced, given the general description provided above.

### Example 1

### Preparation of Antigens and Cloning of IL2v

Recombinant DNA Techniques

Standard methods were used to manipulate DNA as described in Sambrook, J. et al, Molecular cloning: A laboratory manual; Cold Spring Harbor Laboratory press, Cold spring Harbor, New York, 1989. The molecular biological reagents were used according to the manufacturer's instructions. General information regarding the nucleotide sequences of human immunoglobulin light and heavy chains is given in: Kabat, E. A. et al. (1991) Sequences of Proteins of Immunological Interest, Fifth Ed., NIH Publication No 91-3242.

### Gene Synthesis

Desired gene segments, where required, were either generated by PCR using appropriate templates or were synthesized at Geneart AG (Regensburg, Germany) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the sub-cloned gene fragments was confirmed by DNA sequencing. Gene segments were designed with suitable restriction sites to allow sub-cloning into the respective expression vectors.

Cloning of the Antigen Expression Vector

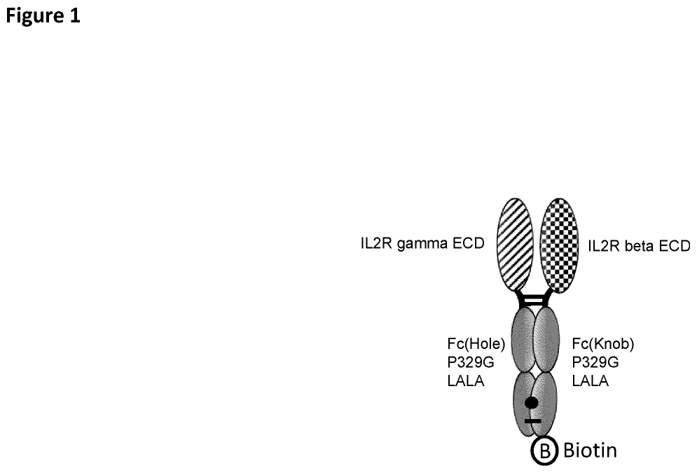

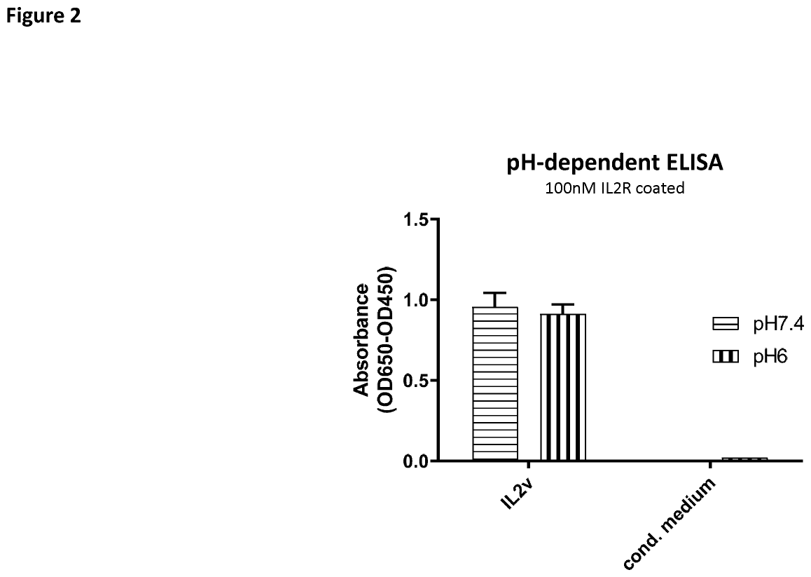

For the generation of an IL2 receptor construct consisting of the beta and the gamma subunit (IL2Rbg), a Fc-based knob-into-hole fusion construct was generated and expressed. While the extracellular domain (ECD) of the IL2R beta unit was fused to the N-terminal end the Fc-knob chain, the ECD of the gamma unit was fused to N-terminal end of the Fc-hole chain (SEQ ID NOs 2 and 3). An avi tag (GLNDIFEAQKIEWHE) fused to the C-terminal end of the Fc-knob chain allowed specific biotinylation during co-expression with BirA biotin ligase. FIG. 1 shows a schematic representation of the construct.

Production and Purification of Fc Fusion Constructs in Eukaryotic Cells

The IL2R(bg)-Fc construct (SEQ ID NOs 2 and 3) was generated by transient transfection of HEK293 EBNA cells. Cells were centrifuged and medium was replaced by pre-warmed CD CHO medium (Thermo Fisher, Cat No 10743029). Expression vectors were mixed in CD CHO medium, PEI (Polyethylenimine, Polysciences, Inc, Cat No 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% CO₂ atmosphere. A simultaneously co-transfected plasmid encoding the biotin ligase BirA allowed avi-tag-specific biotinylation in vivo. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter).

The protein was purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art. Nr.: UFC903096)), and aggregated protein was separated from monomeric protein by size exclusion chromatography in 20 mM histidine, 140 mM sodium chloride, pH 6.0. Biotinylation was confirmed by adding streptavidin. The resulting biotinylated protein/streptavidin complex showed a shift of the retention time in the analytical SEC chromatogram

Cloning of IL2v into Phagemid Vector

The DNA sequence encoding IL2v (SEQ ID NO: 4) was amplified by PCR and cloned into our phagemid vector using the restriction enzymes NcoI/NotI. This cloning resulted in the transcription/translation of the bacterial PelB signal sequence followed by IL2v sequence and the C-terminally fused His tag and Flag tag. An amber stop codon between the tags and the phage-derived DNA sequence encoding the p3 protein allowed expression of both soluble IL2v and IL2v displayed on the M13 phage.

Evaluation of IL2v Binding to IL2R at pH 7.4 and pH 6

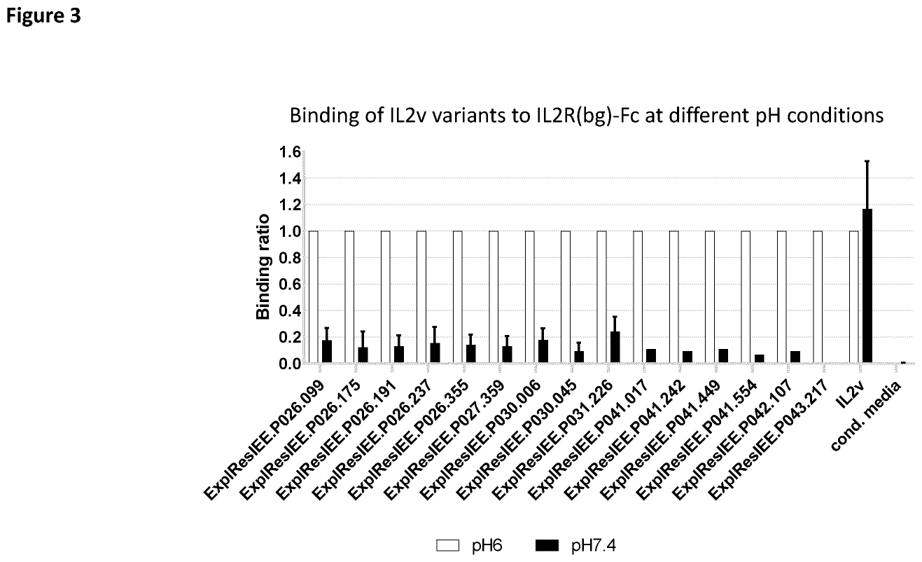

In order to evaluate if IL2v shows any pH-dependent behavior in an ELISA, bacterial supernatant containing soluble IL2v was generated by IPTG induction of exponentially growing TG1 bacteria harboring the previously generated IL2v phagemid. Bacterial supernatant was harvested by centrifugation and pH was adjusted to either pH 6 or pH 7.4 using Bis-Tris buffer (final concentration: 50 mM BisTris, 140 mM NaCL). Supernatant was incubated in Neutravidin plates that were previously coated with the biotinylated IL2R(bg)-Fc construct and blocked with BSA. While extensive washing after 1 h was performed with Bis-Tris washing buffer at the respective pH 6 or 7.4 (50 mM BisTris, 140 mM NaCL, 0.1% Tween), all subsequent steps were performed at pH 6 to avoid any pH-dependent secondary effects. Binding of IL2v to the IL2R(bg)-Fc construct was detected via Flag tag by using an anti-Flag/HRP secondary antibody. Comparison of the signal between the two conditions (pH 6 vs pH 7.4) revealed an equally strong signal concluding that the parental IL2v protein shows a pH-independent behavior to its receptor (FIG. 2).

### Example 2

Design of a pH-Dependent IL2v Library Based on IL2v

The crystal structures of IL-2 (PDB codes: 2b5i and 5m5e) were used as a basis. The interface between the IL-2 and the IL2 receptor beta and gamma chains (IL2Rbg) was analyzed in PyMol. Special emphasis was put on amino acids of the receptor that carry sidechains with pH titratable properties, particularly Hydrogen bond donors or acceptors. IL2 residues positioned within a radius of <8 Angstrom of those pH titratable residues on the receptor subunits were considered for pH engineering. Since IL2 was previously displayed on a bacteriophage (Buchli et al., Arch Biochem Biophys 1997; 339:79-84; Vispo et al., Immunotechnology Int J Immunol Eng 1997; 3:185-93), a similar approach was applied to introduce pH dependent binding properties.

We confirmed beforehand that IL2v (SEQ ID NO: 4) does not show intrinsic pH dependent binding properties. Since this would affect the library design and phage selection scheme. FIG. 2 shows that IL2v behaves identical at pH 7.4 and 6.0 with respect to in vitro IL2Rbg binding. In contrast, Fallon and coworkers have identified pH dependency in IL2, such that IL2 binds with higher affinity at pH 7.2 over pH 6 (Fallon et al., J. Biol. Chem. 275, 6790-6797 (2000).

An initial combinatorial library was generated for phage display that include randomized amino acids at the following positions: K8, Q11, L12, L19, D20, M23, R81, D84, S87, N88, V91, E95, R120, T123, Q126, S130, T133 (Numbering as in UniProt entry P60568 having the signal peptide cleft off). Randomization was chosen in a way that the resulting residues might form hydrogen bonds or salt bridges to the IL2 receptor chains in a pH-dependent fashion.

Generation of Two pH-Dependent IL2v Libraries

For the generation of two phage display libraries based on the sequence of IL2v and randomization in the interface of the receptor sub-units beta and gamma, the respective DNA fragments encompassing the randomized IL2v sequences and flanked by the restriction sites NcoI and NotI were synthesized. The fragments were digested with NcoI/NotI alongside with equally cleaved a parental IL2v acceptor phagemid vector. Library inserts were ligated with the phagemid vector over night at 4° C. Purified ligation products were used for bacterial transformations resulting in 5×10⁵ transformants for library 1 and 1.66×10¹⁰ transformant for library 2. Phagemid particles displaying the IL2v library were rescued and purified by PEG/NaCl purification to be further used for selections.

### Example 3

Selection of pH-Dependent IL2v Variants by Phage Display

The selection for pH-dependent IL2v variants was conducted using the recombinant biotinylated IL2R(bg)-Fc construct (SEQ ID NOs: 2 and 3). The selection steps were performed using Bis-Tris based buffer (50 mM BisTris, 140 mM NaCL) either at pH 6.0 or pH 7.4.

Panning rounds were performed in solution according to the following pattern: 1.) binding of approx. 10¹² phagemid particles to 100 nM biotinylated antigen protein for 0.5 h in a total volume of 1 ml; 2.) capture of biotinylated antigen and attachment of specifically binding phage by addition of 5.4×10⁷ streptavidin-coated magnetic beads for 10 min; 3.) washing of the beads using Bis-Tris based buffer: 4.) elution of phage particles. While incubation was performed at either pH 7.4 or pH 6, washing steps were always processed at pH 6. The bound phage particles were eluted by addition of either 1 ml 100 mM triethylamine (TEA) for 10 min or through a buffer change to pH 7.4.

The eluted phage was used for re-infection of exponentially growing E. coli TG1 cells. After super-infection with helperphage VCSM13 and subsequent PEG/NaCl precipitation, the isolated phagemid particles were used in subsequent selection rounds. In total, 4 selections rounds were carried out.

Identification of pH-Dependent IL2v Variants by ELISA

For the identification of pH-dependent IL2v variants, soluble IL2v-containing bacterial supernatants were generated and tested by ELISA as follows:

25 μl of 50 nM biotinylated antigen per well were coated at pH 6 on 384-well neutravidin plates and blocked with 2% BSA (50 mM BisTris 140 mM NaCl, 2% BSA, pH 6). Bacterial supernatants containing soluble variants of IL2v were re-buffered to pH 6, added to the plates and incubated for 1 h. Subsequently, several washing steps were performed at either pH 6 or pH 7.4 followed by an incubation step in washing buffer for 30 min at the respective pH. Binding of pH-dependent IL2v variants to the IL2R(bg)-Fc construct was detected via Flag tag by using an anti-Flag/HRP secondary antibody. Comparison of the signal between the two conditions (pH 6 vs pH 7.4) allowed identifying constructs that bind in a pH-dependent manner. The phagemid expressing the parental IL2v construct was used as an internal pH-independent control. Constructs showing the greatest binding ratio at pH 6 versus pH 7, i.e. strong binding at pH 6.0 and significantly reduced or no binding at pH 7.4. (FIG. 3), were selected (SEQ ID NOs 5-19) and converted into one-armed (OA) targeted conjugates.

Conversion of pH-Dependent IL2v Variants into One-Armed CD8-Targeted IgG IL2v Fusions—First Set of Variants from the First Phage Display Campaign

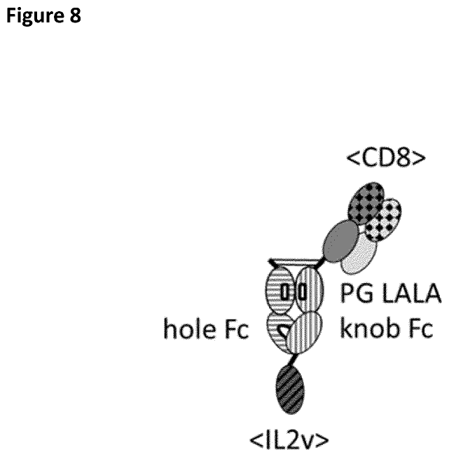

Fifteen pH-dependent IL2 variants (SEQ ID NOs 5-19) that had been selected by phage display for preferential binding at slightly acidic pH in the first campaign were converted into one-armed CD8-targeted IgG PG LALA IL2v fusions for expression in mammalian cells (CHO K1) and subsequent purification by protein A affinity chromatography and preparative size exclusion chromatography (SEC). The pH-dependent IL2v variants were fused to the C-terminus of the ‘knob’ heavy chain without terminal lysine (K) and connected via a (G45)3 glycine-serine linker. The resulting CD8-targeted IgG IL2v fusions were composed of SEQ ID NOs 62, 63 and 65; SEQ ID NOs 62, 63 and 66; SEQ ID NOs 62, 63 and 67; SEQ ID NOs 62, 63 and 68; SEQ ID NOs 62, 63 and 69; SEQ ID NOs 62, 63 and 70; SEQ ID NOs 62, 63 and 71; SEQ ID NOs 62, 63 and 72; SEQ ID NOs 62, 63 and 73; SEQ ID NOs 62, 63 and 74; SEQ ID NOs 62, 63 and 75; SEQ ID NOs 62, 63 and 76; SEQ ID NOs 62, 63 and 77; SEQ ID NOs 62, 63 and 78; or SEQ ID NOs 62, 63 and 79. FIG. 8 schematically shows the molecule format of the generated fusions. The parental IL2v was included as a control in the set of constructs (SEQ ID NOs 62, 63 and 64). The corresponding cDNAs were cloned into evitria's vector system using conventional (non-PCR based) cloning techniques. The evitria vector plasmids were gene synthesized. Plasmid DNA was prepared under low-endotoxin conditions based on anion exchange chromatography. DNA concentration was determined by measuring the absorption at a wavelength of 260 nm. Correctness of the sequences was confirmed by Sanger sequencing (with up to two sequencing reactions per plasmid depending on the size of the cDNA.) Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter). The one-armed CD8-targeted IgG IL2v fusion constructs were subsequently purified by protein A affinity chromatography (Mab Select SuRe) and preparative size exclusion chromatography (HiLoad 26/60 Superdex 200) and formulated into 20 mM His, 140 mM Nacl, 0.01% Tween20 pH 6.0. The concentration was determined by measuring absorption at a wavelength of 280 nm. Monomer content was determined by analytical size exclusion chromatography and purity by capillary electrophoresis (CE-SDS). Endotoxin levels were determined and <0.231 EU/ml for all samples.

Determination of Binding Kinetics of pH-Dependent OA CD8-Targeted IgG IL2v Fusions to IL2Rbg Using Surface Plasmon Resonance (SPR)

Binding of the one-armed CD8-targeted IgG IL2v fusion constructs to the human heterodimeric IL-2 receptor beta/gamma (IL2Rbg) was investigated by surface plasmon resonance (SPR) using a Biacore T200 instrument (Cytiva). Briefly, a mouse anti-PG LALA antibody (Roche Diagnostics) was immobilized on a series s sensor chip CM3 (Cytiva, 29104990) using standard amine coupling chemistry according to the manufacturer's instructions. A final surface density of approx. 4000 resonance units (RU) was obtained. Subsequently, the IgG IL2v fusion constructs were injected on the second flow cell for 30 s at a concentration of 0.25 μg/ml each. The first flow cell was used as the reference surface. After increasing the flow rate to 30 μl/min, IL2Rbg was injected on both flow cells for 90 s at concentrations ranging from 300 to 11.1 nM (1:3 dilution series) in a single cycle kinetics mode (kinetic titration). The dissociation after the last analyte injection was monitored for 900 s. Subsequently, the surface was regenerated by injecting 10 mM NaOH for 60 s at a flow rate of 5 μl/min. Bulk refractive index differences were corrected by subtracting the response obtained from flow cell 1 (reference surface) as well as by subtracting buffer injections (double referencing). The derived sensorgrams were fitted to a 1:1 Langmuir binding model using the BIAevaluation software (Cytiva). All experiments were performed at 25° C. using HBS-P+(10 mM HEPES, 150 mM NaCl pH 7.4, 0.05% Surfactant P-20), PIPES pH 6.1, PIPES pH 6.5 and PIPES pH 7.0 (20 mM PIPES, 154 mM NaCl, 0.05% Tween-20 each) as running and dilution buffer. The results are summarized in the tables 1-4 below.

TABLE 1
Kinetic rate constants, affinities and t_{1/2} of pH-dependent
IL2v variants binding to IL2Rbg at pH 7.4

| | | ka | kd | KD | t½ |
|---|---|---|---|---|---|
| ID | | (1/Ms) | (1/s) | (nM) | (min) |
| P1AF0551 | OA CD8 IgG IL2v | 4.44E+05 | 2.77E−05 | 0.6 | 417 |
| P1AF0552 | OA CD8 IgG IL2v | 7.75E+05 | 5.70E−03 | 7 | 2 |
| | P026.099 | | | | |
| P1AF0553 | OA CD8 IgG IL2v | 2.37E+05 | 1.92E−02 | 81 | 1 |
| | P026.175 | | | | |
| P1AF0554 | OA CD8 IgG IL2v | 7.81E+05 | 2.48E−02 | 32 | 0 |
| | P026.191 | | | | |
| P1AF0555 | OA CD8 IgG IL2v | 2.20E+05 | 3.69E−03 | 17 | 3 |
| | P026.237 | | | | |
| P1AF0556 | OA CD8 IgG IL2v | 5.36E+05 | 8.97E−03 | 17 | 1 |
| | P026.355 | | | | |
| P1AF0557 | OA CD8 IgG IL2v | 1.48E+06 | 1.13E−01 | 76 | 0 |
| | P027.359 | | | | |
| P1AF0558 | OA CD8 IgG IL2v | 1.78E+05 | 1.60E−03 | 9 | 7 |
| | P030.006 | | | | |
| P1AF0559 | OA CD8 IgG IL2v | 8.27E+04 | 5.73E−03 | 69 | 2 |
| | P030.045 | | | | |
| P1AF0560 | OA CD8 IgG IL2v | 4.24E+05 | 6.88E−04 | 2 | 17 |
| | P031.226 | | | | |
| P1AF0561 | OA CD8 IgG IL2v | 5.23E+05 | 2.07E−02 | 40 | 1 |
| | P041.017 | | | | |
| P1AF0562 | OA CD8 IgG IL2v | 4.30E+05 | 7.14E−03 | 17 | 2 |
| | P041.242 | | | | |
| P1AF0563 | OA CD8 IgG IL2v | 3.26E+05 | 4.85E−03 | 15 | 2 |
| | P041.449 | | | | |
| P1AF0564 | OA CD8 IgG IL2v | 5.33E+05 | 1.53E−02 | 29 | 1 |
| | P041.554 | | | | |
| P1AF0565 | OA CD8 IgG IL2v | 1.85E+05 | 5.46E−03 | 30 | 2 |
| | P042.107 | | | | |
| P1AF0566 | OA CD8 IgG IL2v | 3.98E+05 | 7.53E−05 | 0.2 | 153 |
| | P043.217 | | | | |

TABLE 2
Kinetic rate constants, affinities and t_{1/2} of pH-dependent
IL2v variants binding to IL2Rbg at pH 7.0

| | | ka | kd | KD | t½ |
|---|---|---|---|---|---|
| ID | | (1/Ms) | (1/s) | (nM) | (min) |
| P1AF0551 | OA CD8 IgG IL2v | 2.85E+05 | 1.30E−04 | 0.5 | 89 |
| P1AF0552 | OA CD8 IgG IL2v | 3.75E+05 | 1.77E−03 | 5 | 7 |
| | P026.099 | | | | |
| P1AF0553 | OA CD8 IgG IL2v | 1.45E+05 | 3.43E−03 | 24 | 3 |
| | P026.175 | | | | |
| P1AF0554 | OA CD8 IgG IL2v | 3.19E+05 | 3.56E−03 | 11 | 3 |
| | P026.191 | | | | |
| P1AF0555 | OA CD8 IgG IL2v | 1.49E+05 | 1.45E−03 | 10 | 8 |
| | P026.237 | | | | |
| P1AF0556 | OA CD8 IgG IL2v | 3.54E+05 | 2.40E−03 | 7 | 5 |
| | P026.355 | | | | |
| P1AF0557 | OA CD8 IgG IL2v | 2.49E+05 | 4.10E−03 | 17 | 3 |
| | P027.359 | | | | |
| P1AF0558 | OA CD8 IgG IL2v | 1.10E+05 | 8.47E−04 | 8 | 14 |
| | P030.006 | | | | |
| P1AF0559 | OA CD8 IgG IL2v | 2.84E+05 | 5.85E−03 | 21 | 2 |
| | P030.045 | | | | |
| P1AF0560 | OA CD8 IgG IL2v | 3.37E+05 | 5.61E−04 | 2 | 21 |
| | P031.226 | | | | |
| P1AF0561 | OA CD8 IgG IL2v | 2.45E+05 | 3.10E−03 | 13 | 4 |
| | P041.017 | | | | |
| P1AF0562 | OA CD8 IgG IL2v | 2.36E+05 | 2.10E−03 | 9 | 6 |
| | P041.242 | | | | |
| P1AF0563 | OA CD8 IgG IL2v | 2.05E+05 | 1.47E−03 | 7 | 8 |
| | P041.449 | | | | |
| P1AF0564 | OA CD8 IgG IL2v | 2.31E+05 | 4.13E−03 | 18 | 3 |
| | P041.554 | | | | |
| P1AF0565 | OA CD8 IgG IL2v | 8.11E+04 | 1.60E−03 | 20 | 7 |
| | P042.107 | | | | |
| P1AF0566 | OA CD8 IgG IL2v | 3.21E+05 | 7.76E−05 | 0.2 | 149 |
| | P043.217 | | | | |

TABLE 3
Kinetic rate constants, affinities and t_{1/2} of pH-dependent
IL2v variants binding to IL2Rbg at pH 6.5

| | | ka | kd | KD | t½ |
|---|---|---|---|---|---|
| ID | | (1/Ms) | (1/s) | (nM) | (min) |
| P1AF0551 | OA CD8 IgG IL2v | 1.48E+05 | 3.94E−04 | 3 | 29 |
| P1AF0552 | OA CD8 IgG IL2v | 3.85E+05 | 1.90E−03 | 5 | 6 |
| | P026.099 | | | | |
| P1AF0553 | OA CD8 IgG IL2v | 1.02E+05 | 1.36E−03 | 13 | 9 |
| | P026.175 | | | | |
| P1AF0554 | OA CD8 IgG IL2v | 1.73E+05 | 2.22E−03 | 13 | 5 |
| | P026.191 | | | | |
| P1AF0555 | OA CD8 IgG IL2v | 1.23E+05 | 6.27E−04 | 5 | 18 |
| | P026.237 | | | | |
| P1AF0556 | OA CD8 IgG IL2v | 2.15E+05 | 9.20E−04 | 4 | 13 |
| | P026.355 | | | | |
| P1AF0557 | OA CD8 IgG IL2v | 1.84E+05 | 6.45E−04 | 4 | 18 |
| | P027.359 | | | | |
| P1AF0558 | OA CD8 IgG IL2v | 1.27E+05 | 2.86E−04 | 2 | 40 |
| | P030.006 | | | | |
| P1AF0559 | OA CD8 IgG IL2v | 2.02E+05 | 6.63E−04 | 3 | 17 |
| | P030.045 | | | | |
| P1AF0560 | OA CD8 IgG IL2v | 2.66E+05 | 4.50E−04 | 2 | 26 |
| | P031.226 | | | | |
| P1AF0561 | OA CD8 IgG IL2v | 1.37E+05 | 9.04E−04 | 7 | 13 |
| | P041.017 | | | | |
| P1AF0562 | OA CD8 IgG IL2v | 1.60E+05 | 1.26E−03 | 8 | 9 |
| | P041.242 | | | | |
| P1AF0563 | OA CD8 IgG IL2v | 1.49E+05 | 1.14E−03 | 8 | 10 |
| | P041.449 | | | | |
| P1AF0564 | OA CD8 IgG IL2v | 1.78E+05 | 2.03E−03 | 11 | 6 |
| | P041.554 | | | | |
| P1AF0565 | OA CD8 IgG IL2v | 1.29E+05 | 6.49E−04 | 5 | 18 |
| | P042.107 | | | | |
| P1AF0566 | OA CD8 IgG IL2v | 2.60E+05 | 3.10E−04 | 1 | 37 |
| | P043.217 | | | | |

TABLE 4
Kinetic rate constants, affinities and t_{1/2} of pH-dependent
IL2v variants binding to IL2Rbg at pH 6.1

| | | ka | kd | KD | t½ |
|---|---|---|---|---|---|
| ID | | (1/Ms) | (1/s) | (nM) | (min) |
| P1AF0551 | OA CD8 IgG IL2v | 1.62E+05 | 4.00E−04 | 2 | 29 |
| P1AF0552 | OA CD8 IgG IL2v | 2.76E+05 | 1.07E−03 | 4 | 11 |
| | P026.099 | | | | |
| P1AF0553 | OA CD8 IgG IL2v | 1.55E+05 | 1.05E−03 | 7 | 11 |
| | P026.175 | | | | |
| P1AF0554 | OA CD8 IgG IL2v | 1.59E+05 | 7.99E−04 | 5 | 14 |
| | P026.191 | | | | |
| P1AF0555 | OA CD8 IgG IL2v | 1.09E+05 | 7.73E−04 | 7 | 15 |
| | P026.237 | | | | |
| P1AF0556 | OA CD8 IgG IL2v | 2.56E+05 | 8.29E−04 | 3 | 14 |
| | P026.355 | | | | |
| P1AF0557 | OA CD8 IgG IL2v | 1.00E+05 | 8.92E−04 | 9 | 13 |
| | P027.359 | | | | |
| P1AF0558 | OA CD8 IgG IL2v | 6.02E+04 | 6.46E−04 | 11 | 18 |
| | P030.006 | | | | |
| P1AF0559 | OA CD8 IgG IL2v | 8.53E+04 | 8.04E−04 | 9 | 14 |
| | P030.045 | | | | |
| P1AF0560 | OA CD8 IgG IL2v | 2.62E+05 | 6.55E−04 | 3 | 18 |
| | P031.226 | | | | |
| P1AF0561 | OA CD8 IgG IL2v | 1.33E+05 | 1.20E−03 | 9 | 10 |
| | P041.017 | | | | |
| P1AF0562 | OA CD8 IgG IL2v | 1.26E+05 | 8.09E−04 | 6 | 14 |
| | P041.242 | | | | |
| P1AF0563 | OA CD8 IgG IL2v | 1.80E+05 | 8.37E−04 | 5 | 14 |
| | P041.449 | | | | |
| P1AF0564 | OA CD8 IgG IL2v | 9.11E+04 | 1.07E−03 | 12 | 11 |
| | P041.554 | | | | |
| P1AF0565 | OA CD8 IgG IL2v | 1.59E+05 | 7.46E−04 | 5 | 15 |
| | P042.107 | | | | |
| P1AF0566 | OA CD8 IgG IL2v | 2.64E+05 | 4.77E−04 | 2 | 24 |
| | P043.217 | | | | |

For the parental IL2v (OA CD8 IgG IL2v), a slight reduction in affinity between binding to human IL2Rbg at pH 7.4 and pH 7.0 versus binding to human IL2Rbg at pH 6.5 and pH 6.1 (0.6 nM at pH 7.4, 0.5 nM at pH 7.0, 3 nM at pH 6.5, and 2 nM at pH 6.1, respectively) can be observed using SPR. Its dissociation rate from IL2Rbg becomes faster as the pH decreases. In contrast, most of the pH-dependent IL2v variants (except for e.g. OA CD8 IgG IL2v P031.226 and OA CD8 IgG IL2v P043.217) exhibit significantly weaker binding than the parental IL2v (OA CD8 IgG IL2v) at pH 7.4 and pH 7.0 but their affinities increase and their dissociation rates from human IL2Rbg become slower as the pH decreases. Thus, compared to the parental IL2v (OA CD8 IgG IL2v), most of the selected pH-dependent IL2v variants from the first phage display campaign bind more weakly to human IL2Rbg at neutral pH but with increased affinity at slightly acidic pH (pH 6.5 and pH 6.1).

pSTAT5 Induction by pH-Dependent OA CD8 IgG IL2v Constructs in Human NK Cells

The activity of the pH-dependent OA CD8 IgG IL2v constructs was determined using human PBMCs. Briefly, pH-dependent OA CD8 IgG IL2v construct dilutions (end concentrations 100 nM, 1 nM, 0.01 nM, negative control: untreated; positive control: proleukin) were incubated with fresh human PBMCs for 20 min at 37° C. in the incubator before adding Cytofix (BD #554655) for fixation of the cells (10 min at 37° C.). Cells were centrifuged (350×g, 5 min), re-suspended in PhosflowPerm buffer (BD #558050) and incubated for 30 min at 4° C. Afterwards, cells were washed with FACS Buffer (PBS+2% FCS+5 mM EDTA+0.25% sodium azide) and flow cytometry (FACS) staining was performed for 30 min at 4° C. using the following antibodies: anti-human CD3-Pacific Blue, anti-human CD4 PECy7, anti-human CD8 PerCPCy5.5, anti-human CD25-PE, anti-human CD56-APC, anti-human FoxP3-PE-CF594, anti-pSTAT5-AlexaFluor 488 (all abs BD, only anti-human CD56 derived from Biolegend). Samples were been measured using a BD FACS Fortessa and the percentage of positive cells as well as mean fluorescence intensity of the pSTAT5 signal was determined in the NK cell population (CD3 neg CD56pos).

TABLE 5
pH-dependent OA CD8 IgG IL2v constructs for pSTAT5 assay

| construct | ID |
|---|---|
| OA CD8 IgG IL2v | P1AF0551_ctrl |
| OA CD8 IgG IL2v P026.099 | P1AF0552 |
| OA CD8 IgG IL2v P026.175 | P1AF0553 |
| OA CD8 IgG IL2v P026.191 | P1AF0554 |
| OA CD8 IgG IL2v P026.237 | P1AF0555 |
| OA CD8 IgG IL2v P026.355 | P1AF0556 |
| OA CD8 IgG IL2v P027.359 | P1AF0557 |
| OA CD8 IgG IL2v P030.006 | P1AF0558 |
| OA CD8 IgG IL2v P030.045 | P1AF0559 |
| OA CD8 IgG IL2v P031.226 | P1AF0560 |
| OA CD8 IgG IL2v P041.017 | P1AF0561 |
| OA CD8 IgG IL2v P041.242 | P1AF0562 |
| OA CD8 IgG IL2v P041.449 | P1AF0563 |
| OA CD8 IgG IL2v P041.554 | P1AF0564 |
| OA CD8 IgG IL2v P042.107 | P1AF0565 |
| OA CD8 IgG IL2v P043.217 | P1AF0566 |
| Proleukin | Proleukin |

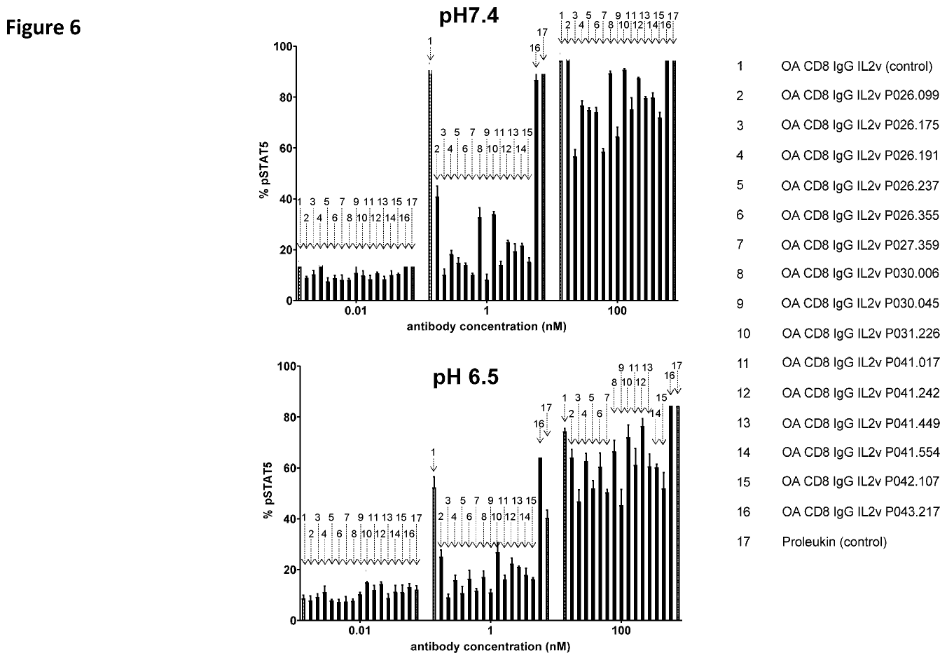

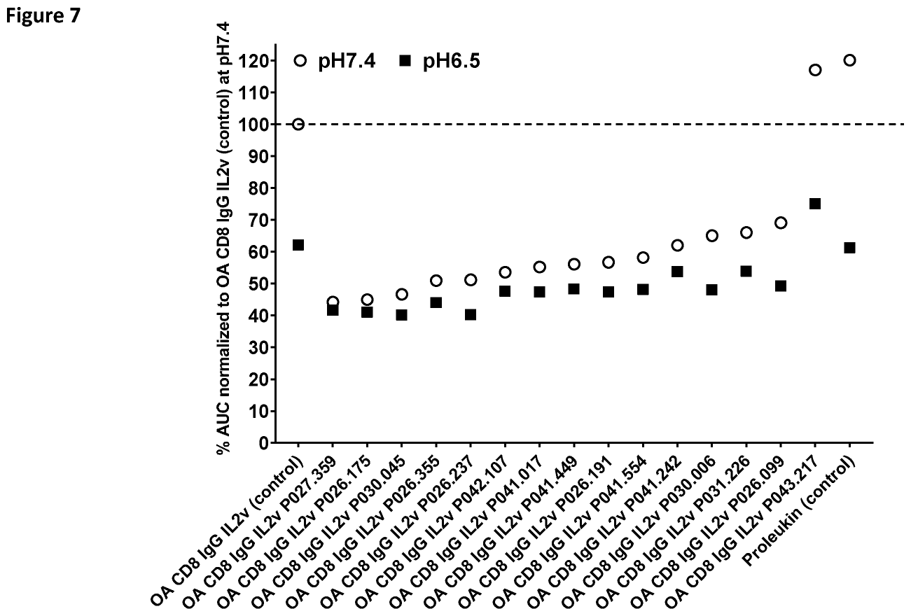

The results are shown in FIGS. 6 and 7. FIG. 6 shows the percentages of NK cells which are positive for phosphorylated STAT5 (Signal Transducer And Activators Of Transcription, pSTAT), a transcription factor activated upon IL-2 signaling, mediating the biological activity of the cytokine. Proleukin as well as the OA CD8 IgG IL2v control construct and also the variant OA CD8 IgG IL2v P043.217 show comparable and concentration-dependent activation of pSTAT5 at pH 7.4. In contrast to this, all other pH-dependent OA CD8 IgG IL2v constructs show reduced activity at pH 7.4 depending on the antibody concentration. At pH 6.5 a general reduction of pSTAT5 induction can be observed upon IL-2 stimulation. FIG. 7 shows the calculated area under the curves (AUC), normalized to the induction of pSTAT5 by OA CD8 IgG IL2v (control) (set to 100% activity at pH 7.4). All pH-dependent constructs showed a reduction in pSTAT5 activation by 31-66% at pH 7.4 (Table 2, middle column) with exception of OA CD8 IgG IL2v P043.217 which performed similar to proleukin showing >100% activity in comparison to CD8 IgG IL2v. At pH 6.5, all constructs show only 40-75% of activity compared to CD8 IgG IL2v at pH 7.4 (Table 6, right column).

TABLE 6
% of AUC values of pH-dependent OA CD8 IgG IL2v constructs
normalized to CD8 IgG IL2v at pH 7.4 in the pSTAT5 assay

| | % AUC | % AUC |
|---|---|---|
| Construct | at pH 7.4 | at pH 6.5 |
| OA CD8 IgG IL2v | 100 | 62.18787 |
| OA CD8 IgG IL2v P027.359 | 44.15577 | 41.71819 |
| OA CD8 IgG IL2v P026.175 | 44.96433 | 41.08205 |
| OA CD8 IgG IL2v P030.045 | 46.61118 | 40.20809 |
| OA CD8 IgG IL2v P026.355 | 50.93341 | 44.07253 |
| OA CD8 IgG IL2v P026.237 | 51.18906 | 40.26754 |
| OA CD8 IgG IL2v P042.107 | 53.54935 | 47.69917 |
| OA CD8 IgG IL2v P041.017 | 55.2497 | 47.4019 |
| OA CD8 IgG IL2v P041.449 | 56.10583 | 48.35315 |
| OA CD8 IgG IL2v P026.191 | 56.71819 | 47.4019 |
| OA CD8 IgG IL2v P041.554 | 58.1629 | 48.21641 |
| OA CD8 IgG IL2v P041.242 | 62.00951 | 53.76338 |
| OA CD8 IgG IL2v P030.006 | 65.04162 | 48.05589 |
| OA CD8 IgG IL2v P031.226 | 66.05232 | 53.90606 |
| OA CD8 IgG IL2v P026.099 | 69.08442 | 49.28656 |
| OA CD8 IgG IL2v P043.217 | 117.063 | 75.08918 |
| Proleukin | 120.1546 | 61.29608 |

### Example 4

### Generation and Characterization of a Second pH-Dependent IL2 Library Based on the Consensus Sequence of IL2v Variants Selected from the First pH-Dependent IL2v Library

A second library was designed based on certain enrichment patterns observed in the first campaign. For example at position K11 we observed only recovery of the initial lysine residue after phage selections. Therefore, for the next round, this residue was back mutated to lysine again. Residues considered for the refined library are as follows: Q11, L12, Q13, E15, H16, L19, D20, Q22, M23, R81, D84, S87, E95, R120, Q126, 5130, and T133. Some positions showed a clear enrichment of one particular amino acid that is not the parental one. For example, Q11 showed an enrichment for E, after the first campaign. R81 was clearly replaced by D. Such patterns were included in the design of the second version of the library. The second library was handled identical to the first campaign.

For the generation of the new pH-dependent IL2v phage display library, the respective DNA fragment were synthesized. It consists of the randomized IL2v sequences and is flanked by the restriction sites NcoI and NotI. The fragment was digested with NcoI/NotI alongside with equally cleaved parental IL2v acceptor phagemid vector. In contrast to the first phagemid vector, the sequence of the V5 tag (GKPIPNPLLGLDST) was inserted between the flag tag and the his tag region of the phagemid. The V5 tag allows specific capturing of the molecules during SPR experiments using an anti-V5 antibody. The library insert was ligated with the phagemid vector over night at 4° C. The purified ligation was used for bacterial transformations resulting in a library with a complexity of more than 2×10¹⁰ transformants. Phagemid particles displaying the IL2v library were rescued and purified by PEG/NaCl purification to be used for selections.

### Example 5

### Selection and Characterization of Improved pH-Dependent IL2v Variants

Selection of pH-Dependent IL2v Variants by Phage Display

The selection for pH-dependent IL2v variants was performed according to a similar procedure for the selection of the first library above. In brief, the recombinant biotinylated IL2R(bg)-Fc construct was used as an antigen for the selection. All selection steps were performed using Bis-Tris-based buffer (50 mM BisTris 140 mM NaCl, final concentration, pH 6 or pH 7.4). Panning rounds were performed in solution according to the following pattern: 1.) binding of approx. 10¹² phagemid particles to 100 nM biotinylated antigen protein for 0.5 h in a total volume of 1 ml; 2.) capture of biotinylated antigen and attachment of specifically binding phage by addition of 5.4×10⁷ streptavidin-coated magnetic beads for 10 min; 3.) washing of the beads using Bis-Tris based buffer; 4.) elution of phage particles. While incubation steps were performed at either pH 7.4 or pH 6, washing steps were always processed at pH 6. Bound phage particles were eluted by addition of either 1 ml 100 mM triethylamine (TEA) for 10 min or by a buffer change to pH 7.4.

Eluted phage was used for re-infection of exponentially growing E. coli TG1 cells. After super-infection with helperphage VCSM13 and subsequent PEG/NaCl precipitation, the isolated phagemid particles were used in subsequent selection rounds. Selections were carried out over 4 rounds using decreasing (from 100 nM to 20 nM) antigen concentrations.

Screening and Characterization of pH-Dependent IL2v Variants by ELISA

For the identification of pH-dependent IL2v variants, soluble IL2v-containing bacterial supernatants were generated and tested by ELISA as follows:

25 μl of 50 nM biotinylated antigen per well were coated at pH 6 on 384-well neutravidin plates and blocked with 2% BSA (50 mM BisTris 140 mM NaCL, 2% BSA, pH 6). Bacterial supernatants containing soluble variants of IL2v were re-buffered to pH 6, added to the plates and incubated for 1 h. Subsequently, several washing steps were performed at either pH 6 or pH 7.4 followed by an incubation step in washing buffer for 30 min at the respective pH. Binding of pH-dependent IL2v variants to the IL2R(bg)-Fc construct was detected via Flag tag by using an anti-Flag/HRP secondary antibody. Comparison of the signal between the two conditions (pH6 vs pH7.4) allowed identifying clones that bind in a pH-dependent manner. The phagemid expressing the parental IL2v construct was used as an internal control for a pH-independent signal. Clones with a binding ratio at pH 6 versus pH 7 that greater than 10 were short-listed and re-tested in order to confirm their pH-dependency.

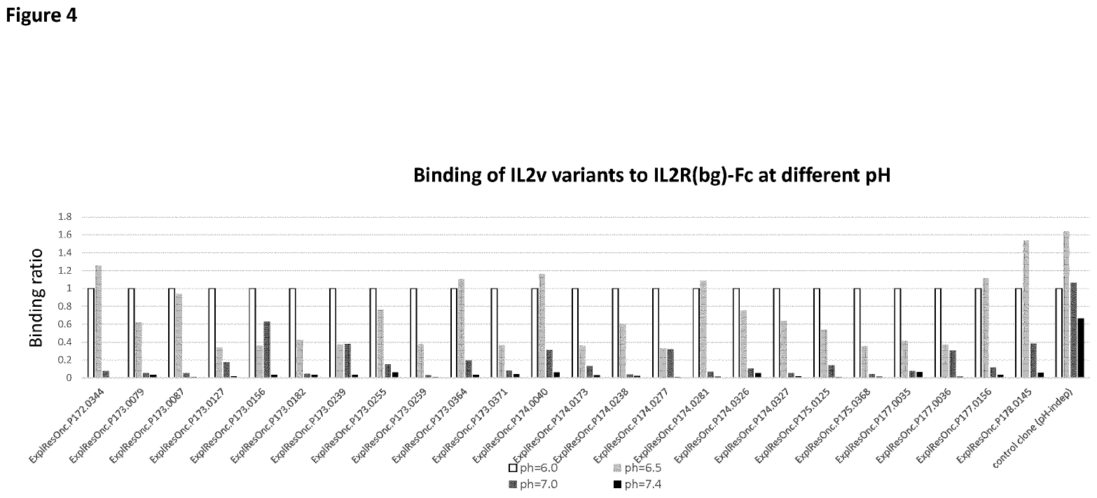

In order to further characterize the selected pH-dependent IL2v variants, they were analyzed for their binding to IL2R(bg)-Fc under four different pH conditions including pH 6, pH 6.5, pH 7, and pH 7.4. The ELISA with the respective pH values was performed as described in Example 3 above and the results are shown in FIG. 4. Binding at pH 6 was normalized to 1 in order to better compare binding among all clones. Interestingly, many binders bind similarly strongly at pH 6.5 as compared to pH 6, e.g. P172.0344, P173.0087, P173.0364, P174.0040, P174.0281, P177.0156, while other clones already show reduced binding, e.g. P173.0127, P173.0156, P173.0239, P173.0259, P173.0371, P0174.0277, P175.0368, P177.0035, P177.0036. In general, binding intensity at pH 7 and pH 7.4 was reduced between 10-100-fold

Screening and Characterization of pH-Dependent IL2v Variants by SPR

In order to examine the individual binding behavior of the selected pH-dependent IL2v-clones, surface plasmon resonance was performed using a ProteOn XPR36 instrument (Biorad): Commercially available anti-VS tag antibody (20 μg/ml) was immobilized on a GLM Chip by amine coupling to achieve immobilization levels of about 8000 response units (RU) in vertical orientation. The bacterial supernatants containing soluble IL2v clone variants were diluted 1:5 with Bis-Tris (pH 7.4) and captured as ligands to the anti-VS mAb on the chip in vertical orientation resulting in captures levels between 100 and 300 RUs. Bacterial supernatant without protein expression was used for referencing.

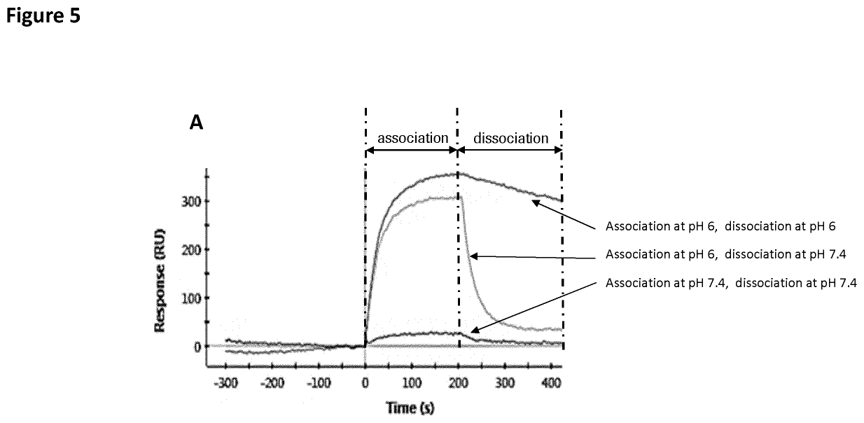

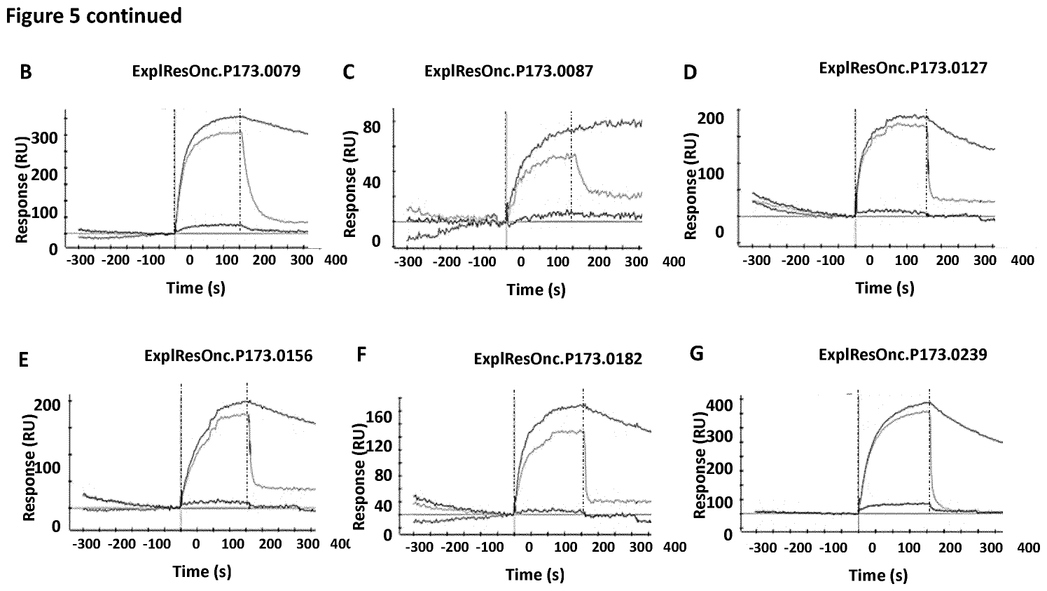

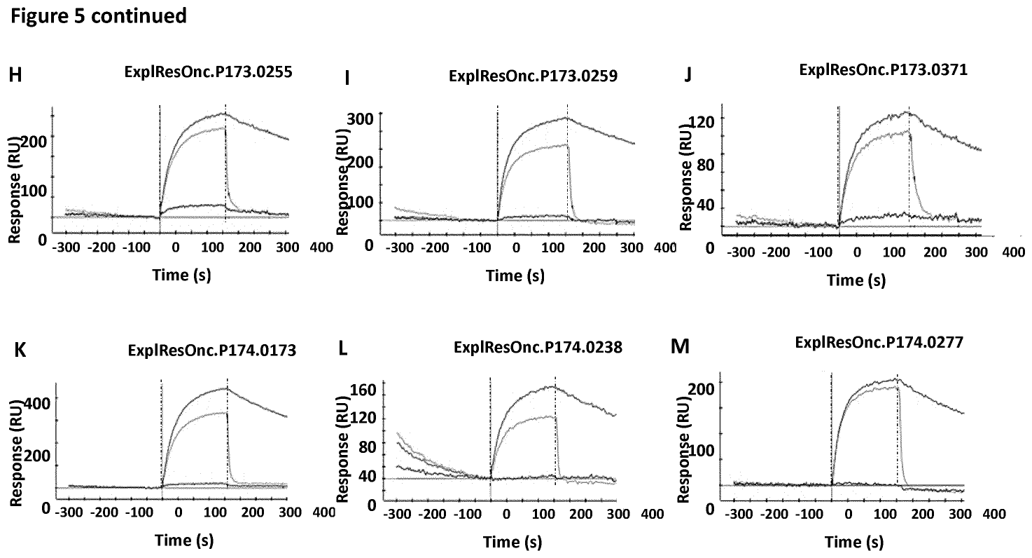

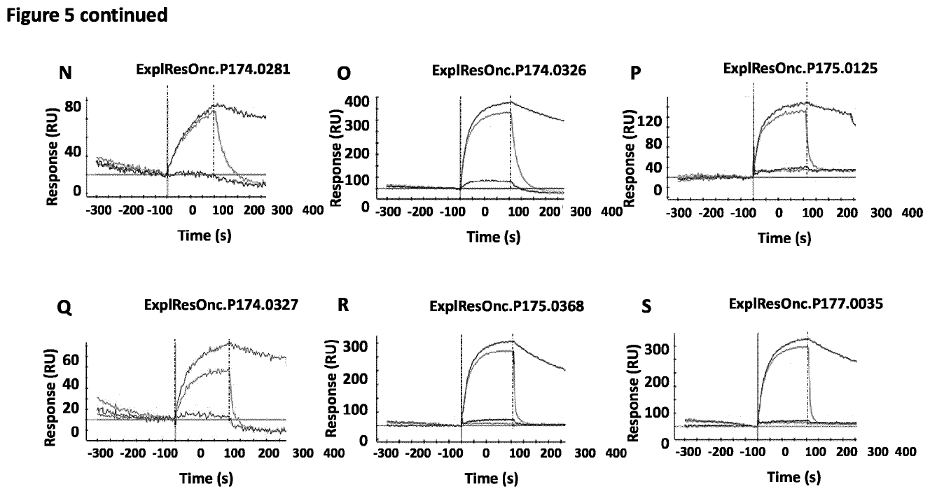

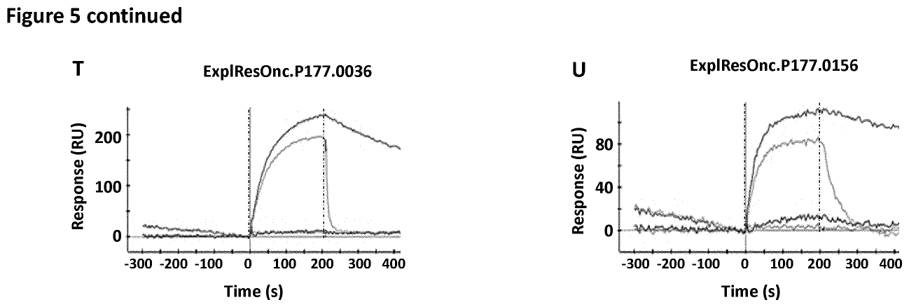

In a co-injection experiment, recombinant IL2R(bg)-Fc (50 nM) was used as an analyte in horizontal orientation to investigate the binding properties of the pH-dependent IL2v clone variants at 3 different conditions simultaneously: 1) association with Bis-Tris buffer (pH 6) and dissociation with Bis-Tris buffer (pH 6); 2) association with Bis-Tris buffer (pH 6) and dissociation with Bis-Tris buffer (pH 7.4); and 3) association with Bis-Tris buffer (pH 7.4) and dissociation with Bis-Tris buffer (pH 7.4). An illustrative example that illustrates the experimental conditions is shown in FIG. 5A. Both association and dissociation lasted 200 sec at a flow rate of 50 μl per second. While condition 1 (association and dissociation at pH 6) resulted in a strong binding of IL2R(bg)-Fc to the IL2v binder variants, the buffer change from pH 6 (association) to pH 7.4 (dissociation) induced a rapid loss in binding and the interaction between IL2R(bg)-Fc and the immobilized pH-dependent IL2v variants disappeared within a few seconds. For many clone variants, the interaction during the association phase at pH 7.4 was not or only weakly detectable. The sensorgrams of the best twenty clones at three conditions tested are shown in FIGS. 5B-U. Based on very clear pH-dependent binding results coming from the qualitative SPR measurements, twenty four clones (SEQ ID NOs 20-43) were selected for the final conversion of the IL2v variants into anti-CD8 targeted IgG fusion constructs.

Conversion of pH-Dependent IL2v Variants into One-Armed CD8-Targeted IgG IL2v Fusions—Second Set of Variants from the Second Phage Display Campaign

Twenty four pH-dependent IL2v variants (SEQ ID NOs 20-43) that had been selected by phage display for preferential binding at slightly acidic pH in the second campaign were converted into one-armed CD8-targeted IgG PG LALA IL2v fusions for expression in mammalian cells (CHO K1) and subsequent purification by protein A affinity chromatography and preparative size exclusion chromatography (SEC). The pH-dependent IL2v variants were fused to the C-terminus of the ‘knob’ heavy chain without terminal lysine (K) and connected via a (G4S)2 G5 glycine-serine linker (FIG. 8 shows schematically the molecule format of the generated fusions). Additionally, this set not only includes selected pH-dependent IL2v variants but also IL2v variants with single mutations or combined consensus mutations. The resulting CD8-targeted IgG IL2v fusions were composed of SEQ ID NOs 62, 80 and 81; SEQ ID NOs 62, 80 and 82; SEQ ID NOs 62, 80 and 83; SEQ ID NOs 62, 80 and 84; SEQ ID NOs 62, 80 and 85; SEQ ID NOs 62, 80 and 86; SEQ ID NOs 62, 80 and 87; SEQ ID NOs 62, 80 and 88; SEQ ID NOs 62, 80 and 89; SEQ ID NOs 62, 80 and 90; SEQ ID NOs 62, 80 and 91; SEQ ID NOs 62, 80 and 92; SEQ ID NOs 62, 80 and 93; SEQ ID NOs 62, 80 and 94; SEQ ID NOs 62, 80 and 95; SEQ ID NOs 62, 80 and 96; SEQ ID NOs 62, 80 and 97; SEQ ID NOs 62, 80 and 98; SEQ ID NOs 62, 80 and 99; SEQ ID NOs 62, 80 and 100; SEQ ID NOs 62, 80 and 101; SEQ ID NOs 62, 80 and 102; SEQ ID NOs 62, 80 and 103; SEQ ID NOs 62, 80 and 104; SEQ ID NOs 62, 80 and 105; SEQ ID NOs 62, 80 and 106; SEQ ID NOs 62, 80 and 107; SEQ ID NOs 62, 80 and 108; SEQ ID NOs 62, 80 and 109; SEQ ID NOs 62, 80 and 110; SEQ ID NOs 62, 80 and 111; SEQ ID NOs 62, 80 and 112; SEQ ID NOs 62, 80 and 113; SEQ ID NOs 62, 80 and 114; SEQ ID NOs 62, 80 and 115; SEQ ID NOs 62, 80 and 116; SEQ ID NOs 62, 80 and 117; SEQ ID NOs 62, 80 and 118; SEQ ID NOs 62, 80 and 119; SEQ ID NOs 62, 80 and 120; SEQ ID NOs 62, 80 and 121; SEQ ID NOs 62, 80 and 122; SEQ ID NOs 62, 80 and 124; SEQ ID NOs 62, 80 and 125; SEQ ID NOs 62, 80 and 126; SEQ ID NOs 62, 80 and 127; SEQ ID NOs 62, 80 and 128; SEQ ID NOs 62, 80 and 129; SEQ ID NOs 62, 80 and 130; SEQ ID NOs 62, 80 and 131; SEQ ID NOs 62, 80 and 132; SEQ ID NOs 62, 80 and 133; SEQ ID NOs 62, 80 and 134; SEQ ID NOs 62, 80 and 135; SEQ ID NOs 62, 80 and 136; SEQ ID NOs 62, 80 and 137; SEQ ID NOs 62, 80 and 138; SEQ ID NOs 62, 80 and 139; SEQ ID NOs 62, 80 and 140 SEQ ID NOs 62, 80 and 141; SEQ ID NOs 62, 80 and 142; or SEQ ID NOs 62, 80 and 143.

### Example 6

Determination of Binding Kinetics of One-Armed CD8-Targeted IgG IL2v Fusions (2nd Set of Variants from 2nd Phage Display Campaign) to IL2Rbg Using Biolayer Interferometry (BLI)

Binding of the one-armed CD8-targeted IgG IL2v fusions to IL-2Rbg at different pH values was investigated by biolayer interferometry using an Octet RED384 instrument (Sartorius). Briefly, the antibody—IL2v fusions were captured onto anti-human Fab CH1 sensor tips (FAB2G, Sartorius no. 18-5125) for 90s at a concentration of 5 μg/ml in 20 mM PIPES, 140 mM NaCl pH 7.4, 0.05% Tween 20. After an additional washing/baseline step using already the corresponding dilution buffer, 100 nM of human IL2Rbg (P1AA4193) or murine IL2Rbg (P1AD6727) were bound to the captured antibody—IL2v fusions with 180s association and 300s dissociation time, diluted in 20 mM PIPES, 140 mM NaCl, 0.05% Tween 20 at pH 6.0, 6.2, 6.5, 6.8, 7.1, or 7.4, respectively. Subsequently, the sensor tips were regenerated with 10 mM Glycine pH 1.7 for three times 30s. The obtained binding curves were fitted to a 1:1 binding model using the Data Analysis Software (Sartorius). The results are summarized in the Table 7.

TABLE 7
Binding kinetics of one-armed CD8-targeted IgG IL2v
fusions to human IL-2Rbg at different pH values

| pH | concept ID | construct | kon(1/Ms) | kdis(1/s) | KD (M) | KD (nM) |
|---|---|---|---|---|---|---|
| 7.4 | P1AG0697 | one-armed CD8 IgG-pH dep | 3.07E+05 | 1.26E−02 | 4.09E−08 | 40.9 |
| | | IL2v_ExplResOnc.P172.0344 | | | | |
| 7.1 | P1AG0697 | one-armed CD8 IgG-pH dep | 3.05E+05 | 5.28E−03 | 1.73E−08 | 17.3 |
| | | IL2v_ExplResOnc.P172.0344 | | | | |
| 6.8 | P1AG0697 | one-armed CD8 IgG-pH dep | 2.60E+05 | 2.79E−03 | 1.07E−08 | 10.7 |
| | | IL2v_ExplResOnc.P172.0344 | | | | |
| 6.5 | P1AG0697 | one-armed CD8 IgG-pH dep | 2.15E+05 | 1.44E−03 | 6.70E−09 | 6.7 |
| | | IL2v_ExplResOnc.P172.0344 | | | | |
| 6.2 | P1AG0697 | one-armed CD8 IgG-pH dep | 2.07E+05 | 7.60E−04 | 3.67E−09 | 3.7 |
| | | IL2v_ExplResOnc.P172.0344 | | | | |
| 6.0 | P1AG0697 | one-armed CD8 IgG-pH dep | 2.00E+05 | 5.72E−04 | 2.86E−09 | 2.9 |
| | | IL2v_ExplResOnc.P172.0344 | | | | |
| 7.4 | P1AG0698 | one-armed CD8 IgG-pH dep | 2.30E+03 | 4.34E−01 | 1.89E−04 | low |
| | | IL2v_ExplResOnc.P173.0079 | | | | |
| 7.1 | P1AG0698 | one-armed CD8 IgG-pH dep | 5.51E+03 | 4.58E−02 | 8.31E−06 | 8312.0 |
| | | IL2v_ExplResOnc.P173.0079 | | | | |
| 6.8 | P1AG0698 | one-armed CD8 IgG-pH dep | 1.90E+05 | 1.16E−02 | 6.07E−08 | 60.7 |
| | | IL2v_ExplResOnc.P173.0079 | | | | |
| 6.5 | P1AG0698 | one-armed CD8 IgG-pH dep | 2.81E+05 | 4.77E−03 | 1.70E−08 | 17.0 |
| | | IL2v_ExplResOnc.P173.0079 | | | | |
| 6.2 | P1AG0698 | one-armed CD8 IgG-pH dep | 3.55E+05 | 2.63E−03 | 7.40E−09 | 7.4 |
| | | IL2v_ExplResOnc.P173.0079 | | | | |
| 6.0 | P1AG0698 | one-armed CD8 IgG-pH dep | 3.73E+05 | 1.95E−03 | 5.23E−09 | 5.2 |
| | | IL2v_ExplResOnc.P173.0079 | | | | |
| 7.4 | P1AG0699 | one-armed CD8 IgG-pH dep | 3.79E+06 | 2.59E+01 | 6.84E−06 | 6836.0 |
| | | IL2v_ExplResOnc.P173.0087 | | | | |
| 7.1 | P1AG0699 | one-armed CD8 IgG-pH dep | 1.57E+05 | 5.02E−02 | 3.21E−07 | 320.5 |
| | | IL2v_ExplResOnc.P173.0087 | | | | |
| 6.8 | P1AG0699 | one-armed CD8 IgG-pH dep | 1.96E+05 | 1.45E−02 | 7.40E−08 | 74.0 |
| | | IL2v_ExplResOnc.P173.0087 | | | | |
| 6.5 | P1AG0699 | one-armed CD8 IgG-pH dep | 2.38E+05 | 4.97E−03 | 2.09E−08 | 20.9 |
| | | IL2v_ExplResOnc.P173.0087 | | | | |
| 6.2 | P1AG0699 | one-armed CD8 IgG-pH dep | 2.51E+05 | 2.47E−03 | 9.83E−09 | 9.8 |
| | | IL2v_ExplResOnc.P173.0087 | | | | |
| 6.0 | P1AG0699 | one-armed CD8 IgG-pH dep | 2.53E+05 | 1.78E−03 | 7.02E−09 | 7.0 |
| | | IL2v_ExplResOnc.P173.0087 | | | | |
| 7.4 | P1AG0701 | one-armed CD8 IgG-pH dep | 1.77E+04 | 3.01E−01 | 1.70E−05 | low |
| | | IL2v_ExplResOnc.P173.0156 | | | | |
| 7.1 | P1AG0701 | one-armed CD8 IgG-pH dep | 1.14E+06 | 1.39E−01 | 1.22E−07 | 121.6 |
| | | IL2v_ExplResOnc.P173.0156 | | | | |
| 6.8 | P1AG0701 | one-armed CD8 IgG-pH dep | 1.90E+05 | 4.53E−02 | 2.38E−07 | 238.2 |
| | | IL2v_ExplResOnc.P173.0156 | | | | |
| 6.5 | P1AG0701 | one-armed CD8 IgG-pH dep | 1.60E+05 | 2.07E−02 | 1.29E−07 | 129.0 |
| | | IL2v_ExplResOnc.P173.0156 | | | | |
| 6.2 | P1AG0701 | one-armed CD8 IgG-pH dep | 2.05E+05 | 9.36E−03 | 4.57E−08 | 45.7 |
| | | IL2v_ExplResOnc.P173.0156 | | | | |
| 6.0 | P1AG0701 | one-armed CD8 IgG-pH dep | 2.03E+05 | 6.08E−03 | 2.99E−08 | 29.9 |
| | | IL2v_ExplResOnc.P173.0156 | | | | |
| 7.4 | P1AG0702 | one-armed CD8 IgG-pH dep | 5.48E+06 | 1.60E+00 | 2.92E−07 | 291.6 |
| | | IL2v_ExplResOnc.P173.0182 | | | | |
| 7.1 | P1AG0702 | one-armed CD8 IgG-pH dep | 1.77E+06 | 1.09E−01 | 6.15E−08 | 61.5 |
| | | IL2v_ExplResOnc.P173.0182 | | | | |
| 6.8 | P1AG0702 | one-armed CD8 IgG-pH dep | 2.99E+05 | 2.09E−02 | 6.99E−08 | 69.9 |
| | | IL2v_ExplResOnc.P173.0182 | | | | |
| 6.5 | P1AG0702 | one-armed CD8 IgG-pH dep | 4.14E+05 | 7.22E−03 | 1.74E−08 | 17.4 |
| | | IL2v_ExplResOnc.P173.0182 | | | | |
| 6.2 | P1AG0702 | one-armed CD8 IgG-pH dep | 4.67E+05 | 4.71E−03 | 1.01E−08 | 10.1 |
| | | IL2v_ExplResOnc.P173.0182 | | | | |
| 6.0 | P1AG0702 | one-armed CD8 IgG-pH dep | 4.48E+05 | 3.60E−03 | 8.03E−09 | 8.0 |
| | | IL2v_ExplResOnc.P173.0182 | | | | |
| 7.4 | P1AG0703 | one-armed CD8 IgG-pH dep | 3.31E+04 | 2.66E−01 | 8.03E−06 | low |
| | | IL2v_ExplResOnc.P173.0239 | | | | |
| 7.1 | P1AG0703 | one-armed CD8 IgG-pH dep | 3.04E+05 | 2.83E−02 | 9.33E−08 | 93.3 |
| | | IL2v_ExplResOnc.P173.0239 | | | | |
| 6.8 | P1AG0703 | one-armed CD8 IgG-pH dep | 3.71E+05 | 9.50E−03 | 2.56E−08 | 25.6 |
| | | IL2v_ExplResOnc.P173.0239 | | | | |
| 6.5 | P1AG0703 | one-armed CD8 IgG-pH dep | 3.86E+05 | 4.75E−03 | 1.23E−08 | 12.3 |
| | | IL2v_ExplResOnc.P173.0239 | | | | |
| 6.2 | P1AG0703 | one-armed CD8 IgG-pH dep | 3.68E+05 | 2.51E−03 | 6.83E−09 | 6.8 |
| | | IL2v_ExplResOnc.P173.0239 | | | | |
| 6.0 | P1AG0703 | one-armed CD8 IgG-pH dep | 3.60E+05 | 1.69E−03 | 4.71E−09 | 4.7 |
| | | IL2v_ExplResOnc.P173.0239 | | | | |
| 7.4 | P1AG0704 | one-armed CD8 IgG-pH dep | 9.36E+07 | 3.72E+01 | 3.98E−07 | 398.0 |
| | | IL2v_ExplResOnc.P173.0255 | | | | |
| 7.1 | P1AG0704 | one-armed CD8 IgG-pH dep | 1.36E+05 | 1.17E−01 | 8.60E−07 | 859.8 |
| | | IL2v_ExplResOnc.P173.0255 | | | | |
| 6.8 | P1AG0704 | one-armed CD8 IgG-pH dep | 2.12E+05 | 3.15E−02 | 1.49E−07 | 148.6 |
| | | IL2v_ExplResOnc.P173.0255 | | | | |
| 6.5 | P1AG0704 | one-armed CD8 IgG-pH dep | 3.11E+05 | 9.63E−03 | 3.10E−08 | 31.0 |
| | | IL2v_ExplResOnc.P173.0255 | | | | |
| 6.2 | P1AG0704 | one-armed CD8 IgG-pH dep | 3.36E+05 | 4.41E−03 | 1.31E−08 | 13.1 |
| | | IL2v_ExplResOnc.P173.0255 | | | | |
| 6.0 | P1AG0704 | one-armed CD8 IgG-pH dep | 3.35E+05 | 3.59E−03 | 1.07E−08 | 10.7 |
| | | IL2v_ExplResOnc.P173.0255 | | | | |
| 7.4 | P1AG0705 | one-armed CD8 IgG-pH dep | 5.71E+10 | 7.92E+01 | 1.39E−09 | 1.4 |
| | | IL2v_ExplResOnc.P173.0259 | | | | |
| 7.1 | P1AG0705 | one-armed CD8 IgG-pH dep | 5.17E+06 | 1.58E−01 | 3.06E−08 | 30.6 |
| | | IL2v_ExplResOnc.P173.0259 | | | | |
| 6.8 | P1AG0705 | one-armed CD8 IgG-pH dep | 2.43E+05 | 2.59E−02 | 1.07E−07 | 106.6 |
| | | IL2v_ExplResOnc.P173.0259 | | | | |
| 6.5 | P1AG0705 | one-armed CD8 IgG-pH dep | 3.33E+05 | 1.05E−02 | 3.15E−08 | 31.5 |
| | | IL2v_ExplResOnc.P173.0259 | | | | |
| 6.2 | P1AG0705 | one-armed CD8 IgG-pH dep | 4.26E+05 | 5.39E−03 | 1.27E−08 | 12.7 |
| | | IL2v_ExplResOnc.P173.0259 | | | | |
| 6.0 | P1AG0705 | one-armed CD8 IgG-pH dep | 4.29E+05 | 3.39E−03 | 7.90E−09 | 7.9 |
| | | IL2v_ExplResOnc.P173.0259 | | | | |
| 7.4 | P1AG0706 | one-armed CD8 IgG-pH dep | 6.43E+04 | 3.19E−02 | 4.96E−07 | 496.2 |
| | | IL2v_ExplResOnc.P173.0364 | | | | |
| 7.1 | P1AG0706 | one-armed CD8 IgG-pH dep | 1.48E+05 | 1.09E−02 | 7.41E−08 | 74.1 |
| | | IL2v_ExplResOnc.P173.0364 | | | | |
| 6.8 | P1AG0706 | one-armed CD8 IgG-pH dep | 1.92E+05 | 4.25E−03 | 2.21E−08 | 22.1 |
| | | IL2v_ExplResOnc.P173.0364 | | | | |
| 6.5 | P1AG0706 | one-armed CD8 IgG-pH dep | 1.38E+05 | 2.95E−03 | 2.13E−08 | 21.3 |
| | | IL2v_ExplResOnc.P173.0364 | | | | |
| 6.2 | P1AG0706 | one-armed CD8 IgG-pH dep | 1.86E+05 | 2.20E−03 | 1.18E−08 | 11.8 |
| | | IL2v_ExplResOnc.P173.0364 | | | | |
| 6.0 | P1AG0706 | one-armed CD8 IgG-pH dep | 1.74E+05 | 1.81E−03 | 1.04E−08 | 10.4 |
| | | IL2v_ExplResOnc.P173.0364 | | | | |
| 7.4 | P1AG0707 | one-armed CD8 IgG-pH dep | 1.04E+04 | 1.01E−01 | 9.66E−06 | low |
| | | IL2v_ExplResOnc.P173.0371 | | | | |
| 7.1 | P1AG0707 | one-armed CD8 IgG-pH dep | 6.82E+04 | 2.68E−02 | 3.92E−07 | 392.4 |
| | | IL2v_ExplResOnc.P173.0371 | | | | |
| 6.8 | P1AG0707 | one-armed CD8 IgG-pH dep | 1.82E+05 | 9.70E−03 | 5.32E−08 | 53.2 |
| | | IL2v_ExplResOnc.P173.0371 | | | | |
| 6.5 | P1AG0707 | one-armed CD8 IgG-pH dep | 2.39E+05 | 5.00E−03 | 2.09E−08 | 20.9 |
| | | IL2v_ExplResOnc.P173.0371 | | | | |
| 6.2 | P1AG0707 | one-armed CD8 IgG-pH dep | 2.87E+05 | 2.63E−03 | 9.16E−09 | 9.2 |
| | | IL2v_ExplResOnc.P173.0371 | | | | |
| 6.0 | P1AG0707 | one-armed CD8 IgG-pH dep | 3.03E+05 | 2.04E−03 | 6.74E−09 | 6.7 |
| | | IL2v_ExplResOnc.P173.0371 | | | | |
| 7.4 | P1AG0708 | one-armed CD8 IgG-pH dep | 9.53E+05 | 1.38E−01 | 1.45E−07 | 145.0 |
| | | IL2v_ExplResOnc.P174.0040 | | | | |
| 7.1 | P1AG0708 | one-armed CD8 IgG-pH dep | 1.74E+05 | 2.77E−02 | 1.60E−07 | 159.8 |
| | | IL2v_ExplResOnc.P174.0040 | | | | |
| 6.8 | P1AG0708 | one-armed CD8 IgG-pH dep | 3.63E+05 | 6.96E−03 | 1.92E−08 | 19.2 |
| | | IL2v_ExplResOnc.P174.0040 | | | | |
| 6.5 | P1AG0708 | one-armed CD8 IgG-pH dep | 3.01E+05 | 4.33E−03 | 1.44E−08 | 14.4 |
| | | IL2v_ExplResOnc.P174.0040 | | | | |
| 6.2 | P1AG0708 | one-armed CD8 IgG-pH dep | 3.19E+05 | 2.96E−03 | 9.25E−09 | 9.3 |
| | | IL2v_ExplResOnc.P174.0040 | | | | |
| 6.0 | P1AG0708 | one-armed CD8 IgG-pH dep | 3.03E+05 | 2.23E−03 | 7.35E−09 | 7.4 |
| | | IL2v_ExplResOnc.P174.0040 | | | | |
| 7.4 | P1AG0709 | one-armed CD8 IgG-pH dep | 5.36E+05 | 1.14E+00 | 2.12E−06 | low |
| | | IL2v_ExplResOnc.P174.0173 | | | | |
| 7.1 | P1AG0709 | one-armed CD8 IgG-pH dep | 1.99E+06 | 3.69E−01 | 1.86E−07 | 185.7 |
| | | IL2v_ExplResOnc.P174.0173 | | | | |
| 6.8 | P1AG0709 | one-armed CD8 IgG-pH dep | 2.86E+05 | 4.55E−02 | 1.59E−07 | 159.3 |
| | | IL2v_ExplResOnc.P174.0173 | | | | |
| 6.5 | P1AG0709 | one-armed CD8 IgG-pH dep | 2.43E+05 | 1.90E−02 | 7.79E−08 | 77.9 |
| | | IL2v_ExplResOnc.P174.0173 | | | | |
| 6.2 | P1AG0709 | one-armed CD8 IgG-pH dep | 3.75E+05 | 7.36E−03 | 1.96E−08 | 19.6 |
| | | IL2v_ExplResOnc.P174.0173 | | | | |
| 6.0 | P1AG0709 | one-armed CD8 IgG-pH dep | 3.70E+05 | 5.04E−03 | 1.36E−08 | 13.6 |
| | | IL2v_ExplResOnc.P174.0173 | | | | |
| 7.4 | P1AG0710 | one-armed CD8 IgG-pH dep | 6.07E−02 | 6.25E−01 | 1.03E+01 | low |
| | | IL2v_ExplResOnc.P174.0238 | | | | |
| 7.1 | P1AG0710 | one-armed CD8 IgG-pH dep | 3.45E+06 | 4.08E−01 | 1.18E−07 | 118.1 |
| | | IL2v_ExplResOnc.P174.0238 | | | | |
| 6.8 | P1AG0710 | one-armed CD8 IgG-pH dep | 3.22E+05 | 7.35E−02 | 2.28E−07 | 227.9 |
| | | IL2v_ExplResOnc.P174.0238 | | | | |
| 6.5 | P1AG0710 | one-armed CD8 IgG-pH dep | 2.13E+05 | 2.06E−02 | 9.66E−08 | 96.6 |
| | | IL2v_ExplResOnc.P174.0238 | | | | |
| 6.2 | P1AG0710 | one-armed CD8 IgG-pH dep | 3.50E+05 | 6.82E−03 | 1.95E−08 | 19.5 |
| | | IL2v_ExplResOnc.P174.0238 | | | | |
| 6.0 | P1AG0710 | one-armed CD8 IgG-pH dep | 3.44E+05 | 4.73E−03 | 1.38E−08 | 13.8 |
| | | IL2v_ExplResOnc.P174.0238 | | | | |
| 7.4 | P1AG0711 | one-armed CD8 IgG-pH dep | 2.49E+04 | 8.28E+00 | 3.33E−04 | low |
| | | IL2v_ExplResOnc.P174.0277 | | | | |
| 7.1 | P1AG0711 | one-armed CD8 IgG-pH dep | 1.78E+06 | 1.34E−01 | 7.53E−08 | 75.3 |
| | | IL2v_ExplResOnc.P174.0277 | | | | |
| 6.8 | P1AG0711 | one-armed CD8 IgG-pH dep | 2.24E+05 | 3.33E−02 | 1.49E−07 | 148.8 |
| | | IL2v_ExplResOnc.P174.0277 | | | | |
| 6.5 | P1AG0711 | one-armed CD8 IgG-pH dep | 3.02E+05 | 1.20E−02 | 3.95E−08 | 39.5 |
| | | IL2v_ExplResOnc.P174.0277 | | | | |
| 6.2 | P1AG0711 | one-armed CD8 IgG-pH dep | 3.80E+05 | 5.52E−03 | 1.45E−08 | 14.5 |
| | | IL2v_ExplResOnc.P174.0277 | | | | |
| 6.0 | P1AG0711 | one-armed CD8 IgG-pH dep | 3.75E+05 | 4.17E−03 | 1.11E−08 | 11.1 |
| | | IL2v_ExplResOnc.P174.0277 | | | | |
| 7.4 | P1AG0712 | one-armed CD8 IgG-pH dep | 6.93E+03 | 7.06E−02 | 1.02E−05 | low |
| | | IL2v_ExplResOnc.P174.0281 | | | | |
| 7.1 | P1AG0712 | one-armed CD8 IgG-pH dep | 1.00E+05 | 1.19E−02 | 1.19E−07 | 118.7 |
| | | IL2v_ExplResOnc.P174.0281 | | | | |
| 6.8 | P1AG0712 | one-armed CD8 IgG-pH dep | 1.36E+05 | 4.37E−03 | 3.21E−08 | 32.1 |
| | | IL2v_ExplResOnc.P174.0281 | | | | |
| 6.5 | P1AG0712 | one-armed CD8 IgG-pH dep | 1.29E+05 | 2.38E−03 | 1.85E−08 | 18.5 |
| | | IL2v_ExplResOnc.P174.0281 | | | | |
| 6.2 | P1AG0712 | one-armed CD8 IgG-pH dep | 1.39E+05 | 1.16E−03 | 8.30E−09 | 8.3 |
| | | IL2v_ExplResOnc.P174.0281 | | | | |
| 6.0 | P1AG0712 | one-armed CD8 IgG-pH dep | 1.52E+05 | 9.74E−04 | 6.41E−09 | 6.4 |
| | | IL2v_ExplResOnc.P174.0281 | | | | |
| 7.4 | P1AG0713 | one-armed CD8 IgG-pH dep | 2.26E+04 | 8.29E−02 | 3.67E−06 | low |
| | | IL2v_ExplResOnc.P174.0326 | | | | |
| 7.1 | P1AG0713 | one-armed CD8 IgG-pH dep | 7.15E+04 | 3.53E−02 | 4.93E−07 | 493.3 |
| | | IL2v_ExplResOnc.P174.0326 | | | | |
| 6.8 | P1AG0713 | one-armed CD8 IgG-pH dep | 2.73E+05 | 1.06E−02 | 3.87E−08 | 38.7 |
| | | IL2v_ExplResOnc.P174.0326 | | | | |
| 6.5 | P1AG0713 | one-armed CD8 IgG-pH dep | 3.44E+05 | 5.21E−03 | 1.52E−08 | 15.2 |
| | | IL2v_ExplResOnc.P174.0326 | | | | |
| 6.2 | P1AG0713 | one-armed CD8 IgG-pH dep | 4.19E+05 | 2.88E−03 | 6.88E−09 | 6.9 |
| | | IL2v_ExplResOnc.P174.0326 | | | | |
| 6.0 | P1AG0713 | one-armed CD8 IgG-pH dep | 4.13E+05 | 2.30E−03 | 5.56E−09 | 5.6 |
| | | IL2v_ExplResOnc.P174.0326 | | | | |
| 7.4 | P1AG0714 | one-armed CD8 IgG-pH dep | 1.41E+05 | 6.03E−02 | 4.29E−07 | 428.5 |
| | | IL2v_ExplResOnc.P174.0327 | | | | |
| 7.1 | P1AG0714 | one-armed CD8 IgG-pH dep | 2.96E+05 | 1.29E−02 | 4.35E−08 | 43.5 |
| | | IL2v_ExplResOnc.P174.0327 | | | | |
| 6.8 | P1AG0714 | one-armed CD8 IgG-pH dep | 2.95E+05 | 4.97E−03 | 1.68E−08 | 16.8 |
| | | IL2v_ExplResOnc.P174.0327 | | | | |
| 6.5 | P1AG0714 | one-armed CD8 IgG-pH dep | 2.28E+05 | 2.71E−03 | 1.19E−08 | 11.9 |
| | | IL2v_ExplResOnc.P174.0327 | | | | |
| 6.2 | P1AG0714 | one-armed CD8 IgG-pH dep | 2.32E+05 | 1.72E−03 | 7.41E−09 | 7.4 |
| | | IL2v_ExplResOnc.P174.0327 | | | | |
| 6.0 | P1AG0714 | one-armed CD8 IgG-pH dep | 2.04E+05 | 1.14E−03 | 5.60E−09 | 5.6 |
| | | IL2v_ExplResOnc.P174.0327 | | | | |
| 7.4 | P1AG0715 | one-armed CD8 IgG-pH dep | 5.63E+06 | 9.19E+00 | 1.63E−06 | low |
| | | IL2v_ExplResOnc.P175.0125 | | | | |
| 7.1 | P1AG0715 | one-armed CD8 IgG-pH dep | 1.24E+05 | 3.03E−02 | 2.44E−07 | 244.3 |
| | | IL2v_ExplResOnc.P175.0125 | | | | |
| 6.8 | P1AG0715 | one-armed CD8 IgG-pH dep | 2.50E+05 | 7.91E−03 | 3.16E−08 | 31.6 |
| | | IL2v_ExplResOnc.P175.0125 | | | | |
| 6.5 | P1AG0715 | one-armed CD8 IgG-pH dep | 2.92E+05 | 3.72E−03 | 1.27E−08 | 12.7 |
| | | IL2v_ExplResOnc.P175.0125 | | | | |
| 6.2 | P1AG0715 | one-armed CD8 IgG-pH dep | 3.41E+05 | 2.04E−03 | 5.98E−09 | 6.0 |
| | | IL2v_ExplResOnc.P175.0125 | | | | |
| 6.0 | P1AG0715 | one-armed CD8 IgG-pH dep | 3.52E+05 | 1.16E−03 | 3.31E−09 | 3.3 |
| | | IL2v_ExplResOnc.P175.0125 | | | | |
| 7.4 | P1AG0716 | one-armed CD8 IgG-pH dep | 4.40E+06 | 3.45E−01 | 7.85E−08 | 78.5 |
| | | IL2v_ExplResOnc.P175.0368 | | | | |
| 7.1 | P1AG0716 | one-armed CD8 IgG-pH dep | 2.19E+05 | 3.79E−02 | 1.73E−07 | 173.4 |
| | | IL2v_ExplResOnc.P175.0368 | | | | |
| 6.8 | P1AG0716 | one-armed CD8 IgG-pH dep | 3.70E+05 | 1.20E−02 | 3.23E−08 | 32.3 |
| | | IL2v_ExplResOnc.P175.0368 | | | | |
| 6.5 | P1AG0716 | one-armed CD8 IgG-pH dep | 3.91E+05 | 6.19E−03 | 1.58E−08 | 15.8 |
| | | IL2v_ExplResOnc.P175.0368 | | | | |
| 6.2 | P1AG0716 | one-armed CD8 IgG-pH dep | 4.18E+05 | 4.00E−03 | 9.58E−09 | 9.6 |
| | | IL2v_ExplResOnc.P175.0368 | | | | |
| 6.0 | P1AG0716 | one-armed CD8 IgG-pH dep | 3.89E+05 | 2.85E−03 | 7.33E−09 | 7.3 |
| | | IL2v_ExplResOnc.P175.0368 | | | | |
| 7.4 | P1AG0717 | one-armed CD8 IgG-pH dep | 2.92E+05 | 5.74E−01 | 1.97E−06 | low |
| | | IL2v_ExplResOnc.P177.0035 | | | | |
| | | (IL2v (consensus all)) | | | | |
| 7.1 | P1AG0717 | one-armed CD8 IgG-pH dep | 2.57E+04 | 1.97E−01 | 7.67E−06 | 7666.0 |
| | | IL2v_ExplResOnc.P177.0035 | | | | |
| | | (IL2v (consensus all) | | | | |
| 6.8 | P1AG0717 | one-armed CD8 IgG-pH dep | 1.24E+04 | 5.51E−02 | 4.44E−06 | 4436.0 |
| | | IL2v_ExplResOnc.P177.0035 | | | | |
| | | (IL2v (consensus all) | | | | |
| 6.5 | P1AG0717 | one-armed CD8 IgG-pH dep | 3.37E+05 | 1.18E−02 | 3.50E−08 | 35.0 |
| | | IL2v_ExplResOnc.P177.0035 | | | | |
| | | (IL2v (consensus all)) | | | | |
| 6.2 | P1AG0717 | one-armed CD8 IgG-pH dep | 4.51E+05 | 4.71E−03 | 1.05E−08 | 10.5 |
| | | IL2v_ExplResOnc.P177.0035 | | | | |
| | | (IL2v (consensus all)) | | | | |
| 6.0 | P1AG0717 | one-armed CD8 IgG-pH dep | 4.73E+05 | 3.61E−03 | 7.63E−09 | 7.6 |
| | | IL2v_ExplResOnc.P177.0035 | | | | |
| | | (IL2v (consensus all)) | | | | |
| 7.4 | P1AG0718 | one-armed CD8 IgG-pH dep | 1.58E+05 | 2.65E+03 | 1.67E−02 | low |
| | | IL2v_ExplResOnc.P177.0036 | | | | |
| 7.1 | P1AG0718 | one-armed CD8 IgG-pH dep | 1.27E+06 | 7.13E−02 | 5.61E−08 | 56.1 |
| | | IL2v_ExplResOnc.P177.0036 | | | | |
| 6.8 | P1AG0718 | one-armed CD8 IgG-pH dep | 2.10E+05 | 4.90E−02 | 2.34E−07 | 233.7 |
| | | IL2v_ExplResOnc.P177.0036 | | | | |
| 6.5 | P1AG0718 | one-armed CD8 IgG-pH dep | 1.63E+05 | 2.09E−02 | 1.28E−07 | 128.0 |
| | | IL2v_ExplResOnc.P177.0036 | | | | |
| 6.2 | P1AG0718 | one-armed CD8 IgG-pH dep | 3.17E+05 | 7.86E−03 | 2.48E−08 | 24.8 |
| | | IL2v_ExplResOnc.P177.0036 | | | | |
| 6.0 | P1AG0718 | one-armed CD8 IgG-pH dep | 3.28E+05 | 4.47E−03 | 1.36E−08 | 13.6 |
| | | IL2v_ExplResOnc.P177.0036 | | | | |
| 7.4 | P1AG0719 | one-armed CD8 IgG-pH dep | 8.95E+04 | 5.65E+00 | 6.32E−05 | low |
| | | IL2v_ExplResOnc.P177.0156 | | | | |
| 7.1 | P1AG0719 | one-armed CD8 IgG-pH dep | 7.06E+04 | 4.70E−02 | 6.66E−07 | 665.9 |
| | | IL2v_ExplResOnc.P177.0156 | | | | |
| 6.8 | P1AG0719 | one-armed CD8 IgG-pH dep | 2.10E+05 | 1.17E−02 | 5.57E−08 | 55.7 |
| | | IL2v_ExplResOnc.P177.0156 | | | | |
| 6.5 | P1AG0719 | one-armed CD8 IgG-pH dep | 2.51E+05 | 5.60E−03 | 2.23E−08 | 22.3 |
| | | IL2v_ExplResOnc.P177.0156 | | | | |
| 6.2 | P1AG0719 | one-armed CD8 IgG-pH dep | 3.38E+05 | 3.74E−03 | 1.11E−08 | 11.1 |
| | | IL2v_ExplResOnc.P177.0156 | | | | |
| 6.0 | P1AG0719 | one-armed CD8 IgG-pH dep | 3.42E+05 | 2.37E−03 | 6.92E−09 | 6.9 |
| | | IL2v_ExplResOnc.P177.0156 | | | | |
| 7.4 | P1AG0720 | one-armed CD8 IgG-pH dep | 3.74E+05 | 1.46E−02 | 3.90E−08 | 39.0 |
| | | IL2v_ExplResOnc.P178.0145 | | | | |
| 7.1 | P1AG0720 | one-armed CD8 IgG-pH dep | 1.73E+05 | 1.12E−02 | 6.43E−08 | 64.3 |
| | | IL2v_ExplResOnc.P178.0145 | | | | |
| 6.8 | P1AG0720 | one-armed CD8 IgG-pH dep | 1.83E+05 | 5.27E−03 | 2.88E−08 | 28.8 |
| | | IL2v_ExplResOnc.P178.0145 | | | | |
| 6.5 | P1AG0720 | one-armed CD8 IgG-pH dep | 1.59E+05 | 3.26E−03 | 2.05E−08 | 20.5 |
| | | IL2v_ExplResOnc.P178.0145 | | | | |
| 6.2 | P1AG0720 | one-armed CD8 IgG-pH dep | 1.81E+05 | 1.92E−03 | 1.06E−08 | 10.6 |
| | | IL2v_ExplResOnc.P178.0145 | | | | |
| 6.0 | P1AG0720 | one-armed CD8 IgG-pH dep | 1.86E+05 | 1.65E−03 | 8.91E−09 | 8.9 |
| | | IL2v_ExplResOnc.P178.0145 | | | | |
| 7.4 | P1AG0721 | one-armed CD8 IgG-pH dep | low response | | | |
| | | IL2v-pH 1 (Q11E) | | | | |
| 7.1 | P1AG0721 | one-armed CD8 IgG-pH dep | | | | |
| | | IL2v-pH 1 (Q11E) | | | | |
| 6.8 | P1AG0721 | one-armed CD8 IgG-pH dep | | | | |
| | | IL2v-pH 1 (Q11E) | | | | |
| 6.5 | P1AG0721 | one-armed CD8 IgG-pH dep | | | | |
| | | IL2v-pH 1 (Q11E) | | | | |
| 6.2 | P1AG0721 | one-armed CD8 IgG-pH dep | | | | |
| | | IL2v-pH 1 (Q11E) | | | | |
| 6.0 | P1AG0721 | one-armed CD8 IgG-pH dep | | | | |
| | | IL2v-pH 1 (Q11E) | | | | |
| 7.4 | P1AG0722 | one-armed CD8 IgG-pH dep | 9.24E+04 | 2.72E−04 | 2.94E−09 | 2.9 |
| | | IL2v-pH 2 (E15Q) | | | | |
| 7.1 | P1AG0722 | one-armed CD8 IgG-pH dep | 6.86E+04 | 8.91E−05 | 1.30E−09 | 1.3 |
| | | IL2v-pH 2 (E15Q) | | | | |
| 6.8 | P1AG0722 | one-armed CD8 IgG-pH dep | 8.36E+04 | 1.63E−04 | 1.95E−09 | 1.9 |
| | | IL2v-pH 2 (E15Q) | | | | |
| 6.5 | P1AG0722 | one-armed CD8 IgG-pH dep | 6.34E+04 | 3.17E−04 | 5.00E−09 | 5.0 |
| | | IL2v-pH 2 (E15Q) | | | | |
| 6.2 | P1AG0722 | one-armed CD8 IgG-pH dep | 1.07E+05 | 1.41E−04 | 1.32E−09 | 1.3 |
| | | IL2v-pH 2 (E15Q) | | | | |
| 6.0 | P1AG0722 | one-armed CD8 IgG-pH dep | 1.27E+05 | 2.36E−04 | 1.85E−09 | 1.9 |
| | | IL2v-pH 2 (E15Q) | | | | |
| 7.4 | P1AG0723 | one-armed CD8 IgG-pH dep | 1.44E+05 | 1.47E−03 | 1.02E−08 | 10.2 |
| | | IL2v-pH 3 (H16E) | | | | |
| 7.1 | P1AG0723 | one-armed CD8 IgG-pH dep | 1.60E+05 | 6.33E−04 | 3.96E−09 | 4.0 |
| | | IL2v-pH 3 (H16E) | | | | |
| 6.8 | P1AG0723 | one-armed CD8 IgG-pH dep | 1.74E+05 | 4.15E−04 | 2.39E−09 | 2.4 |
| | | IL2v-pH 3 (H16E) | | | | |
| 6.5 | P1AG0723 | one-armed CD8 IgG-pH dep | 1.47E+05 | 3.58E−04 | 2.44E−09 | 2.4 |
| | | IL2v-pH 3 (H16E) | | | | |
| 6.2 | P1AG0723 | one-armed CD8 IgG-pH dep | 1.95E+05 | 2.31E−04 | 1.19E−09 | 1.2 |
| | | IL2v-pH 3 (H16E) | | | | |
| 6.0 | P1AG0723 | one-armed CD8 IgG-pH dep | 1.86E+05 | 1.52E−04 | 8.17E−10 | 0.8 |
| | | IL2v-pH 3 (H16E) | | | | |
| 7.4 | P1AG0724 | one-armed CD8 IgG-pH dep | 1.01E+05 | 1.90E−03 | 1.88E−08 | 18.8 |
| | | IL2v-pH 4 (L19D) | | | | |
| 7.1 | P1AG0724 | one-armed CD8 IgG-pH dep | 6.97E+04 | 1.56E−03 | 2.24E−08 | 22.4 |
| | | IL2v-pH 4 (L19D) | | | | |
| 6.8 | P1AG0724 | one-armed CD8 IgG-pH dep | 7.84E+04 | 1.16E−03 | 1.48E−08 | 14.8 |
| | | IL2v-pH 4 (L19D) | | | | |
| 6.5 | P1AG0724 | one-armed CD8 IgG-pH dep | 7.10E+04 | 1.24E−03 | 1.74E−08 | 17.4 |
| | | IL2v-pH 4 (L19D) | | | | |
| 6.2 | P1AG0724 | one-armed CD8 IgG-pH dep | 9.39E+04 | 9.72E−04 | 1.04E−08 | 10.4 |
| | | IL2v-pH 4 (L19D) | | | | |
| 6.0 | P1AG0724 | one-armed CD8 IgG-pH dep | 9.14E+04 | 9.70E−04 | 1.06E−08 | 10.6 |
| | | IL2v-pH 4 (L19D) | | | | |
| 7.4 | P1AG0725 | one-armed CD8 IgG-pH dep | low response | | | |
| | | IL2v-pH 5 (Q22E) | | | | |
| 7.1 | P1AG0725 | one-armed CD8 IgG-pH dep | | | | |
| | | IL2v-pH 5 (Q22E) | | | | |
| 6.8 | P1AG0725 | one-armed CD8 IgG-pH dep | | | | |
| | | IL2v-pH 5 (Q22E) | | | | |
| 6.5 | P1AG0725 | one-armed CD8 IgG-pH dep | | | | |
| | | IL2v-pH 5 (Q22E) | | | | |
| 6.2 | P1AG0725 | one-armed CD8 IgG-pH dep | | | | |
| | | IL2v-pH 5 (Q22E) | | | | |
| 6.0 | P1AG0725 | one-armed CD8 IgG-pH dep | | | | |
| | | IL2v-pH 5 (Q22E) | | | | |
| 7.4 | P1AG0726 | one-armed CD8 IgG-pH dep | 1.05E+05 | 3.98E−04 | 3.79E−09 | 3.8 |
| | | IL2v-pH 6 (M23Q) | | | | |
| 7.1 | P1AG0726 | one-armed CD8 IgG-pH dep | 8.88E+04 | 1.44E−04 | 1.63E−09 | 1.6 |
| | | IL2v-pH 6 (M23Q) | | | | |
| 6.8 | P1AG0726 | one-armed CD8 IgG-pH dep | 8.49E+04 | 3.17E−04 | 3.74E−09 | 3.7 |
| | | IL2v-pH 6 (M23Q) | | | | |
| 6.5 | P1AG0726 | one-armed CD8 IgG-pH dep | 8.36E+04 | 4.87E−04 | 5.83E−09 | 5.8 |
| | | IL2v-pH 6 (M23Q) | | | | |
| 6.2 | P1AG0726 | one-armed CD8 IgG-pH dep | 1.07E+05 | 7.57E−05 | 7.06E−10 | 0.7 |
| | | IL2v-pH 6 (M23Q) | | | | |
| 6.0 | P1AG0726 | one-armed CD8 IgG-pH dep | 8.82E+04 | 2.07E−04 | 2.35E−09 | 2.3 |
| | | IL2v-pH 6 (M23Q) | | | | |
| 7.4 | P1AG0727 | one-armed CD8 IgG-pH dep | 2.04E+05 | 2.49E−04 | 1.22E−09 | 1.2 |
| | | IL2v-pH 7_(R81D) | | | | |
| 7.1 | P1AG0727 | one-armed CD8 IgG-pH dep | 2.04E+05 | 5.30E−05 | 2.59E−10 | 0.3 |
| | | IL2v-pH 7_(R81D) | | | | |
| 6.8 | P1AG0727 | one-armed CD8 IgG-pH dep | 2.18E+05 | 5.89E−05 | 2.71E−10 | 0.3 |
| | | IL2v-pH 7_(R81D) | | | | |
| 6.5 | P1AG0727 | one-armed CD8 IgG-pH dep | 1.43E+05 | 1.13E−04 | 7.87E−10 | 0.8 |
| | | IL2v-pH 7_(R81D) | | | | |
| 6.2 | P1AG0727 | one-armed CD8 IgG-pH dep | 1.87E+05 | <1.0E−07 | <1.0E−12 | <0.1 |
| | | IL2v-pH 7_(R81D) | | | | |
| 6.0 | P1AG0727 | one-armed CD8 IgG-pH dep | 1.73E+05 | <1.0E−07 | <1.0E−12 | <0.1 |
| | | IL2v-pH 7_(R81D) | | | | |
| 7.4 | P1AG0728 | one-armed CD8 IgG-pH dep | 9.87E+04 | 2.99E−04 | 3.03E−09 | 3.0 |
| | | IL2v-pH 8_(D84E) | | | | |
| 7.1 | P1AG0728 | one-armed CD8 IgG-pH dep | 1.00E+05 | <1.0E−07 | <1.0E−12 | <0.1 |
| | | IL2v-pH 8_(D84E) | | | | |
| 6.8 | P1AG0728 | one-armed CD8 IgG-pH dep | 1.05E+05 | <1.0E−07 | <1.0E−12 | <0.1 |
| | | IL2v-pH 8_(D84E) | | | | |
| 6.5 | P1AG0728 | one-armed CD8 IgG-pH dep | 6.86E+04 | 6.09E−05 | 8.88E−10 | 0.9 |
| | | IL2v-pH 8_(D84E) | | | | |
| 6.2 | P1AG0728 | one-armed CD8 IgG-pH dep | 1.05E+05 | <1.0E−07 | <1.0E−12 | <0.1 |
| | | IL2v-pH 8_(D84E) | | | | |
| 6.0 | P1AG0728 | one-armed CD8 IgG-pH dep | 8.72E+04 | <1.0E−07 | <1.0E−12 | <0.1 |
| | | IL2v-pH 8_(D84E) | | | | |
| 7.4 | P1AG0729 | one-armed CD8 IgG-pH dep | 1.11E+05 | 3.52E−04 | 3.18E−09 | 3.2 |
| | | IL2v-pH 9 (S87E) | | | | |
| 7.1 | P1AG0729 | one-armed CD8 IgG-pH dep | 1.21E+05 | 5.86E−05 | 4.83E−10 | 0.5 |
| | | IL2v-pH 9 (S87E) | | | | |
| 6.8 | P1AG0729 | one-armed CD8 IgG-pH dep | 1.47E+05 | 5.69E−05 | 3.88E−10 | 0.4 |
| | | IL2v-pH 9 (S87E) | | | | |
| 6.5 | P1AG0729 | one-armed CD8 IgG-pH dep | 1.01E+05 | 1.22E−04 | 1.20E−09 | 1.2 |
| | | IL2v-pH 9 (S87E) | | | | |
| 6.2 | P1AG0729 | one-armed CD8 IgG-pH dep | 1.51E+05 | 5.04E−05 | 3.34E−10 | 0.3 |
| | | IL2v-pH 9 (S87E) | | | | |
| 6.0 | P1AG0729 | one-armed CD8 IgG-pH dep | 1.25E+05 | <1.0E−07 | <1.0E−12 | <0.1 |
| | | IL2v-pH 9 (S87E) | | | | |
| 7.4 | P1AG0730 | one-armed CD8 IgG-pH dep | low response | | | |
| | | IL2v-pH 11 (Q126H) | | | | |
| 7.1 | P1AG0730 | one-armed CD8 IgG-pH dep | | | | |
| | | IL2v-pH 11 (Q126H) | | | | |
| 6.8 | P1AG0730 | one-armed CD8 IgG-pH dep | | | | |
| | | IL2v-pH 11 (Q126H) | | | | |
| 6.5 | P1AG0730 | one-armed CD8 IgG-pH dep | | | | |
| | | IL2v-pH 11 (Q126H) | | | | |
| 6.2 | P1AG0730 | one-armed CD8 IgG-pH dep | | | | |
| | | IL2v-pH 11 (Q126H) | | | | |
| 6.0 | P1AG0730 | one-armed CD8 IgG-pH dep | | | | |
| | | IL2v-pH 11 (Q126H) | | | | |
| 7.4 | P1AG0731 | one-armed CD8 IgG-pH dep | 6.25E+04 | 8.99E−04 | 1.44E−08 | 14.4 |
| | | IL2v-pH 12 (S130E) | | | | |
| 7.1 | P1AG0731 | one-armed CD8 IgG-pH dep | 1.02E+05 | 6.37E−04 | 6.24E−09 | 6.2 |
| | | IL2v-pH 12 (S130E) | | | | |
| 6.8 | P1AG0731 | one-armed CD8 IgG-pH dep | 8.27E+04 | 4.33E−04 | 5.23E−09 | 5.2 |
| | | IL2v-pH 12 (S130E) | | | | |
| 6.5 | P1AG0731 | one-armed CD8 IgG-pH dep | 1.82E+04 | 4.36E−04 | 2.39E−08 | 23.9 |
| | | IL2v-pH 12 (S130E) | | | | |
| 6.2 | P1AG0731 | one-armed CD8 IgG-pH dep | 7.67E+04 | 6.92E−04 | 9.01E−09 | 9.0 |
| | | IL2v-pH 12 (S130E) | | | | |
| 6.0 | P1AG0731 | one-armed CD8 IgG-pH dep | 3.59E+04 | <1.0E−07 | <1.0E−12 | <0.1 |
| | | IL2v-pH 12 (S130E) | | | | |
| 7.4 | P1AG0733 | one-armed CD8 IgG-pH dep | 1.02E+05 | 3.42E−04 | 3.35E−09 | 3.4 |
| | | IL2v-pH 14_(T133D) | | | | |
| 7.1 | P1AG0733 | one-armed CD8 IgG-pH dep | 1.24E+05 | 2.78E−04 | 2.25E−09 | 2.2 |
| | | IL2v-pH 14_(T133D) | | | | |
| 6.8 | P1AG0733 | one-armed CD8 IgG-pH dep | 1.33E+05 | 1.64E−04 | 1.23E−09 | 1.2 |
| | | IL2v-pH 14_(T133D) | | | | |
| 6.5 | P1AG0733 | one-armed CD8 IgG-pH dep | 8.35E+04 | 1.99E−04 | 2.39E−09 | 2.4 |
| | | IL2v-pH 14_(T133D) | | | | |
| 6.2 | P1AG0733 | one-armed CD8 IgG-pH dep | 1.42E+05 | 3.19E−04 | 2.24E−09 | 2.2 |
| | | IL2v-pH 14_(T133D) | | | | |
| 6.0 | P1AG0733 | one-armed CD8 IgG-pH dep | 1.22E+05 | 1.68E−04 | 1.38E−09 | 1.4 |
| | | IL2v-pH 14_(T133D) | | | | |
| 7.4 | P1AG0700 | one-armed CD8 IgG-pH dep | low response | | | |
| | | IL2v_ExplResOnc.P173.0127 | | | | |
| 7.1 | P1AG0700 | one-armed CD8 IgG-pH dep | 3.27E+05 | 9.61E−02 | 2.94E−07 | 293.50 |
| | | IL2v_ExplResOnc.P173.0127 | | | | |
| 6.8 | P1AG0700 | one-armed CD8 IgG-pH dep | 5.44E+05 | 1.07E−02 | 1.96E−08 | 19.58 |
| | | IL2v_ExplResOnc.P173.0127 | | | | |
| 6.5 | P1AG0700 | one-armed CD8 IgG-pH dep | 4.83E+05 | 4.84E−03 | 1.00E−08 | 10.02 |
| | | IL2v_ExplResOnc.P173.0127 | | | | |
| 6.2 | P1AG0700 | one-armed CD8 IgG-pH dep | 4.97E+05 | 2.16E−03 | 4.35E−09 | 4.35 |
| | | IL2v_ExplResOnc.P173.0127 | | | | |
| 6.0 | P1AG0700 | one-armed CD8 IgG-pH dep | 4.63E+05 | 1.69E−03 | 3.65E−09 | 3.65 |
| | | IL2v_ExplResOnc.P173.0127 | | | | |
| 7.4 | P1AG0735 | one-armed CD8 IgG-pH dep | 3.85E+05 | 7.25E−03 | 1.88E−08 | 18.8 |
| | | IL2v-pH 17 (consensus beta) | | | | |
| 7.1 | P1AG0735 | one-armed CD8 IgG-pH dep | 4.30E+05 | 4.47E−03 | 1.04E−08 | 10.4 |
| | | IL2v-pH 17 (consensus beta) | | | | |
| 6.8 | P1AG0735 | one-armed CD8 IgG-pH dep | 4.68E+05 | 3.08E−03 | 6.58E−09 | 6.6 |
| | | IL2v-pH 17 (consensus beta) | | | | |
| 6.5 | P1AG0735 | one-armed CD8 IgG-pH dep | 4.21E+05 | 2.23E−03 | 5.30E−09 | 5.3 |
| | | IL2v-pH 17 (consensus beta) | | | | |
| 6.2 | P1AG0735 | one-armed CD8 IgG-pH dep | 4.57E+05 | 1.58E−03 | 3.45E−09 | 3.5 |
| | | IL2v-pH 17 (consensus beta) | | | | |
| 6.0 | P1AG0735 | one-armed CD8 IgG-pH dep | 4.44E+05 | 1.43E−03 | 3.23E−09 | 3.2 |
| | | IL2v-pH 17 (consensus beta) | | | | |
| 7.4 | P1AG0736 | one-armed CD8 IgG-pH dep | 1.19E+05 | 1.12E−03 | 9.42E−09 | 9.4 |
| | | IL2v-pH 18 (consensus | | | | |
| | | gamma) | | | | |
| 7.1 | P1AG0736 | one-armed CD8 IgG-pH dep | 1.04E+05 | 5.74E−04 | 5.49E−09 | 5.5 |
| | | IL2v-pH 18 (consensus | | | | |
| | | gamma) | | | | |
| 6.8 | P1AG0736 | one-armed CD8 IgG-pH dep | 1.11E+05 | 4.00E−04 | 3.60E−09 | 3.6 |
| | | IL2v-pH 18 (consensus | | | | |
| | | gamma) | | | | |
| 6.5 | P1AG0736 | one-armed CD8 IgG-pH dep | 6.97E+04 | 3.68E−04 | 5.29E−09 | 5.3 |
| | | IL2v-pH 18 (consensus | | | | |
| | | gamma) | | | | |
| 6.2 | P1AG0736 | one-armed CD8 IgG-pH dep | 9.78E+04 | 2.74E−04 | 2.80E−09 | 2.8 |
| | | IL2v-pH 18 (consensus | | | | |
| | | gamma) | | | | |
| 6.0 | P1AG0736 | one-armed CD8 IgG-pH dep | 1.05E+05 | 2.80E−04 | 2.66E−09 | 2.7 |
| | | IL2v-pH 18 (consensus | | | | |
| | | gamma) | | | | |
| 7.4 | P1AG0737 | one-armed CD8 IgG-pH dep | 4.89E+05 | 5.34E−04 | 1.09E−09 | 1.1 |
| | | IL2v-pH_2nd library template | | | | |
| 7.1 | P1AG0737 | one-armed CD8 IgG-pH dep | 4.39E+05 | 3.30E−04 | 7.53E−10 | 0.8 |
| | | IL2v-pH_2nd library template | | | | |
| 6.8 | P1AG0737 | one-armed CD8 IgG-pH dep | 4.06E+05 | 2.93E−04 | 7.22E−10 | 0.7 |
| | | IL2v-pH_2nd library template | | | | |
| 6.5 | P1AG0737 | one-armed CD8 IgG-pH dep | 3.15E+05 | 2.79E−04 | 8.86E−10 | 0.9 |
| | | IL2v-pH_2nd library template | | | | |
| 6.2 | P1AG0737 | one-armed CD8 IgG-pH dep | 2.93E+05 | 1.83E−04 | 6.24E−10 | 0.6 |
| | | IL2v-pH_2nd library template | | | | |
| 6.0 | P1AG0737 | one-armed CD8 IgG-pH dep | 2.66E+05 | 1.86E−04 | 7.01E−10 | 0.7 |
| | | IL2v-pH_2nd library template | | | | |
| 7.4 | P1AG0738 | one-armed CD8 IgG-Parental | 7.03E+04 | 3.98E−04 | 5.66E−09 | 5.7 |
| | | IL2v | | | | |
| 7.1 | P1AG0738 | one-armed CD8 IgG-Parental | 7.24E+04 | 1.96E−04 | 2.71E−09 | 2.7 |
| | | IL2v | | | | |
| 6.8 | P1AG0738 | one-armed CD8 IgG-Parental | 6.78E+04 | 8.37E−05 | 1.24E−09 | 1.2 |
| | | IL2v | | | | |
| 6.5 | P1AG0738 | one-armed CD8 IgG-Parental | 2.72E+04 | 1.29E−04 | 4.74E−09 | 4.7 |
| | | IL2v | | | | |
| 6.2 | P1AG0738 | one-armed CD8 IgG-Parental | 6.55E+04 | <1.0E−07 | <1.0E−12 | <0.1 |
| | | IL2v | | | | |
| 6.0 | P1AG0738 | one-armed CD8 IgG-Parental | 5.16E+04 | <1.0E−07 | <1.0E−12 | <0.1 |
| | | IL2v | | | | |
| 7.4 | P1AG0739 | one-armed CD8 IgG-pH dep | 1.71E+05 | 2.82E−04 | 1.65E−09 | 1.7 |
| | | IL2v-pH 10 (R120H) | | | | |
| 7.1 | P1AG0739 | one-armed CD8 IgG-pH dep | 1.83E+05 | 1.97E−04 | 1.08E−09 | 1.1 |
| | | IL2v-pH 10 (R120H) | | | | |
| 6.8 | P1AG0739 | one-armed CD8 IgG-pH dep | 1.89E+05 | 1.88E−04 | 9.95E−10 | 1.0 |
| | | IL2v-pH 10 (R120H) | | | | |
| 6.5 | P1AG0739 | one-armed CD8 IgG-pH dep | 1.07E+05 | 3.06E−04 | 2.87E−09 | 2.9 |
| | | IL2v-pH 10 (R120H) | | | | |
| 6.2 | P1AG0739 | one-armed CD8 IgG-pH dep | 1.44E+05 | 3.99E−04 | 2.78E−09 | 2.8 |
| | | IL2v-pH 10 (R120H) | | | | |
| 6.0 | P1AG0739 | one-armed CD8 IgG-pH dep | 1.22E+05 | 4.90E−04 | 4.01E−09 | 4.0 |
| | | IL2v-pH 10 (R120H) | | | | |

For the majority of the selected IL2v variants, the BLI data show that they bind very poorly at pH 7.4 and pH 7.0 and that their receptor binding strength increases as the pH decreases. Overall, there is an inverse correlation between affinity (KD) and pH and the improved affinities as the pH decreases from pH 7.4 to pH 6.0 are mainly caused by increasingly slower dissociation rate constants (kd). In contrast to the IL2v variants that have been selected for pH-dependency, parental IL2v (P1AG0738, one-armed CD8 IgG-Parental IL2v) exhibits higher affinities and slower dissociation rate constants at all pH values tested but in particular at the higher pH values at around neutral pH. Importantly, the IL2v variants that have been selected for pH-dependency exhibit much weaker affinities to the human IL2Rbg at around neutral pH (pH 7.4, pH 7.0, pH 6.8) compared to parental IL2v and should thus lead to reduced systemic receptor recruitment. In the slightly acidic tumor microenvironment (TME), however, these variants are expected to exhibit better receptor recruitment and subsequent signaling via the IL2R pathway than in the periphery, outside the TME. The introduction of single mutations into IL2v does not lead to the same extent of pH-dependency. In general, their affinity range between pH 7.4 and pH 6.0 is more comparable to parental IL2v than to the IL2v variants that have been selected for pH-dependency. Most of the selected pH-dependent IL2v variants are cross-reactive to murine IL2Rbg, however, they do not exhibit pH-dependency to the same extent as on the human IL2Rbg. The results are summarized in the Table 8.

TABLE 8
Binding kinetics of one-armed CD8-targeted IgG IL2v
fusions to murine IL-2Rbg at different pH values

| Sample ID | concept ID | construct | kon(1/Ms) | kdis(1/s) | KD (M) | KD (nM) |
|---|---|---|---|---|---|---|
| 7.4 | P1AG0697 | one-armed CD8 IgG-pH dep | 1.58E+05 | 1.00E−03 | 6.34E−09 | 6.3 |
| | | IL2v_ExplResOnc.P172.0344 | | | | |
| 7.1 | P1AG0697 | one-armed CD8 IgG-pH dep | 1.62E+05 | 9.50E−04 | 5.88E−09 | 5.9 |
| | | IL2v_ExplResOnc.P172.0344 | | | | |
| 6.8 | P1AG0697 | one-armed CD8 IgG-pH dep | 1.39E+05 | 1.05E−03 | 7.56E−09 | 7.6 |
| | | IL2v_ExplResOnc.P172.0344 | | | | |
| 6.5 | P1AG0697 | one-armed CD8 IgG-pH dep | 1.31E+05 | 1.03E−03 | 7.82E−09 | 7.8 |
| | | IL2v_ExplResOnc.P172.0344 | | | | |
| 6.2 | P1AG0697 | one-armed CD8 IgG-pH dep | 1.26E+05 | 1.02E−03 | 8.17E−09 | 8.2 |
| | | IL2v_ExplResOnc.P172.0344 | | | | |
| 6.0 | P1AG0697 | one-armed CD8 IgG-pH dep | 1.37E+05 | 1.15E−03 | 8.42E−09 | 8.4 |
| | | IL2v_ExplResOnc.P172.0344 | | | | |
| 7.4 | P1AG0698 | one-armed CD8 IgG-pH dep | 1.89E+05 | 1.18E−03 | 6.24E−09 | 6.2 |
| | | IL2v_ExplResOnc.P173.0079 | | | | |
| 7.1 | P1AG0698 | one-armed CD8 IgG-pH dep | 2.05E+05 | 1.05E−03 | 5.13E−09 | 5.1 |
| | | IL2v_ExplResOnc.P173.0079 | | | | |
| 6.8 | P1AG0698 | one-armed CD8 IgG-pH dep | 2.04E+05 | 1.34E−03 | 6.55E−09 | 6.6 |
| | | IL2v_ExplResOnc.P173.0079 | | | | |
| 6.5 | P1AG0698 | one-armed CD8 IgG-pH dep | 2.22E+05 | 1.36E−03 | 6.12E−09 | 6.1 |
| | | IL2v_ExplResOnc.P173.0079 | | | | |
| 6.2 | P1AG0698 | one-armed CD8 IgG-pH dep | 2.39E+05 | 1.27E−03 | 5.32E−09 | 5.3 |
| | | IL2v_ExplResOnc.P173.0079 | | | | |
| 6.0 | P1AG0698 | one-armed CD8 IgG-pH dep | 2.53E+05 | 1.42E−03 | 5.62E−09 | 5.6 |
| | | IL2v_ExplResOnc.P173.0079 | | | | |
| 7.4 | P1AG0699 | one-armed CD8 IgG-pH dep | 1.78E+05 | 2.02E−03 | 1.13E−08 | 11.3 |
| | | IL2v_ExplResOnc.P173.0087 | | | | |
| 7.1 | P1AG0699 | one-armed CD8 IgG-pH dep | 1.63E+05 | 1.57E−03 | 9.59E−09 | 9.6 |
| | | IL2v_ExplResOnc.P173.0087 | | | | |
| 6.8 | P1AG0699 | one-armed CD8 IgG-pH dep | 1.80E+05 | 1.57E−03 | 8.72E−09 | 8.7 |
| | | IL2v_ExplResOnc.P173.0087 | | | | |
| 6.5 | P1AG0699 | one-armed CD8 IgG-pH dep | 1.88E+05 | 1.20E−03 | 6.38E−09 | 6.4 |
| | | IL2v_ExplResOnc.P173.0087 | | | | |
| 6.2 | P1AG0699 | one-armed CD8 IgG-pH dep | 1.92E+05 | 5.90E−04 | 3.08E−09 | 3.1 |
| | | IL2v_ExplResOnc.P173.0087 | | | | |
| 6.0 | P1AG0699 | one-armed CD8 IgG-pH dep | 2.24E+05 | 9.79E−04 | 4.36E−09 | 4.4 |
| | | IL2v_ExplResOnc.P173.0087 | | | | |
| 7.4 | P1AG0701 | one-armed CD8 IgG-pH dep | low response | | | |
| | | IL2v_ExplResOnc.P173.0156 | | | | |
| 7.1 | P1AG0701 | one-armed CD8 IgG-pH dep | | | | |
| | | IL2v_ExplResOnc.P173.0156 | | | | |
| 6.8 | P1AG0701 | one-armed CD8 IgG-pH dep | | | | |
| | | IL2v_ExplResOnc.P173.0156 | | | | |
| 6.5 | P1AG0701 | one-armed CD8 IgG-pH dep | | | | |
| | | IL2v_ExplResOnc.P173.0156 | | | | |
| 6.2 | P1AG0701 | one-armed CD8 IgG-pH dep | | | | |
| | | IL2v_ExplResOnc.P173.0156 | | | | |
| 6.0 | P1AG0701 | one-armed CD8 IgG-pH dep | | | | |
| | | IL2v_ExplResOnc.P173.0156 | | | | |
| 7.4 | P1AG0702 | one-armed CD8 IgG-pH dep | low response | | | |
| | | IL2v_ExplResOnc.P173.0182 | | | | |
| 7.1 | P1AG0702 | one-armed CD8 IgG-pH dep | | | | |
| | | IL2v_ExplResOnc.P173.0182 | | | | |
| 6.8 | P1AG0702 | one-armed CD8 IgG-pH dep | | | | |
| | | IL2v_ExplResOnc.P173.0182 | | | | |
| 6.5 | P1AG0702 | one-armed CD8 IgG-pH dep | | | | |
| | | IL2v_ExplResOnc.P173.0182 | | | | |
| 6.2 | P1AG0702 | one-armed CD8 IgG-pH dep | | | | |
| | | IL2v_ExplResOnc.P173.0182 | | | | |
| 6.0 | P1AG0702 | one-armed CD8 IgG-pH dep | | | | |
| | | IL2v_ExplResOnc.P173.0182 | | | | |
| 7.4 | P1AG0703 | one-armed CD8 IgG-pH dep | 2.39E+05 | 2.73E−03 | 1.14E−08 | 11.4 |
| | | IL2v_ExplResOnc.P173.0239 | | | | |
| 7.1 | P1AG0703 | one-armed CD8 IgG-pH dep | 2.33E+05 | 2.18E−03 | 9.36E−09 | 9.4 |
| | | IL2v_ExplResOnc.P173.0239 | | | | |
| 6.8 | P1AG0703 | one-armed CD8 IgG-pH dep | 2.60E+05 | 3.09E−03 | 1.19E−08 | 11.9 |
| | | IL2v_ExplResOnc.P173.0239 | | | | |
| 6.5 | P1AG0703 | one-armed CD8 IgG-pH dep | 2.67E+05 | 2.58E−03 | 9.66E−09 | 9.7 |
| | | IL2v_ExplResOnc.P173.0239 | | | | |
| 6.2 | P1AG0703 | one-armed CD8 IgG-pH dep | 2.81E+05 | 2.13E−03 | 7.60E−09 | 7.6 |
| | | IL2v_ExplResOnc.P173.0239 | | | | |
| 6.0 | P1AG0703 | one-armed CD8 IgG-pH dep | 2.97E+05 | 1.89E−03 | 6.37E−09 | 6.4 |
| | | IL2v_ExplResOnc.P173.0239 | | | | |
| 7.4 | P1AG0704 | one-armed CD8 IgG-pH dep | 2.09E+05 | 2.20E−03 | 1.05E−08 | 10.5 |
| | | IL2v_ExplResOnc.P173.0255 | | | | |
| 7.1 | P1AG0704 | one-armed CD8 IgG-pH dep | 1.93E+05 | 1.62E−03 | 8.42E−09 | 8.4 |
| | | IL2v_ExplResOnc.P173.0255 | | | | |
| 6.8 | P1AG0704 | one-armed CD8 IgG-pH dep | 2.10E+05 | 1.90E−03 | 9.06E−09 | 9.1 |
| | | IL2v_ExplResOnc.P173.0255 | | | | |
| 6.5 | P1AG0704 | one-armed CD8 IgG-pH dep | 1.87E+05 | 1.07E−03 | 5.74E−09 | 5.7 |
| | | IL2v_ExplResOnc.P173.0255 | | | | |
| 6.2 | P1AG0704 | one-armed CD8 IgG-pH dep | 2.29E+05 | 1.73E−03 | 7.58E−09 | 7.6 |
| | | IL2v_ExplResOnc.P173.0255 | | | | |
| 6.0 | P1AG0704 | one-armed CD8 IgG-pH dep | 2.55E+05 | 1.98E−03 | 7.75E−09 | 7.7 |
| | | IL2v_ExplResOnc.P173.0255 | | | | |
| 7.4 | P1AG0705 | one-armed CD8 IgG-pH dep | low response | | | |
| | | IL2v_ExplResOnc.P173.0259 | | | | |
| 7.1 | P1AG0705 | one-armed CD8 IgG-pH dep | | | | |
| | | IL2v_ExplResOnc.P173.0259 | | | | |
| 6.8 | P1AG0705 | one-armed CD8 IgG-pH dep | | | | |
| | | IL2v_ExplResOnc.P173.0259 | | | | |
| 6.5 | P1AG0705 | one-armed CD8 IgG-pH dep | | | | |
| | | IL2v_ExplResOnc.P173.0259 | | | | |
| 6.2 | P1AG0705 | one-armed CD8 IgG-pH dep | | | | |
| | | IL2v_ExplResOnc.P173.0259 | | | | |
| 6.0 | P1AG0705 | one-armed CD8 IgG-pH dep | | | | |
| | | IL2v_ExplResOnc.P173.0259 | | | | |
| 7.4 | P1AG0706 | one-armed CD8 IgG-pH dep | 2.90E+05 | 4.71E−04 | 1.63E−09 | 1.6 |
| | | IL2v_ExplResOnc.P173.0364 | | | | |
| 7.1 | P1AG0706 | one-armed CD8 IgG-pH dep | 2.74E+05 | 5.33E−04 | 1.94E−09 | 1.9 |
| | | IL2v_ExplResOnc.P173.0364 | | | | |
| 6.8 | P1AG0706 | one-armed CD8 IgG-pH dep | 2.67E+05 | 6.08E−04 | 2.28E−09 | 2.3 |
| | | IL2v_ExplResOnc.P173.0364 | | | | |
| 6.5 | P1AG0706 | one-armed CD8 IgG-pH dep | 2.66E+05 | 6.19E−04 | 2.32E−09 | 2.3 |
| | | IL2v_ExplResOnc.P173.0364 | | | | |
| 6.2 | P1AG0706 | one-armed CD8 IgG-pH dep | 2.27E+05 | 7.62E−04 | 3.35E−09 | 3.4 |
| | | IL2v_ExplResOnc.P173.0364 | | | | |
| 6.0 | P1AG0706 | one-armed CD8 IgG-pH dep | 2.34E+05 | 1.08E−03 | 4.61E−09 | 4.6 |
| | | IL2v_ExplResOnc.P173.0364 | | | | |
| 7.4 | P1AG0707 | one-armed CD8 IgG-pH dep | 2.34E+05 | 1.53E−03 | 6.52E−09 | 6.5 |
| | | IL2v_ExplResOnc.P173.0371 | | | | |
| 7.1 | P1AG0707 | one-armed CD8 IgG-pH dep | 2.34E+05 | 1.32E−03 | 5.63E−09 | 5.6 |
| | | IL2v_ExplResOnc.P173.0371 | | | | |
| 6.8 | P1AG0707 | one-armed CD8 IgG-pH dep | 2.42E+05 | 1.59E−03 | 6.58E−09 | 6.6 |
| | | IL2v_ExplResOnc.P173.0371 | | | | |
| 6.5 | P1AG0707 | one-armed CD8 IgG-pH dep | 2.63E+05 | 1.65E−03 | 6.29E−09 | 6.3 |
| | | IL2v_ExplResOnc.P173.0371 | | | | |
| 6.2 | P1AG0707 | one-armed CD8 IgG-pH dep | 2.80E+05 | 1.68E−03 | 5.99E−09 | 6.0 |
| | | IL2v_ExplResOnc.P173.0371 | | | | |
| 6.0 | P1AG0707 | one-armed CD8 IgG-pH dep | 2.61E+05 | 1.72E−03 | 6.61E−09 | 6.6 |
| | | IL2v_ExplResOnc.P173.0371 | | | | |
| 7.4 | P1AG0708 | one-armed CD8 IgG-pH dep | 2.25E+05 | 2.99E−03 | 1.33E−08 | 13.3 |
| | | IL2v_ExplResOnc.P174.0040 | | | | |
| 7.1 | P1AG0708 | one-armed CD8 IgG-pH dep | 2.11E+05 | 2.52E−03 | 1.19E−08 | 11.9 |
| | | IL2v_ExplResOnc.P174.0040 | | | | |
| 6.8 | P1AG0708 | one-armed CD8 IgG-pH dep | 2.16E+05 | 2.32E−03 | 1.07E−08 | 10.7 |
| | | IL2v_ExplResOnc.P174.0040 | | | | |
| 6.5 | P1AG0708 | one-armed CD8 IgG-pH dep | 2.32E+05 | 2.10E−03 | 9.06E−09 | 9.1 |
| | | IL2v_ExplResOnc.P174.0040 | | | | |
| 6.2 | P1AG0708 | one-armed CD8 IgG-pH dep | 2.36E+05 | 1.59E−03 | 6.74E−09 | 6.7 |
| | | IL2v_ExplResOnc.P174.0040 | | | | |
| 6.0 | P1AG0708 | one-armed CD8 IgG-pH dep | 2.80E+05 | 2.03E−03 | 7.25E−09 | 7.3 |
| | | IL2v_ExplResOnc.P174.0040 | | | | |
| 7.4 | P1AG0709 | one-armed CD8 IgG-pH dep | low response | | | |
| | | IL2v_ExplResOnc.P174.0173 | | | | |
| 7.1 | P1AG0709 | one-armed CD8 IgG-pH dep | | | | |
| | | IL2v_ExplResOnc.P174.0173 | | | | |
| 6.8 | P1AG0709 | one-armed CD8 IgG-pH dep | | | | |
| | | IL2v_ExplResOnc.P174.0173 | | | | |
| 6.5 | P1AG0709 | one-armed CD8 IgG-pH dep | | | | |
| | | IL2v_ExplResOnc.P174.0173 | | | | |
| 6.2 | P1AG0709 | one-armed CD8 IgG-pH dep | | | | |
| | | IL2v_ExplResOnc.P174.0173 | | | | |
| 6.0 | P1AG0709 | one-armed CD8 IgG-pH dep | | | | |
| | | IL2v_ExplResOnc.P174.0173 | | | | |
| 7.4 | P1AG0710 | one-armed CD8 IgG-pH dep | 2.18E+05 | 1.04E−03 | 4.78E−09 | 4.8 |
| | | IL2v_ExplResOnc.P174.0238 | | | | |
| 7.1 | P1AG0710 | one-armed CD8 IgG-pH dep | 2.20E+05 | 8.22E−04 | 3.73E−09 | 3.7 |
| | | IL2v_ExplResOnc.P174.0238 | | | | |
| 6.8 | P1AG0710 | one-armed CD8 IgG-pH dep | 2.51E+05 | 1.03E−03 | 4.12E−09 | 4.1 |
| | | IL2v_ExplResOnc.P174.0238 | | | | |
| 6.5 | P1AG0710 | one-armed CD8 IgG-pH dep | 2.91E+05 | 1.04E−03 | 3.58E−09 | 3.6 |
| | | IL2v_ExplResOnc.P174.0238 | | | | |
| 6.2 | P1AG0710 | one-armed CD8 IgG-pH dep | 3.16E+05 | 9.02E−04 | 2.85E−09 | 2.9 |
| | | IL2v_ExplResOnc.P174.0238 | | | | |
| 6.0 | P1AG0710 | one-armed CD8 IgG-pH dep | 3.47E+05 | 9.15E−04 | 2.63E−09 | 2.6 |
| | | IL2v_ExplResOnc.P174.0238 | | | | |
| 7.4 | P1AG0711 | one-armed CD8 IgG-pH dep | 7.50E+04 | 3.66E−04 | 4.88E−09 | 4.9 |
| | | IL2v_ExplResOnc.P174.0277 | | | | |
| 7.1 | P1AG0711 | one-armed CD8 IgG-pH dep | 9.94E+04 | 1.42E−04 | 1.43E−09 | 1.4 |
| | | IL2v_ExplResOnc.P174.0277 | | | | |
| 6.8 | P1AG0711 | one-armed CD8 IgG-pH dep | 1.05E+05 | 3.02E−04 | 2.87E−09 | 2.9 |
| | | IL2v_ExplResOnc.P174.0277 | | | | |
| 6.5 | P1AG0711 | one-armed CD8 IgG-pH dep | 7.08E+04 | 1.68E−04 | 2.38E−09 | 2.4 |
| | | IL2v_ExplResOnc.P174.0277 | | | | |
| 6.2 | P1AG0711 | one-armed CD8 IgG-pH dep | 4.56E+04 | <1.0E−07 | <1.0E−12 | <0.1 |
| | | IL2v_ExplResOnc.P174.0277 | | | | |
| 6.0 | P1AG0711 | one-armed CD8 IgG-pH dep | 7.70E+04 | <1.0E−07 | <1.0E−12 | <0.1 |
| | | IL2v_ExplResOnc.P174.0277 | | | | |
| 7.4 | P1AG0712 | one-armed CD8 IgG-pH dep | 1.42E+05 | 1.15E−03 | 8.12E−09 | 8.1 |
| | | IL2v_ExplResOnc.P174.0281 | | | | |
| 7.1 | P1AG0712 | one-armed CD8 IgG-pH dep | 1.49E+05 | 9.69E−04 | 6.49E−09 | 6.5 |
| | | IL2v_ExplResOnc.P174.0281 | | | | |
| 6.8 | P1AG0712 | one-armed CD8 IgG-pH dep | 1.45E+05 | 1.12E−03 | 7.77E−09 | 7.8 |
| | | IL2v_ExplResOnc.P174.0281 | | | | |
| 6.5 | P1AG0712 | one-armed CD8 IgG-pH dep | 1.39E+05 | 9.79E−04 | 7.05E−09 | 7.1 |
| | | IL2v_ExplResOnc.P174.0281 | | | | |
| 6.2 | P1AG0712 | one-armed CD8 IgG-pH dep | 1.49E+05 | 8.17E−04 | 5.49E−09 | 5.5 |
| | | IL2v_ExplResOnc.P174.0281 | | | | |
| 6.0 | P1AG0712 | one-armed CD8 IgG-pH dep | 1.56E+05 | 6.42E−04 | 4.12E−09 | 4.1 |
| | | IL2v_ExplResOnc.P174.0281 | | | | |
| 7.4 | P1AG0713 | one-armed CD8 IgG-pH dep | 2.44E+05 | 4.50E−03 | 1.84E−08 | 18.4 |
| | | IL2v_ExplResOnc.P174.0326 | | | | |
| 7.1 | P1AG0713 | one-armed CD8 IgG-pH dep | 2.78E+05 | 4.30E−03 | 1.55E−08 | 15.5 |
| | | IL2v_ExplResOnc.P174.0326 | | | | |
| 6.8 | P1AG0713 | one-armed CD8 IgG-pH dep | 2.57E+05 | 3.61E−03 | 1.41E−08 | 14.1 |
| | | IL2v_ExplResOnc.P174.0326 | | | | |
| 6.5 | P1AG0713 | one-armed CD8 IgG-pH dep | 2.67E+05 | 3.15E−03 | 1.18E−08 | 11.8 |
| | | IL2v_ExplResOnc.P174.0326 | | | | |
| 6.2 | P1AG0713 | one-armed CD8 IgG-pH dep | 2.78E+05 | 2.90E−03 | 1.04E−08 | 10.4 |
| | | IL2v_ExplResOnc.P174.0326 | | | | |
| 6.0 | P1AG0713 | one-armed CD8 IgG-pH dep | 2.79E+05 | 2.88E−03 | 1.03E−08 | 10.3 |
| | | IL2v_ExplResOnc.P174.0326 | | | | |
| 7.4 | P1AG0714 | one-armed CD8 IgG-pH dep | 1.96E+05 | 1.19E−03 | 6.06E−09 | 6.1 |
| | | IL2v_ExplResOnc.P174.0327 | | | | |
| 7.1 | P1AG0714 | one-armed CD8 IgG-pH dep | 1.94E+05 | 1.04E−03 | 5.37E−09 | 5.4 |
| | | IL2v_ExplResOnc.P174.0327 | | | | |
| 6.8 | P1AG0714 | one-armed CD8 IgG-pH dep | 2.22E+05 | 1.30E−03 | 5.87E−09 | 5.9 |
| | | IL2v_ExplResOnc.P174.0327 | | | | |
| 6.5 | P1AG0714 | one-armed CD8 IgG-pH dep | 2.50E+05 | 1.19E−03 | 4.75E−09 | 4.7 |
| | | IL2v_ExplResOnc.P174.0327 | | | | |
| 6.2 | P1AG0714 | one-armed CD8 IgG-pH dep | 2.84E+05 | 1.17E−03 | 4.11E−09 | 4.1 |
| | | IL2v_ExplResOnc.P174.0327 | | | | |
| 6.0 | P1AG0714 | one-armed CD8 IgG-pH dep | 3.39E+05 | 1.34E−03 | 3.95E−09 | 4.0 |
| | | IL2v_ExplResOnc.P174.0327 | | | | |
| 7.4 | P1AG0715 | one-armed CD8 IgG-pH dep | 2.12E+05 | 1.30E−03 | 6.16E−09 | 6.2 |
| | | IL2v_ExplResOnc.P175.0125 | | | | |
| 7.1 | P1AG0715 | one-armed CD8 IgG-pH dep | 1.91E+05 | 1.05E−03 | 5.48E−09 | 5.5 |
| | | IL2v_ExplResOnc.P175.0125 | | | | |
| 6.8 | P1AG0715 | one-armed CD8 IgG-pH dep | 2.00E+05 | 1.27E−03 | 6.37E−09 | 6.4 |
| | | IL2v_ExplResOnc.P175.0125 | | | | |
| 6.5 | P1AG0715 | one-armed CD8 IgG-pH dep | 2.06E+05 | 1.26E−03 | 6.12E−09 | 6.1 |
| | | IL2v_ExplResOnc.P175.0125 | | | | |
| 6.2 | P1AG0715 | one-armed CD8 IgG-pH dep | 2.02E+05 | 1.06E−03 | 5.22E−09 | 5.2 |
| | | IL2v_ExplResOnc.P175.0125 | | | | |
| 6.0 | P1AG0715 | one-armed CD8 IgG-pH dep | 2.41E+05 | 1.19E−03 | 4.96E−09 | 5.0 |
| | | IL2v_ExplResOnc.P175.0125 | | | | |
| 7.4 | P1AG0716 | one-armed CD8 IgG-pH dep | 9.44E+04 | 3.26E−04 | 3.46E−09 | 3.5 |
| | | IL2v_ExplResOnc.P175.0368 | | | | |
| 7.1 | P1AG0716 | one-armed CD8 IgG-pH dep | 1.04E+05 | 2.61E−04 | 2.52E−09 | 2.5 |
| | | IL2v_ExplResOnc.P175.0368 | | | | |
| 6.8 | P1AG0716 | one-armed CD8 IgG-pH dep | 1.03E+05 | 4.57E−04 | 4.44E−09 | 4.4 |
| | | IL2v_ExplResOnc.P175.0368 | | | | |
| 6.5 | P1AG0716 | one-armed CD8 IgG-pH dep | 1.09E+05 | 6.21E−04 | 5.69E−09 | 5.7 |
| | | IL2v_ExplResOnc.P175.0368 | | | | |
| 6.2 | P1AG0716 | one-armed CD8 IgG-pH dep | 1.04E+05 | 5.12E−04 | 4.95E−09 | 4.9 |
| | | IL2v_ExplResOnc.P175.0368 | | | | |
| 6.0 | P1AG0716 | one-armed CD8 IgG-pH dep | 1.24E+05 | 2.83E−04 | 2.28E−09 | 2.3 |
| | | IL2v_ExplResOnc.P175.0368 | | | | |
| 7.4 | P1AG0717 | one-armed CD8 IgG-pH dep | 3.77E+05 | 4.52E−03 | 1.20E−08 | 12.0 |
| | | IL2v_ExplResOnc.P177.0035 | | | | |
| | | (IL2v (consensus all)) | | | | |
| 7.1 | P1AG0717 | one-armed CD8 IgG-pH dep | 3.68E+05 | 3.46E−03 | 9.41E−09 | 9.4 |
| | | IL2v_ExplResOnc.P177.0035 | | | | |
| | | (IL2v (consensus all)) | | | | |
| 6.8 | P1AG0717 | one-armed CD8 IgG-pH dep | 3.16E+05 | 3.38E−03 | 1.07E−08 | 10.7 |
| | | IL2v_ExplResOnc.P177.0035 | | | | |
| | | (IL2v (consensus all)) | | | | |
| 6.5 | P1AG0717 | one-armed CD8 IgG-pH dep | 3.44E+05 | 2.50E−03 | 7.28E−09 | 7.3 |
| | | IL2v_ExplResOnc.P177.0035 | | | | |
| | | (IL2v (consensus all)) | | | | |
| 6.2 | P1AG0717 | one-armed CD8 IgG-pH dep | 2.99E+05 | 2.04E−03 | 6.84E−09 | 6.8 |
| | | IL2v_ExplResOnc.P177.0035 | | | | |
| | | (IL2v (consensus all)) | | | | |
| 6.0 | P1AG0717 | one-armed CD8 IgG-pH dep | 3.28E+05 | 2.75E−03 | 8.38E−09 | 8.4 |
| | | IL2v_ExplResOnc.P177.0035 | | | | |
| | | (IL2v (consensus all)) | | | | |
| 7.4 | P1AG0718 | one-armed CD8 IgG-pH dep | 3.86E+05 | 4.31E−03 | 1.12E−08 | 11.2 |
| | | IL2v_ExplResOnc.P177.0036 | | | | |
| 7.1 | P1AG0718 | one-armed CD8 IgG-pH dep | 3.28E+05 | 4.11E−03 | 1.25E−08 | 12.5 |
| | | IL2v_ExplResOnc.P177.0036 | | | | |
| 6.8 | P1AG0718 | one-armed CD8 IgG-pH dep | 3.13E+05 | 3.59E−03 | 1.15E−08 | 11.5 |
| | | IL2v_ExplResOnc.P177.0036 | | | | |
| 6.5 | P1AG0718 | one-armed CD8 IgG-pH dep | 3.14E+05 | 3.45E−03 | 1.10E−08 | 11.0 |
| | | IL2v_ExplResOnc.P177.0036 | | | | |
| 6.2 | P1AG0718 | one-armed CD8 IgG-pH dep | 2.74E+05 | 2.12E−03 | 7.76E−09 | 7.8 |
| | | IL2v_ExplResOnc.P177.0036 | | | | |
| 6.0 | P1AG0718 | one-armed CD8 IgG-pH dep | 2.64E+05 | 2.31E−03 | 8.73E−09 | 8.7 |
| | | IL2v_ExplResOnc.P177.0036 | | | | |
| 7.4 | P1AG0719 | one-armed CD8 IgG-pH dep | 1.63E+05 | 1.50E−03 | 9.24E−09 | 9.2 |
| | | IL2v_ExplResOnc.P177.0156 | | | | |
| 7.1 | P1AG0719 | one-armed CD8 IgG-pH dep | 1.58E+05 | 9.62E−04 | 6.11E−09 | 6.1 |
| | | IL2v_ExplResOnc.P177.0156 | | | | |
| 6.8 | P1AG0719 | one-armed CD8 IgG-pH dep | 1.60E+05 | 1.04E−03 | 6.50E−09 | 6.5 |
| | | IL2v_ExplResOnc.P177.0156 | | | | |
| 6.5 | P1AG0719 | one-armed CD8 IgG-pH dep | 1.52E+05 | 7.97E−04 | 5.24E−09 | 5.2 |
| | | IL2v_ExplResOnc.P177.0156 | | | | |
| 6.2 | P1AG0719 | one-armed CD8 IgG-pH dep | 1.64E+05 | 7.15E−04 | 4.37E−09 | 4.4 |
| | | IL2v_ExplResOnc.P177.0156 | | | | |
| 6.0 | P1AG0719 | one-armed CD8 IgG-pH dep | 1.66E+05 | 5.60E−04 | 3.36E−09 | 3.4 |
| | | IL2v_ExplResOnc.P177.0156 | | | | |
| 7.4 | P1AG0720 | one-armed CD8 IgG-pH dep | 1.95E+05 | 2.38E−03 | 1.22E−08 | 12.2 |
| | | IL2v_ExplResOnc.P178.0145 | | | | |
| 7.1 | P1AG0720 | one-armed CD8 IgG-pH dep | 2.19E+05 | 2.08E−03 | 9.53E−09 | 9.5 |
| | | IL2v_ExplResOnc.P178.0145 | | | | |
| 6.8 | P1AG0720 | one-armed CD8 IgG-pH dep | 1.98E+05 | 1.76E−03 | 8.88E−09 | 8.9 |
| | | IL2v_ExplResOnc.P178.0145 | | | | |
| 6.5 | P1AG0720 | one-armed CD8 IgG-pH dep | 2.03E+05 | 1.59E−03 | 7.83E−09 | 7.8 |
| | | IL2v_ExplResOnc.P178.0145 | | | | |
| 6.2 | P1AG0720 | one-armed CD8 IgG-pH dep | 2.14E+05 | 1.68E−03 | 7.86E−09 | 7.9 |
| | | IL2v_ExplResOnc.P178.0145 | | | | |
| 6.0 | P1AG0720 | one-armed CD8 IgG-pH dep | 2.43E+05 | 2.16E−03 | 8.90E−09 | 8.9 |
| | | IL2v_ExplResOnc.P178.0145 | | | | |
| 7.4 | P1AG0721 | one-armed CD8 IgG-pH dep | low response | | | |
| | | IL2v-pH 1 (Q11E) | | | | |
| 7.1 | P1AG0721 | one-armed CD8 IgG-pH dep | | | | |
| | | IL2v-pH 1 (Q11E) | | | | |
| 6.8 | P1AG0721 | one-armed CD8 IgG-pH dep | | | | |
| | | IL2v-pH 1 (Q11E) | | | | |
| 6.5 | P1AG0721 | one-armed CD8 IgG-pH dep | | | | |
| | | IL2v-pH 1 (Q11E) | | | | |
| 6.2 | P1AG0721 | one-armed CD8 IgG-pH dep | | | | |
| | | IL2v-pH 1 (Q11E) | | | | |
| 6.0 | P1AG0721 | one-armed CD8 IgG-pH dep | | | | |
| | | IL2v-pH 1 (Q11E) | | | | |
| 7.4 | P1AG0722 | one-armed CD8 IgG-pH dep | low response | | | |
| | | IL2v-pH 2 (E15Q) | | | | |
| 7.1 | P1AG0722 | one-armed CD8 IgG-pH dep | | | | |
| | | IL2v-pH 2 (E15Q) | | | | |
| 6.8 | P1AG0722 | one-armed CD8 IgG-pH dep | | | | |
| | | IL2v-pH 2 (E15Q) | | | | |
| 6.5 | P1AG0722 | one-armed CD8 IgG-pH dep | | | | |
| | | IL2v-pH 2 (E15Q) | | | | |
| 6.2 | P1AG0722 | one-armed CD8 IgG-pH dep | | | | |
| | | IL2v-pH 2 (E15Q) | | | | |
| 6.0 | P1AG0722 | one-armed CD8 IgG-pH dep | | | | |
| | | IL2v-pH 2 (E15Q) | | | | |
| 7.4 | P1AG0723 | one-armed CD8 IgG-pH dep | 1.08E+05 | 5.93E−04 | 5.49E−09 | 5.5 |
| | | IL2v-pH 3 (H16E) | | | | |
| 7.1 | P1AG0723 | one-armed CD8 IgG-pH dep | 1.06E+05 | 3.70E−04 | 3.50E−09 | 3.5 |
| | | IL2v-pH 3 (H16E) | | | | |
| 6.8 | P1AG0723 | one-armed CD8 IgG-pH dep | 1.23E+05 | 8.13E−04 | 6.62E−09 | 6.6 |
| | | IL2v-pH 3 (H16E) | | | | |
| 6.5 | P1AG0723 | one-armed CD8 IgG-pH dep | 1.13E+05 | 8.05E−04 | 7.14E−09 | 7.1 |
| | | IL2v-pH 3 (H16E) | | | | |
| 6.2 | P1AG0723 | one-armed CD8 IgG-pH dep | 1.04E+05 | 6.21E−04 | 5.97E−09 | 6.0 |
| | | IL2v-pH 3 (H16E) | | | | |
| 6.0 | P1AG0723 | one-armed CD8 IgG-pH dep | 1.20E+05 | 4.86E−04 | 4.07E−09 | 4.1 |
| | | IL2v-pH 3 (H16E) | | | | |
| 7.4 | P1AG0724 | one-armed CD8 IgG-pH dep | low response | | | |
| | | IL2v-pH 4 (L19D) | | | | |
| 7.1 | P1AG0724 | one-armed CD8 IgG-pH dep | | | | |
| | | IL2v-pH 4 (L19D) | | | | |
| 6.8 | P1AG0724 | one-armed CD8 IgG-pH dep | | | | |
| | | IL2v-pH 4 (L19D) | | | | |
| 6.5 | P1AG0724 | one-armed CD8 IgG-pH dep | | | | |
| | | IL2v-pH 4 (L19D) | | | | |
| 6.2 | P1AG0724 | one-armed CD8 IgG-pH dep | | | | |
| | | IL2v-pH 4 (L19D) | | | | |
| 6.0 | P1AG0724 | one-armed CD8 IgG-pH dep | | | | |
| | | IL2v-pH 4 (L19D) | | | | |
| 7.4 | P1AG0725 | one-armed CD8 IgG-pH dep | low response | | | |
| | | IL2v-pH 5 (Q22E) | | | | |
| 7.1 | P1AG0725 | one-armed CD8 IgG-pH dep | | | | |
| | | IL2v-pH 5 (Q22E) | | | | |
| 6.8 | P1AG0725 | one-armed CD8 IgG-pH dep | | | | |
| | | IL2v-pH 5 (Q22E) | | | | |
| 6.5 | P1AG0725 | one-armed CD8 IgG-pH dep | | | | |
| | | IL2v-pH 5 (Q22E) | | | | |
| 6.2 | P1AG0725 | one-armed CD8 IgG-pH dep | | | | |
| | | IL2v-pH 5 (Q22E) | | | | |
| 6.0 | P1AG0725 | one-armed CD8 IgG-pH dep | | | | |
| | | IL2v-pH 5 (Q22E) | | | | |
| 7.4 | P1AG0726 | one-armed CD8 IgG-pH dep | low response | | | |
| | | IL2v-pH 6 (M23Q) | | | | |
| 7.1 | P1AG0726 | one-armed CD8 IgG-pH dep | | | | |
| | | IL2v-pH 6 (M23Q) | | | | |
| 6.8 | P1AG0726 | one-armed CD8 IgG-pH dep | | | | |
| | | IL2v-pH 6 (M23Q) | | | | |
| 6.5 | P1AG0726 | one-armed CD8 IgG-pH dep | | | | |
| | | IL2v-pH 6 (M23Q) | | | | |
| 6.2 | P1AG0726 | one-armed CD8 IgG-pH dep | | | | |
| | | IL2v-pH 6 (M23Q) | | | | |
| 6.0 | P1AG0726 | one-armed CD8 IgG-pH dep | | | | |
| | | IL2v-pH 6 (M23Q) | | | | |
| 7.4 | P1AG0727 | one-armed CD8 IgG-pH dep | 1.26E+05 | 4.39E−04 | 3.49E−09 | 3.5 |
| | | IL2v-pH 7_(R81D) | | | | |
| 7.1 | P1AG0727 | one-armed CD8 IgG-pH dep | 1.06E+05 | 4.62E−05 | 4.34E−10 | 0.4 |
| | | IL2v-pH 7_(R81D) | | | | |
| 6.8 | P1AG0727 | one-armed CD8 IgG-pH dep | 1.15E+05 | 3.86E−04 | 3.36E−09 | 3.4 |
| | | IL2v-pH 7_(R81D) | | | | |
| 6.5 | P1AG0727 | one-armed CD8 IgG-pH dep | 9.78E+04 | 6.18E−04 | 6.33E−09 | 6.3 |
| | | IL2v-pH 7_(R81D) | | | | |
| 6.2 | P1AG0727 | one-armed CD8 IgG-pH dep | 8.98E+04 | 5.32E−04 | 5.93E−09 | 5.9 |
| | | IL2v-pH 7_(R81D) | | | | |
| 6.0 | P1AG0727 | one-armed CD8 IgG-pH dep | 8.14E+04 | <1.0E−07 | <1.0E−12 | <0.1 |
| | | IL2v-pH 7_(R81D) | | | | |
| 7.4 | P1AG0728 | one-armed CD8 IgG-pH dep | 9.54E+04 | 3.22E−04 | 3.37E−09 | 3.4 |
| | | IL2v-pH 8_(D84E) | | | | |
| 7.1 | P1AG0728 | one-armed CD8 IgG-pH dep | 9.15E+04 | <1.0E−07 | <1.0E−12 | <0.1 |
| | | IL2v-pH 8_(D84E) | | | | |
| 6.8 | P1AG0728 | one-armed CD8 IgG-pH dep | 8.88E+04 | <1.0E−07 | <1.0E−12 | <0.1 |
| | | IL2v-pH 8_(D84E) | | | | |
| 6.5 | P1AG0728 | one-armed CD8 IgG-pH dep | 6.58E+04 | <1.0E−07 | <1.0E−12 | <0.1 |
| | | IL2v-pH 8_(D84E) | | | | |
| 6.2 | P1AG0728 | one-armed CD8 IgG-pH dep | 4.92E+04 | <1.0E−07 | <1.0E−12 | <0.1 |
| | | IL2v-pH 8_(D84E) | | | | |
| 6.0 | P1AG0728 | one-armed CD8 IgG-pH dep | 4.46E+04 | <1.0E−07 | <1.0E−12 | <0.1 |
| | | IL2v-pH 8_(D84E) | | | | |
| 7.4 | P1AG0729 | one-armed CD8 IgG-pH dep | 6.20E+04 | 8.43E−05 | 1.36E−09 | 1.4 |
| | | IL2v-pH 9 (S87E) | | | | |
| 7.1 | P1AG0729 | one-armed CD8 IgG-pH dep | 6.54E+04 | <1.0E−07 | <1.0E−12 | <0.1 |
| | | IL2v-pH 9 (S87E) | | | | |
| 6.8 | P1AG0729 | one-armed CD8 IgG-pH dep | 5.76E+04 | <1.0E−07 | <1.0E−12 | <0.1 |
| | | IL2v-pH 9 (S87E) | | | | |
| 6.5 | P1AG0729 | one-armed CD8 IgG-pH dep | 4.21E+04 | <1.0E−07 | <1.0E−12 | <0.1 |
| | | IL2v-pH 9 (S87E) | | | | |
| 6.2 | P1AG0729 | one-armed CD8 IgG-pH dep | 3.19E+04 | <1.0E−07 | <1.0E−12 | <0.1 |
| | | IL2v-pH 9 (S87E) | | | | |
| 6.0 | P1AG0729 | one-armed CD8 IgG-pH dep | 4.47E+04 | <1.0E−07 | <1.0E−12 | <0.1 |
| | | IL2v-pH 9 (S87E) | | | | |
| 7.4 | P1AG0730 | one-armed CD8 IgG-pH dep | low response | | | |
| | | IL2v-pH 11 (Q126H) | | | | |
| 7.1 | P1AG0730 | one-armed CD8 IgG-pH dep | | | | |
| | | IL2v-pH 11 (Q126H) | | | | |
| 6.8 | P1AG0730 | one-armed CD8 IgG-pH dep | | | | |
| | | IL2v-pH 11 (Q126H) | | | | |
| 6.5 | P1AG0730 | one-armed CD8 IgG-pH dep | | | | |
| | | IL2v-pH 11 (Q126H) | | | | |
| 6.2 | P1AG0730 | one-armed CD8 IgG-pH dep | | | | |
| | | IL2v-pH 11 (Q126H) | | | | |
| 6.0 | P1AG0730 | one-armed CD8 IgG-pH dep | | | | |
| | | IL2v-pH 11 (Q126H) | | | | |
| 7.4 | P1AG0731 | one-armed CD8 IgG-pH dep | 2.61E+05 | 1.45E−03 | 5.56E−09 | 5.6 |
| | | IL2v-pH 12 (S130E) | | | | |
| 7.1 | P1AG0731 | one-armed CD8 IgG-pH dep | 2.45E+05 | 1.19E−03 | 4.86E−09 | 4.9 |
| | | IL2v-pH 12 (S130E) | | | | |
| 6.8 | P1AG0731 | one-armed CD8 IgG-pH dep | 2.62E+05 | 1.46E−03 | 5.58E−09 | 5.6 |
| | | IL2v-pH 12 (S130E) | | | | |
| 6.5 | P1AG0731 | one-armed CD8 IgG-pH dep | 2.73E+05 | 1.49E−03 | 5.44E−09 | 5.4 |
| | | IL2v-pH 12 (S130E) | | | | |
| 6.2 | P1AG0731 | one-armed CD8 IgG-pH dep | 2.62E+05 | 1.32E−03 | 5.03E−09 | 5.0 |
| | | IL2v-pH 12 (S130E) | | | | |
| 6.0 | P1AG0731 | one-armed CD8 IgG-pH dep | 2.79E+05 | 1.44E−03 | 5.18E−09 | 5.2 |
| | | IL2v-pH 12 (S130E) | | | | |
| 7.4 | P1AG0733 | one-armed CD8 IgG-pH dep | 2.33E+05 | 1.15E−03 | 4.95E−09 | 5.0 |
| | | IL2v-pH 14_(T133D) | | | | |
| 7.1 | P1AG0733 | one-armed CD8 IgG-pH dep | 2.23E+05 | 9.29E−04 | 4.16E−09 | 4.2 |
| | | IL2v-pH 14_(T133D) | | | | |
| 6.8 | P1AG0733 | one-armed CD8 IgG-pH dep | 2.35E+05 | 1.14E−03 | 4.84E−09 | 4.8 |
| | | IL2v-pH 14 (T133D) | | | | |
| 6.5 | P1AG0733 | one-armed CD8 IgG-pH dep | 2.68E+05 | 1.22E−03 | 4.54E−09 | 4.5 |
| | | IL2v-pH 14_(T133D) | | | | |
| 6.2 | P1AG0733 | one-armed CD8 IgG-pH dep | 2.76E+05 | 1.16E−03 | 4.20E−09 | 4.2 |
| | | IL2v-pH 14_(T133D) | | | | |
| 6.0 | P1AG0733 | one-armed CD8 IgG-pH dep | 2.99E+05 | 1.19E−03 | 3.97E−09 | 4.0 |
| | | IL2v-pH 14_(T133D) | | | | |
| 7.4 | P1AG0735 | one-armed CD8 IgG-pH dep | 2.65E+05 | 1.23E−03 | 4.65E−09 | 4.7 |
| | | IL2v-pH 17 (consensus beta) | | | | |
| 7.1 | P1AG0735 | one-armed CD8 IgG-pH dep | 2.71E+05 | 1.02E−03 | 3.76E−09 | 3.8 |
| | | IL2v-pH 17 (consensus beta) | | | | |
| 6.8 | P1AG0735 | one-armed CD8 IgG-pH dep | 2.59E+05 | 1.18E−03 | 4.55E−09 | 4.6 |
| | | IL2v-pH 17 (consensus beta) | | | | |
| 6.5 | P1AG0735 | one-armed CD8 IgG-pH dep | 2.49E+05 | 1.18E−03 | 4.73E−09 | 4.7 |
| | | IL2v-pH 17 (consensus beta) | | | | |
| 6.2 | P1AG0735 | one-armed CD8 IgG-pH dep | 2.38E+05 | 1.01E−03 | 4.25E−09 | 4.2 |
| | | IL2v-pH 17 (consensus beta) | | | | |
| 6.0 | P1AG0735 | one-armed CD8 IgG-pH dep | 2.35E+05 | 1.07E−03 | 4.58E−09 | 4.6 |
| | | IL2v-pH 17 (consensus beta) | | | | |
| 7.4 | P1AG0736 | one-armed CD8 IgG-pH dep | 9.67E+04 | 4.68E−04 | 4.84E−09 | 4.8 |
| | | IL2v-pH 18 (consensus | | | | |
| | | gamma) | | | | |
| 7.1 | P1AG0736 | one-armed CD8 IgG-pH dep | 1.13E+05 | 4.21E−04 | 3.73E−09 | 3.7 |
| | | IL2v-pH 18 (consensus | | | | |
| | | gamma) | | | | |
| 6.8 | P1AG0736 | one-armed CD8 IgG-pH dep | 9.91E+04 | 5.58E−04 | 5.63E−09 | 5.6 |
| | | IL2v-pH 18 (consensus | | | | |
| | | gamma) | | | | |
| 6.5 | P1AG0736 | one-armed CD8 IgG-pH dep | 9.90E+04 | 5.80E−04 | 5.86E−09 | 5.9 |
| | | IL2v-pH 18 (consensus | | | | |
| | | gamma) | | | | |
| 6.2 | P1AG0736 | one-armed CD8 IgG-pH dep | 1.11E+05 | 5.32E−04 | 4.79E−09 | 4.8 |
| | | IL2v-pH 18 (consensus | | | | |
| | | gamma) | | | | |
| 6.0 | P1AG0736 | one-armed CD8 IgG-pH dep | 1.69E+05 | 5.37E−04 | 3.18E−09 | 3.2 |
| | | IL2v-pH 18 (consensus | | | | |
| | | gamma) | | | | |
| 7.4 | P1AG0737 | one-armed CD8 IgG-pH dep | 2.28E+05 | 1.58E−04 | 6.95E−10 | 0.7 |
| | | IL2v-pH_2nd library template | | | | |
| 7.1 | P1AG0737 | one-armed CD8 IgG-pH dep | 2.36E+05 | 2.06E−04 | 8.70E−10 | 0.9 |
| | | IL2v-pH_2nd library template | | | | |
| 6.8 | P1AG0737 | one-armed CD8 IgG-pH dep | 2.06E+05 | 2.89E−04 | 1.40E−09 | 1.4 |
| | | IL2v-pH_2nd library template | | | | |
| 6.5 | P1AG0737 | one-armed CD8 IgG-pH dep | 1.88E+05 | 4.10E−04 | 2.18E−09 | 2.2 |
| | | IL2v-pH_2nd library template | | | | |
| 6.2 | P1AG0737 | one-armed CD8 IgG-pH dep | 1.81E+05 | 7.15E−04 | 3.94E−09 | 3.9 |
| | | IL2v-pH_2nd library template | | | | |
| 6.0 | P1AG0737 | one-armed CD8 IgG-pH dep | 1.78E+05 | 1.13E−03 | 6.34E−09 | 6.3 |
| | | IL2v-pH_2nd library template | | | | |
| 7.4 | P1AG0738 | one-armed CD8 IgG-Parental | 7.06E+04 | 4.04E−04 | 5.72E−09 | 5.7 |
| | | IL2v | | | | |
| 7.1 | P1AG0738 | one-armed CD8 IgG-Parental | 7.66E+04 | 2.39E−04 | 3.13E−09 | 3.1 |
| | | IL2v | | | | |
| 6.8 | P1AG0738 | one-armed CD8 IgG-Parental | 5.96E+04 | 1.94E−04 | 3.24E−09 | 3.2 |
| | | IL2v | | | | |
| 6.5 | P1AG0738 | one-armed CD8 IgG-Parental | 3.87E+04 | 5.40E−05 | 1.40E−09 | 1.4 |
| | | IL2v | | | | |
| 6.2 | P1AG0738 | one-armed CD8 IgG-Parental | 5.56E+04 | 2.53E−05 | 4.55E−10 | 0.5 |
| | | IL2v | | | | |
| 6.0 | P1AG0738 | one-armed CD8 IgG-Parental | 6.18E+04 | <1.0E−07 | <1.0E−12 | <0.1 |
| | | IL2v | | | | |
| 7.4 | P1AG0739 | one-armed CD8 IgG-pH dep | 1.10E+05 | 6.74E−04 | 6.14E−09 | 6.1 |
| | | IL2v-pH 10 (R120H) | | | | |
| 7.1 | P1AG0739 | one-armed CD8 IgG-pH dep | 1.10E+05 | 7.72E−04 | 7.03E−09 | 7.0 |
| | | IL2v-pH 10 (R120H) | | | | |
| 6.8 | P1AG0739 | one-armed CD8 IgG-pH dep | 1.16E+05 | 1.10E−03 | 9.46E−09 | 9.5 |
| | | IL2v-pH 10 (R120H) | | | | |
| 6.5 | P1AG0739 | one-armed CD8 IgG-pH dep | 7.78E+04 | 1.05E−03 | 1.35E−08 | 13.5 |
| | | IL2v-pH 10 (R120H) | | | | |
| 6.2 | P1AG0739 | one-armed CD8 IgG-pH dep | 4.03E+04 | <1.0E−07 | <1.0E−12 | <0.1 |
| | | IL2v-pH 10 (R120H) | | | | |
| 6.0 | P1AG0739 | one-armed CD8 IgG-pH dep | 8.89E+04 | 1.81E−04 | 2.03E−09 | 2.0 |
| | | IL2v-pH 10 (R120H) | | | | |

### Example 7

Determination of Binding Kinetics of One-Armed CD8-Targeted IgG IL2v Fusions (2nd Set of Variants from 2nd Phage Display Campaign) to IL2Rbg Using BIACORE Single Cycle Kinetics

A set of eleven shortlisted IL2v variants was additionally tested for pH-dependent binding to IL-2Rbg by surface plasmon resonance (single cycle kinetics) using a Biacore T200 instrument (Cytiva). Experiments were performed in PBS-T (10 mM H₃PO₄, 140 mM NaCl, 0.05% Tween-20) at pH 7.4, pH 7.1, pH 6.8, and pH 6.5, respectively. For capturing of the IL2v variants, a biotinylated anti-human Fab (anti-CH1) antibody (ThermoScientific no. 7103202100) was immobilized on a Series S Sensor Chip SA (Cytiva no. BR-1005-31) at a surface density of −2500 resonance units (RU) on two flow cells. The IL2v variants were injected into flow cell 2 and the obtained surface densities ranged from −10 to 30 RU whereas the flow cell 1 was used as a reference surface. Subsequently, a dilution series of 11.1, 33.3, and 100 nM of human IL-2Rbg (P1AA4193) was injected for 90s and the dissociation was monitored for 900s for the highest concentration. Bulk refractive index differences were corrected by subtracting the response obtained from flow cell 1 (mock surface) as well as by subtracting buffer injections (double referencing). The derived curves were fitted to a 1:1 Langmuir binding model using the BIAevaluation software (Cytiva).

All of the eleven IL2v variants exhibit pH-dependent binding which continuously decreases from pH 6.5 to pH 7.4. Driver for this decrease in affinity is the dissociation rate constant (kd) for all variants, whereas for some of the variants there is also a clear reduction of the association rate constant (ka) from pH 6.5 to pH 7.4, e.g. for P1AG0709, P1AG0710, P1AG0712, P1AG0713, P1AG0715, P1AG0717, and P1AG0720. The results are summarized in Table 9.

TABLE 9
SPR kinetic data

| | | ka | kd | KD | KD |
|---|---|---|---|---|---|
| pH | Concept ID | (1/Ms) | (1/s) | (M) | (nM) |
| 6.5 | P1AG0697 | 2.62E+05 | 4.55E−04 | 1.74E−09 | 2 |
| 6.8 | P1AG0697 | 4.10E+05 | 1.19E−03 | 2.90E−09 | 3 |
| 7.1 | P1AG0697 | 4.72E+05 | 4.24E−03 | 8.97E−09 | 9 |
| 7.4 | P1AG0697 | 3.82E+06 | 4.04E−02 | 1.06E−08 | 11 |
| 6.5 | P1AG0706 | 3.67E+05 | 1.51E−03 | 4.12E−09 | 4 |
| 6.8 | P1AG0706 | 4.90E+05 | 3.22E−03 | 6.57E−09 | 7 |
| 7.1 | P1AG0706 | 5.54E+05 | 1.82E−02 | 3.28E−08 | 33 |
| 7.4 | P1AG0706 | 9.69E+04 | 1.04E−02 | 1.08E−07 | 108 |
| 6.5 | P1AG0708 | 5.49E+05 | 1.32E−03 | 2.41E−09 | 2 |
| 6.8 | P1AG0708 | 9.63E+05 | 4.84E−03 | 5.03E−09 | 5 |
| 7.1 | P1AG0708 | 3.39E+05 | 1.28E−02 | 3.78E−08 | 38 |
| 7.4 | P1AG0708 | 2.15E+05 | 2.61E−02 | 1.21E−07 | 121 |
| 6.5 | P1AG0709 | 1.33E+06 | 1.40E−02 | 1.05E−08 | 11 |
| 6.8 | P1AG0709 | 2.87E+05 | 2.38E−02 | 8.29E−08 | 83 |
| 7.1 | P1AG0709 | low response | | | |
| 7.4 | P1AG0709 | | | | |
| 6.5 | P1AG0710 | 6.24E+05 | 1.71E−02 | 2.74E−08 | 27 |
| 6.8 | P1AG0710 | 1.73E+05 | 1.58E−02 | 9.13E−08 | 91 |
| 7.1 | P1AG0710 | 1.06E+05 | 2.02E−02 | 1.90E−07 | 190 |
| 7.4 | P1AG0710 | low response | | | |
| 6.5 | P1AG0712 | 1.25E+05 | 7.11E−04 | 5.68E−09 | 6 |
| 6.8 | P1AG0712 | 1.22E+05 | 1.49E−03 | 1.23E−08 | 12 |
| 7.1 | P1AG0712 | low response | | | |
| 7.4 | P1AG0712 | | | | |
| 6.5 | P1AG0713 | 1.52E+06 | 2.04E−03 | 1.34E−09 | 1 |
| 6.8 | P1AG0713 | 7.85E+05 | 7.42E−03 | 9.45E−09 | 9 |
| 7.1 | P1AG0713 | 1.46E+05 | 1.54E−02 | 1.05E−07 | 105 |
| 7.4 | P1AG0713 | low response | | | |
| 6.5 | P1AG0714 | 3.16E+05 | 6.88E−04 | 2.17E−09 | 2 |
| 6.8 | P1AG0714 | 4.86E+05 | 2.17E−03 | 4.46E−09 | 4 |
| 7.1 | P1AG0714 | 4.02E+05 | 8.01E−03 | 1.99E−08 | 20 |
| 7.4 | P1AG0714 | 3.06E+05 | 1.31E−02 | 4.29E−08 | 43 |
| 6.5 | P1AG0715 | 5.32E+05 | 1.20E−03 | 2.26E−09 | 2 |
| 6.8 | P1AG0715 | 7.80E+05 | 7.07E−03 | 9.06E−09 | 9 |
| 7.1 | P1AG0715 | 1.17E+05 | 1.47E−02 | 1.26E−07 | 126 |
| 7.4 | P1AG0715 | low response | | | |
| 6.5 | P1AG0717 | 7.99E+05 | 8.41E−03 | 1.05E−08 | 11 |
| 6.8 | P1AG0717 | 3.00E+05 | 1.23E−02 | 4.10E−08 | 41 |
| 7.1 | P1AG0717 | 1.85E+05 | 4.57E−02 | 2.47E−07 | 247 |
| 7.4 | P1AG0717 | low response | | | |
| 6.5 | P1AG0720 | 3.29E+05 | 1.50E−03 | 4.54E−09 | 5 |
| 6.8 | P1AG0720 | 5.28E+05 | 4.57E−03 | 8.66E−09 | 9 |
| 7.1 | P1AG0720 | 8.71E+04 | 6.70E−03 | 7.68E−08 | 77 |
| 7.4 | P1AG0720 | 6.37E+04 | 1.54E−02 | 2.42E−07 | 242 |

### Example 8

Activation and Proliferation Induction of Human PBMCs by One-Armed CD8-Targeted IgG IL2v Fusions

The impact of anti-CD8-IL2v (pH) constructs on human PBMC activation and proliferation was determined at pH7.4 and pH6.5. Briefly, anti-CD8-IL2v (pH) dilutions (end concentrations 100 nM, 10 nM, 1 nM, 0.1 nM) were incubated with fresh CF SE-labeled human PBMCs for 5 days at 37° C., 5% CO₂ in the incubator in custom RPMI medium (Gibco/Thermofisher) with either pH6.5 or pH7.4. After 5 days, cells were harvested and stained for flow cytometry (Live/Dead (life technologies #L34976), anti-human CD4 BV605, anti-human CD8 BV711, anti-human CD56 BV421, anti-human CD25 PE-Dazzle 594 and anti-human CD69 PECy7. The percentages of CD25, CD69 or 4-1BB expressing CD4+ T cells, CD8+ T cells or NK cells and the median fluorescence intensities (MFI) of the activation marker on the three immune populations were determined as well as the percentage of proliferating cells.

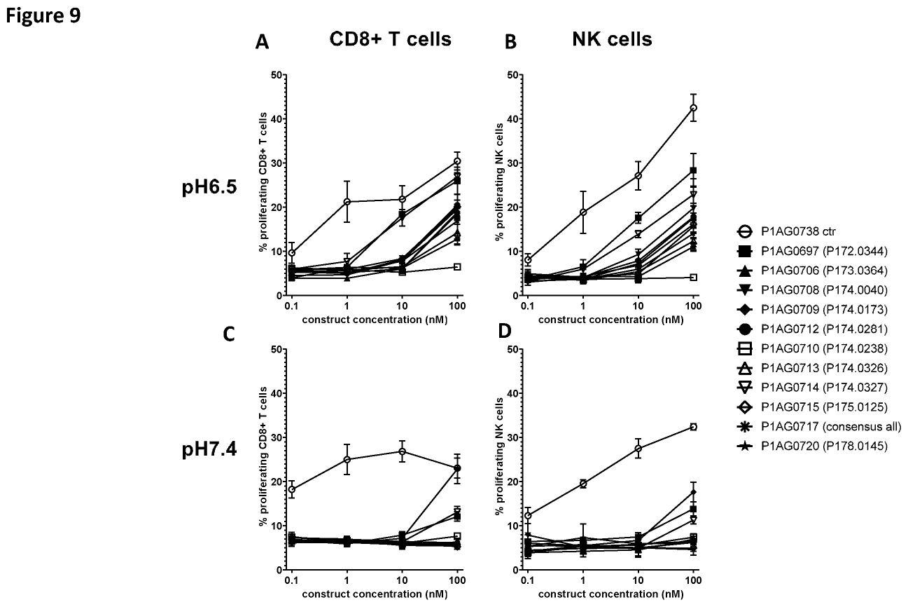

The results for the induction of CD8+ T cell and NK cell proliferation by the anti-CD8-IL2v (pH) constructs are shown in FIG. 9. CD4+ T cells showed no significant induction of proliferation in the absence of further stimulation (data not shown). All constructs induced concentration-dependent proliferation of CD8+ T cells and NK cells at pH6.5 whereas only the highest concentration of some of the pH-dependent constructs induced some proliferation at pH7.4 compared to the pH-independent control construct P1AG0738 ctr. which induced significant proliferation under all tested conditions (FIG. 9).

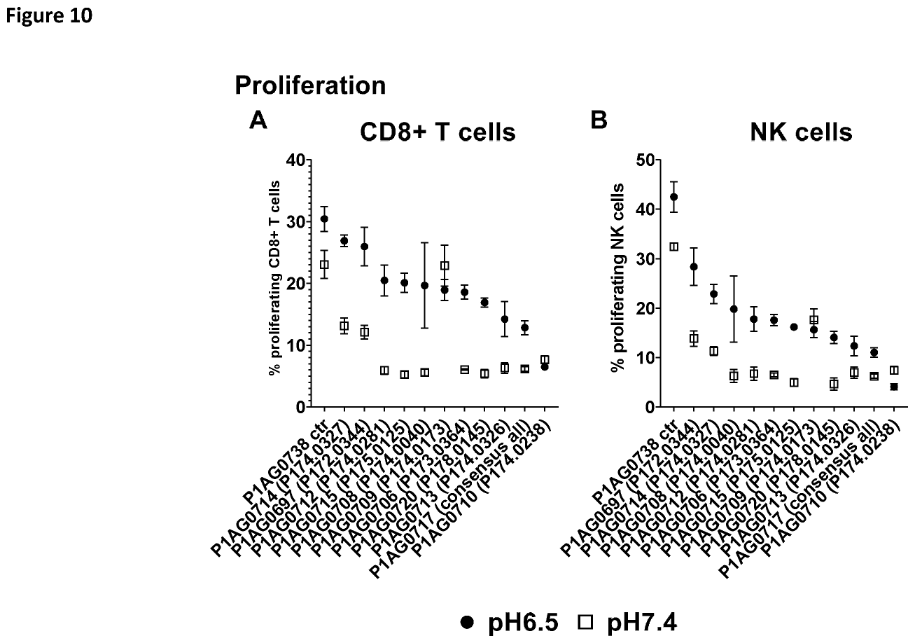

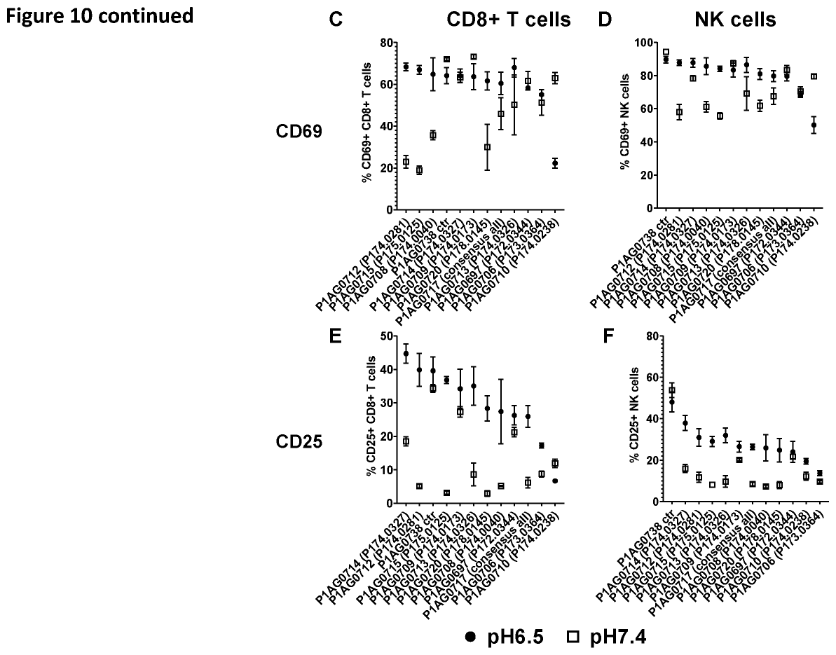

In addition to proliferation, anti-CD8-IL2v (pH) constructs induce also T and NK cell activation (CD25, CD69) as shown in FIG. 10. Here, the percentage of proliferating cells as well as percent positive cells for CD25 and CD69 expression is shown dependent on the pH of the culture medium. There is a clear pH-dependency observed for the majority of the constructs in contrast to the pH-independent control P1AG0738 ctr.

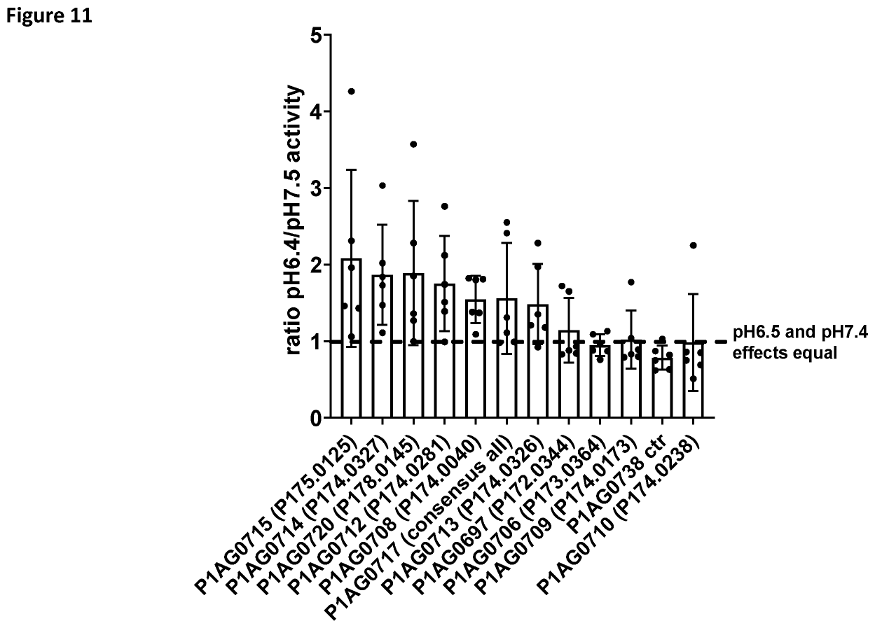

In order to select the most promising candidates with higher activity at pH6.5 but low to no activity at pH7.4 across different immune subsets and activation/proliferation marker, the area under the curve (AUC) values were determined for all readouts based on the titration curves of the constructs. The ratio of the AUCs at pH6.5 to the AUCs at pH7.4 was calculated (FIG. 11). Based on this, the top 5 molecules with the highest average ratio of activity at pH6.5 to pH7.4 have been selected for in vitro testing upon conversion to PD1-targeted IL2v (pH) constructs (Table 10).

### Example 9

Conversion of 2nd Generation pH-Dep IL2v Variants into a Bivalently PD1-Targeted Format

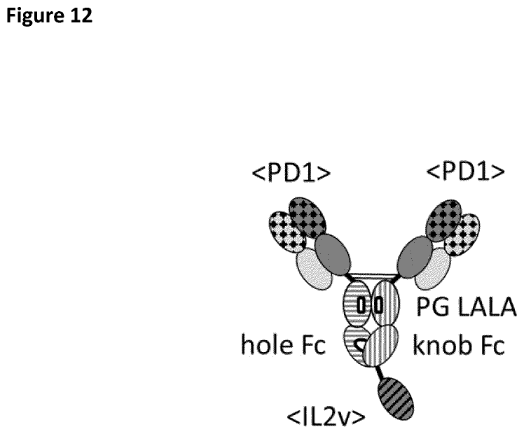

A short-list of eleven pH-dependent IL2v variants have been selected for conversion into a bivalently PD1-targeted complex format as depicted in FIG. 12. These eleven pH-dependent IL2v variants were P172.0344 (SEQ ID NOs 145, 146 and 147), P173.0364 (SEQ ID NOs 145, 146 and 148), P174.0040 (SEQ ID NOs 145, 146 and 149), P174.0173 (SEQ ID NOs 145, 146 and 150), P174.0238 (SEQ ID NOs 145, 146 and 151), P174.0281(SEQ ID NOs 145, 146 and 152), P174.0326 (SEQ ID NOs 145, 146 and 153), P174.0327 (SEQ ID NOs 145, 146 and 154), P175.0125 (SEQ ID NOs 145, 146 and 155), P177.0035 (IL2v (consensus all)) (SEQ ID NOs 145, 146 and 156), and P178.0145 (SEQ ID NOs 145, 146 and 157)). Five of these PD1-targeted pH-dependent IL2v variants have been tested in cell-based assays in vitro and the table below shows the corresponding one-armed CD8-targeted vs. bivalently PD1-targeted pH-dependent IL2v variant constructs.

TABLE 10
The most pH-dependent IL2v variants with the highest average
ratio of activity at pH 6.5 to pH 7.4 have been selected for
in vitro testing as bivalently PD1-targeted constructs

| CD8-targeted IL2v (pH) | corresponding PD1-targeted IL2v (pH) |
|---|---|
| P1AG0715 (P175.0125) | P1AG6052 (P175.0125) |
| P1AG0714 (P174.0327) | P1AG6053 (P174.0327) |
| P1AG0720 (P178.0145) | P1AG6056 (P178.0145) |
| P1AG0712 (P174.0281) | P1AG6059 (P174.0281) |
| P1AG0708 (P174.0040) | P1AG6061 (P174.0040) |

Activation and Proliferation Induction of Human PBMCs by 2nd Generation pH-Dep IL2v Variants Bivalently Targeted to PD1

The impact of anti-PD1-IL2v (pH) constructs on human PBMC activation and proliferation was determined at pH7.4, pH6.8 and pH6.5. Briefly, anti-PD1-IL2v (pH) dilutions (end concentrations 10 nM, 1 nM) were incubated with fresh CFSE-labeled human PBMCs for 5 days at 37° C., 5% CO2 in the incubator in custom RPMI medium (Gibco/Thermofisher) with either pH6.5 or pH7.4 in the absence or presence of coated anti-human CD3 antibody (1 μg/ml). After 5 days, cells were harvested and stained for flow cytometry (Live/Dead (life technologies #L34976), anti-human CD4 BV605, anti-human CD8 BV711, anti-human CD56 BV421, anti-human CD25 PE-Dazzle 594, anti-human CD69 PECy7 and anti-human 4-1BB APC). The percentages of CD25, CD69 or 4-1BB expressing CD4+ T cells, CD8+ T cells or NK cells and the median fluorescence intensities (MFI) of the activation marker on the three immune populations were determined as well as the percentage of proliferating cells.

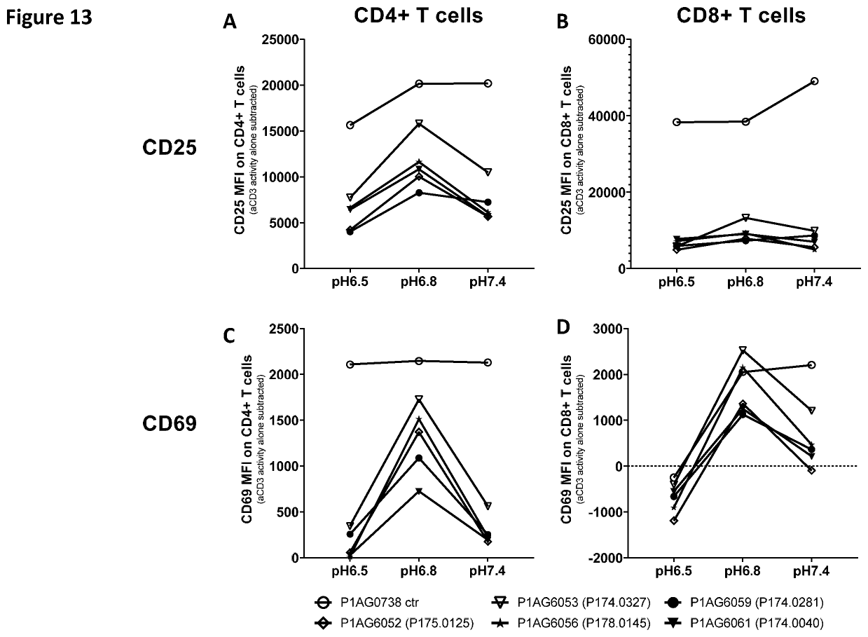

In contrast to CD8-targeted-IL2v (pH) constructs, the activity of anti-PD1-IL2v (pH) constructs was low to absent on fresh human PBMCs, which do not express PD1, in the absence of other stimuli (data not shown). In contrast to this, significant enhancement of aCD3-induced activation and proliferation could be observed especially at pH6.8 upon 5 days of incubation (a time at which activated T cells also upregulate PD1 expression) (FIG. 13).

### Example 10

Introduction of pH-Dependency Conferring Mutations into Wild-Type IL2

In order to test whether the mutations that confer pH-dependency to IL2v would also confer pH-dependency to wild-type IL2, selected sets of mutations were introduced into the wild-type IL2 sequence in fusion to the C-terminus of a human Fc knob chain (SEQ ID NO: 158 (‘empty’ HC hole for non-targeted Fc—wild-type IL2 fusions), SEQ ID NOs 159-164 (HC knob for non-targeted Fc—wild-type IL2 fusions). These selected sets of mutations correspond to P174.0040 (relating to the construct with SEQ ID NOs 158 and 159), P174.0326 (relating to the construct with SEQ ID NOs 158 and 160), P172.0344 (relating to the construct with SEQ ID NOs 158 and 162), P175.0125 (relating to the construct with SEQ ID NOs 158 and 163), and P174.0238 (relating to the construct with SEQ ID NOs 158 and 164), respectively, and a similar construct comprising wild-type IL2 has been used as comparator (P1AG7463 (relating to the construct with SEQ ID NOs 158 and 161)). As wild-type IL2 can bind in cis to CD25, targeting to CD8 or PD1 was omitted in these constructs. The format is shown in FIG. 14.

Determination of Binding Kinetics of pH-Dependent Non-Targeted Fc—Wt IL2 Fusions to IL2Rbg Using BIACORE Single Cycle Kinetics

pH-dependent binding of non-targeted Fc—wt IL2 fusions to IL2Rbg in comparison to one-armed CD8-targeted IgG IL2v fusions was investigated by surface plasmon resonance using a Biacore T200 instrument (Cytiva). Experiments were performed in PBS-T (10 mM H3PO4, 140 mM NaCl, 0.05% Tween-20) pH 7.4, 7.1, 6.8 and pH 6.5, respectively. For capturing of the IL2 fusion constructs, a mouse anti-human Fc PGLALA antibody was immobilized on a Series S Sensor Chip CM3 (Cytiva, 29104990) at a surface density of appr. 5000 resonance units (RU). The IL2 fusion constructs were injected onto the flow cells, the obtained surface densities reached from appr. 10 to 100 RU. The first flow cell was kept as reference surface. Subsequently, a dilution series of 33.3, 100 and 300 nM (11.1, 33.3 and 100 nM when using the parental IL2 fusion constructs as controls) of human IL-2Rbg (P1AA4193) was injected for 90s, dissociation was monitored for up to 600s. Bulk refractive index differences were corrected by subtracting the response obtained from flow cell one (reference surface) as well as by subtracting buffer injections (double referencing). The derived curves were fitted to a 1:1 Langmuir binding model using the BIAevaluation software (Cytiva) and the resulting KDs at the four different pH-values are summarized in Table 11. As can be appreciated by comparison of the IL2v variants (P1AG0697, P1AG0708, P1AG0710, P1AG0713, and P1AG0715) to the wild-type IL2 variants (P1AG7464, P1AG7461, P1AG7466, P1AG7462, and P1AG7465), the selected sets of mutations confer pH-dependency not only to IL2v but also to wild-type IL2.

TABLE 11
Summary table of the dissociation equilibrium constants (KD)
of pH-dependent IL2v and wild-type IL2 variants at different
pH (low = only low binding signal and very low affinity)

| | Concept | huIL2Rbg: KD (nM) at pH | | | |
|---|---|---|---|---|---|
| Concept name | ID | 7.4 | 7.1 | 6.8 | 6.5 |
| one-armed CD8 IgG-pH dep | P1AG0697 | 25 | 8 | 5 | 2 |
| IL2v_ExplResOnc.P172.0344 | | | | | |
| one-armed CD8 IgG-pH dep | P1AG0708 | 220 | 27 | 11 | 4 |
| IL2v_ExplResOnc.P174.0040 | | | | | |
| one-armed CD8 IgG-pH dep | P1AG0710 | low | 571 | 143 | 32 |
| IL2v_ExplResOnc.P174.0238 | | | | | |
| one-armed CD8 IgG-pH dep | P1AG0713 | 314 | 45 | 16 | 5 |
| IL2v_ExplResOnc.P174.0326 | | | | | |
| one-armed CD8 IgG-pH dep | P1AG0715 | 1070 | 54 | 15 | 4 |
| IL2v_ExplResOnc.P175.0125 | | | | | |
| one-armed CD8 IgG- | P1AG0738 | 0.1 | 0.1 | 0.1 | 0.1 |
| Parental IL2v | | | | | |
| Fc-IL2wt_pH_P172-0344 | P1AG7464 | 50 | 17 | 11 | 6 |
| Fc-IL2wt_pH_P174-0040 | P1AG7461 | 868 | 82 | 33 | 15 |
| Fc-IL2wt_pH_P174-0238 | P1AG7466 | low | 1530 | 443 | 121 |
| Fc-IL2wt_pH_P174-0326 | P1AG7462 | 797 | 96 | 37 | 12 |
| Fc-IL2wt_pH_P175-0125 | P1AG7465 | low | 182 | 45 | 11 |
| Fc-IL2wt | P1AG7463 | 0.1 | 0.1 | 0.1 | 0.1 |

Activation and Proliferation Induction of Human PBMCs by pH-Dependent Non-Targeted Fc—Wt IL2 Fusions

The impact of pH-dependent non-targeted Fc—wt IL2 fusions (Table 12) on human PBMC activation and proliferation was determined at pH7.4 and pH6.5. Briefly, IL2 wt (pH) dilutions were incubated with fresh CFSE-labeled human PBMCs for 5 days at 37° C., 5% CO2 in the incubator in custom RPMI medium (Gibco/Thermofisher) with either pH6.5 or pH7.4. After 5 days, cells were harvested and stained for flow cytometry. The percentages of CD69 expressing CD8+ T cells or NK cells and the median fluorescence intensities (MFI) of the activation marker on the two immune populations were determined as well as the percentage of proliferating cells.

TABLE 12
pH-dependent non-targeted Fc - wt IL2 fusions variant

| non-targeted Fc - | pH-dependent IL2 |
|---|---|
| wt IL2 fusion | variant identifier |
| P1AG7461 | P174.0040 |
| P1AG7462 | P174.0326 |
| P1AG7463_ctr | n.a. (wild-type IL2) |
| P1AG7464 | P172.0344 |
| P1AG7465 | P175.0125 |
| P1AG7466 | P174.0238 |

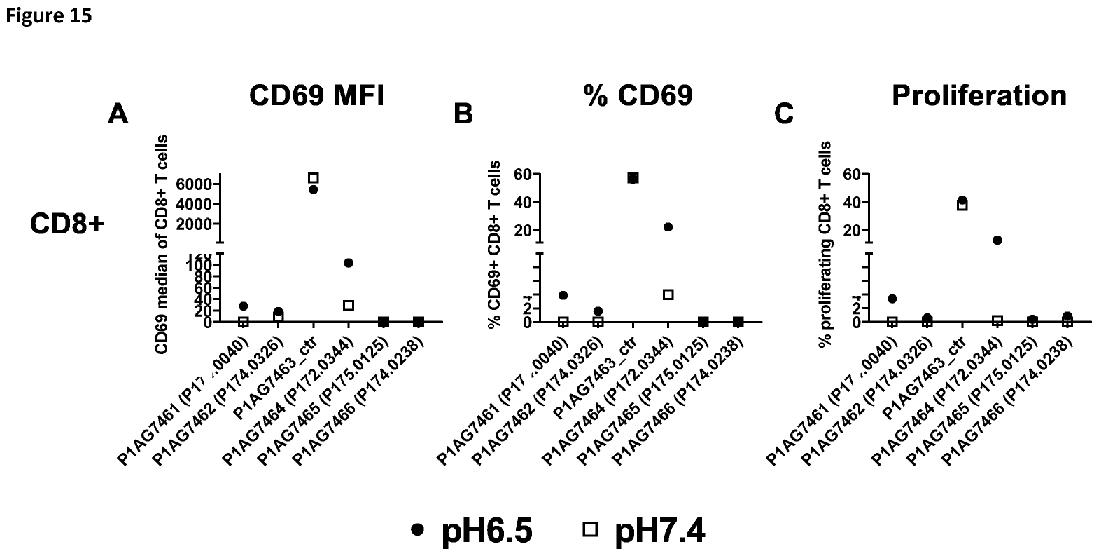

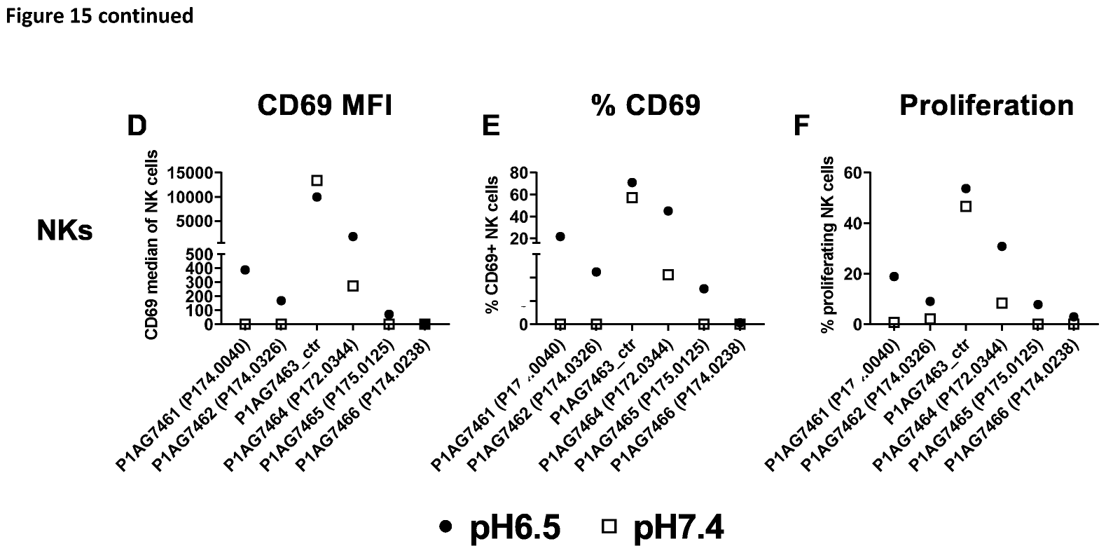

FIG. 15 shows the activity of the 100 nM pH-dependent non-targeted Fc—wt IL2 fusions at pH6.5 compared to pH7.4. From all pH-dependent IL2 wt constructs tested, P1AG7464 (P172.0344) showed the highest activity at pH6.5 and significantly lower activity at pH7.4 compared to the pH-independent control construct P1AG7463 ctr. P1AG7461 (P174.0040) and P1AG7462 (P174.0326) showed some activity at pH6.5 and hardly any activity at pH7.4 whereas P1AG7465 (P175.0125) and P1AG7466 (P174.0238) showed hardly any activity at all.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A mutant interleukin-2 (IL-2) polypeptide comprising one or more amino acid substitutions, each compared to a wilde-type IL-2, preferably human IL-2 according to SEQ ID NO: 144, wherein the one or more amino acid substitutions abolishes or reduces binding to the IL-2 receptor, preferably to the intermediate-affinity IL-2 receptor (IL2Rfβγ), at neutral pH and facilitate binding to the IL-2 receptor, preferably to the intermediate-affinity IL-2 receptor (IL2Rfβγ), at decreased pH.

2. The mutant IL-2 polypeptide of claim 1, wherein said one or more amino acid substitutions is at a position selected from the group of positions corresponding to residue 6, 8, 11, 12, 13, 15, 16, 19, 20, 22, 23, 81, 84, 87, 91, 95, 120, 123, 126, 130, 133 of human IL-2 according to SEQ ID NO: 144.

3. The mutant IL-2 polypeptide of claim 1 or 2, wherein said one or more amino acid substitutions is selected from the group of S6Y, K8E, Q11E, Q11T, L12D, L12E, L12Q, L12S, L12T, Q13H, Q13R, E15Q, H16D, H16E, H16N, H16Q, L19D, L19Q, D20E, D20Q, Q22D, Q22E, Q22H, M23E, M23N, M23Q, R81D, R81E, R81H, R81N, R81Q, D84E, D84Q, S87D, S87E, 587N, S87Q, V91D, V91E, V91N, E95D, E95Q, R120E, R120H, T123E, T123Q, Q126E, Q126H, S130E, T133D, T133E, T133N, T133Q.

4. The mutant IL-2 polypetide of any of claims 1 to 3, wherein the mutant IL-2 polypeptide comprises the amino acid substitutions (i) L12E, D20E, M23N, R81N, D84E, S87E, R120E, T123E, S130E, T133N;
(ii) Q11E, D20Q, M23E, R81D, D84E, S87E, S130E, T133N;
(iii) L12E, L19D, R81D, R120E, T123E, S130E, T133E;
(iv) R81Q, S87E, V91D, T123E, S130E, T133D;
(v) Q11E, L12E, M23Q, R81D, S87E, V91D, S130E, T133Q;
(vi) Q11E, L19D, R81D, D84E, S130E, T133D;
(vii) R81D, D84Q, S87D, V91N, T123Q, S130E, T133D;
(viii) L19D, R81E, D84E, S87Q, R120H, S130E, T133E;
(iix) L19D, M23N, R81D, T133E;
(ix) Q11E, L12E, M23Q, R81Q, S87D, V91N, E95Q, R120H, T123E, S130E, T133E;
(x) L19D, R81E, S130E, T133D;
(xi) R81Q, S87E, V91D, R120E, S130E, T133D;
(xii) L12Q, L19Q, R81H, V91E, T123E, S130E, T133E;
(xiii) K8E, D20E, M23N, R81H, D84Q, S87E, R120H, S130E, T133D;
(xiv) L12E, L19Q, R81H, R120E, T133D;
(xv) H16E, L19D, Q22E, M23Q, R81D, D84E, S87D, R120H, S130E, T133E;
(xvi) Q11E, L12E, H16Q, L19D, Q22E, M23N, R81E, D84E, S87E, R120H, S130E, T133E;
(xvii) Q11E, H16E, L19D, M23E, R81D, S87E, R120H, Q126E, T133D;
(xviii) Q11E, L12S, E15Q, H16N, L19D, M23E, R81E, D84E, S87D, R120H, S130E, T133E;
(xix) Q11E, H16E, M23E, R81N, D84E, S87E, R120H, Q126E, S130E, T133E;
(xx) Q11E, E15Q, H16E, Q22E, M23E, R81H, D84E, S87E, R120H, S130E, T133D;
(xxi) Q11E, L12D, Q13H, E15Q, H16E, Q22E, M23E, R81N, D84E, S87E, R120H, S130E, T133E;
(xxii) Q11E, E15Q, H16E, L19D, R81E, S87E, R120H, S130E, T133E;
(xxiii) H16E, L19D, M23Q, R81N, D84E, S87D, R120H, S130E, T133D;
(xxiv) Q11E, L12T, E15Q, H16E, L19D, Q22H, R81D, D84E, S87E, R120H, S130E, T133E;
(xxv) Q11E, L12T, E15Q, H16E, L19D, Q22H, M23E, R81E, D84E, S87E, R120H, S130E, T133E;
(xxvi) Q11E, E15Q, H16E, L19D, R81E, D84E, S87E, R120H, S130E, T133E;
(xxvii) H16E, Q22E, M23Q, S87N, R120H, S130E, T133E;
(xxviii) H16E, L19D, Q22D, M23Q, R81D, D84E, S87E, R120H, S130E, T133E;
(xxiix) Q11E, L12E, Q13H, E15Q, H16N, L19D, Q22E, M23Q, R81E, D84E, S87D, E95D, R120H, T133E;
(xxix) Q11E, L12T, H16E, L19D, Q22E, R81D, D84E, S87E, R120H, S130E, T133D;
(xxx) Q11T, L12E, E15Q, H16E, L19D, R81D, D84E, S87E, R120E, S130E, T133D;
(xxxi) Q11E, E15Q, H16E, L19D, R81D, D84E, S87E, R120H, S130E, T133E;
(xxxii) Q11E, E15Q, H16E, L19D, R81Q, D84E, S87E, R120H, S130E, T133E;
(xxxiii) Q11E, L12S, H16E, L19D, M23Q, R81D, D84E, S87E, R120H, S130E, T133E;
(xxxiv) S6Y, L12E, Q13R, H16Q, Q22E, M23Q, R81N, D84E, S87E, R120H, S130E, T133D;
(xxxv) H16D, M23N, R81D, D84E, R120H, S130E, T133E;
(xxxvi) Q11E, L12T, H16Q, L19D, M23E, R81D, D84E, S87E, R120H, S130E, T133E;
(xxxvii) Q11E, L12E, H16N, M23N, R81E, D84E, R120H, S130E, T133E;
(xxxviii) E15Q, H16E, L19D, R81D, D84E, S87E;
(xxxix) Q11E, R120H, S130E, T133D; or
(xl) Q11E, R81D, D84E, S87E, R120H, S130E, T133D.

5. The mutant IL-2 polypeptide of any of claims 1 to 4, wherein the mutant IL-2 polypeptide comprises any amino acid substitution selected from the group T3A, F42A, Y45A, L72G, C125A.

6. The mutant IL-2 polypeptide of any of claims 1 to 5, wherein the mutant IL-2 polypeptide comprises the amino acid substitutions F42A, Y45A and L72G.

7. The mutant IL-2 polypeptide of any of claims 1 to 6, wherein the mutant IL-2 polypeptide comprises the amino acid substitutions T3A, F42A, Y45A, L72G and C125A.

8. The mutant IL-2 polypeptide of any one of claims 1 to 7, wherein said mutant IL-2 polypeptide is linked to a non-IL-2 moiety.

9. The mutant IL-2 polypeptide of any one of claims 1 to 8, wherein said mutant IL-2 polypeptide is linked to a first and a second non-IL-2 moiety.

10. The mutant IL-2 polypeptide of claim 9, wherein said mutant IL-2 polypeptide shares a carboxy-terminal peptide bond with said first non-IL-2 moiety and an amino-terminal peptide bond with said second non-IL-2 moiety.

11. The mutant IL-2 polypeptide of any one of claims 8 to 10, wherein said non-IL-2 moiety is an antigen binding moiety or an effector cell binding moiety.

12. An immunoconjugate comprising a mutant IL-2 polypeptide of any one of claims 1 to 7 and an antigen binding moiety or an effector cell binding moiety.

13. The immunoconjugate of claim 12, wherein said mutant IL-2 polypeptide shares an amino- or carboxy-terminal peptide bond with said antigen binding moiety or the effector cell binding moiety.

14. The immunoconjugate of claim 12 or 13, wherein said immunoconjugate comprises a first and a second antigen binding moiety or a first and a second effector cell antigen binding moiety or an antigen binding moiety and an effector cell binding moiety.

15. The immunoconjugate of claim 14,
(i) wherein said mutant IL-2 polypeptide shares an amino- or carboxy-terminal peptide bond with said first antigen binding moiety and said second antigen binding moiety shares an amino- or carboxy-terminal peptide bond with either a) said mutant IL-2 polypeptide or b) said first antigen binding moiety;
(ii) wherein said mutant IL-2 polypeptide shares an amino- or carboxy-terminal peptide bond with said first effector cell binding moiety and said second effector cell binding moiety shares an amino- or carboxy-terminal peptide bond with either a) said mutant IL-2 polypeptide or b) said first effector cell binding moiety;
(iii) wherein said mutant IL-2 polypeptide shares an amino- or carboxy-terminal peptide bond with the antigen binding moiety and the effector cell binding moiety shares an amino- or carboxy-terminal peptide bond with either a) said mutant IL-2 polypeptide or b) said antigen binding moiety; or
(iv) wherein said mutant IL-2 polypeptide shares an amino- or carboxy-terminal peptide bond with the effector cell binding moiety and the antigen binding moiety shares an amino- or carboxy-terminal peptide bond with either a) said mutant IL-2 polypeptide or b) said effector cell binding moiety.

16. The mutant IL-2 polypeptide of claim 11 or the immunoconjugate of any one of claims 12 to 15, wherein said antigen binding moiety o is an antibody or an antibody fragment.

17. The mutant IL-2 polypeptide of claim 11 or the immunoconjugate of any one of claims 12 to 16, wherein said antigen binding moiety and/or said effector cell binding moiety is selected from a Fab molecule and a scFv molecule.

18. The mutant IL-2 polypeptide of claim 11 or the immunoconjugate of any one of claims 12 to 17, wherein said antigen binding moiety and/or said effector cell binding moiety is an immunoglobulin molecule, particularly an IgG molecule.

19. The mutant IL-2 polypeptide of claim 11 or the immunoconjugate of any one of claims 12 to 18, wherein said antigen binding moiety is directed to an antigen presented on a tumor cell or in a tumor cell environment and/or wherein said effector cell binding moiety is directed to an effector cell present in a tumor cell environment in order to achieve cis-targeting.

20. An isolated polynucleotide encoding the mutant IL-2 polypeptide or immunoconjugate of any one of claims 1 to 19.

21. An expression vector comprising the polynucleotide of claim 20.

22. A host cell comprising the polynucleotide of claim 20 or the expression vector of claim 21.

23. A method of producing a mutant IL-2 polypeptide or an immunoconjugate thereof, comprising culturing the host cell of claim 22 under conditions suitable for the expression of the mutant IL-2 polypeptide or the immunoconjugate.

24. A mutant IL-2 polypeptide or immunoconjugate produced by the method of claim 23.

25. A pharmaceutical composition comprising the mutant IL-2 polypeptide or immunoconjugate of any one of claim 1 to 19 or 24 and a pharmaceutically acceptable carrier.

26. The mutant IL-2 polypeptide or immunoconjugate of any one of claim 1 to 19 or 24 for use in the treatment of a disease in an individual in need thereof.

27. The mutant IL-2 polypeptide or immunoconjugate of claim 26, wherein said disease is cancer.

28. Use of the mutant IL-2 polypeptide or immunoconjugate of any one of claim 1 to 19 or 24 for manufacture of a medicament for treating a disease in an individual in need thereof.

29. A method of treating disease in an individual, comprising administering to said individual a therapeutically effective amount of a composition comprising the mutant IL-2 polypeptide or immunoconjugate of any one of claim 1 to 19 or 24 in a pharmaceutically acceptable form.

30. The method of claim 29, wherein said disease is cancer.

31. A method of stimulating the immune system of an individual, comprising administering to said individual a effective amount of a composition comprising the mutant IL-2 polypeptide or immunoconjugate of any one of claim 1 to 19 or 24 in a pharmaceutically acceptable form.

32. The invention as described hereinbefore.


SEQUENCE LISTING (164 sequences)

SEQ ID NO: 1 - protein, 132 aa, Artificial Sequence
  Synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 470 aa, Artificial Sequence
  Synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 461 aa, Artificial Sequence
  Synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 4 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 5 - protein, 133 aa, Artificial Sequence
  Synthetic construct
APASSSTKKTQEQLEHLLLELQNILNGINNYKNPKLTRMLTAKFAMPKKATELKHLQCLEEELKPLEEVLNGAQSKNFHLNPRELIENINVIVLELKGSETTFMCEYADETATIVEFLNEWIEFAQSIIETLN

SEQ ID NO: 6 - protein, 133 aa, Artificial Sequence
  Synthetic construct
APASSSTKKTELQLEHLLLQLQEILNGINNYKNPKLTRMLTAKFAMPKKATELKHLQCLEEELKPLEEVLNGAQSKNFHLDPRELIENINVIVLELKGSETTFMCEYADETATIVEFLNRWITFAQSIIETLN

SEQ ID NO: 7 - protein, 133 aa, Artificial Sequence
  Synthetic construct
APASSSTKKTQEQLEHLLDDLQMILNGINNYKNPKLTRMLTAKFAMPKKATELKHLQCLEEELKPLEEVLNGAQSKNFHLDPRDLISNINVIVLELKGSETTFMCEYADETATIVEFLNEWIEFAQSIIETLE

SEQ ID NO: 8 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLQPRDLIENINDIVLELKGSETTFMCEYADETATIVEFLNRWIEFAQSIIETLD

SEQ ID NO: 9 - protein, 133 aa, Artificial Sequence
  Synthetic construct
APASSSTKKTEEQLEHLLLDLQQILNGINNYKNPKLTRMLTAKFAMPKKATELKHLQCLEEELKPLEEVLNGAQSKNFHLDPRDLIENINDIVLELKGSETTFMCEYADETATIVEFLNRWITFAQSIIETLQ

SEQ ID NO: 10 - protein, 133 aa, Artificial Sequence
  Synthetic construct
APASSSTKKTELQLEHLLDDLQMILNGINNYKNPKLTRMLTAKFAMPKKATELKHLQCLEEELKPLEEVLNGAQSKNFHLDPRELISNINVIVLELKGSETTFMCEYADETATIVEFLNRWITFAQSIIETLD

SEQ ID NO: 11 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLDPRQLIDNINNIVLELKGSETTFMCEYADETATIVEFLNRWIQFAQSIIETLD

SEQ ID NO: 12 - protein, 133 aa, Artificial Sequence
  Synthetic construct
APASSSTKKTQLQLEHLLDDLQMILNGINNYKNPKLTRMLTAKFAMPKKATELKHLQCLEEELKPLEEVLNGAQSKNFHLEPRELIQNINVIVLELKGSETTFMCEYADETATIVEFLNHWITFAQSIIETLE

SEQ ID NO: 13 - protein, 133 aa, Artificial Sequence
  Synthetic construct
APASSSTKKTQLQLEHLLDDLQNILNGINNYKNPKLTRMLTAKFAMPKKATELKHLQCLEEELKPLEEVLNGAQSKNFHLDPRDLISNINVIVLELKGSETTFMCEYADETATIVEFLNRWITFAQSIISTLE

SEQ ID NO: 14 - protein, 133 aa, Artificial Sequence
  Synthetic construct
APASSSTKKTEEQLEHLLLDLQQILNGINNYKNPKLTRMLTAKFAMPKKATELKHLQCLEEELKPLEEVLNGAQSKNFHLQPRDLIDNINNIVLQLKGSETTFMCEYADETATIVEFLNHWIEFAQSIIETLE

SEQ ID NO: 15 - protein, 133 aa, Artificial Sequence
  Synthetic construct
APASSSTKKTQLQLEHLLDDLQMILNGINNYKNPKLTRMLTAKFAMPKKATELKHLQCLEEELKPLEEVLNGAQSKNFHLEPRDLISNINVIVLELKGSETTFMCEYADETATIVEFLNRWITFAQSIIETLD

SEQ ID NO: 16 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLQPRDLIENINDIVLELKGSETTFMCEYADETATIVEFLNEWITFAQSIIETLD

SEQ ID NO: 17 - protein, 133 aa, Artificial Sequence
  Synthetic construct
APASSSTKKTQQQLEHLLQDLQMILNGINNYKNPKLTRMLTAKFAMPKKATELKHLQCLEEELKPLEEVLNGAQSKNFHLHPRDLISNINEIVLELKGSETTFMCEYADETATIVEFLNRWIEFAQSIIETLE

SEQ ID NO: 18 - protein, 133 aa, Artificial Sequence
  Synthetic construct
APASSSTEKTQLQLEHLLLELQNILNGINNYKNPKLTRMLTAKFAMPKKATELKHLQCLEEELKPLEEVLNGAQSKNFHLHPRQLIENINVIVLELKGSETTFMCEYADETATIVEFLNHWITFAQSIIETLD

SEQ ID NO: 19 - protein, 133 aa, Artificial Sequence
  Synthetic construct
APASSSTKKTQEQLEHLLQDLQMILNGINNYKNPKLTRMLTAKFAMPKKATELKHLQCLEEELKPLEEVLNGAQSKNFHLHPRDLISNINVIVLELKGSETTFMCEYADETATIVEFLNEWITFAQSIISTLD

SEQ ID NO: 20 - protein, 133 aa, Artificial Sequence
  Synthetic construct
APASSSTKKTQLQLEELLDDLEQILNGINNYKNPKLTRMLTAKFAMPKKATELKHLQCLEEELKPLEEVLNGAQSKNFHLDPRELIDNINVIVLELKGSETTFMCEYADETATIVEFLNHWITFAQSIIETLE

SEQ ID NO: 21 - protein, 133 aa, Artificial Sequence
  Synthetic construct
APASSSTKKTEEQLEQLLDDLENILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 22 - protein, 133 aa, Artificial Sequence
  Synthetic construct
APASSSTKKTELQLEELLDDLQEILNGINNYKNPKLTRMLTAKFAMPKKATELKHLQCLEEELKPLEEVLNGAQSKNFHLDPRDLIENINVIVLELKGSETTFMCEYADETATIVEFLNHWITFAESIISTLD

SEQ ID NO: 23 - protein, 133 aa, Artificial Sequence
  Synthetic construct
APASSSTKKTESQLQNLLDDLQEILNGINNYKNPKLTRMLTAKFAMPKKATELKHLQCLEEELKPLEEVLNGAQSKNFHLEPRELIDNINVIVLELKGSETTFMCEYADETATIVEFLNHWITFAQSIIETLE

SEQ ID NO: 24 - protein, 133 aa, Artificial Sequence
  Synthetic construct
APASSSTKKTELQLEELLLDLQEILNGINNYKNPKLTRMLTAKFAMPKKATELKHLQCLEEELKPLEEVLNGAQSKNFHLNPRELIENINVIVLELKGSETTFMCEYADETATIVEFLNHWITFAESIIETLE

SEQ ID NO: 25 - protein, 133 aa, Artificial Sequence
  Synthetic construct
APASSSTKKTELQLQELLLDLEEILNGINNYKNPKLTRMLTAKFAMPKKATELKHLQCLEEELKPLEEVLNGAQSKNFHLHPRELIENINVIVLELKGSETTFMCEYADETATIVEFLNHWITFAQSIIETLD

SEQ ID NO: 26 - protein, 133 aa, Artificial Sequence
  Synthetic construct
APASSSTKKTEDHLQELLLDLEEILNGINNYKNPKLTRMLTAKFAMPKKATELKHLQCLEEELKPLEEVLNGAQSKNFHLNPRELIENINVIVLELKGSETTFMCEYADETATIVEFLNHWITFAQSIIETLE

SEQ ID NO: 27 - protein, 133 aa, Artificial Sequence
  Synthetic construct
APASSSTKKTELQLQELLDDLQMILNGINNYKNPKLTRMLTAKFAMPKKATELKHLQCLEEELKPLEEVLNGAQSKNFHLEPRDLIENINVIVLELKGSETTFMCEYADETATIVEFLNHWITFAQSIIETLE

SEQ ID NO: 28 - protein, 133 aa, Artificial Sequence
  Synthetic construct
APASSSTKKTQLQLEELLDDLQQILNGINNYKNPKLTRMLTAKFAMPKKATELKHLQCLEEELKPLEEVLNGAQSKNFHLNPRELIDNINVIVLELKGSETTFMCEYADETATIVEFLNHWITFAQSIIETLD

SEQ ID NO: 29 - protein, 133 aa, Artificial Sequence
  Synthetic construct
APASSSTKKTETQLQELLDDLHMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 30 - protein, 133 aa, Artificial Sequence
  Synthetic construct
APASSSTKKTETQLQELLDDLHEILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 31 - protein, 133 aa, Artificial Sequence
  Synthetic construct
APASSSTKKTELQLQELLDDLQMILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 32 - protein, 133 aa, Artificial Sequence
  Synthetic construct
APASSSTKKTQLQLEELLLDLEQILNGINNYKNPKLTRMLTAKFAMPKKATELKHLQCLEEELKPLEEVLNGAQSKNFHLRPRDLINNINVIVLELKGSETTFMCEYADETATIVEFLNHWITFAQSIIETLE

SEQ ID NO: 33 - protein, 133 aa, Artificial Sequence
  Synthetic construct
APASSSTKKTQLQLEELLDDLDQILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 34 - protein, 133 aa, Artificial Sequence
  Synthetic construct
APASSSTKKTEEHLQNLLDDLEQILNGINNYKNPKLTRMLTAKFAMPKKATELKHLQCLEEELKPLEEVLNGAQSKNFHLEPRELIDNINVIVLDLKGSETTFMCEYADETATIVEFLNHWITFAQSIISTLE

SEQ ID NO: 35 - protein, 133 aa, Artificial Sequence
  Synthetic construct
APASSSTKKTETQLEELLDDLEMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLD

SEQ ID NO: 36 - protein, 133 aa, Artificial Sequence
  Synthetic construct
APASSSTKKTTEQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNEWITFAQSIIETLD

SEQ ID NO: 37 - protein, 133 aa, Artificial Sequence
  Synthetic construct
APASSSTKKTELQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 38 - protein, 133 aa, Artificial Sequence
  Synthetic construct
APASSSTKKTELQLQELLDDLQMILNGINNYKNPKLTRMLTAKFAMPKKATELKHLQCLEEELKPLEEVLNGAQSKNFHLQPRELIENINVIVLELKGSETTFMCEYADETATIVEFLNHWITFAQSIIETLE

SEQ ID NO: 39 - protein, 133 aa, Artificial Sequence
  Synthetic construct
APASSSTKKTESQLEELLDDLQQILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 40 - protein, 133 aa, Artificial Sequence
  Synthetic construct
APASSYTKKTQERLEQLLLDLEQILNGINNYKNPKLTRMLTAKFAMPKKATELKHLQCLEEELKPLEEVLNGAQSKNFHLNPRELIENINVIVLELKGSETTFMCEYADETATIVEFLNHWITFAQSIIETLD

SEQ ID NO: 41 - protein, 133 aa, Artificial Sequence
  Synthetic construct
APASSSTKKTQLQLEDLLLDLQNILNGINNYKNPKLTRMLTAKFAMPKKATELKHLQCLEEELKPLEEVLNGAQSKNFHLDPRELISNINVIVLELKGSETTFMCEYADETATIVEFLNHWITFAQSIIETLE

SEQ ID NO: 42 - protein, 133 aa, Artificial Sequence
  Synthetic construct
APASSSTKKTETQLEQLLDDLQEILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 43 - protein, 133 aa, Artificial Sequence
  Synthetic construct
APASSSTKKTEEQLENLLLDLQNILNGINNYKNPKLTRMLTAKFAMPKKATELKHLQCLEEELKPLEEVLNGAQSKNFHLEPRELISNINVIVLELKGSETTFMCEYADETATIVEFLNHWITFAQSIIETLE

SEQ ID NO: 44 - protein, 133 aa, Artificial Sequence
  Synthetic construct
APASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 45 - protein, 133 aa, Artificial Sequence
  Synthetic construct
APASSSTKKTQLQLQHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 46 - protein, 133 aa, Artificial Sequence
  Synthetic construct
APASSSTKKTQLQLEELLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 47 - protein, 133 aa, Artificial Sequence
  Synthetic construct
APASSSTKKTQLQLEHLLDDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 48 - protein, 133 aa, Artificial Sequence
  Synthetic construct
APASSSTKKTQLQLEHLLLDLEMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 49 - protein, 133 aa, Artificial Sequence
  Synthetic construct
APASSSTKKTQLQLEHLLLDLQQILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 50 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLDPRDLISNINVIVLELKGSETTFMCEYADETATIVEFLNRWITFAQSIISTLT

SEQ ID NO: 51 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRELISNINVIVLELKGSETTFMCEYADETATIVEFLNRWITFAQSIISTLT

SEQ ID NO: 52 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLIENINVIVLELKGSETTFMCEYADETATIVEFLNRWITFAQSIISTLT

SEQ ID NO: 53 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIISTLT

SEQ ID NO: 54 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAESIISTLT

SEQ ID NO: 55 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAHSIISTLT

SEQ ID NO: 56 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIIETLT

SEQ ID NO: 57 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLE

SEQ ID NO: 58 - protein, 133 aa, Artificial Sequence
  Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLD

SEQ ID NO: 59 - protein, 133 aa, Artificial Sequence
  Synthetic construct
APASSSTKKTQLQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNRWITFAQSIISTLT

SEQ ID NO: 60 - protein, 133 aa, Artificial Sequence
  Synthetic construct
APASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIIETLD

SEQ ID NO: 61 - protein, 133 aa, Artificial Sequence
  Synthetic construct
APASSSTKKTELQLEHLLLDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLD

SEQ ID NO: 62 - protein, 214 aa, Artificial Sequence
  Synthetic construct
DVQITQSPSSLSASVGDRVTITCRTSRSISQYLAWYQEKPGKTNKLLIYSGSTLQSGIPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 227 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 64 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 65 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQEQLEHLLLELQNILNGINNYKNPKLTRMLTAKFAMPKKATELKHLQCLEEELKPLEEVLNGAQSKNFHLNPRELIENINVIVLELKGSETTFMCEYADETATIVEFLNEWIEFAQSIIETLN

SEQ ID NO: 66 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTELQLEHLLLQLQEILNGINNYKNPKLTRMLTAKFAMPKKATELKHLQCLEEELKPLEEVLNGAQSKNFHLDPRELIENINVIVLELKGSETTFMCEYADETATIVEFLNRWITFAQSIIETLN

SEQ ID NO: 67 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQEQLEHLLDDLQMILNGINNYKNPKLTRMLTAKFAMPKKATELKHLQCLEEELKPLEEVLNGAQSKNFHLDPRDLISNINVIVLELKGSETTFMCEYADETATIVEFLNEWIEFAQSIIETLE

SEQ ID NO: 68 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLQPRDLIENINDIVLELKGSETTFMCEYADETATIVEFLNRWIEFAQSIIETLD

SEQ ID NO: 69 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTEEQLEHLLLDLQQILNGINNYKNPKLTRMLTAKFAMPKKATELKHLQCLEEELKPLEEVLNGAQSKNFHLDPRDLIENINDIVLELKGSETTFMCEYADETATIVEFLNRWITFAQSIIETLQ

SEQ ID NO: 70 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTELQLEHLLDDLQMILNGINNYKNPKLTRMLTAKFAMPKKATELKHLQCLEEELKPLEEVLNGAQSKNFHLDPRELISNINVIVLELKGSETTFMCEYADETATIVEFLNRWITFAQSIIETLD

SEQ ID NO: 71 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLDPRQLIDNINNIVLELKGSETTFMCEYADETATIVEFLNRWIQFAQSIIETLD

SEQ ID NO: 72 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLDDLQMILNGINNYKNPKLTRMLTAKFAMPKKATELKHLQCLEEELKPLEEVLNGAQSKNFHLEPRELIQNINVIVLELKGSETTFMCEYADETATIVEFLNHWITFAQSIIETLE

SEQ ID NO: 73 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLDDLQNILNGINNYKNPKLTRMLTAKFAMPKKATELKHLQCLEEELKPLEEVLNGAQSKNFHLDPRDLISNINVIVLELKGSETTFMCEYADETATIVEFLNRWITFAQSIISTLE

SEQ ID NO: 74 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTEEQLEHLLLDLQQILNGINNYKNPKLTRMLTAKFAMPKKATELKHLQCLEEELKPLEEVLNGAQSKNFHLQPRDLIDNINNIVLQLKGSETTFMCEYADETATIVEFLNHWIEFAQSIIETLE

SEQ ID NO: 75 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLDDLQMILNGINNYKNPKLTRMLTAKFAMPKKATELKHLQCLEEELKPLEEVLNGAQSKNFHLEPRDLISNINVIVLELKGSETTFMCEYADETATIVEFLNRWITFAQSIIETLD

SEQ ID NO: 76 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLQPRDLIENINDIVLELKGSETTFMCEYADETATIVEFLNEWITFAQSIIETLD

SEQ ID NO: 77 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQQQLEHLLQDLQMILNGINNYKNPKLTRMLTAKFAMPKKATELKHLQCLEEELKPLEEVLNGAQSKNFHLHPRDLISNINEIVLELKGSETTFMCEYADETATIVEFLNRWIEFAQSIIETLE

SEQ ID NO: 78 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTEKTQLQLEHLLLELQNILNGINNYKNPKLTRMLTAKFAMPKKATELKHLQCLEEELKPLEEVLNGAQSKNFHLHPRQLIENINVIVLELKGSETTFMCEYADETATIVEFLNHWITFAQSIIETLD

SEQ ID NO: 79 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQEQLEHLLQDLQMILNGINNYKNPKLTRMLTAKFAMPKKATELKHLQCLEEELKPLEEVLNGAQSKNFHLHPRDLISNINVIVLELKGSETTFMCEYADETATIVEFLNEWITFAQSIISTLD

SEQ ID NO: 80 - protein, 227 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLDDLEQILNGINNYKNPKLTRMLTAKFAMPKKATELKHLQCLEEELKPLEEVLNGAQSKNFHLDPRELIDNINVIVLELKGSETTFMCEYADETATIVEFLNHWITFAQSIIETLE

SEQ ID NO: 82 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEEQLEQLLDDLENILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 83 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEELLDDLQEILNGINNYKNPKLTRMLTAKFAMPKKATELKHLQCLEEELKPLEEVLNGAQSKNFHLDPRDLIENINVIVLELKGSETTFMCEYADETATIVEFLNHWITFAESIISTLD

SEQ ID NO: 84 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTESQLQNLLDDLQEILNGINNYKNPKLTRMLTAKFAMPKKATELKHLQCLEEELKPLEEVLNGAQSKNFHLEPRELIDNINVIVLELKGSETTFMCEYADETATIVEFLNHWITFAQSIIETLE

SEQ ID NO: 85 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEELLLDLQEILNGINNYKNPKLTRMLTAKFAMPKKATELKHLQCLEEELKPLEEVLNGAQSKNFHLNPRELIENINVIVLELKGSETTFMCEYADETATIVEFLNHWITFAESIIETLE

SEQ ID NO: 86 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLLDLEEILNGINNYKNPKLTRMLTAKFAMPKKATELKHLQCLEEELKPLEEVLNGAQSKNFHLHPRELIENINVIVLELKGSETTFMCEYADETATIVEFLNHWITFAQSIIETLD

SEQ ID NO: 87 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEDHLQELLLDLEEILNGINNYKNPKLTRMLTAKFAMPKKATELKHLQCLEEELKPLEEVLNGAQSKNFHLNPRELIENINVIVLELKGSETTFMCEYADETATIVEFLNHWITFAQSIIETLE

SEQ ID NO: 88 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLEPRDLIENINVIVLELKGSETTFMCEYADETATIVEFLNHWITFAQSIIETLE

SEQ ID NO: 89 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLDDLQQILNGINNYKNPKLTRMLTAKFAMPKKATELKHLQCLEEELKPLEEVLNGAQSKNFHLNPRELIDNINVIVLELKGSETTFMCEYADETATIVEFLNHWITFAQSIIETLD

SEQ ID NO: 90 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLQELLDDLHMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 91 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLQELLDDLHEILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 92 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 93 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLLDLEQILNGINNYKNPKLTRMLTAKFAMPKKATELKHLQCLEEELKPLEEVLNGAQSKNFHLRPRDLINNINVIVLELKGSETTFMCEYADETATIVEFLNHWITFAQSIIETLE

SEQ ID NO: 94 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLDDLDQILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 95 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEEHLQNLLDDLEQILNGINNYKNPKLTRMLTAKFAMPKKATELKHLQCLEEELKPLEEVLNGAQSKNFHLEPRELIDNINVIVLDLKGSETTFMCEYADETATIVEFLNHWITFAQSIISTLE

SEQ ID NO: 96 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEELLDDLEMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLD

SEQ ID NO: 97 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTTEQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNEWITFAQSIIETLD

SEQ ID NO: 98 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 99 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLQPRELIENINVIVLELKGSETTFMCEYADETATIVEFLNHWITFAQSIIETLE

SEQ ID NO: 100 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTESQLEELLDDLQQILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 101 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSYTKKTQERLEQLLLDLEQILNGINNYKNPKLTRMLTAKFAMPKKATELKHLQCLEEELKPLEEVLNGAQSKNFHLNPRELIENINVIVLELKGSETTFMCEYADETATIVEFLNHWITFAQSIIETLD

SEQ ID NO: 102 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEDLLLDLQNILNGINNYKNPKLTRMLTAKFAMPKKATELKHLQCLEEELKPLEEVLNGAQSKNFHLDPRELISNINVIVLELKGSETTFMCEYADETATIVEFLNHWITFAQSIIETLE

SEQ ID NO: 103 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEQLLDDLQEILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 104 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEEQLENLLLDLQNILNGINNYKNPKLTRMLTAKFAMPKKATELKHLQCLEEELKPLEEVLNGAQSKNFHLEPRELISNINVIVLELKGSETTFMCEYADETATIVEFLNHWITFAQSIIETLE

SEQ ID NO: 105 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 106 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLQHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 107 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 108 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLDDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 109 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLEMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 110 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQQILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 111 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLDPRDLISNINVIVLELKGSETTFMCEYADETATIVEFLNRWITFAQSIISTLT

SEQ ID NO: 112 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRELISNINVIVLELKGSETTFMCEYADETATIVEFLNRWITFAQSIISTLT

SEQ ID NO: 113 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLIENINVIVLELKGSETTFMCEYADETATIVEFLNRWITFAQSIISTLT

SEQ ID NO: 114 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIISTLT

SEQ ID NO: 115 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAESIISTLT

SEQ ID NO: 116 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAHSIISTLT

SEQ ID NO: 117 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIIETLT

SEQ ID NO: 118 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLE

SEQ ID NO: 119 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLD

SEQ ID NO: 120 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNRWITFAQSIISTLT

SEQ ID NO: 121 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIIETLD

SEQ ID NO: 122 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLD

SEQ ID NO: 123 - protein, 595 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 124 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 125 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLQHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 126 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 127 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLDDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 128 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLEMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 129 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQQILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 130 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLDPRDLISNINVIVLELKGSETTFMCEYADETATIVEFLNRWITFAQSIISTLTA

SEQ ID NO: 131 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRELISNINVIVLELKGSETTFMCEYADETATIVEFLNRWITFAQSIISTLTA

SEQ ID NO: 132 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLIENINVIVLELKGSETTFMCEYADETATIVEFLNRWITFAQSIISTLTA

SEQ ID NO: 133 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIISTLTA

SEQ ID NO: 134 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAESIISTLTA

SEQ ID NO: 135 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAHSIISTLTA

SEQ ID NO: 136 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIIETLTA

SEQ ID NO: 137 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLEA

SEQ ID NO: 138 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLDA

SEQ ID NO: 139 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNRWITFAQSIISTLTA

SEQ ID NO: 140 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNHWITFAQSIIETLDA

SEQ ID NO: 141 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLEHLLLDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLDA

SEQ ID NO: 142 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLEA

SEQ ID NO: 143 - protein, 596 aa, Artificial Sequence
  Synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTA

SEQ ID NO: 144 - protein, 133 aa, Homo sapiens [sequence omitted]

SEQ ID NO: 145 - protein, 218 aa, Artificial Sequence
  Synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 449 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 147 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSYTKKTQERLEQLLLDLEQILNGINNYKNPKLTRMLTAKFAMPKKATELKHLQCLEEELKPLEEVLNGAQSKNFHLNPRELIENINVIVLELKGSETTFMCEYADETATIVEFLNHWITFAQSIIETLD

SEQ ID NO: 148 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEDLLLDLQNILNGINNYKNPKLTRMLTAKFAMPKKATELKHLQCLEEELKPLEEVLNGAQSKNFHLDPRELISNINVIVLELKGSETTFMCEYADETATIVEFLNHWITFAQSIIETLE

SEQ ID NO: 149 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEQLLDDLQEILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 150 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLQELLDDLHMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 151 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLQELLDDLHEILNGINNYKNPKLTRMLTAKFAMPKKATELKHLQCLEEELKPLEEVLNGAQSKNFHLEPRELIENINVIVLELKGSETTFMCEYADETATIVEFLNHWITFAQSIIETLE

SEQ ID NO: 152 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLLDLEQILNGINNYKNPKLTRMLTAKFAMPKKATELKHLQCLEEELKPLEEVLNGAQSKNFHLRPRDLINNINVIVLELKGSETTFMCEYADETATIVEFLNHWITFAQSIIETLE

SEQ ID NO: 153 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLDDLDQILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 154 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEEHLQNLLDDLEQILNGINNYKNPKLTRMLTAKFAMPKKATELKHLQCLEEELKPLEEVLNGAQSKNFHLEPRELIDNINVIVLDLKGSETTFMCEYADETATIVEFLNHWITFAQSIISTLE

SEQ ID NO: 155 - protein, 597 aa, Artificial Sequence
  Synthetic constuct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEELLDDLEMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLD

SEQ ID NO: 156 - protein, 597 aa, Artificial Sequence
  Synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTELQLQELLDDLQMILNGINNYKNPKLTRMLTAKFAMPKKATELKHLQCLEEELKPLEEVLNGAQSKNFHLDPRELIENINVIVLELKGSETTFMCEYADETATIVEFLNHWITFAQSIIETLE

SEQ ID NO: 157 - protein, 597 aa, Artificial Sequence
  Synthetic constuct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTEEQLENLLLDLQNILNGINNYKNPKLTRMLTAKFAMPKKATELKHLQCLEEELKPLEEVLNGAQSKNFHLEPRELISNINVIVLELKGSETTFMCEYADETATIVEFLNHWITFAQSIIETLE

SEQ ID NO: 158 - protein, 226 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 159 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEQLLDDLQEILNGINNYKNPKLTRMLTFKFYMPKKATELKHLQCLEEELKPLEEVLNLAQSKNFHLDPRELIENINVIVLELKGSETTFMCEYADETATIVEFLNHWITFAQSIIETLE

SEQ ID NO: 160 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEELLDDLDQILNGINNYKNPKLTRMLTFKFYMPKKATELKHLQCLEEELKPLEEVLNLAQSKNFHLDPRELIENINVIVLELKGSETTFMCEYADETATIVEFLNHWITFAQSIIETLE

SEQ ID NO: 161 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 162 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSYTKKTQERLEQLLLDLEQILNGINNYKNPKLTRMLTFKFYMPKKATELKHLQCLEEELKPLEEVLNLAQSKNFHLNPRELIENINVIVLELKGSETTFMCEYADETATIVEFLNHWITFAQSIIETLD

SEQ ID NO: 163 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLEELLDDLEMILNGINNYKNPKLTRMLTFKFYMPKKATELKHLQCLEEELKPLEEVLNLAQSKNFHLDPRELIENINVIVLELKGSETTFMCEYADETATIVEFLNHWITFAQSIIETLD

SEQ ID NO: 164 - protein, 374 aa, Artificial Sequence
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTETQLQELLDDLHEILNGINNYKNPKLTRMLTFKFYMPKKATELKHLQCLEEELKPLEEVLNLAQSKNFHLEPRELIENINVIVLELKGSETTFMCEYADETATIVEFLNHWITFAQSIIETLE